(19) United States
(12) Patent Application Publication
(10) Pub. No.: US 2005/0222146 A1
(43) Pub. Date: Oct. 6, 2005

(54) N-substituted-n-sulfonylaminocyclopropane compounds and pharmaceutical use thereof

(76) Inventors: Andrew M. Fryer, Erie, CO (US); Makoto Shiozaki, Osaka (JP); Nicole M. Littmann, Erie, CO (US); Takashi Inaba, Osaka (JP); Steven W. Andrews, Longmont, CO (US); Katsutaka Yasue, Osaka (JP); Ellen R. Laird, Longmont, CO (US); Masahiro Yokota, Osaka (JP); Julia Haas, Boulder, CO (US); Hiroto Imai, Osaka (JP); Katsuya Maeda, Osaka (JP); Yuichi Shinozaki, Osaka (JP); Yoshikazu Hori, Osaka (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/011,781

(22) Filed: Dec. 15, 2004

Related U.S. Application Data
(60) Provisional application No. 60/529,117, filed on Dec. 15, 2003.

(51) Int. Cl. A61K 31/5377; A61K 31/496; A61K 31/381; C07D 49/02; C07D 413/02

(52) U.S. Cl. 514/232.2; 514/252.13; 514/445; 544/145; 549/71; 544/379

(57) ABSTRACT

The present invention provides a compound having aggrecanase inhibitory activity and MMP-13 inhibitory activity, and useful as a therapeutic agent for osteoarthritis, rheumatoid arthritis and the like, more specifically, a N-substituted-N-sulfonylaminocyclopropane compound of formula (1): 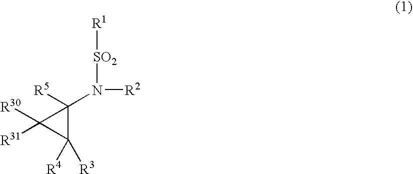
wherein R¹ is —W-A¹—W₁-A², W is —(CH₂)ₘ—X—(CH₂)ₙ—, wherein W₁ is —(CH₂)ₘ₁—X₁—(CH₂)ₙ₁—, m, m1, n and n1 are the same or different and each is 0 to 6, X and X₁ are the same or different and each is a single bond, etc., A¹ is an optionally substituted C₃₋₁₄ hydrocarbon ring group, etc. and A² is a substituted C₃₋₁₄ hydrocarbon ring group etc.; R² is —(CH₂)ᵣ—CO—R⁸, etc., wherein r is 0 to 6 and R⁸ is a C₁₋₆ alkoxy group, etc.; R³ and R⁴ are the same or different and each is a hydrogen atom, a C₁₋₆ alkyl group, etc.; and R⁵ is —CO₂R²¹ etc.; R³⁰ and R³¹ are the same or different and each is a hydrogen atom, etc.; or a prodrug thereof or a pharmaceutically acceptable salt thereof.

This Application claims benefit of priority of U.S. provisional Application No. 60/529,117, filed Dec. 15, 2003, the contents of which are hereby incorporated by reference.

The present invention relates to a novel N-substituted-N-sulfonylaminocyclopropane compound. In further detail, the present invention relates to a N-substituted-N-sulfonylaminocyclopropane compound or a pharmaceutically acceptable salt thereof having an aggrecanase inhibitory activity or matrix metalloproteinase (MMP) inhibitory activity, a pharmaceutical composition which comprises this compound and a pharmaceutical use thereof.

Aggrecan is a main proteoglycan in cartilage, and decomposition of its core protein by protease is one of the early signs of a joint disorder associated with arthrodial cartilage destruction, such as rheumatoid arthritis and osteoarthritis. This process of decomposition leading to the cartilage destruction begins with the disappearance of aggrecan on the surface of cartilage, and progresses to the decomposition of collagen type II fiber. MMPs (Matrix metalloproteinases) that cleave Asn 341—Phe 342 and aggrecanase that cleaves Glu 373-Ala 374 are known as enzymes involved in this decomposition of aggrecan, and both are metal-proteases having zinc in the catalytic active center. The latter was determined to be ADAMTS (A Disintegrin and Metalloproteinase with Thrombospondin Motifs) in 1999. ADAMTS 1 to 20 have been identified so far, and ADAMTS 4 and 5 correspond to aggrecanase-1 and aggrecanase-2, respectively. Conventionally, MMP have been considered to mainly cause cartilage destruction, but many reports have documented that the aggrecan fragments found in the joint of osteoarthritis (OA) patients are predominantly the fragments cleaved by aggrecanases. Thus, aggrecanase is also considered to be a significant vicious factor for the disease state.

At present, conservative treatments and surgical treatments are available for treating OA. The conservative therapy includes body weight control, exercise therapy, physical therapy, drug therapy (administration of anti-inflammatory drug), hyperthermia, and the like. It is a general practice to inject hyaluronic acid into the joint in the course of these treatments to smoothen movement of the joint.

When improvement of conditions by the conservative treatments such as drug therapy, physical therapy, etc., is not achieved, a surgical treatment is performed. When the joint is highly deformed and causes a strong pain, an arthroplasty for embedding an artificial joint is performed as the final option. However, artificial joints have a life of only about 15 to 20 years, after which the QOL (Quality of Life) of the patient deteriorates.

At present, no drug that suppresses enzyme involved in cartilage destruction is available for OA treatment. When no improvement is made by a conservative treatment, cartilage destruction progresses and a surgical treatment will be required. Therefore, prevention of cartilage destruction before reaching the stage requiring a surgical treatment is important. A drug that inhibits aggrecanases involved in the destruction of cartilage is acknowledged to be an anti-OA drug having a sufficient cartilage destruction inhibitory activity. Without a surgical treatment, and moreover, such drug is expected to improve the QOL of patients.

Aggrecanase inhibitors have been developed as shown in the reports by DuPont (WO99/0900), Pfizer (JP-A-2001-114765) and the like, in which poor oral availability is a concern.

In addition, the MMP inhibitors under development include a compound that causes systemic connective tissue toxicity due to nonselective collagenase inhibition. It is proposed that the cause thereof is suppression of turnover of normal connective tissue collagen due to inhibition of collagenase-1 (MMP-1). It is clear, therefore, that the conventional products are not entirely satisfactory from the aspects of effective inhibition and occurrence of side effects.

The compound of the present invention possesses improved oral availability and shows strong aggrecanase inhibitory activity. While the compound is free of an MMP-1 inhibitory activity, it also has selective inhibitory activity of MMP-13, involved in joint destruction. Therefore, the compound is expected to suppress progress of joint diseases without causing side effects.

In addition, expressed in glioma, aggrecanase is suggested to be also involved in metastasis or tissue infiltration of tumor cells, like MMP, and in view of the current development of MMP inhibitor as an antimetastatic drug, the compound of the present invention having an inhibitory activity on both aggrecanase and MMP is expected to be a highly effective antitumor agent.

In bone metabolism, MMP suppresses decomposition of bone matrix and has a major part in bone resorption. In respiratory diseases, protease plays a key role in the course of destruction and remodeling of lung structure. MMP that uses an extracellular matrix (ECM), which is an architectural component of the protease, as a substrate is considered to be an important factor. Therefore, the compound of the present invention having MMP inhibitory activity is expected to be applicable to the bone resorption disorders and lung diseases, in which MMP is involved.

Various reports on compounds aiming at therapy of disorders such as OA, rheumatoid arthritis and the like by inhibition of aggrecanase have been published recently.

For example, JP-A-2002-284686 discloses a sulfonamide derivative having MMP-13 inhibitory activity and aggrecanase inhibitory activity. However, this publication does not include the compound having a structure, such as the structure of the compound of the present invention, or a disclosure suggestive thereof.

JP-A-2001-114765 discloses a hydroxamic acid derivative represented by the following formula: 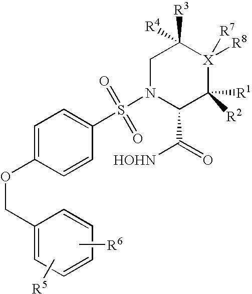
wherein X is carbon atom or nitrogen atom; R¹ and R² are each independently hydrogen atom, hydroxy or methyl, and at least one of R¹ and R² is methyl; R³ and R⁴ are each independently hydrogen atom, hydroxy or methyl, or R³ and R⁴ may be taken together to form carbonyl group; R⁵ and R⁶ are each independently hydrogen atom, halogen, cyano, methyl or ethyl; with the proviso that when X is carbon atom, R⁷ and R⁸ are both hydrogen atom and at least one of R¹, R², R³ and R⁴ is hydroxy; when X is carbon atom and R⁵ is para-halo, at least one of R⁶, R³ and R⁴ is not hydrogen atom; when X is nitrogen atom, R⁸ is not present and R⁷ is hydrogen atom or the group of the formula: 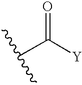
wherein Y is —CH₂—NH₂ or —NH—CH₃; when X is nitrogen atom and R⁷ is H, R³ and R⁴ may be taken together to form carbonyl group, which has aggrecanase inhibitory activity. However, the compound of this publication has a piperidine ring or piperazine ring having substituent(s) as a skeletal structure. This publication does not include the compound having a cyclopropane structure, such as the structure of the compound of the present invention, or a disclosure suggestive thereof.

WO03/053915 discloses a cyclopropane derivative represented by the formula: 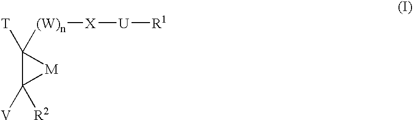
wherein M is —(C(R³⁰)(R⁴⁰))m- wherein m is 1 to 6; T is R²¹-substituted alkyl, cycloalkyl, heterocycloalkyl, cycloalkenyl, heterocycloalkenyl, aryl, heteroaryl, —OR³, —C(O)R⁴, —C(O)OR³, —C(O)NR²⁴R²⁵, —C(O)NR²⁴OR³, —C(O)SR³, —NR²⁴R²⁵R²⁵C(O)R⁴, —NR²⁵C(O)OR³, —NR²⁵C(O)NR²⁴R²⁵—NR²⁵C(O)NR²⁴OR³, —SR³, —S(O)ₓNR²⁴R²⁵, —S(O)ₓNR²⁵OR³ etc.; V is alkyl, R²¹-substituted alkyl, cycloalkyl, heterocycloalkyl, cycloalkenyl, heterocycloalkenyl, aryl, heteroaryl, —OR³, —C(O)R⁴, —(CR²³R²⁴)ₙ₁C(O)OR³, —C(O)NR²⁴R²⁵—(CR²³R²⁴)ₙ₁C(O)NR²⁵OR³, —C(O)SR³, —NR²⁴R²⁵, —NR²⁵C(O)R⁴, —NR²⁵C(O)OR³—NR²⁵C(O)NR²⁴R²⁵, —NR²⁵C(O)NR²⁴R³, —SR³, —S(O)xNR²⁴R²⁵, —S(O)xNR²⁵OR²⁵OR³, etc.; W is a covalent bond, —(C(R³) (R⁴))ₙ₂—, —O—, —S—, etc.; X is alkylene, cycloalkylene, heterocycloalkylene, arylene, heteroarylene, —C≡C—, etc.; U is a covalent bond, —(C(R³) (R⁴))ₚ—, —Y—(C(R³) (R⁴))_{q}—, —(C(R³)(R⁴))ₜ—Y—, —Y—, etc.; Y is —O—, —S(O)ₓ—, etc.; n is 0 to 2; R¹ is alkyl, R²¹-substituted alkyl, cycloalkyl, heterocycloalkyl, cycloalkenyl, heterocycloalkenyl, aryl, heteroaryl, etc.; R², R⁴ and R⁵ are each independently hydrogen atom, halo, alkyl, etc.; R³ is hydrogen atom, alkyl, R²²-substituted alkyl, etc.; R²³ is hydrogen atom, hydroxyl, halo, etc.; R²⁴ is hydrogen atom or alkyl; R²⁵ is hydrogen atom, hydroxyl, alkyl, etc.; R³⁰ is hydrogen atom, etc.; R⁴⁰ is hydrogen atom, etc.; with the proviso that at least one of V and T is —C(O)N(R³) (OR⁴), —C(O)OR or —C(O)NR²⁴R²⁵. However, the compound of the formula disclosed in this publication is structurally different from the compound of the present invention. This publication does not include a compound having a structure of the compound of the present invention, or a disclosure suggestive thereof.

### DISCLOSURE OF INVENTION

The present invention provides a compound having superior aggrecanase inhibitory activity and MMP inhibitory activity (particularly, MMP-13 inhibitory activity), and useful as a prophylactic or therapeutic agent for osteoarthritis, a prophylactic or therapeutic agent for rheumatoid arthritis, a prophylactic or therapeutic agent for a disorder such as joint injury, reactive arthritis, bone resorption disorder, cancer, asthma, allergic reaction, chronic pulmonary emphysema, fibroid lung, acute respiratory distress (ARDS), lung infection, interstitial pneumonia, etc. compound.

Some embodiments of the present invention provide an aggrecanase inhibitor, a MMP inhibitor, a prophylactic or therapeutic agent for osteoarthritis and a prophylactic or therapeutic agent for rheumatoid arthritis.

The present inventors have conducted intensive studies to obtain the above objects and found a N-substituted-N-sulfonylaminocyclopropane compound represented by the following formula (1) has superior aggrecanase inhibitory activity and MMP-13 inhibitory activity, and useful as an aggrecanase inhibitor, a MMP inhibitor, a prophylactic or therapeutic agent for osteoarthritis and a prophylactic or therapeutic agent for rheumatoid arthritis, based on which findings the present invention has been completed.

Accordingly, the present invention relates the compounds [1] to [31] shown below and pharmaceutical use thereof.
[1] An N-substituted-N-sulfonylaminocyclopropane compound represented by the formula (1) 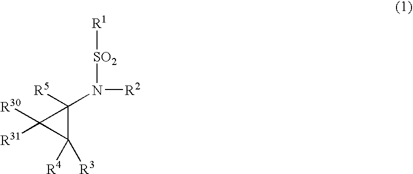
   wherein
R¹ is
   —W-A¹—W₁-A²
   (wherein
W is —(CH₂)ₘ—X—(CH₂)ₙ—,
W₁ is —(CH₂)ₘ₁—X₁—(CH₂)ₙ₁—,
   (wherein
   m, n, m1 and n1 are the same or different and each is selected from 0 and an integer ranging from 1 to 6, X and X₁ are the same or different and each is a linker selected from the following group A,
   group A:
   (a) a single bond,
   (b) a C₁₋₆ alkylene group,
   (c) a C₂₋₆ alkenylene group,
   (d) a C₂₋₆ alkynylene group,
   (e) —O—,
   (f) —N(R⁶)—,
   (g) —S(O)ₘ₃,
   (h) —CO—,
   (i) —COO—,
   (j) —OCO—,
   (k) —CON(R⁶)—,
   (l) —N(R⁶)CO—,
   (m) —SO₂N(R⁶)—,
   (n) —N(R⁶)SO₂—,
   (o) —N(R⁶)CON(R⁷)—,
   (p) —N(R⁶)SO₂N(R⁷)—,
   (q) —OCON(R⁶)—,
   (r) —N(R⁶)COO—and
   (s) —S(O)ₘ₃—(CH₂)ₙ₃—CO—,
      (wherein R⁶ and R⁷ are the same or different and each is selected from a hydrogen atom, a C₁₋₆ alkyl group optionally substituted by halogen atoms or hydroxyl groups, a C₃₋₁₄ hydrocarbon ring group and a heterocyclic group, m3 is selected from 0 and an integer ranging from 1 to 2, and n3 is an integer ranging from 1 to 2),
   A¹ is selected from an optionally substituted C₃₋₁₄ hydrocarbon ring group and an optionally substituted heterocyclic group, and
   A² is selected from a substituted C₃₋₁₄ hydrocarbon ring group and a substituted heterocylic group,
   or A¹ and A² may be taken together with a substituent thereof to form an optionally substituted fused C₆₋₁₄ hydrocarbon ring group);
R² is selected from
   —(CH₂)ₘ₅—X₅—(CH₂)ₙ₅-A⁵ (1)
   and
   —(CH₂)ₘ₅—X₅—(CH₂)ₙ₅—R³² (2)
   (wherein m5 and n5 are the same or different and each is selected from 0 and an integer ranging from 1 to 6,
   X₅ are the same or different and each is a linker selected from the above-mentioned group A,
   A⁵ is selected from an optionally substituted C₃₋₁₄ hydrocarbon ring group and an optionally substituted heterocyclic group, and
   R³² is a substituent selected from the following group B, provided that when m5 and n5 are 0 and X₅ is a single bond, then R³² should not be a hydrogen atom);
   group B:
   (a) a hydrogen atom,
   (b) a halogen atom,
   (c) a hydroxyl group,
   (d) a nitro group,
   (e) a cyano group,
   (f) a carboxyl group,
   (g) an amino group,
   (h) an amide group,
   (i) a C₂₋₆ acyl group,
   (j) a halogenated C₁₋₆ alkyl group,
   (k) a C₁₋₆ alkyl group optionally substituted by hydroxyl groups,
   (l) a C₂₋₆ alkenyl group optionally substituted by halogen atoms,
   (m) a C₂₋₆ alkynyl group,
   (n) a C₁₋₆ alkoxy group optionally substituted by hydroxyl groups
   (o) a C₁₋₆ alkoxy-C₁₋₆alkyl group,
   (p) a C₁₋₆ alkoxy-carbonyl group,
   (q) a C₁₋₆ alkyl-aminocarbonyl group optionally substituted by halogen atoms,
   (r) a mono(C₁₋₆ alkyl)amino group,
   (s) a di(C₁₋₆ alkyl)amino group,
   (t) a C₁₋₆ alkyl-carbonylamino group optionally substituted by halogen atoms,
   (u) a C₁₋₆ alkylsulfonyl group and
   (v) a C₁₋₆ alkylsulfonylamino group,
   or R² and R³ and the cyclopropane ring may be taken together to form an optionally further substituted fused ring);
R³ and R⁴ are the same or different and each is selected from
   —(CH₂)ₘ₂—X₂—(CH₂)ₙ₂-A⁴ (1)
   (wherein m2 and n2 are the same or different and each is selected from 0 and an integer ranging from 1 to 6, X₂ is a linker selected from the above-mentioned group A, and A⁴ is selected from an optionally substituted C₃₋₁₄ hydrocarbon ring group and an optionally substituted heterocyclic group) and
   —(CH₂)ₘ₆—X₆—(CH₂)ₙ₆—R³³ (2)
      (wherein m6 and n6 are the same or different and each is selected from 0 and an integer ranging from 1 to 6, X₆ is a linker selected from the above-mentioned group A, and R³³ is a substituent selected from the above-mentioned group B); or A⁴ and R³³ may be taken together to form an optionally substituted fused ring group,
   and R³ and R⁴ may be taken together with a carbon atom bonded thereto to form the following ring 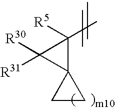
      (wherein m10 is an integer ranging from 1 to 6),
   provided that R³ and R⁴ are not hydrogen atoms at the same time;
R⁵ is selected from
   (1) —CO₂R²¹,
   (2) —C(O)NHOR²¹,
   (3) —C(O)NH—SO₂—R²¹,
   (4) —C(O)NHR²¹,
   (5) —SH,
   (6) —CH₂CO₂R²¹,
   (7) —C(O)R²¹,
   (8) —N(OH)COR²¹,
   (9) —SN₂H₂R²¹,
   (10) —SONHR²¹,
   (11) —CH₂CO₂H,
   (12) —PO(OH)₂,
   (13) —PO(OH)NHR²¹,
   (14) —CH₂SH,
   (15) —CH₂OH,
   (16) —(CH₂)ᵣ₁—PO(OH)—(CH₂)ᵣ₂—R²¹,
   (17) —NHR²¹
   (18) —NH—NHR²¹ and
   (19) —(CH₂)ᵣ₁—R⁵⁰
   (wherein r1 and r2 are the same or different and each is selected from 0 and an integer ranging from 1 to 6,
   R²¹ is selected from
   (1) a hydrogen atom,
   (2) an optionally substituted C₁₋₁₀ alkyl group,
   (3) an optionally substituted C₆₋₁₄ aryl-C₁₋₆ alkyl group and
      —(CH₂)ₘ₇X₇—(CH₂)ₙ₇—R³⁴ (4)
   (wherein m7 and n7 are the same or different and each is selected from 0 and an integer ranging from 1 to 6, X₇ is a linker selected from the above-mentioned group A, R³⁴ is a substituent selected from the following group C));
   group C:
   (a) a hydrogen atom,
   (b) a halogen atom,
   (c) a hydroxyl group,
   (d) a nitro group,
   (e) a cyano group,
   (f) a carboxyl group,
   (g) an amino group,
   (h) an amide group,
   (i) a C₂₋₆ acyl group,
   (j) a halogenated C₁₋₆ alkyl group,
   (k) a C₁₋₆ alkyl group optionally substituted by hydroxyl groups,
   (l) a C₂₋₆ alkenyl group optionally substituted by halogen atoms,
   (m) a C₂₋₆ alkynyl group,
   (n) a C₁₋₆ alkoxy group optionally substituted by hydroxyl groups,
   (o) a C₁₋₆ alkoxy-C₁₋₆ alkyl group,
   (p) a C₁₋₆ alkoxy-carbonyl group,
   (q) a C₁₋₆ alkyl-aminocarbonyl group optionally substituted by halogen atoms,
   (r) a mono(C₁₋₆ alkyl)amino group,
   (s) a di(C₁₋₆alkyl)amino group,
   (t) a C₁₋₆ alkyl-carbonylamino group optionally substituted by halogen atoms,
   (u) a C₁₋₆ alkylsulfonyl group,
   (v) a C₁₋₆ alkylsulfonylamino group,
   (w) a C₃₋₁₄ hydrocarbon ring group optionally substituted by 1 to 5 substituents selected from the above-mentioned group B and
   (x) a heterocyclic group optionally substituted by 1 to 5 substituents selected from the above-mentioned group B), and
   R⁵⁰ is selected from an optionally substituted C₃₋₁₄ hydrocarbon ring group and an optionally substituted heterocyclic group;
   or R²¹ of —C(O)NHR²¹, A⁴ and the cyclopropane ring may be taken together to form an optionally further substituted fused ring;
R³⁰ and R³¹ ae the same or different and each is selected from
   —(CH₂)ₘ₈—X₈—(CH₂)ₙ₈-A⁶ (1)
   (wherein m8 and n8 are the same or different and each is 0 or an integer ranging from 1 to 6, X₈ is a linker selected from the above-mentioned group A, and A⁶ is selected from an optionally substituted C₃₋₁₄ hydrocarbon ring group and an optionally substituted heterocyclic group) and
   —(CH₂)ₘ₉—X₉—(CH₂)ₙ₉—R³⁶ (2)
   (wherein m9 and n9 are the same or different and each is selected from 0 and an integer ranging from 1 to 6, X₉ is a linker selected from the above-mentioned group A, and R³⁶ is a substituent selected from the above-mentioned group B);
   or A⁴, R³⁶ and the cyclopropane ring may be taken together to form an optionally further substituted fused ring,
   or R²¹ of —CO₂R²¹, R³⁰ and the cyclopropane ring may be taken together to form an optionally further substituted fused ring,
   or further, R³⁰ and R³¹ may be taken together with a carbon atom bonded thereto to form the following ring 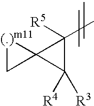
      (wherein m11 is an integer ranging from 1 to 6);
      or a prodrug thereof or a pharmaceutically acceptable salt thereof [hereinafter sometimes referred to as compound (1)];
[2] The compound of [1] above, wherein A² is 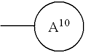
   (wherein ring A¹⁰ is selected from a C₃₋₁₄ hydrocarbon ring group and a heterocyclic group, and further the ring A¹⁰ is substituted by 1 to 5 groups of “−(CH₂)ₘ₁₂—X₁₂—(CH₂)ₙ₁₂—R³⁷”, which are the same or different (wherein m₁₂ and n12 are the same or different and each is selected from 0 and an integer ranging from 1 to 6, X₁₂ is a linker selected from the above-mentioned group A and R³⁷ is a substituent selected from the above-mentioned group C)),
   or the ring A¹⁰ and A¹ may be taken together with a substituent thereof to form an optionally substituted fused C₆₋₁₄ hydrocarbon ring group,
   A⁴, A⁵ and A⁶ may be the same or different and each is 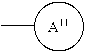
      (wherein ring A¹¹ is selected from a C₃₋₁₄ hydrocarbon ring group and a heterocyclic group, and further the ring A¹¹ is optionally substituted by 1 to 5 groups of “—(CH₂)ₘ₁₃—X₁₃—(CH₂)ₙ₁₃—R³⁸”, which are the same or different (wherein m13 and n13 are the same or different and each is selected from 0 and an integer ranging from 1 to 6, X₁₃ is a linker selected from the above-mentioned group A and R³⁸ is a substituent selected from the above-mentioned group C)); or a prodrug thereof or a pharmaceutically acceptable salt thereof;
[3] The compound of [2] above, wherein m and n are 0 and X is a single bond; or a prodrug thereof or a pharmaceutically acceptable salt thereof;
[4] The compound of [3] above, wherein m1 and n1 are 0 and X₁ is a single bond; or a prodrug thereof or a pharmaceutically acceptable salt thereof;
[5] The compound of [4] above, wherein R⁵ is selected from —CO₂R²¹ and —C(O)NHOR²¹; or a prodrug thereof or a pharmaceutically acceptable salt thereof.
[6] The compound of [5] above, wherein R²¹ is a hydrogen atom; or a prodrug thereof or a pharmaceutically acceptable salt thereof;
[7] The compound of [6] above, wherein R³ is —(CH₂)ₘ2—X₂—(CH₂)ₙ₂-A⁴; or a prodrug thereof or a pharmaceutically acceptable salt thereof;
[8] The compound of [1] above, which is selected from the group consisting of
(1S*,5S*,6R*)-2-(4′-Chloro-biphenyl-4-sulfonyl)-6-phenyl-2-aza-bicyclo[3.1.0]hexane-1-carboxylic acid,
(1S,2R)-1-{[5-(4-Chloro-phenyl)-thiophene-2-sulfonyl]-methyl-amino}-2-phenyl-cyclopropanecarboxylic acid,
(1R*,2S*)-1-[[5-(4-Chloro-phenyl)-thiophene-2-sulfonyl]-(2)-hydroxy-ethyl)-amino]-2-phenyl-cyclopropanecarboxylic acid,
1-(2-{((1R*,2S*)-1-Carboxy-2-phenyl-cyclopropyl)-[4-(4-trifluoromethyl-phenyl)-piperazine-1-sulfonyl]-amino}-ethyl)-1H-pyrazole-4-carboxylic acid,
(1R*,2S*)-1-{[2-(5-Amino-tetrazol-2-yl)-ethyl]-[4-(4-trifluoromethyl-phenyl)-piperazine-1-sulfonyl]-amino}-2-phenyl-cyclopropanecarboxylic acid,
(1R*,2S*)-1-{[2-(5-Amino-tetrazol-1-yl)-ethyl]-[4-(4-trifluoromethyl-phenyl)-piperazine-1-sulfonyl]-amino}-2-phenyl-cyclopropanecarboxylic acid,
(1R,2S)-1-{Carboxymethyl-[5-(4-chloro-phenyl)-thiophene-2-sulfonyl]-amino}-2-phenyl-cyclopropanecarboxylic acid,
(1R,2S)-1-{Carboxymethyl-[5-(5-trifluoromethyl-isoxazol-3-yl)-thiophene-2-sulfonyl]-amino}-2-phenyl-cyclopropanecarboxylic acid,
(1S,2R)-1-{Carboxymethyl-[5-(4-chloro-phenyl)-thiophene-2-sulfonyl]-amino}-2-phenyl-cyclopropanecarboxylic acid,
(1S,2R)-1-{[5-(4-Chloro-phenyl)-thiophene-2-sulfonyl]-[2-(4-methyl-piperazin-1-yl)-2-oxo-ethyl]-amino}-2-phenyl-cyclopropanecarboxylic acid
(1R,2S)-1-{[5-(4-Chloro-phenyl)-thiophene-2-sulfonyl]-methyl-amino}-2-phenyl-cyclopropanecarboxylic acid,
(1R*,6S*)-2-[5-(4-Chloro-phenyl)-thiophene-2-sulfonyl]-6-phenyl-2-aza-bicyclo[4.1.0]heptane-1-carboxylic acid,
(1R,2S)-1-[[5-(4-Chloro-phenyl)-thiophene-2-sulfonyl]-(2-morpholin-4-yl-ethyl)-amino]-2-phenyl-cyclopropanecarboxylic acid,
4-Methyl-piperazine-1-carboxylic acid 2-{((1R,2S)-1-carboxy-2-phenyl-cyclopropyl)-[5-(4-chloro-phenyl)-thiophene-2-sulfonyl]-amino}-ethyl ester,
(1R,2S)-1-[[5-(4-Chloro-phenyl)-thiophene-2-sulfonyl]-(2-morpholin-4-yl-2-oxo-ethyl)-amino]-2-phenyl-cyclopropanecarboxylic acid,
(1R,2S)-1-{[5-(4-Chloro-phenyl)-thiophene-2-sulfonyl]-[2-(4-methyl-piperazin-1-yl)-2-oxo-ethyl]-amino}-2-phenyl-cyclopropanecarboxylic acid,
(1R,2S)-1-[[5-(4-Chloro-phenyl)-thiophene-2-sulfonyl]-(3-hydroxy-propyl)-amino]-2-phenyl-cyclopropanecarboxylic acid,
Morpholine-4-carboxylic acid 2-{((1R,2S)-1-carboxy-2-phenyl-cyclopropyl)-[5-(4-chloro-phenyl)-thiophene-2-sulfonyl]-amino}-ethyl ester,
Morpholine-4-carboxylic acid 3-{((1R,2S)-1-carboxy-2-phenyl-cyclopropyl)-[5-(4-chloro-phenyl)-thiophene-2-sulfonyl]-amino}-propyl ester,
4-Methyl-piperazine-1-carboxylic acid 3-{((1R,2S)-1-carboxy-2-phenyl-cyclopropyl)-[5-(4-chloro-phenyl)-thiophene-2-sulfonyl]-amino}-propyl ester,
(1R*,6R*)-2-[5-(4-Chloro-phenyl)-thiophene-2-sulfonyl]-6-phenyl-4-oxa-2-aza-bicyclo[4.1.0]heptane-1-carboxylic acid,
(1R*,6S*)-6-Phenyl-2-[5-(5-trifluoromethyl-isoxazol-3-yl)-thiophene-2-sulfonyl]-2-aza-bicyclo[4.1.0]heptane-1-carboxylic acid,
(1R*,5S*)-2-[5-(4-Chloro-phenyl)-thiophene-2-sulfonyl]-5-phenyl-2-aza-bicyclo[3.1.0]hexane-1-carboxylic acid,
(1R*,2S*)-1-{Methyl-[5-(5-trifluoromethyl-isoxazol-3-yl)-thiophene-2-sulfonyl]-amino}-2-morpholin-4-ylmethyl-2-phenyl-cyclopropanecarboxylic acid,
1R*,7S*)-2-[5-(4-Chloro-phenyl)-thiophene-2-sulfonyl]-4-oxo-7-phenyl-2,5-diaza-bicyclo[5.1.0]octane-1-carboxylic acid,
(1R*,7R*)-2-[5-(4-Chloro-phenyl)-thiophene-2-sulfonyl]-4-hydroxymethyl-7-phenyl-5-oxa-2-aza-bicyclo[5.1.0]octane-1-carboxylic acid, and
(1R*,7R*)-2-[5-(4-Chloro-phenyl)-thiophene-2-sulfonyl]-5-methyl-4-oxo-7-phenyl-2-,5-diaza-bicyclo[5.1.0]octane-1-carboxylic acid;
   or a prodrug thereof or a pharmaceutically acceptable salt thereof;
[9] The compound of [1] above, which is represented by the formula (1′): 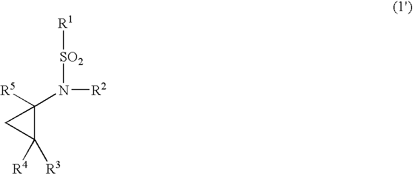
   wherein
R¹ is —W-A¹—W₁-A²,
   wherein
W is —(CH₂)ₘ—X—(CH₂)ₙ—,
W₁ is —(CH₂)ₘ₁—X₁—(CH₂)ₙ₁—,
   wherein
   m, m1, n and n1 are the same or different and each is selected from 0 and an integer ranging from 1 to 6,
   X and X₁ are the same or different and each is selected from a single bond, a C₁₋₆ alkylene group, a C₂₋₆ alkenylene group, a C₂₋₆ alkynylene group, —O—, —N(R⁶)—, —S(O)_{q}—, —CO—, —CON(R⁶)— 1-N(R⁶)CO—, —SO₂N(R⁶)—, —N(R⁶)SO₂—, —N(R⁶)CON(R⁷)—, —N(R⁶)SO₂N(R⁷)—, —OCON(R⁶)— and —N(R⁶)COO—,
   wherein
   R⁶ and R⁷ are the same or different and each is selected from a hydrogen atom, a C₁₋₆ alkyl group, an optionally substituted C₃₋₁₄ hydrocarbon ring group and an optionally substituted heterocyclic group,
   q is selected from 0 and an integer ranging from 1 to 2,
   A¹ is selected from an optionally substituted C₃₋₁₄ hydrocarbon ring group and an optionally substituted heterocyclic group;
A² is selected from a substituted C₃₋₁₄ hydrocarbon ring group and a substituted heterocyclic group;
R² is selected from
   —(CH₂)ᵣ—CO—R⁸ (1)
   wherein
   r is selected from 0 and an integer ranging from 1 to 6,
   R⁸ is selected from a C₁₋₆ alkoxy group and —N(R⁹) (R¹⁰)
   wherein
   R⁹ and R¹⁰ are the same or different and each is selected from a hydrogen atom, a C₁₋₆ alkyl group, a C₁₋₆ alkylsulfonyl group, —SO₂A³ and A³, or may be taken together with a nitrogen atom to form an optionally substituted nitrogen-containing heterocyclic group,
   A³ is selected from an optionally substituted C₃₋₁₄ hydrocarbon ring group and an optionally substituted heterocyclic group;
      —(CH₂)ᵣ—N(R¹¹)(R¹²) (2)
   wherein
   r is as defined above,
   R¹¹ and R¹² are the same or different and each is selected from a hydrogen atom, a C₁₋₆ alkyl group, —CO—R¹³, —SO₂—R¹⁴ and A³, or may be taken together with a nitrogen atom to form an optionally substituted nitrogen-containing heterocyclic group,
   wherein
   R¹³ is selected from a C₁₋₆ alkyl group optionally substituted by C₁₋₆ alkoxy groups or hydroxy groups, and a C₁₋₆ alkoxy group,
   R¹⁴ is selected from a C₁₋₆ alkyl group, a halogenated C₁₋₆ alkyl group, —N(R¹⁵) (R¹⁶) and A³,
   wherein
   R¹⁵ and R¹⁶ are the same or different and each is selected from a hydrogen atom, a C₁₋₆ alkyl group, a C₁₋₆ alkoxy-carbonyl group and A³,
   A³ is as defined above; and
      —(CH₂)ᵣ—R¹⁷ (3)
   wherein
   r is as defined above,
   R¹⁷ is selected from a C₁₋₆ alkyl group optionally substituted by at least one substituent selected from hydroxy groups and —CO₂R¹⁸ groups, and A³,
   wherein
   R¹⁸ is selected from a hydrogen atom and a C₁₋₆ alkyl group,
   A³ is as defined above;
R³ and R⁴ are the same or different and each is selected from
(1) a hydrogen atom,
(2) a C₁₋₆ alkyl group
(3) a halogenated C₁₋₆ alkyl group, and
   —(CH₂)ₘ₂—X₂—(CH₂)ₙ₂-A⁴, (4)
   wherein
   m2 and n2 are the same or different and each is selected from 0 and an integer ranging from 1 to 6,
   X₂ is selected from a single bond, a C₁₋₆ alkylene group, a C₂₋₆ alkenylene group, a C₂₋₆ alkynylene group, —O—, —N(R¹⁹)—, —S(O)_{q1}—, —CO—, —CON(R¹⁹)—, —N(R¹⁹)CO—, —SO₂N(R¹⁹)—, —N(R¹⁹)SO₂—, —N(R¹⁹)CON(R²⁰)—, —N(R¹⁹)SO₂N(R²⁰)—, —OCON(R¹⁹)— and —N(R¹⁹)COO—,
   wherein
   R¹⁹ and R²⁰ are the same or different and each is selected from a hydrogen atom, a C₁₋₆ alkyl group, an optionally substituted C₃₋₁₄ hydrocarbon ring group and an optionally substituted heterocyclic group,
   q1 is selected from 0 and an integer ranging from 1 to 2,
   A⁴ is selected from an optionally substituted C₃₋₁₄ hydrocarbon ring group and an optionally substituted heterocyclic group;
R⁵ is selected from
(1) —CO₂R²¹,
(2) —C(O)NHOR²¹,
(3) —C(O)NH—SO₂—R²¹,
(4) —C(O)NHR²¹,
(5) —SH,
(6) —CH₂CO₂R²¹,
(7) —C(O)R²¹,
(8) —N(OH)COR²¹,
(9) —SN₂H₂R²¹,
(10) —SONHR²¹,
(11) —CH₂CO₂H,
(12) —PO(OH)₂,
(13) —PO(OH)NHR²¹,
(14) —CH₂SH and
(15) —CH₂OH
   wherein
   R²¹ is selected from a hydrogen atom, an optionally substituted C₁₋₁₀ alkyl group and an optionally substituted C₆₋₁₄ aryl-C₁₋₆ alkyl group;
      or a prodrug thereof or a pharmaceutically acceptable salt thereof;
[10] The compound of [9] above, wherein m and n are 0, and X is a single bond; or a prodrug thereof or a pharmaceutically acceptable salt thereof;
[11] The compound of [10] above, wherein m1 and n1 are 0; or a prodrug thereof or a pharmaceutically acceptable salt thereof;
[12] The compound of [11] above, wherein R⁵ is selected from —CO₂R²¹ and —C(O)NHOR²¹; or a prodrug thereof or a pharmaceutically acceptable salt thereof;
[13] The compound of [12] above, wherein R²¹ is a hydrogen atom; or a prodrug thereof or a pharmaceutically acceptable salt thereof;
[14] The compound of [13] above, wherein R³ is —(CH₂)ₘ₂—X₂-(CH₂) n2-A⁴; or a prodrug thereof or a pharmaceutically acceptable salt thereof;
[15] The compound of [9] above, which is selected from the group consisting of
(1S,2R)-1-[carboxymethyl-(4′-chloro-biphenyl-4-sulfonyl)-amino]-2-phenyl-cyclopropanecarboxylic acid,
(1R,2S)-1-[carboxymethyl-(4′-chloro-biphenyl-4-sulfonyl)-amino]-2-phenyl-cyclopropanecarboxylic acid,
(1S,2R)-2-benzyl-1-[carboxymethyl-(4′-chloro-biphenyl-4-sulfonyl)-amino]-cyclopropanecarboxylic acid,
(1R*,2S*)-1-[(2-tert-butoxycarbonylamino-ethyl)-(4′-chloro-biphenyl-4-sulfonyl)-amino]-2-phenyl-cyclopropanecarboxylic acid,
(1R*,2S*)-1-[(4′-chloro-biphenyl-4-sulfonyl)-(5-oxo-2,5-dihydro-1H-pyrazol-3-ylmethyl)-amino]-2-phenyl-cyclopropanecarboxylic acid,
(1R*,2S*)-1-[(4′-chloro-biphenyl-4-sulfonyl)-(2-hydroxy-2-methyl-propyl)-amino]-2-phenyl-cyclopropanecarboxylic acid,
(1R*,2S*)-1-[(4′-chloro-biphenyl-4-sulfonyl)-(2-methanesulfonylamino-ethyl)-amino]-2-phenyl-cyclopropanecarboxylic acid,
(1R*,2S*)-1-[(1H-benzoimidazol-2-ylmethyl)-(4′-chloro-biphenyl-4-sulfonyl)-amino]-2-phenyl-cyclopropanecarboxylic acid,
(1R*, 2S*)-1-[(4′-chloro-biphenyl-4-sulfonyl)-(2-isopropoxycarbonylaminosulfonylamino-ethyl)-amino]-2-phenyl-cyclopropanecarboxylic acid,
(1R*,2S*)-4-{[(1-carboxy-2-phenyl-cyclopropyl)-(4′-chloro-biphenyl-4-sulfonyl)-amino]-methyl}-benzoic acid,
(1R*,2S*)-1-[carboxymethyl-(4′-chloro-biphenyl-4-sulfonyl)-amino]-2-(4-chloro-phenyl)-cyclopropanecarboxylic acid,
(1R*,2S*)-1-[carboxymethyl-(4′-chloro-biphenyl-4-sulfonyl)-amino]-2-(3-chloro-phenyl)-cyclopropanecarboxylic acid,
(1R*,2S*)-1-[carboxymethyl-(4′-chloro-biphenyl-4-sulfonyl)-amino]-2-(2-chloro-phenyl)-cyclopropanecarboxylic acid,
(1R*,2R*)-1-[carboxymethyl-(4′-chloro-biphenyl-4-sulfonyl)-amino]-2-(2-chloro-phenyl)-cyclopropanecarboxylic acid,
(1R*,2S*)-1-[(4′-chloro-biphenyl-4-sulfonyl)-(2H-tetrazol-5-ylmethyl)-amino]-2-phenyl-cyclopropanecarboxylic acid,
(1R*,2S)-1-[benzyl-(4′-chloro-biphenyl-4-sulfonyl)-amino]-2-phenyl-cyclopropanecarboxylic acid,
(1R*,2S*)-1-[(4′-chloro-biphenyl-4-sulfonyl)-(3-hydroxy-benzyl)-amino]-2-phenyl-cyclopropanecarboxylic acid,
(1R*,2S*)-1-[carboxymethyl-(4′-chloro-biphenyl-4-sulfonyl)-amino]-2-(4-methyl-phenyl)-cyclopropanecarboxylic acid,
(1R*,2S*)-1-[carboxymethyl-(4′-chloro-biphenyl-4-sulfonyl)-amino]-2-(2-methyl-phenyl)-cyclopropanecarboxylic acid,
(1R*,2S*)-1-[carboxymethyl-(4′-chloro-biphenyl-4-sulfonyl)-amino]-2-(3-methyl-phenyl)-cyclopropanecarboxylic acid,
(1R*,2S)-1-[carboxymethyl-(4′-chloro-biphenyl-4-sulfonyl)-amino]-2-(3-methoxy-phenyl)-cyclopropanecarboxylic acid,
(1R*,2S*)-1-[carboxymethyl-(4′-chloro-biphenyl-4-sulfonyl)-amino]-2-(2-methoxy-phenyl)-cyclopropanecarboxylic acid,
(1R*,2S*)-1-[carboxymethyl-(4′-chloro-biphenyl-4-sulfonyl)-amino]-2-(3,4-dichloro-phenyl)-cyclopropanecarboxylic acid,
(1R*,2S*)-1-[carboxymethyl-(4′-chloro-biphenyl-4-sulfonyl)-amino]-2-(2,5-dichloro-phenyl)-cyclopropanecarboxylic acid,
(1R*,2S*)-1-[carboxymethyl-(4′-chloro-biphenyl-4-sulfonyl)-amino]-2-(3-phenoxy-phenyl)-cyclopropanecarboxylic acid,
(1R*,2S*)-2-biphenyl-2-yl-1-[carboxymethyl-(4′-chloro-biphenyl-4-sulfonyl)-amino]-cyclopropanecarboxylic acid,
(1R*,2S*)-1-[carboxymethyl-(4′-chloro-biphenyl-4-sulfonyl)-amino]-2-(3-cyano-phenyl)-cyclopropanecarboxylic acid,
(1R*,2S*)-1-[carboxymethyl-(4′-chloro-biphenyl-4-sulfonyl)-amino]-2-(2-cyano-phenyl)-cyclopropanecarboxylic acid,
(1R*,2S*)-1-[carboxymethyl-(4′-chloro-biphenyl-4-sulfonyl)-amino]-2-(2,6-dichloro-phenyl)-cyclopropanecarboxylic acid,
(1R*,2S*)-1-[carboxymethyl-(4′-chloro-biphenyl-4-sulfonyl)-amino]-2-(4-cyano-phenyl)-cyclopropanecarboxylic acid,
(1R*,2S*)-2-(2-benzyl-phenyl)-1-[carboxymethyl-(4′-chloro-biphenyl-4-sulfonyl)-amino]-cyclopropanecarboxylic acid,
(1R*,2S*)-2-biphenyl-4-yl-1-[carboxymethyl-(4′-chloro-biphenyl-4-sulfonyl)-amino]-cyclopropanecarboxylic acid,
(1R*,2S*)-1-[carboxymethyl-(4′-chloro-biphenyl-4-sulfonyl)-amino]-2-(2-trifluoromethyl-phenyl)-cyclopropanecarboxylic acid,
(1R*,2S*)-1-[carboxymethyl-(4′-chloro-biphenyl-4-sulfonyl)-amino]-2-(3-trifluoromethyl-phenyl)-cyclopropanecarboxylic acid,
(1R*,2S*)-1-[carboxymethyl-(4′-chloro-biphenyl-4-sulfonyl)-amino]-2-(5-chloro-2-trifluoromethyl-phenyl)-cyclopropanecarboxylic acid,
(1R*,2S)-1-[carbamoylmethyl-(4′-chloro-biphenyl-4-sulfonyl)-amino]-2-phenyl-cyclopropanecarboxylic acid,
(1R*,2S*)-1-[(2-carboxy-2-methyl-propyl)-(4′-chloro-biphenyl-4-sulfonyl)-amino]-2-phenyl-cyclopropanecarboxylic acid,
(1R*,2S*)-1-[(4′-chloro-biphenyl-4-sulfonyl)-(2-methanesulfonylamino-2-oxo-ethyl)-amino]-2-phenyl-cyclopropanecarboxylic acid,
(1R*,2S*)-2-(3-benzyloxy-phenyl)-1-[carboxymethyl-(4′-chloro-biphenyl-4-sulfonyl)-amino]-cyclopropanecarboxylic acid,
(1R*,2S*)-2-(2-benzyloxy-phenyl)-1-[carboxymethyl-(4′-chloro-biphenyl-4-sulfonyl)-amino]-cyclopropanecarboxylic acid,
(1R*,2S*)-1-[carboxymethyl-(4′-chloro-biphenyl-4-sulfonyl)-amino]-2-(2,3-dichloro-phenyl)-cyclopropanecarboxylic acid,
(1R*,2S*)-1-[carboxymethyl-(4′-chloro-biphenyl-4-sulfonyl)-amino]-2-(2-phenoxy-phenyl)-cyclopropanecarboxylic acid,
(1R*,2S*)-1-{(4′-chloro-biphenyl-4-sulfonyl)-[2-(3,4-dihydroxy-pyrrolidin-1-yl)-2-oxo-ethyl]-amino}-2-phenyl-cyclopropanecarboxylic acid,
(1R*, 2S*)-2-(3-benzyl-phenyl)-1-[carboxymethyl-(4′-chloro-biphenyl-4-sulfonyl)-amino]-cyclopropanecarboxylic acid,
(1R*,2S*)-1-[(4′-chloro-biphenyl-4-sulfonyl)-(2-oxo-2-pyrrolidin-1-yl-ethyl)-amino]-2-phenyl-cyclopropanecarboxylic acid,
(1R*,2S*)-1-[(4′-chloro-biphenyl-4-sulfonyl)-methoxycarbonylmethyl-amino]-2-phenyl-cyclopropanecarboxylic acid,
(1R*,2S*)-1-[carboxymethyl-(4′-chloro-biphenyl-4-sulfonyl)-amino]-2-(3-isobutoxy-phenyl)-cyclopropanecarboxylic acid,
(1R*,2S*)-1-[carboxymethyl-(4′-chloro-biphenyl-4-sulfonyl)-amino]-2-(3-cyclohexyloxy-phenyl)-cyclopropanecarboxylic acid,
(1R*,2S*)-[(4′-chloro-biphenyl-4-sulfonyl)-(1-methanesulfonylaminocarbonyl-2-phenyl-cyclopropyl)-amino]-acetic acid,
(1R*,2S*)-2-(3-benzyloxy-phenyl)-1-[carboxymethyl-(4′-chloro-biphenyl-4-sulfonyl)-amino]-cyclopropanecarboxylic acid,
(1R*,2S*)-3-{[[1-carboxy-2-(3-phenoxy-phenyl)-cyclopropyl]-(4′-chloro-biphenyl-4-sulfonyl)-amino]-methyl}-benzoic acid,
(1R*,2S*)-1-[(4′-chloro-biphenyl-4-sulfonyl)-ethoxycarbonylmethyl-amino]-2-(3-phenoxy-phenyl)-cyclopropanecarboxylic acid,
(1R*,2S*)-1-[(4′-chloro-biphenyl-4-sulfonyl)-methyl-amino]-2-(3-phenoxy-phenyl)-cyclopropanecarboxylic acid,
(1R*,2S*)-1-[(4′-chloro-biphenyl-4-sulfonyl)-(4-methanesulfonylaminocarbonyl-thiazol-2-ylmethyl)-amino]-2-phenyl-cyclopropanecarboxylic acid,
5-{[((1R*,2S*)-1-carboxy-2-phenyl-cyclopropyl)-(4′-chloro-biphenyl-4-sulfonyl)-amino]-methyl}-furan-2-carboxylic acid,
2-{[((1R*,2S*)-1-carboxy-2-phenyl-cyclopropyl)-(4′-chloro-biphenyl-4-sulfonyl)-amino]-methyl}-nicotinic acid,
(1R*,2S*)-1-[(4′-chloro-biphenyl-4-sulfonyl)-pyridin-2-ylmethyl-amino]-2-phenyl-cyclopropanecarboxylic acid,
(1R*,2S*)-1-[(4′-chloro-biphenyl-4-sulfonyl)-pyridin-3-ylmethyl-amino]-2-phenyl-cyclopropanecarboxylic acid,
(1R*,2S*)-1-{benzyl-[4-(2-methyl-2H-tetrazol-5-yl)-benzenesulfonyl]-amino}-2-phenyl-cyclopropanecarboxylic acid,
2-{[((1R*, 2S*)-1-carboxy-2-phenyl-cyclopropyl)-(4′-chloro-biphenyl-4-sulfonyl)-amino]-methyl}-thiazole-4-carboxylic acid,
(1R*,2S*)-1-{(4′-chloro-biphenyl-4-sulfonyl)-[(1H-tetrazol-5-ylcarbamoyl)-methyl]-amino}-2-phenyl-cyclopropanecarboxylic acid,
(1R*,2S*)-1-[(4′-chloro-biphenyl-4-sulfonyl)-(3-methanesulfonylamino-benzyl)-amino]-2-phenyl-cyclopropanecarboxylic acid,
(1R*,2S*)-1-[(4′-chloro-biphenyl-4-sulfonyl)-(2-trifluoromethanesulfonylamino-ethyl)-amino]-2-phenyl-cyclopropanecarboxylic acid,
(1R*,2S*)-1-[(4′-chloro-biphenyl-4-sulfonyl)-(5-oxo-4,5-dihydro-[1,2,4]oxadiazol-3-ylmethyl)-amino]-2-phenyl-cyclopropanecarboxylic acid,
(1R*,2S*)-1-[(4′-chloro-biphenyl-4-sulfonyl)-(5-oxo-4,5-dihydro-1H-[1,2,4]triazol-3-ylmethyl)-amino]-2-phenyl-cyclopropanecarboxylic acid,
(1R*,2S*)-1-[carboxymethyl-(4′-chloro-biphenyl-4-sulfonyl)-amino]-2-(3-hydroxy-phenyl)-cyclopropanecarboxylic acid,
4-(3-{(1R*,2S*)-2-Carboxy-2-[carboxymethyl-(4′-chloro-biphenyl-4-sulfonyl)-amino]-cyclopropyl}-phenoxy)-piperidine-1-carboxylic acid tert-butyl ester,
(1R*,2S*)-1-[carboxymethyl-(4′-chloro-biphenyl-4-sulfonyl)-amino]-2-[3-(piperidin-4-yloxy)-phenyl]-cyclopropanecarboxylic acid,
1-{2-[((1R*,2S*)-1-carboxy-2-phenyl-cyclopropyl)-(4′-chloro-biphenyl-4-sulfonyl)-amino]-ethyl}-1H-pyrrole-2-carboxylic acid,
4-{(1R*,2S*)-2-carboxy-2-[(3-carboxy-benzyl)-(4′-chloro-biphenyl-4-sulfonyl)-amino]-cyclopropyl}-piperidin-1-carboxylic acid tert-butyl ester,
(1R*,2S*)-1-[(4′-chloro-biphenyl-4-sulfonyl)-(5-oxo-4,5-dihydro-[1,2,4]thiadiazol-3-ylmethyl)-amino]-2-phenyl-cyclopropanecarboxylic acid,
(1R*,2S*)-1-[(4′-chloro-biphenyl-4-sulfonyl)-(5-oxo-4,5-dihydro-[1,2,4]oxadiazol-3-ylmethyl)-amino]-2-(3-phenoxy-phenyl)-cyclopropanecarboxylic acid,
3-{[((1R*, 2S*)-1-carboxy-2-phenyl-cyclopropyl)-(4′-chloro-biphenyl-4-sulfonyl)-amino]-methyl}-pyridin-2-carboxylic acid,
(1R*,2S*)-1-{methyl-[4-(4-methyl-thiophen-2-yl)-benzenesulfonyl]-amino}-2-phenyl-cyclopropanecarboxylic acid,
(1R*,2S*)-1-{carboxymethyl-[4-(4-methyl-thiophen-2-yl)-benzenesulfonyl]-amino}-2-phenyl-cyclopropanecarboxylic acid,
4-({((1R*,2S*)-1-carboxy-2-phenyl-cyclopropyl)-[4-(4-methyl-thiophen-2-yl)-benzenesulfonyl]-amino}-methyl)-benzoic acid,
(1R*,2S*)-1-{(4′-chloro-biphenyl-4-sulfonyl)-[2-(1H-tetrazol-5-ylamino)-ethyl]-amino}-2-phenyl-cyclopropanecarboxylic acid,
1-{2-[((1R*,2S*)-1-carboxy-2-phenyl-cyclopropyl)-(4′-chloro-biphenyl-4-sulfonyl)-amino]-ethyl}-1H-imidazole-2-carboxylic acid,
1-{2-[(1R*,2S*)-1-carboxy-2-phenyl-cyclopropyl)-(4′-chloro-biphenyl-4-sulfonyl)-amino]-ethyl}-1H-pyrazol-4-carboxylic acid,
3-{[((1R*,2S*)-1-carboxy-2-piperidin-4-yl-cyclopropyl)-(4′-chloro-biphenyl-4-sulfonyl)-amino]-methyl}-benzoic acid,
(1R*,2S*)-1-[(4′-chloro-biphenyl-4-sulfonyl)-(2-trifluoromethanesulfonylamino-ethyl)-amino]-2-(3-phenoxy-phenyl)-cyclopropanecarboxylic acid,
5-{[((R, 2S*-1-carboxy-2-phenyl-cyclopropyl)-(4′-chloro-biphenyl-4-sulfonyl)-amino]-methyl}-isooxazole-3-carboxylic acid,
(1R*,2S*)-1-{(4′-chloro-biphenyl-4-sulfonyl)-[2-(1,1,3,4-tetraoxo-llambda*6*-[1,2,5]thiadiazolidin-2-yl)-ethyl]-amino}-2-phenyl-cyclopropanecarboxylic acid,
(1R*,2S*)-1-[(4′-chloro-biphenyl-4-sulfonyl)-(2-aminosulfonylamino-ethyl)-amino]-2-(3-phenoxy-phenyl)-cyclopropanecarboxylic acid,
3-{[{(1R*,2S*)-1-carboxy-2-[3-(2-diethylamino-ethylamino)-phenyl]-cyclopropyl}-(4′-chloro-biphenyl-4-sulfonyl)-amino]-methyl}-benzoic acid,
3-{[{(1R*,2S*)-1-carboxy-2-[3-(pyridin-2-ylamino)-phenyl]-cyclopropyl}-(4′-chloro-biphenyl-4-sulfonyl)-amino]-methyl}benzoic acid,
(1R*, 2S*)-1-[(4′-chloro-biphenyl-4-sulfonyl)-(2-trifluoromethanesulfonylamino-ethyl)-amino]-2-[3-(2-piperidin-1-yl-acetylamino)-phenyl]-cyclopropanecarboxylic acid,
3-{[{(1R*,2S*)-1-carboxy-2-[3-(2-hydroxy-ethoxy)-phenyl]-cyclopropyl}-(4′-chloro-biphenyl-4-sulfonyl)-amino]-methyl}-benzoic acid,
(1R*,2S*)-1-[(4′-chloro-biphenyl-4-sulfonyl)-(2-trifluoromethanesulfonylamino-ethyl)-amino]-2-[3-(2-piperidin-1-yl-ethylamino)-phenyl]-cyclopropanecarboxylic acid,
(1R*,2S*)-1-[(4′-chloro-biphenyl-4-sulfonyl)-(2-trifluoromethanesulfonylamino-ethyl)-amino]-2-[3-(2-piperidin-1-yl-ethanesulfonylamino)-phenyl]-cyclopropanecarboxylic acid,
3-{[{(1R*,2S*)-1-carboxy-2-[3-(2-piperidin-1-yl-ethylamino)-phenyl]-cyclopropyl}-(4′-chloro-biphenyl-4-sulfonyl)-amino]-methyl}-benzoic acid,
3-[((4′-chloro-biphenyl-4-sulfonyl)-{(1R*,2S*)-1-methyl carbamoyl-2-[3-(2-piperidin-1-yl-ethylamino)-phenyl]-cyclopropyl}-amino)-methyl]-benzoic acid,
(1R*,2S*)-1-[(3-carboxy-propyl)-(4′-chloro-biphenyl-4-sulfonyl)-amino]-2-phenyl-cyclopropanecarboxylic acid,
1-{2-[((1R*,2S*)-1-carboxy-2-phenyl-cyclopropyl)-(4′-chloro-biphenyl-4-sulfonyl)-amino]-ethyl}-piperidin-4-carboxylic acid,
3-{[{(1R*,2S*-1-carboxy-2-[3-(2-imidazol-1-yl-ethoxy)-phenyl]-cyclopropyl}-(4′-chloro-biphenyl-4-sulfonyl)-amino]-methyl}-benzoic acid,
(1R*,2S*)-1-{(4′-chloro-biphenyl-4-sulfonyl)-[2-(2-hydroxy-acetylamino)-ethyl]-amino}-2-phenyl-cyclopropanecarboxylic acid,
1-{2-[((1R*,2S*)-1-carboxy-2-phenyl-cyclopropyl)-(4′-chloro-biphenyl-4-sulfonyl)-amino]-ethyl}-piperidin-3R-carboxylic acid,
1-{2-[((1R*,2S*)-1-carboxy-2-phenyl-cyclopropyl)-(4′-chloro-biphenyl-4-sulfonyl)-amino]-ethyl}-piperidin-3S-carboxylic acid,
3-{[((1R*, 2S*)-1-carboxy-2-{3-[(pyridin-3-carbonyl)-amino]-phenyl}-cyclopropyl)-(4′-chloro-biphenyl-4-sulfonyl)-amino]-methyl}-benzoic acid,
3-{[{(1R*,2S*)-1-carboxy-2-[3-(2-pyrrolidin-1-yl-ethoxy)-phenyl]-cyclopropyl}-(4′-chloro-biphenyl-4-sulfonyl)-amino]-methyl}-benzoic acid,
3-{[{(1R*,2S*)-1-carboxy-2-[3-(2-morpholin-4-yl-ethoxy)-phenyl]-cyclopropyl}-(4′-chloro-biphenyl-4-sulfonyl)-amino]-methyl}-benzoic acid,
3-{[{(1R*,2S*-1-carboxy-2-[3-(pyridin-3-yloxy)-phenyl cyclopropyl}-(4′-chloro-biphenyl-4-sulfonyl)-amino]-methyl}-benzoic acid,
(1R*,2S*)-1-[(4′-chloro-biphenyl-4-sulfonyl)-(4-oxalyl-benzyl)-amino]-2-phenyl-cyclopropanecarboxylic acid,
1-{2-[(1R*,2S*)-1-carboxy-2-phenyl-cyclopropyl)-(4′-chloro-biphenyl-4-sulfonyl)-amino]-ethyl}-1H-imidazole-4-carboxylic acid,
(1R*,2S*)-1-[(5-carbamoyl-pentyl)-(4′-chloro-biphenyl-4-sulfonyl)-amino]-2-phenyl-cyclopropanecarboxylic acid,
(1R*,2S*)—{(4′-chloro-biphenyl-4-sulfonyl)-[2-(4-methyl carbamoyl-pyrazol-1-yl)-ethyl]-amino}-2-phenyl-cyclopropanecarboxylic acid,
(1R*,2S*)-1-{(4′-chloro-biphenyl-4-sulfonyl)-[2-(2-hydroxy-2-methyl-propionylamino)-ethyl]-amino}-2-phenyl-cyclopropanecarboxylic acid,
3-{[{(1R*,2S*)-1-carboxy-2-[3-(2-pyrazol-1-yl-ethoxy)-phenyl]-cyclopropyl}-(4′-chloro-biphenyl-4-sulfonyl)-amino]-methyl}-benzoic acid,
(1R*,2S*)-1-{(4′-chloro-biphenyl-4-sulfonyl)-[2-(1H-tetrazol-5-ylamino)-ethyl]-amino}-2-(3-phenoxy-phenyl)-cyclopropanecarboxylic acid,
(1R*,2S*)-1-{(4′-chloro-biphenyl-4-sulfonyl)-[3-(2H-tetrazol-5-ylamino)-propyl]-amino}-2-phenyl-cyclopropanecarboxylic acid,
3-{[((1R*,2S*)-1-carboxy-2-phenyl-cyclopropyl)-(4′-chloro-biphenyl-4-sulfonyl)-amino]-methyl}-benzoic acid methyl ester,
1-{2-[((1R*,2S*)-1-carboxy-2-phenyl-cyclopropyl)-(4′-chloro-biphenyl-4-sulfonyl)-amino]-ethyl}-1H-imidazole-4-carboxylic acid methyl ester,
(R*,2S*)-1-{(4′-chloro-biphenyl-4-sulfonyl)-[2-(4-methyl carbamoyl-imidazol-1-yl)-ethyl]-amino}-2-phenyl-cyclopropanecarboxylic acid,
(1R*,2S*)-1-[(4′-chloro-biphenyl-4-sulfonyl)-(2-hydroxy-ethyl)-amino]-2-(3-phenoxy-phenyl)-cyclopropanecarboxylic acid, and
3-({((1R*,2S*)-1-carboxy-2-phenyl-cyclopropyl)-[4-(4-chloro-phenyl)-piperazine-1-sulfonyl]-amino}-methyl)-benzoic acid;
   or a prodrug thereof or a pharmaceutically acceptable salt thereof;
[16] A pharmaceutical composition comprising a compound of any of [1] to [15] above, a prodrug thereof or a pharmaceutically acceptable salt thereof, and a pharmaceutically acceptable carrier;
[17 An aggrecanase inhibitor comprising a compound of any of [1] to [15] above, or a prodrug thereof or a pharmaceutically acceptable salt thereof as an active ingredient;
[18] An MMP inhibitor comprising a compound of any of [1] to [15] above, or a prodrug thereof or a pharmaceutically acceptable salt thereof as an active ingredient;
[19] The MMP inhibitor of [18] above, which is an MMP-13 inhibitor;
[20] A prophylactic or therapeutic agent for osteoarthritis comprising a compound of any of [1] to [15] above, a prodrug thereof or a pharmaceutically acceptable salt thereof as an active ingredient;
[21] A prophylactic or therapeutic agent for rheumatoid arthritis comprising a compound of any of [1] to [15] above, a prodrug thereof or a pharmaceutically acceptable salt thereof as an active ingredient;
[22] A method for preventing or treating osteoarthritis, which comprises administering a compound of any of [1] to [15] above, a prodrug thereof or a pharmaceutically acceptable salt thereof to a mammal;
[23] A method for preventing or treating rheumatoid arthritis, which comprises administering a compound of any of [1]to [15]above, a prodrug thereof or a pharmaceutically acceptable salt thereof to a mammal;
[24] The agent of [20] above, which is used in combination with a different therapeutic agent for osteoarthritis;
[25] The agent of [20] above, which is used in combination with a different therapeutic agent for rheumatoid arthritis;
[26] The agent of [21] above, which is used in combination with a different therapeutic agent for osteoarthritis;
[27] The agent of [21] above, which is used in combination with a different therapeutic agent for rheumatoid arthritis;
[28] The method of [22] above, which is used in combination with a different therapeutic agent for osteoarthritis;
[29] The method of [22] above, which is used in combination with a different therapeutic agent for rheumatoid arthritis;
[30] The method of [23] above, which is used in combination with a different therapeutic agent for osteoarthritis;
[31] The method of [23] above, which is used in combination with a different therapeutic agent for rheumatoid arthritis.

### DETAILED DESCRIPTION OF THE INVENTION

The definitions of respective substituents and moieties used in the present specification are as follows.

In the present specification, “C₁₋₆” means that the number of carbon atoms ranges from 1 to 6.

The “single bond” means a direct connection. In —W-A¹—W₁-A², for example, when W is a “single bond”, it is -A¹-W₁-A^{2.}

The “halogen atom” is a fluorine atom, a chlorine atom, a bromine atom or an iodine atom, preferably a fluorine atom, a chlorine atom or a bromine atom.

The “C₁₋₁₀ alkyl group” is a straight chain or branched chain alkyl group having 1 to 10 carbon atoms, and is exemplified by methyl, ethyl, propyl, isopropyl, butyl, isobutyl, sec-butyl, tert-butyl, pentyl, isopentyl, neo-pentyl, tert-pentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like. In some embodiments of the present invention, it is a straight chain or branched chain alkyl group having 1 to 6 carbon atoms.

The “C₁₋₆ alkyl group” is a straight chain or branched chain alkyl group having 1 to 6 carbon atoms, and is exemplified by methyl, ethyl, propyl, isopropyl, butyl, isobutyl, sec-butyl, tert-butyl, pentyl, isopentyl, tert-pentyl, hexyl and the like. In some embodiments of the present invention, it is a straight chain or branched chain alkyl group having 1 to 4 carbon atoms.

The “C₂₋₆ alkenyl group” is a straight chain or branched chain alkenyl group having 2 to 6 carbon atoms, and is exemplified by ethenyl(vinyl), 1-propenyl, 2-propenyl (allyl), isopropenyl, 1-butenyl, 2-butenyl, 3-butenyl, 1-methyl-1-propenyl, 1-methyl-2-propenyl, 2-methyl-2-propenyl, 1-ethylvinyl, 1-pentenyl, 2-pentenyl, 3-pentenyl, 4-pentenyl, 1,2-dimethyl-1-propenyl, 1,2-dimethyl-2-propenyl, 1-ethyl-1-propenyl, 1-ethyl-2-propenyl, 1-methyl-1-butenyl, 1-methyl-2-butenyl, 2-methyl-1-butenyl, 1-isopropylvinyl, 2,4-pentadienyl, 1-hexenyl, 2-hexenyl, 3-hexenyl, 4-hexenyl, 5-hexenyl, 2,4-hexadienyl, 1-methyl-1-pentenyl and the like. In some embodiments of the present invention, it is a straight chain or branched chain alkenyl group having 2 to 4 carbon atoms.

The “C₂₋₆ alkynyl group” is a straight chain or branched chain alkynyl group having 2 to 6 carbon atoms, and is exemplified by ethynyl, propynyl, butynyl, 2-pentynyl, 3-pentynyl, 2-hexynyl, 3-hexynyl and the like.

The “C₁₋₆ alkoxy group” is an alkyloxy group wherein the alkyl moiety thereof is the above-defined C₁₋₆ alkyl group. Examples thereof include methoxy, ethoxy, propoxy, isopropyloxy, butoxy, isobutyloxy, tert-butyloxy, pentyloxy, hexyloxy and the like. In some embodiments of the present invention, it is an alkoxy group wherein the alkyl moiety thereof is a straight chain or branched chain alkyl group having 1 to 4 carbon atoms.

The “halogenated C₁₋₆ alkyl group” is the above-defined C₁₋₆ alkyl group except that it is substituted by the above-defined halogen atom. Examples thereof include fluoromethyl, difluoromethyl, trifluoromethyl, bromomethyl, chloromethyl, 1,2-dichloromethyl, 2,2-dichloromethyl, 2,2,2-trifluoroethyl and the like. In some embodiments of the present invention, it is a halogenated alkyl group wherein the alkyl moiety thereof is a straight chain or branched chain alkyl group having 1 to 4 carbon atoms.

The “halogenated C₁₋₆ alkoxy group” is the above-defined C₁₋₆ alkoxy group except that it is substituted by the above-defined halogen atom. Examples thereof include fluoromethoxy, difluoromethoxy, trifluoromethoxy, bromomethoxy, chloromethoxy, 1,2-dichloromethoxy, 2,2-dichloromethoxy, 2,2,2-trifluoroethoxy and the like. In some embodiments of the present invention, it is a halogenated alkoxy group wherein the alkoxy moiety thereof is a straight chain or branched chain alkoxy group having 1 to 4 carbon atoms.

The “mono(C₁₋₆ alkyl)amino group” is a mono-alkyl-amino group wherein the alkyl moiety thereof is the above-defined C₁₋₆ alkyl group. Examples thereof include methylamino, ethylamino, propylamino, isopropylamino, butylamino, isobutylamino, tert-butylamino, pentylamino, hexylamino and the like. In some embodiments of the present invention, it is a mono-alkyl-amino group wherein the alkyl moiety thereof is a straight chain or branched chain alkyl group having 1 to 4 carbon atoms.

The “di(C₁₋₆ alkyl)amino group” is a di-alkyl-amino group wherein the alkyl moiety thereof is the above-defined C₁₋₆ alkyl group. Examples thereof include dimethylamino, diethylamino, dipropylamino and the like. In some embodiments of the present invention, it is a di-alkyl-amino group wherein the alkyl moiety thereof is a straight chain or branched chain alkyl group having 1 to 4 carbon atoms.

The “C₁₋₆ alkoxy-carbonyl group” is an alkyloxycarbonyl group wherein the alkoxy moiety thereof is the above-defined C₁₋₆ alkoxy group. Examples thereof include methoxycarbonyl, ethoxycarbonyl, propoxycarbonyl, isopropyloxycarbonyl, butoxycarbonyl, isobutyloxycarbonyl, tert-butyloxycarbonyl, pentyloxycarbonyl, hexyloxycarbonyl and the like. In some embodiments of the present invention, it is an alkoxy-carbonyl group wherein the alkyl moiety thereof is a straight chain or branched chain alkyl group having 1 to 4 carbon atoms.

The “C₁₋₆ alkoxy-C₁₋₆ alkyl group” is an alkoxy-alkyl group wherein the alkoxy moiety thereof is the above-defined C₁₋₆ alkoxy group and the alkyl moiety thereof is the above-defined C₁₋₆ alkyl group. Examples thereof include amethoxymethyl, ethoxymethyl, propoxymethyl, butoxymethyl, pentyloxymethyl, hexyloxymethyl, methoxyethyl, ethoxyethyl, propoxyethyl, butoxyethyl, pentyloxyethyl, hexyloxyethyl and the like. In some embodiments of the present invention, it is a C₁₋₄ alkoxy-C₁₋₄ alkyl group wherein the alkoxy moiety thereof is a straight chain or branched chain alkoxy group having 1 to 4 carbon atoms and the alkyl moiety thereof is a straight chain or branched chain alkyl group having 1 to 4 carbon atoms.

The “C₁₋₆ alkyl-aminocarbonyl group” is a mono-alkyl-amino-carbonyl group wherein the alkyl moiety thereof is the above-defined C₁₋₆ alkyl group. Examples thereof include methylaminocarbonyl, ethylaminocarbonyl, propylaminocarbonyl, isopropylaminocarbonyl, butylaminocarbonyl, isobutylaminocarbonyl, tert-butylaminocarbonyl, pentylaminocarbonyl, hexylaminocarbonyl and the like. In some embodiments of the present invention, it is a C₁₋₄ alkyl-aminocarbonyl group wherein the alkyl moiety thereof is a straight chain or branched chain alkyl group having 1 to 4 carbon atoms.

The “C₁₋₆ alkyl-carbonylamino group” is a mono-alkylcarbonyl-amino group wherein the alkyl moiety thereof is the above-defined C₁₋₆ alkyl group. Examples thereof include methylcarbonylamino, ethylcarbonylamino, propylcarbonylamino, isopropylcarbonylamino, butylcarbonylamino, isobutylcarbonylamino, tert-butylcarbonylamino, pentylcarbonylamino, hexylcarbonylamino and the like. In some embodiments of the present invention, it is a mono-alkylcarbonyl-amino group wherein the alkyl moiety thereof is a straight chain or branched chain alkyl group having 1 to 4 carbon atoms.

The “C₁₋₆ alkylsulfonyl group” is an alkylsulfonyl group wherein the alkyl moiety thereof is the above-defined C₁₋₆ alkyl group. Examples thereof include methanesulfonyl, ethanesulfonyl, propanesulfonyl, butanesulfonyl, penanesulfonyl, hexanesulfonyl and the like. In some embodiments of the present invention, it is an alkylsulfonyl group wherein the alkyl moiety thereof is a straight chain or branched chain alkyl group having 1 to 4 carbon atoms.

The “C₁₋₆ alkylsulfonylamino group” is an alkylsulfonyl-amino group wherein the alkyl moiety thereof is the above-defined C₁₋₆ alkyl group. Examples thereof include methanesulfonylamino, ethanesulfonylamino, propanesulfonylamino, butanesulfonylamino, pentanesulfonylamino, hexanesulfonylamino and the like. In some embodiments of the present invention, it is an alkylsulfonylamino group wherein the alkyl moiety thereof is a straight a chain or branched chain alkyl group having 1 to 4 carbon atoms.

The “C₁₋₆ alkylene group” is a straight chain or branched chain alkenylene group having 1 to 6 carbon atoms, and is exemplified by methylene, ethylene, trimethylene, tetramethylene, pentamethylene, hexamethylene and the like. In some embodiments of the present invention, it is a straight chain or branched chain alkylene group having 1 to 4 carbon atoms.

The “C₂₋₆ alkenylene group” is a straight chain or branched chain alkenylene group having 2 to 6 carbon atoms, and is exemplified by vinylene, propenylene, 1-butenylene, 1,3-butadienylene and the like.

The “C₂₋₆ alkynylene group” is a straight chain or branched chain alkynylene group having 2 to 6 carbon atoms, such as a straight chain or branched chain alkynylene group having 2 to 4 carbon atoms, for example ethynylene.

The “C₂₋₆ acyl group” is an alkanoyl group having 2 to 6 carbon atoms, and is exemplified by, acetyl, propionyl, butyryl, pivaloyl and the like. In some embodiments of the present invention, it is acetyl, pivaloyl and the like.

The “optionally substituted C₁₋₁₀ alkyl group” is that wherein the above-defined C₁₋₁₀ alkyl group is optionally substituted by 1 to 5, for example 1 to 3, substituent(s) and includes an unsubstituted C₁₋₁₀ alkyl group. The substituent of the substituted C₃₋₁₄ hydrocarbon ring group include
(i) a halogen atom,
(ii) a nitro group,
(iii) a cyano group,
(iv) a C₁₋₆ alkoxy group,
(v) a hydroxyl group,
(vi) a halogenated C₁₋₆ alkoxy group,
(vii) a carboxyl group,
(vii) a C₁₋₆ alkoxy-carbonyl group,
(ix) an amino group,
(x) a mono(C₁₋₆ alkyl)amino group,
(xi) a di(C₁₋₆ alkyl)amino group,
(xii) an optionally substituted C₃₋₁₄ hydrocarbon ring group,
(xiii) an optionally substituted heterocyclic group,
(ix) a group selected from the above-mentioned group B,
(x) a group selected from the above-mentioned group C, and the like.

In one embodiment of the present invention, the optionally substituted C₁₋₁₀ alkyl group is a straight chain or branched chain alkyl group having 1 to 6 carbon atoms, which is substituted or unsubstituted by the above-mentioned substituents.

The “optionally substituted C₁₋₆ alkyl group” is that wherein the above-defined C₁₋₆ alkyl group is optionally substituted by 1 to 5, for example 1 to 3, substituent(s) and includes an unsubstituted C₁₋₆ alkyl group. Examples of substituent of the “optionally substituted C₁₋₆ alkyl group” include substituents similar to those mentioned above for the substituted C₁₋₁₀ alkyl group.

The “C₃₋₁₄ hydrocarbon ring group” is a saturated or unsaturated cyclic hydrocarbon group having 3 to 14 carbon atoms and includes a C₆₋₁₄ aryl group, a C₃₋₁₀ cycloalkyl group, a C₃₋₈ cycloalkenyl group and the like.

The “C₆₋₁₄ aryl group” is an aromatic hydrocarbon group having 6 to 14 carbon atoms. Examples thereof include phenyl, naphthyl, azulenyl, anthryl, phenanthryl and the like, for example, some embodiments include phenyl.

The “C₃₋₁₀ cycloalkyl group” is a saturated cycloalkyl group having 3 to 10 carbon atoms. Examples thereof include cyclopropyl, cyclobutyl, cyclopentyl, cyclohexyl, cycloheptyl, cyclooctyl, adamantyl, norbornanyl and the like, for example, some embodiments include cyclopentyl, cyclohexyl and cycloheptyl.

The “C₃₋₈ cycloalkenyl group” is a cycloalkenyl group having at least 1, preferably 1 or 2, double bond(s) and 3 to 8 carbon atoms. Examples thereof include cyclopropenyl, cyclobutenyl, cyclopentenyl, cyclopentadienyl, cyclohexenyl (e.g., 2,4-cyclohexadien-1-yl, 2,5-cyclohexadien-1-yl, etc.), cycloheptenyl, cyclooctenyl and the like.

The “substituted C₃₋₁₄ hydrocarbon ring group” is the above-defined C₃₋₁₄ hydrocarbon ring group except that it is substituted by 1 to 5, for example 1 to 3, substituent(s). The substituent of the substituted C₃₋₁₄ hydrocarbon ring group include
(i) an optionally substituted C₁₋₆ alkyl group,
(ii) a halogen atom,
(iii) a nitro group,
(iv) a cyano group,
(v) a C₁₋₆ alkoxy group,
(vi) a hydroxyl group,
(vii) a halogenated C₁₋₆ alkyl group,
(viii) a halogenated C₁₋₆ alkoxy group,
(ix) a carboxyl group,
(x) a C₁₋₆ alkoxy-carbonyl group,
(xi) an amino group,
(xii) a mono(C₁₋₆ alkyl)amino group,
(xiii) a di(C₁₋₆alkyl)amino group,
(xiv) an optionally substituted C₃₋₁₄ hydrocarbon ring group,
(xv) an optionally substituted heterocyclic group,
(xvi) —W₂-Z₂
wherein
W₂ is —(CH₂)ₘ₃—X₃-(CH₂)ₙ₃—;
wherein
m3 and n3 are the same or different and each is selected from 0 and an integer ranging from 1 to 6,
X₃ is selected from a single bond, a C₁₋₆ alkylene group, a C₂₋₆ alkenylene group, a C₂₋₆ alkynylene group, —O—, —N(R²²)—, —S(O)ₘ₄—, —CO—, —CON(R²²)—, —N(R²²)CO— —SO₂N(R²²)—, —N(R²²)SO₂—, —N(R²²)CON(R²³)—, —N(R²²)SO₂N(R²)—, —OCON(R²²)— and —N(R²²)COO—,
wherein
R²² and R²³ are the same or different and each is selected from a hydrogen atom and a C₁₋₆ alkyl group,
m4 is selected from 0 and an integer ranging from 1 to 2,
Z₂ is selected from an optionally substituted C₁₋₆ alkyl group, a halogen atom, a nitro group, a cyano group, a C₁₋₆ alkoxy group, hydroxyl group, a halogenated C₁₋₆ alkyl group, a halogenated C₁₋₆ alkoxy group, a carboxyl group, a C₁₋₆ alkoxy-carbonyl group, an amino group, a mono(C₁₋₆ alkyl)amino group, a di(C₁₋₆ alkyl)amino group, an optionally substituted C₃₋₁₄ hydrocarbon ring group and an optionally substituted heterocyclic group,
(xvii) a group of the formula —(CH₂)ₘ₁₂—X₁₂—(CH₂)ₙ₁₂—R³⁷ wherein each symbol is as defined above,
(xviii) a group selected from the above-mentioned group B,
(xix) a group selected from the above-mentioned group C, and the like.

The “substituted C₃₋₁₄ hydrocarbon ring group” may take together with the substituent(s) to form an “optionally substituted fused C₆₋₁₄ hydrocarbon ring group” or an “optionally substituted fused heterocyclic group”.

The “fused C₆₋₁₄ hydrocarbon ring group” in the “optionally substituted fused C₆₋₁₄ hydrocarbon ring group” includes, for example, a saturated or unsaturated (including partially unsaturated and completely unsaturated) fused hydrocarbon ring group having 6 to 14 carbon atoms, wherein C₃₋₁₄ hydrocarbon ring groups defined above have been fused. Examples thereof include indenyl, indanyl, 1,4-dihydronaphthyl, fluorenyl, 9-oxo-fluorenyl, 1,2,3,4-tetrahydronaphthyl(1,2,3,4-tetrahydro-2-naphthyl, 5,6,7,8-tetrahydro-2-naphthyl and the like), perhydronaphthyl and the like. For example, it is a fused ring of phenyl and a different ring and includes fluorenyl, 9-oxo-fluorenyl and the like.

Examples of substituent of the “optionally substituted fused C₆₋₁₄ hydrocarbon ring group” include substituents similar to those mentioned above for “substituted C₃₋₁₄ hydrocarbon ring group”.

The “optionally substituted C₃₋₁₄ hydrocarbon ring group” includes the “substituted C₃₋₁₄ hydrocarbon ring group” and an unsubstituited C₃₋₁₄ hydrocarbon ring group.

The “heterocyclic group” is a 5-membered or 6-membered saturated or unsaturated (including partially unsaturated and completely unsaturated) monocyclic heterocyclic group having, as an atom constituting the ring, at least 1, for example 1 to 4, heteroatoms selected from an oxygen atom, a nitrogen atom and a sulfur atom, besides a carbon atom.

The “saturated monocyclic heterocyclic group” include, for example, pyrrolidinyl, 2-oxo-pyrrolidinyl, tetrahydrofuryl, tetrahydrothienyl, imidazolidinyl, 2-oxo-imidazolidinyl, 2,4-dioxo-imidazolidinyl, pyrazolydinyl, 1,3-dioxolanyl, 1,3-oxathiolanyl, oxazolidinyl, 2-oxo-oxazolidinyl, thiazolidinyl, 2-oxo-thiazolidinyl, 2,4-dioxo-thiazolidinyl, 4-oxo-2-thioxo-thiazolidinyl, piperidinyl, 2-oxopiperidinyl, piperazinyl, 2,5-dioxopiperazinyl, hexahydropyridazinyl, 3-oxotetrahydropyridazinyl, 2-oxotetrahydropyrimidinyl, tetrahydropyranyl, tetrahydrothiopyranyl, dioxanyl, morpholinyl, 3-oxomorpholinyl, thiomorpholinyl, 3-oxothiomorpholinyl, 2-oxopyrrolidinyl, 2-oxopiperidinyl, 4-oxopiperidinyl, 2,6-dioxopiperidinyl, 2-oxo-1,3-oxazinanyl, 2-oxo-1,3-thiazinanyl, azetidinyl, 1,4-diazepanyl, 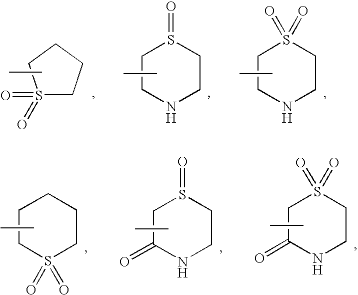
and the like, such as pyrrolidinyl, piperidinyl and morpholinyl.

The “unsaturated monocyclic heterocyclic group” includes, for example, pyrrolyl, 1,5-dihydro-2-oxopyrrolyl, furyl, thienyl, imidazolyl, 1,2-dihydro-2-oxoimidazolyl, 1,3-dihydro-2-oxoimidazolyl, pyrazolyl, 1,2-dihydro-3-oxopyrazolyl, oxazolyl, 2-oxo-oxazolyl, isoxazolyl, thiazolyl, 2-oxothiazolyl, isothiazolyl, 1,2,4-triazolyl, 3-oxo-1,2,4-triazolyl, 1,2,3-triazolyl, tetrazolyl, 1,3,4-oxadiazolyl, 1,2,4-oxadiazolyl, 5-oxo-1,2,4-oxadiazolyl, 1,3,4-thiadiazinyl, 1,3,4-thiadiazolyl, 2-thioxo-1,3,4-thiadiazolyl, 3-oxo-1,4-oxazinyl, 1,2,4-thiadiazolyl, 5-oxo-1,2,4-thiadiazolyl, furazanyl, pyridyl, 2-oxopyridyl, 4-oxopyridyl, 2-thioxopyridyl, 4-thioxopyridyl, pyrimidinyl, 2-oxopyrimidinyl, 3,4-dihydro-4-oxopyrimidinyl, 2,4,6-trioxopyrimidinyl, pyridazinyl, 3-oxopyridazinyl, pyrazinyl, 1,3,5-triazinyl, imidazolinyl, pyrazolinyl, oxazolinyl(2-oxazolinyl, 3-oxazolinyl, 4-oxazolinyl), isoxazolinyl, thiazolinyl, isothiazolinyl, pyranyl, 2-oxopyranyl, 4-oxopyranyl, 4-thioxopyranyl and the like, such as, imidazolyl, pyrazolyl, isoxazolyl, thiazolyl, 1,2,4-triazolyl, tetrazolyl, 1,3,4-oxadiazolyl, pyridyl, pyrimidinyl, pyridazinyl, pyrazinyl and oxazolinyl.

The “substituted heterocyclic group” is the above-defined heterocyclic group except that it is substituted by 1 to 5, for example 1 to 3, substituent(s). As the substituent thereof, examples include substituents similar to those mentioned above for “substituted C₃₋₁₄ hydrocarbon ring group”.

The “substituted heterocyclic group” may take together with the substituent(s) to form an “optionally substituted fused heterocyclic group”.

The “fused heterocyclic group” in the “optionally substituted fused heterocyclic group” includes, for example, a 6-membered to 14-membered saturated or unsaturated (including partially unsaturated and completely unsaturated) fused heterocyclic group having, as an atom constituting the ring, at least 1, for example 1 to 4, heteroatoms selected from an oxygen atom, a nitrogen atom and a sulfur atom, besides a carbon atom. The fused heterocyclic group may be a fused ring group of a saturated or unsaturated heterocyclic group defined above and a C₃₋₁₄ hydrocarbon ring group defined above, or may be a fused ring group of saturated or unsaturated heterocyclic groups defined above. Examples thereof include indolyl, isoindolyl, 2,3-dihydroindolyl, 2,3-dihydroisoindolyl, 1,3-dihydro-2-oxoisoindolyl, 2,3-dihydro-1-oxoisoindolyl, 1,3-dihydro-1,3-dioxoisoindolyl, benzimidazolyl, indazolyl, 4,5,6,7-tetrahydroindazolyl, benzotriazolyl, benzothiazolyl, benzoisothiazolyl, 4,5,6,7-tetrahydrobenzoisothiazolyl, 2-oxobenzothiazolyl, benzothiophenyl, dibenzothiophenyl, 4,5,6,7-tetrahydrobenzothiophenyl, benzofuranyl, dibenzofuranyl, isobenzofuranyl, 4,5,6,7-tetrahydrobenzofuranyl, 4,5,6,7-tetrahydroisobenzofuranyl, benzoxazolyl, benzoisooxazolyl, 2-oxobenzoxazolyl, 4,5,6,7-tetrahydroisobenzoxazolyl, indolizinyl, quinolyl, isoquinolyl, 1,2-dihydro-2-oxoquinolyl, quinazolinyl, quinoxalinyl, cinnolinyl, phthalazinyl, quinolidinyl, carbazolyl, puryl, pteridinyl, indolinyl, isoindolinyl, 4,5,6,7-tetrahydroindolyl, 4,5,6,7-tetrahydroisoindolyl, 5,6,7,8-tetrahydroquinolyl, 1,2,3,4-tetrahydroquinolyl, 2-oxo-1,2,3,4-tetrahydroquinolyl, 1,2,3,4-tetrahydroisoquinolyl, 2-oxo-1,2,3,4-tetrahydroisoquinolyl, 1,3-benzodioxolyl, 3,4-methylenedioxypyridyl, 4,5-ethylenedioxypyrimidinyl, chromenyl, chromanyl, isochromanyl, 1,2,4-benzotriazinyl, 6,7-dihydropyrindinyl, 6,7-dihydrocyclopentapyrazinyl, 6,7-dihydrocyclopentapyridazinyl, 6,7-dihydrocyclopentapyrimidinyl, 2,3,4,5-tetrahydrobenzoazepinyl, 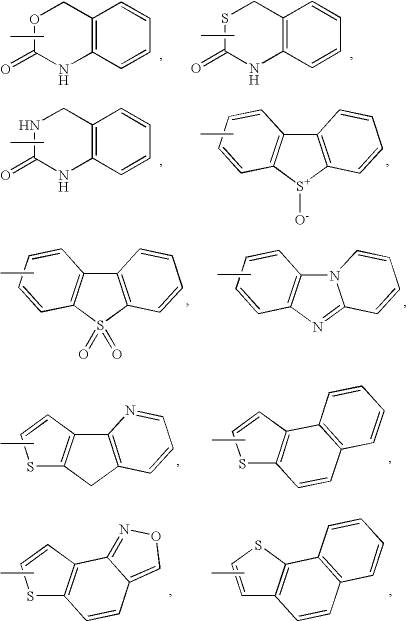
and the like, for example, some embodiments include benzofuranyl, dibenzofuranyl and isoquinolyl.

Example of substituents of the “optionally substituted fused heterocyclic group” include substituents similar to those mentioned above for “substituted heterocyclic group”.

The “optionally substituted heterocyclic group” includes the above-defined “substituted heterocyclic group” and the unsubstituted heterocyclic group.

The “optionally substituted nitrogen-containing heterocyclic group” is a 5-membered or 6-membered saturated or unsaturated (including partially unsaturated and completely unsaturated) monocyclic heterocyclic group having, as an atom constituting the ring, at least one nitrogen atom and, for example, 1 to 4 heteroatoms selected from an oxygen atom, a nitrogen atom and a sulfur atom, and includes a fused ring group of such heterocyclic ring groups above, and a fused ring group of a heterocyclic ring group and a hydrocarbon ring group selected from benzene, cyclopentane and cyclohexane. Examples thereof include pyrrolidine, piperazine, piperidine, pyrrole, pyrazole, imidazole, triazole, tetrazole, pyridine, quinoline, benzoimidazole, thiazole, oxadiazole, morpholine and the like. Examples of substituents of the optionally substituted nitrogen-containing heterocyclic group, include substituents similar to those mentioned above for “substituted C₃₋₁₄ hydrocarbon ring group”.

The “C₆₋₁₄ aryl-C₁₋₆ alkyl group” is an arylalkyl group wherein the alkyl moiety thereof is the above-defined C₁₋₆ alkyl group and the aryl moiety is the above-defined C₆₋₁₄ aryl group. Examples thereof include benzyl, phenethyl, 3-phenylpropyl, 2-phenylpropyl, 4-phenylbutyl and the like. For example, it may be an arylalkyl group wherein the alkyl moiety thereof is a straight chain alkyl group having 1 to 4 carbon atoms and the aryl moiety is phenyl.

The “optionally substituted C₆₋₁₄ aryl-C₁₋₆ alkyl group” is that wherein the above-defined C₆₋₁₄ aryl-C₁₋₆ alkyl group is optionally substituted by 1 to 5, for example 1 to 3, substituent(s) and includes unsubstituted C₆₋₁₄ aryl-C₁₋₆ alkyl group. Examples of substituents of the optionally substituted C₆₋₁₄ aryl-C₁₋₆ alkyl group include substituents similar to those mentioned above for the substituted C₃₋₁₄ hydrocarbon ring group. In one embodiment, it is a phenyl-C₁₋₄ alkyl group substituted or unsubstituted by the above-mentioned substituents.

Each symbol in the formula (1) of preferable compounds of the formula (1) is explained in the following.

In some embodiments of the inventive compounds of formula (1), R¹ is —W-A¹-W₁-A², W is —(CH₂)ₘ—X—(CH₂)ₙ, and W₁ is —(CH₂)ₘ₁—X₁—(CH₂)ₙ₁—, wherein each symbol is as defined above.
m, n, m1 and n1 are for example 0.
X and X₁ are for example a single bond.

The optionally substituted C₃₋₁₄ hydrocarbon ring group at A¹ is for example an optionally substituted C₆₋₁₄ aryl group, preferably an optionally substituted phenyl group. The substituent thereof is for example a substituent selected from the above-mentioned group B. The number of substituents is for example a integer ranging from 1 to 3.

The optionally substituted heterocyclic group at A¹ is for example an optionally substituted saturated monocyclic heterocyclic group (e.g., piperazinyl) or an optionally substituted unsaturated monocyclic heterocyclic group (e.g., thienyl). The substituent thereof is for example a substituent selected from the above-mentioned group B. The number of substituents is for example a integer ranging from 1 to 3.

A² is for example, a group of the following formula

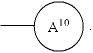

The C₃₋₁₄ hydrocarbon ring group at the ring A¹⁰ is for example a C₆₋₁₄ aryl group, preferably phenyl group.

The heterocyclic group at the ring A¹⁰ is for example an unsaturated monocyclic heterocyclic group, preferably tetrazolyl, thienyl or isooxazolyl.

The ring A¹⁰ is substituted by 1 to 5 (preferably 1) groups of “—(CH₂)ₘ₁₂—X₁₂—(CH₂)ₙ₁₂—R³⁷”, wherein each symbol is as defined above, which are the same or different.

m12 and n12 are the same or different and each is for example 0.

X₁₂ is for example a single bond.

R³⁷ is for example a halogen atom (e.g., chlorine atom), a halogenated C₁₋₆ alkyl group (e.g., trifluoromethyl) or a C₁₋₆ alkyl group optionally substituted by hydroxyl groups (e.g., methyl).

A¹ and A² may be taken together with a substituent thereof to form an optionally substituted fused C₆₋₁₄ hydrocarbon ring group. The A₁₀ and A¹ may be taken together with a substituent thereof to form an optionally substituted fused C₆₋₁₄ hydrocarbon ring group. The optionally substituted fused ring group is for example the above-defined “optionally substituted fused C₆₋₁₄ hydrocarbon ring group” or the like.

The “fused C₆₋₁₄ hydrocarbon ring group” in the “optionally substituted fused C₆₋₁₄ hydrocarbon ring group” is for example 9H-fluorenyl or 9-oxo-9H-fluorenyl. The substituent thereof is for example a substituent selected from the above-mentioned group B. The number of substituents is for example 1.

In some embodiments of the inventive compounds of formula (1), R² is (1) —(CH₂)ₘ₅—X₅—(CH₂)ₙ₅-A⁵, wherein each symbol is as defined above, or (2) —(CH₂)ₘ₅—X₅-(CH₂)ₙ₅—R³², wherein each symbol is as defined above, provided that when m5 and n5 are 0 and X₅ is a single bond, then R³² should not be a hydrogen atom.
m5 and n5 are for example 1 or 2.

X₅ is for example a single bond, a C₁₋₆ alkylene group (e.g., dimethylmethylene), —N(R⁶)—, —CO—, —COO—, —CON(R⁶)—, —N(R⁶)CO—, —N(R⁶)SO₂—, —N(R⁶)SO₂N(R⁷)—, wherein R⁶ is for example a hydrogen atom and R⁷ is for example a hydrogen atom, or the like.

A⁵ is for example, a group of the following formula

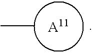

The C₃₋₁₄ hydrocarbon ring group at the ring A¹¹ is for example a C₆₋₁₄ aryl group, preferably phenyl group.

The heterocyclic group in the ring A¹¹ is for example a saturated monocyclic heterocyclic group (e.g., pyrrolidinyl, piperidinyl, 1,2,5-thiadiazolidinyl, 1,1,3,4-tetraoxo-llambda*6*-[1,2,5]thiadiazolidinyl) or an unsaturated monocyclic heterocyclic group (e.g., pyrrolyl, furyl, pyridyl, thiazolyl, 1,2,4-thiadiazolyl, 5-oxo-1,2,4-thiadiazolyl, oxazolyl, 1,2,4-oxadiazolyl, 5-oxo-1,2,4-oxadiazolyl, imidazolyl, 1,2,4-triazolyl, 5-oxo-1,2,4-triazolyl, tetrazolyl, pyrazolyl, 5-oxo-pyrazolyl).

The ring A¹¹ is optionally substituted by 1 to 5 (preferably 1 or 2) groups of “—(CH₂)ₘ₁₃—X₁₃—(CH₂)ₙ₁₃—R³⁸”, wherein each symbol is as defined above, which are the same or different.

m13 and n13 are the same or different and each is for example 0.

X₁₃ is for example a single bond, —CO—, —COO—, —CON(R⁶)—, —N(R⁶)SO₂—, wherein R⁶ is for example hydrogen atom, or the like.

R³⁸ is for example a hydrogen atom, a hydroxyl group, a carboxyl group, a C₁₋₆ alkyl group optionally substituted by hydroxyl groups (e.g., methyl), a C₁₋₆ alkylsulfonyl group (e.g., methanesulfonyl) or the like.

R³² is for example a hydrogen atom, a hydroxyl group, a carboxyl group, an amino group, a halogenated C₁₋₆ alkyl group (e.g., trifluoromethyl), a C₁₋₆ alkyl group optionally substituted by hyroxyl groups (e.g., methyl, ethyl, hydroxymethyl, 1-hydroxy-1-methylethyl), a C₁₋₆ alkoxy group optionally substituted by hydroxyl groups (e.g., t-butoxy), a C₁₋₆ alkoxy-carbonyl group (e.g., isopropoxycarbonyl), a C₁₋₆ alkylsulfonyl group (e.g., methanesulfonyl) or the like.

The ring A¹¹ may be taken together with a group of “—(CH₂)ₘ₁₃—X₁₃—(CH₂)ₙ₁₃—R³⁸”, wherein each symbol is as defined above, to form an optionally substituted fused ring group. The “optionally substituted fused ring group” is for example the above-defined “optionally substituted fused C₆₋₁₄ hydrocarbon ring group”, the above-defined “optionally substituted fused heterocyclic group” or the like.

The “fused C₆₋₁₄ hydrocarbon ring group” in the “optionally substituted fused C₆₋₁₄ hydrocarbon ring group” is for example 9H-fluorenyl or 9-oxo-9H-fluorenyl. The substituent thereof is for example a substituent selected from the above-mentioned group B. The number of substituents is for example 1.

The “fused heterocyclic group” in the “optionally substituted fused heterocyclic group” is for example benzoimidazolyl. The substituent thereof is for example a substituent selected from the above-mentioned group B. The number of substituents is for example 1.

R², R³ and the cyclopropane ring may be taken together to form an optionally further substituted fused ring. The “fused ring” is for example a fused C₆₋₁₄ hydrocarbon ring in the above-defined fused C₆₋₁₄ hydrocarbon ring group or a fused heterocyclic ring in the above-defined fused heterocyclic group, wherein the above-defined C₃₋₁₄ hydrocarbon ring group and/or the above-defined heterocyclic group are/is fused with the cyclopropane ring, or the like. Examples thereof include is 2-aza-bicyclo[3.1.0]hexane, 2-aza-bicyclo[4.1.0]heptane, 4-oxa-2-aza-bicyclo[4.1.0]heptane, 4-oxo-2,5-diaza-bicyclo[5.1.0]octane, 5-oxa-2-aza-bicyclo[5.1.0]octane and the like. The “fused ring” is optionally further substituted, and the substituent thereof is for example a substituent selected from the above-mentioned group C. The number of substituents is for example 1.

In some embodiments of the inventive compounds of formula (1), R³ and R⁴ are the same or different and each is (1) —(CH₂)ₘ₂—X₂—(CH₂)ₙ₂-A⁴, wherein each symbol is as defined above, or (2) —(CH₂)ₘ₆—X₆—(CH₂)ₙ₆—R³³, wherein each symbol is as defined above, and for example, one of them is a hydrogen atom and the other is —(CH₂)ₘ₂—X₂—(CH₂)ₙ₂-A⁴, wherein each symbol is as defined above.

m2 and n2 are the same or different and each is for example 0 or 1.

X₂ is for example a single bond.

A⁴ is for example, a group of the following formula

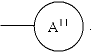

The C₃₋₁₄ hydrocarbon ring group at the ring A¹¹ is for example a C₆₋₁₄ aryl group, preferably phenyl group.

The heterocyclic group at the ring A¹¹ is for example a saturated monocyclic heterocyclic group, preferably piperidinyl.

The ring A¹¹ is optionally substituted by 1 to 5 (preferably 1 or 2) groups of “—(CH₂)ₘ₁₃—X₁₃—(CH₂)ₙ₁₃—R³⁸”, wherein each symbol is as defined above, which are the same or different.

m₁₃ and n₁₃ are the same or different and each is for example 0 or an integer ranging from 1 to 2.

X₁₃ is for example a single bond, —O—, —N(R⁶)—, —N(R⁶)CO—, —N(R⁶)SO₂—, wherein R⁶ is for example a hydrogen atom, or the like.

R³⁸ is for example a hydrogen atom, a halogen atom (e.g., a chlorine atom), a hydroxyl group, a cyano group, a carboxyl group, a C₁₋₆ alkyl group optionally substituted by hydroxyl groups (e.g., methyl, 2-hydroxyethyl), a C₁₋₆ alkoxy group optionally substituted by hydroxyl groups (e.g., methoxy, isobutoxy), a di(C₁₋₆ alkyl)amino group (e.g., diethylamino), a C₃₋₁₄ hydrocarbon ring group optionally substituted by 1 to 5 substituent(s) selected from the above-mentioned group B (e.g., a C₆₋₁₄ aryl group (e.g., phenyl group), a C₃₋₈ cycloalkyl group (e.g., cyclohexyl)), a heterocyclic group optionally substituted by 1 to 5 substituent(s) selected from the above-mentioned group B (e.g., an saturated monocyclic heterocyclic group (e.g., pyrrolidinyl, piperidinyl, morpholinyl) optionally substituted by a C₁₋₆ alkoxy-carbonyl group (e.g., t-butoxycarbonyl), an optionally substituted unsaturated monocyclic heterocyclic group (e.g., pyrazolyl, pyridyl, imidazolyl)) or the like.

m6 and n6 in —(CH₂)ₘ₆—X₆—(CH₂)ₙ₆—R³³ is for example 0.

X₆ is for example a single bond.

R³³ is for example a hydrogen atom.

A⁴ and R³³ may be taken together to form an optionally substituted fused ring group. The optionally substituted fused ring group is for example the above-defined “optionally substituted fused C₆₋₁₄ hydrocarbon ring group”, the above-defined “optionally substituted fused heterocyclic group” or the like. Examples thereof include 1,2,3,4-tetrahydroisoquinoline and the like. The substituent thereof is for example a substituent selected from the above-mentioned group C, preferably a C₂₋₆ acyl group (e.g., acetyl). The number of substituents is for example 1.

R³ and R⁴ may be taken together with a carbon atom bonded thereto to form the following ring 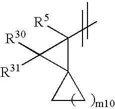
wherein each symbol is as defined above.

m10 is for example an integer ranging from 1 to 4, preferably 1.

Provided that R³ and R⁴ are not hydrogen atoms at the same time.

In some embodiments of the inventive compounds of formula (1), R⁵ is for example (1) —CO₂R²¹, (2) —C(O)NHOR²¹ (3) —C(O)NH—SO₂—R²¹, (4) —C(O)NHR²¹ or (5) —(CH₂)ᵣ₁—R⁵⁰, wherein each symbol is as defined above.

R²¹ is for example a hydrogen atom, an optionally substituted C₁₋₁₀ alkyl group (e.g., methyl) or —(CH₂)ₘ₇—X₇-(CH₂)ₙ₇—R³⁴, wherein each symbol is as defined above.

m7 and n7 are the same or different and each is for example 0 or an integer ranging from 1 to 2.

X₇ is for example a single bond.

R³⁴ is for example a C₃₋₁₄ hydrocarbon ring group optionally substituted by 1 to 5 substituent(s) selected from the above-mentioned group B, a heterocyclic group optionally substituted by 1 to 5 substituent(s) selected from the above-mentioned group B or the like.
r1 is for example 0 or an integer ranging from 1 to 2.

The “optionally substituted C₃₋₁₄ hydrocarbon ring group” at R³⁴ and R⁵⁰ is for example the above-defined “optionally substituted C₃₋₁₄ hydrocarbon ring group” or the like.

The “optionally substituted heterocyclic group” at R³⁴ and R⁵⁰ is for example the above-defined “optionally substituted heterocyclic group” or the like. Examples thereof include 1-hydroxy-1H-pyridin-2-one, 3-hydroxy-1H-pyridin-2-one, 3-hydroxy-1,2-dimethyl-1H-pyridin-4-one, 3-hydroxy-pyran-4-one, 3-hydroxy-2-methyl-pyran-4-one, 3-hydroxy-1H-pyridin-2-one, 1-hydroxy-1H-pyridine-2-thione, 3-hydroxy-1,2-dimethyl-1H-pyridine-4-thione, 3-hydroxy-1H-pyridine-2-thione, 3-hydroxy-pyran-4-thione, 3-hydroxy-2-methyl-pyran-4-thione, 3H-[1,3,4]thiadiazole-2-thione, barbituric acid, 2-thioxo-thiazolidin-4-one, thiazolidine-2,4-dione, imidazolidine-2,4-dione, 6H-1,3,4-thiazine, nitropyrimidine and the like.

R²¹ of —C(O)NHR²¹, A⁴ and the cyclopropane ring may be taken together to form an optionally further substituted fused ring. The “fused ring” is for example a fused C₆₋₁₄ hydrocarbon ring in the above-defined fused C₆₋₁₄ hydrocarbon ring group or a fused heterocyclic ring in the above-defined fused heterocyclic group, wherein the above-defined C₃₋₁₄ hydrocarbon ring group and/or the above-defined heterocyclic group are/is fused with the cyclopropane ring, or the like. Examples thereof include 2-oxo-1,2,3,7b-tetrahydro-3-aza-cyclopropa [a] naphthalene, 2-oxo-2,3,4,8b-tetrahydro-1H-3-aza-benzo [a] cyclopropa[c] and the like. The “fused ring” is optionally further substituted, and the substituent thereof is for example a substituent selected from the above-mentioned group C. The number of substituents is for example 1.

In some embodiments of the inventive compounds of formula (1), R³⁰ and R³¹ are the same or different and each is —(CH₂)ₘ8—X₈—(CH₂)ₙ₈-A⁶, wherein each symbol is as defined above, or —(CH₂)ₘ₉—X₉—(CH₂)ₙ₉—R³⁶, wherein each symbol is as defined above, preferably —(CH₂)ₘ₉—X₉—(CH₂)ₙ₉—R³⁶, more preferably a hydrogen atom or a C₁₋₆ alkyl group optionally substituted by hydroxyl groups.

m8 and n8 are the same or different and each is for example 0 or an integer ranging from 1 to 2, preferably 0.

X₈ is for example a single bond.

A⁶ is for example, a group of the following formula 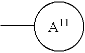
wherein each symbol is as defined above.

m9 and n9 are the same or different and each is for example 0 or an integer ranging from 1 to 2, preferably 0.

X₉ is preferably a single bond.

R³⁶ is for example
(a) a hydrogen atom
(b) a C₁₋₆ alkyl group optionally substituted by hydroxyl groups (e.g., methyl, ethyl, 2-hydroxymethyl) or
   (c) a C₁₋₆ alkoxy-C₁₋₆ alkyl group (e.g., methoxymethyl).

A⁴, R³⁶ and the cyclopropane ring may be taken together to form an optionally further substituted fused ring. The “fused ring” is for example a fused C₆₋₁₄ hydrocarbon ring in the above-defined fused C₆₋₁₄ hydrocarbon ring group or a fused heterocyclic ring in the above-defined fused heterocyclic group, wherein the above-defined C₃₋₁₄ hydrocarbon ring group and/or the above-defined heterocyclic group are/is fused with the cyclopropane ring, or the like. Examples thereof include 1,1a,2,3,4,8b-hexahydro-benzo[a]cyclopropa[c]cycloheptene, 1,1a,6,6a-tetrahydro-cyclopropa [a] indene, 1a,2,3,7b-tetrahydro-1H-cyclopropa[a]naphthalene, 1a,2,3,8b-tetrahydro-1H-4-oxa-benzo[a]cyclopropa[c]cycloheptene, 1,1a,2,3,4,8b-hexahydro-4-aza-benzo[a]cyclopropa[c]cycloheptene and the like. The “fused ring” is optionally further substituted, and the substituent thereof is for example a substituent selected from the above-mentioned group C, preferably a hydroxyl group and a C₂₋₆ acyl group (e.g., acetyl). The number of substituents is for example 1.

R²¹ of —CO₂R²¹, R³⁰ and the cyclopropane ring may be taken together to form an optionally further substituted fused ring. The “fused ring” is for example a fused C₆₋₁₄ hydrocarbon ring in the above-defined fused C₆₋₁₄ hydrocarbon ring or a fused heterocyclic ring in the above-defined fused heterocyclic group, wherein the above-defined C₃₋₁₄ hydrocarbon ring group and/or the above-defined heterocyclic group are/is fused with the cyclopropane ring, or the like. Examples thereof include 2-oxo-3-oxa-bicyclo[3.1.0]hexyl and the like. The “fused ring” is optionally further substituted, and the substituent thereof is for example a substituent selected from the above-mentioned group C. The number of substituents is for example 1.

R³⁰ and R³¹ may be taken together with a carbon atom bonded thereto to form the following ring, 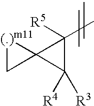
wherein each symbol is as defined above.

m11 is for example an integer ranging from 1 to 4, preferably 1.

As the compound represented by the formula (1), the following compound is preferable.

[Compound A]

A compound wherein
R¹ is —W-A₁-W₁-A
   wherein W is —(CH₂)ₘ—X— (CH₂)ₙ— and W₁ is —(CH₂)ₘ₁—X₁—(CH₂)ₙ₁— (wherein m, n, m1 and n1 are 0, and X and X₁ are single bonds), and
A² is 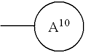
   wherein each symbol is as defined above;
R³ is —(CH₂)ₘ₂—X₂—(CH₂)ₙ₂-A⁴, wherein each symbol is as defined above;
A⁴, A⁵ and A⁶ are the same or different and each is 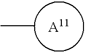
   wherein each symbol is as defined above; and
R⁵ is —CO₂R²¹ or —C(O)NHOR²¹ wherein R²¹ is a hydrogen atom.

As the compound represented by the formula (1), the compound represented by the following formula (1′) is also preferable: 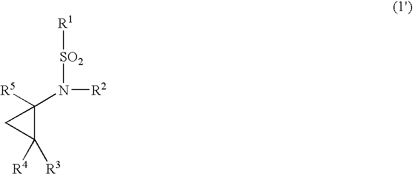
wherein
R¹ is —W-A¹-W₁-A
   wherein
W is —(CH₂)ₘ—X—(CH₂)ₙ—,
W₁ is —(CH₂)ₘ₁—X₁-(CH₂)ₙ₁—,
   wherein
   m, m1, n and n1 are the same or different and each is selected from 0 and an integer ranging from 1 to 6,
   X and X₁ are the same or different and each is selected from a single bond, a C₁₋₆ alkylene group, a C₂₋₆ alkenylene group, a C₂₋₆ alkynylene group, —O—, —N(R⁶)—, S(O)_{q}-r —CO—, —CON(R⁶)—, —N(R⁶)CO—, —SO₂N(R⁶)—, —N(R⁶)SO₂—, —N(R⁶)CON(R⁷)—, —N(R⁶)SO₂N(R⁷)—, —OCON(R⁶)— and —N(R⁶)COO—,
   wherein
   R⁶ and R⁷ are the same or different and each is selected from a hydrogen atom, a C₁₋₆ alkyl group, an optionally substituted C₃₋₁₄ hydrocarbon ring group and an optionally substituted heterocyclic group,
   q is selected from 0 and an integer ranging from 1 to 2,
   A¹ is selected from an optionally substituted C₃₋₁₄ hydrocarbon ring group and an optionally substituted heterocyclic group;
   A² is selected from a substituted C₃₋₁₄ hydrocarbon ring group and a substituted heterocyclic group;
R² is selected from
   —(CH₂)ᵣ—CO—R⁸ (1)
   wherein
   r is selected from 0 and an integer ranging from 1 to 6,
   R⁸ is selected from a C₁₋₆ (alkoxy group and —N(R⁹) (R¹⁰)
   wherein
   R⁹ and R¹⁰ are the same or different and each is selected from a hydrogen atom, a C₁₋₆ alkyl group, a C₁₋₆ alkylsulfonyl group, —SO₂A³ and A³, or may be taken together with a nitrogen atom to form an optionally substituted nitrogen-containing heterocyclic group,
   A³ is selected from an optionally substituted C₃₋₁₄ hydrocarbon ring group and an optionally substituted heterocyclic group;
      —(CH₂)ᵣ—N(R¹¹)(R¹²) (2)
   wherein
   r is as defined above,
   R¹¹ and R¹² are the same or different and each is selected from a hydrogen atom, a C₁₋₆ alkyl group, —CO—R¹³, —SO₂—R¹⁴ and A³, or may be taken together with a nitrogen atom to form an optionally substituted nitrogen-containing heterocyclic group,
   wherein
   R¹³ is selected from a C₁₋₆ alkyl group optionally substituted by C₁₋₆ alkoxy groups or hydroxy groups, and a C₁₋₆ alkoxy group,
   R¹⁴ is selected from a C₁₋₆ alkyl group, a halogenated C₁₋₆ alkyl group, —N(R¹⁵)(R¹⁶) and A³,
   wherein
   R¹⁵ and R¹⁶ are the same or different and each is selected from a hydrogen atom, a C₁₋₆ alkyl group, a C₁₋₆ alkoxy-carbonyl group and A³,
   A³ is as defined above; and
      —(CH₂)ᵣ—R¹⁷ (3)
   wherein
   r is as defined above,
   R¹⁷ is selected from a C₁₋₆ alkyl group optionally substituted by at least one substituent selected from hydroxy groups and —CO₂R¹⁸ groups, and A³,
   wherein
   R¹⁸ is selected from a hydrogen atom and a C₁₋₆ alkyl group,
   A³ is as defined above;
R³ and R⁴ are the same or different and each is selected from
(1) a hydrogen atom,
(2) a C₁₋₆ alkyl group
(3) a halogenated C₁₋₆ alkyl group, and
   —(CH₂)ₘ₂—X₂—(CH₂)ₙ₂-A⁴, (4)
   wherein
   m2 and n2 are the same or different and each is selected from 0 and an integer ranging from 1 to 6,
   X₂ is selected from a single bond, a C₁₋₆ alkylene group, a C₂₋₆ alkenylene group, a C₂₋₆ alkynylene group, —O—, —N(R¹⁹)—, —S(O)_{q1}, —CO—, —CON(R¹⁹)—, —N(R¹⁹)CO—, —SO₂N(R¹⁹)—, —N(R¹⁹)SO₂—, —N(R¹⁹)CON(R²⁰)—, —N(R¹⁹)SO₂N(R²⁰)—, —OCON(R¹⁹)— and —N(R¹⁹)COO—,
   wherein
   R¹⁹ and R²⁰ are the same or different and each is selected from a hydrogen atom, a C₁₋₆ alkyl group, an optionally substituted C₃₋₁₄ hydrocarbon ring group and an optionally substituted heterocyclic group,
   q1 is selected from 0 and an integer ranging from 1 to 2,
   A⁴ is selected from an optionally substituted C₃₋₁₄ hydrocarbon ring group and an optionally substituted heterocyclic group;
R⁵ is selected from
(1) —CO₂R²¹,
(2) —C(O)NHOR²¹,
(3) —C(O)NH—SO₂—R²¹,
(4) —C(O)NHR²¹,
(5) —SH,
(6) —CH₂CO₂R²¹,
(7) —C(O)R²¹,
(8) —N(OH)COR²¹,
(9) —SN₂H₂R²¹,
(10) —SONHR²¹,
(11) —CH₂CO₂H,
(12) —PO(OH)₂,
(13) —PO(OH)NHR²¹,
(14) —CH₂SH and
(15) —CH₂OH
   wherein
   R²¹ is selected from a hydrogen atom, an optionally substituted C₁₋₁₀ alkyl group and an optionally substituted C₆₋₁₄ aryl-C₁₋₆ alkyl group.

In some embodiments of the inventive compounds of formula (1′), R¹ is for example that wherein A¹ is an optionally substituted C₆₋₁₄ aryl group (e.g., phenyl), an optionally substituted saturated monocyclic heterocyclic group (e.g., piperazinyl), or an optionally substituted unsaturated monocyclic heterocyclic group (e.g., thienyl) and A² is a substituted C₆₋₁₄ aryl (e.g., phenyl) an optionally substituted fused C₆₋₁₄ hydrocarbon ring group (e.g., fluorenyl), a substituted saturated monocyclic heterocyclic group (e.g., thienyl, isooxazolyl, pyridyl, tetrazolyl) or an optionally substituted heterocyclic group (e.g., benzofuranyl, benzothiophenyl).

For R¹, 4-chlorobiphenyl, 4-(4-methylthiophen-2-yl)phenyl, 4-(4-chlorophenyl)piperazin-1-yl, 7-bromo-9H-fluoren-2-yl, 7-fluoro-9H-fluoren-2-yl, 7-chloro-9H-fluoren-2-yl, 5-(5-trifluoromethyl-isooxazol-3-yl)-thiophen-2-yl, 5-(5-chloro-pyridin-2-yl)-thiophen-2-yl, 5′-methyl-[2,2′]bithiophenyl-5-yl, 5-benzofuran-2-yl-thiophen-2-yl, 5-benzo[b]thiophen-2-yl-thiophen-2-yl, 2-methyl-2H-tetrazol-5-yl and the like are examples of embodiments of the present invention.

In some embodiments of the inventive compounds of formula (1′), R² is for example
(1) —(CH₂)ᵣ—CO—R⁸ wherein each symbol is as defined above:
   such as carbamoylmethyl, methanesulfonylaminocarbonylmethyl, pyrrolidin-1-ylcarbonylmethyl, 3,4-dihydroxypyrrolidin-1-ylcarbonylmethyl, methoxycarbonylmethyl, ethoxycarbonylmethyl, 1H-tetrazol-5-ylcarbamoylmethyl, 5-carbamoylpentyl and the like;
(2) —(CH₂)ᵣ—N(R¹¹) (R¹²) wherein each symbol is as defined above:
   such as 2-tert-butoxycarbonylaminoethyl, 2-methanesulfonylaminoethyl, 2-isopropoxycarbonylaminosulfonylaminoethyl, 2-trifluoromethanesulfonylaminoethyl, 1H-tetrazol-5-ylaminoethyl, 1H-tetrazol-5-ylaminopropyl, aminosulfonylaminoethyl, 2-hydroxyacetylaminoethyl, 2-hydroxy-2-methyl-propionylaminoethyl and the like; or
(3) —(CH₂)ᵣ—R¹⁷ wherein each symbol is as defined above:
   such as carboxymethyl, 5-oxo-2,5-dihydro-1H-pyrazol-3-ylmethyl, 2-hydroxy-2-methyl-propyl, 1H-benzoimidazol-2-ylmethyl, 3-carboxybenzyl, 4-carboxybenzyl, 2H-tetrazol-5-ylmethyl, benzyl, 3-hydroxybenzyl, 2-carboxy-2-methyl-propyl, methyl, 4-methanesulfonylaminocarbonyl-thiazol-2-ylmethyl, 5-carboxy-furan-2-ylmethyl, 3-carboxy-pyridin-2-ylmethyl, pyridin-2-ylmethyl, pyridin-3-ylmethyl, 4-carboxy-thiazol-2-yl, 3-methanesulfonylamino-benzyl, 5-oxo-4,5-dihydro-[1,2,4]oxadiazol-3-ylmethyl, 5-oxo-4,5-dihydro-1H-[1,2,4]triazol-3-ylmethyl, 2-carboxy-pyrrol-1-ylethyl, 5-oxo-4,5-dihydro-[1,2,4]thiadiazol-3-ylmethyl, 2-carboxy-pyridin-3-yl, 2-(1H-tetrazol-5-ylamino)-ethyl, 5-carboxy-imidazol-1-yl, 4-carboxy-pyrazol-1-ylethyl, 3-carboxy-isoxazol-5-ylmethyl, 2-(1,1,3,4-tetraoxo-1 lambda*6* -[1,2,5]thiadiazolidin-2-yl)-ethyl, 3-carboxypropyl, 4-carboxy-piperidin-1-ylethyl, 3-carboxy-piperidin-1-ylethyl 4-oxalyl-benzyl, 4-carboxy-imidazol-1-ylethyl, 2-(4-methylcarbamoyl-pyrazol-1-yl)-ethyl, 3-methoxycarbonylbenzyl, 2-(4-methoxycarbonyl-imidazol-1-yl)-ethyl, 2-(4-methylcarbamoyl-imidazol-1-yl)-ethyl and the like.

In some embodiments of the inventive compounds of formula (1′), R³ and R⁴ are for example, one of them is a hydrogen atom and the other is for example —(CH₂)ₘ₂—X₂—(CH₂)ₙ₂-A⁴ wherein each symbol is as defined above, such as phenyl, benzyl, 2-,3- or 4-chlorophenyl, 2,3-dichlorophenyl, 2,5-dichlorophenyl, 2,6-dichlorophenyl, 3,4-dichlorophenyl, 2-,3- or 4-methylphenyl, 2-methoxyphenyl,3-methoxyphenyl, 2-phenoxyphenyl,3-phenoxyphenyl, biphenyl-2-yl, biphenyl-4-yl, 2-,3- or 4-cyanophenyl, 2-benzylphenyl, 3-benzylphenyl, 2-trifluoromethylphenyl, 3-trifluoromethylphenyl, 5-chloro-2-trifluoromethylphenyl, 3-isobutoxyphenyl, 3-cyclohexyloxyphenyl, 3-hydroxyphenyl, 3-(tert-butoxycarbonyl-piperidin-4-yloxy)phenyl, 3-(piperidin-4-yloxy)phenyl, tert-butoxycarbonyl-piperidin-4-yl, piperidin-4-yl, 3-(2-diethylaminoethylamino)phenyl, 3-(pyridin-2-ylamino)phenyl, 3-(2-piperidin-1-ylacetylamino)phenyl, 3-(2-hydroxyethoxy)phenyl, 3-(2-piperidin-1-ylethylamino)phenyl, 3-(2-piperidin-1-ylethanesulfonylamino)phenyl, 3-(2-imidazol-1-ylethoxy)phenyl, 3-[(pyridin-3-ylcarbonyl)amino]phenyl, 3-(2-pyrrolidin-1-ylethoxy)phenyl, 3-(2-morpholin-4-ylethoxy)phenyl, 3-(pyridin-3-yloxy)phenyl, 3-(2-pyrazol-1-ylethoxy)phenyl and the like.

In some embodiments of the inventive compounds of formula (1′), R⁵ is for example (1) —CO₂R²¹ (e.g., a carboxyl group, etc.), (2) —C(O)NHOR²¹ (e.g., hydroxyaminocarbonyl, etc.), (3) —C(O)NH—SO₂—R²¹ (e.g., a C₁₋₆ alkyl-sulfonylaminocarbonyl group such as methylsulfonylaminocarbonyl, etc.), (4) —C(O)NHR²¹ (e.g., a C₁₋₆ alkyl-aminocarbonyl group such as methylaminocarbonyl, etc.) or the like.

The “pharmaceutically acceptable salt” may be any as long as it forms a non-toxic salt with a compound of the above-mentioned formula (1). Such salt can be obtained by reacting the compound with an inorganic acid such as hydrochloric acid, sulfuric acid, phosphoric acid, hydrobromic acid and the like; or an organic acid such as oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, benzylsulfonic acid and the like; or an inorganic base such as sodium, potassium, lithium, calcium, magnesium, ammonium and the like; or an organic base such as methylamine, diethylamine, triethylamine, triethanolamine, ethylenediamine, tris(hydroxymethyl)methylamine, guanidine, choline, cinchonine N-methyl-D-glucamine and the like; or an amino acid such as lysine, histidine, arginine, alanine and the like. The present invention encompasses water-retaining product, hydrate and solvate of each compound.

The compounds of the above-mentioned formula (1) have various isomers. For example, E compound and Z compound are present as geometric isomers, and when the compound has an asymmetric carbon, an enantiomer and a diastereomer are present due to the asymmetric carbon. A tautomer may be also present. The present invention encompasses all of these isomers and mixtures thereof.

The present invention also encompasses prodrug and metabolite of the compound represented by the formula (1).

The “prodrug” means a derivative having a chemically modified drug molecule, which does not show physiological activity by itself, but which shows inherent efficacy by reverting to the original compound in a body after administration. The “prodrug” in the present invention means a derivative of N-substituted-N-sulfonylaminocyclopropane compound (1) having a group capable of chemical or metabolic decomposition and showing a pharmaceutical activity by hydrolysis or solvolysis or by decomposition under physiological condition. For example, those wherein a hydroxyl group of the compound is substituted by —CO-alkyl, —CO₂-alkyl, —CONH-alkyl, —CO-alkenyl, —CO₂-alkenyl, —CONH-alkenyl, —CO-aryl, —CO₂-aryl, —CONH-aryl, —CO-heterocyclic ring, —CO₂-heterocyclic ring, —COHN-heterocyclic ring (the alkyl, alkenyl, aryl, heterocyclic are optionally substituted by halogen atom, alkyl group, hydroxyl group, alkoxy group, carboxyl group, amino group, amino acid residue, —PO₃H₂, —SO₃H, —OPO₃H₂, —OSO₃H, and the like.), or —CO-polyethylene glycol residue, —CO₂-polyethylene glycol residue, —CO-polyethylene glycol mono alkyl ether residue, —CO₂-polyethylene glycol mono alkyl ether residue, —PO₃H₂, saccharides (e.g., glucose), or other known macromolecule for a prodrug and the like;
those wherein an amino group of the compound is substituted by —CO-alkyl, —CO₂-alkyl, —CO-alkenyl, —CO₂-alkenyl, —CO₂-aryl, —CO-aryl, —CO-heterocyclic ring, —CO₂-heterocyclic ring (the alkyl, alkenyl, aryl, heterocyclic ring are optionally substituted by halogen atom, alkyl group, hydroxyl group, alkoxy group, carboxyl group, amino group, amino acid residue, —PO₃H₂, —SO₃H, —OPO₃H₂, —OSO₃H, and the like.), or —CO-polyethylene glycol residue, —CO₂-polyethylene glycol residue, —CO-polyethylene glycol mono alkyl ether residue, —CO₂-polyethylene glycol mono alkyl ether residue, —PO₃H₂, saccharides (e.g., glucose), or other known macromolecule for a prodrug and the like; and
those wherein a carboxyl group of the compound is substituted by alkoxy group, aryloxy group (the alkoxy group, aryloxy group are optionally substituted by halogen atom, alkyl group, hydroxyl group, alkoxy group, carboxyl group, amino group, amino acid residue, —PO₃H₂, —SO₃H, —OPO₃H₂, —OSO₃H, and the like.), or polyethylene glycol residue, polyethylene glycol mono alkyl ether residue, saccharides (e.g., glucose), or other known macromolecule for a prodrug and the like are mentioned as examples of embodiments of the present invention.

These prodrugs can be produced, for example, according to a method known per se by one of skill in the pertinent field, such as esterification, acylation, alkoxycarbonylation, and the like.

When the inventive compound is used as a pharmaceutical preparation, the inventive compound is generally admixed with pharmaceutically acceptable carriers, excipients, diluents, fillers, disintegrators, stabilizers, preservatives, buffers, emulsifiers, aromatics, coloring agents, sweeteners, thickeners, correctives, solubilizers, and other additives such as water, vegetable oil, alcohol such as ethanol, benzyl alcohol and the like, polyethylene glycol, glycerol triacetate, gelatin, lactose, carbohydrate such as starch and the like, magnesium stearate, talc, lanolin, petrolatum and the like, and prepared into a dosage form, for example, of tablets, pills, powders, granules, suppositories, injections, eye drops, liquids, capsules, troches, aerosols, elixirs, suspensions, emulsions, syrups and the like, which can be administered systemically or topically and orally or parenterally.

While the dose of the inventive compound varies depending on the age, body weight, general condition, treatment effect, administration route and the like, it is generally from 1 mg to 1000 mg for an adult per dose, which is given one to several times a day.

The inventive compound (1) can be administered to mammals (human, mouse, rat, rabbit, dog, cat, cattle, pig, monkey, etc.) as an aggrecanase inhibitor, an MMP inhibitor, a prophylactic or therapeutic agent for osteoarthritis (OA), a prophylactic or therapeutic agent for rheumatoid arthritis (RA), a prophylactic or therapeutic agent for a disorder mediated by aggrecanase, such as joint injury, reactive arthritis, cancer, asthma, allergic reaction, chronic pulmonary emphysema, fibroid lung, acute respiratory distress (ARDS), lung infection, interstitial pneumonia, bone resorption disorder, and the like.

The compound (1) of the present invention can be administered to mammals along with other therapeutic agents for osteoarthritis, for the purpose of prevention or treatment of osteoarthritis. The compound (1) of the present invention can be also administered to mammals along with other therapeutic agents for arthritis rheumatoides, for the purpose of prevention or treatment of arthritis rheumatoides.

“Prevention” include, for example, both preventing recurrence of the disease and preventing initial occurrence of the disease.

In the case of combined administration, the compound of the present invention can be administered simultaneously with other therapeutic agents for osteoarthritis or other therapeutic agents for rheumatoid arthritis (hereinafter combination drug) or administered at certain time intervals. In the case of combined administration, a pharmaceutical composition containing the compound of the present invention and a combination drug can be administered. Alternatively, a pharmaceutical composition containing the compound of the present invention and a pharmaceutical composition containing a combination drug may be administered separately. The administration route may be the same or different.

In the case of a combined administration, the compound of the present invention can be administered once a day or several times a day in a single dose of 1 mg to 1000 mg, or may be administered in a smaller dose. The combination drug can be administered in a dose generally used for the prevention or treatment of osteoarthritis or rheumatoid arthritis or in a smaller dose.

In addition, a compound having aggrecanase inhibitory activity or MMP inhibitory activity as does the compound (1) of the present invention, a prodrug thereof and a pharmaceutically acceptable salt thereof can be used as prophylactic or therapeutic agents for diseases mediated by aggrecanase, such as osteoarthritis, arthritis rheumatoides, and the like.

Examples of the production method of the compound (1) of the present invention are given in the following. However, the production method of the compound of the present invention is not limited to these examples.

It is also possible to previously protect, as necessary, the functional groups other than those involved in the reactions to be mentioned below, and to deprotect them at a later stage.

The treatment after reaction in each step may be a conventional one, for which typical methods, such as isolation and purification, crystallization, recrystallization, column chromatography, preparative HPLC and the like, can be appropriately selected and combined.

The compound (2), which is a starting material in the following production methods, is commercially available or can be easily synthesized by a method known per se by one of skill in the art.

Production Method 1

This production method is a production method for compound (1) wherein R⁵ is a caboxyl group or a hydroxyaminocarbonyl group. 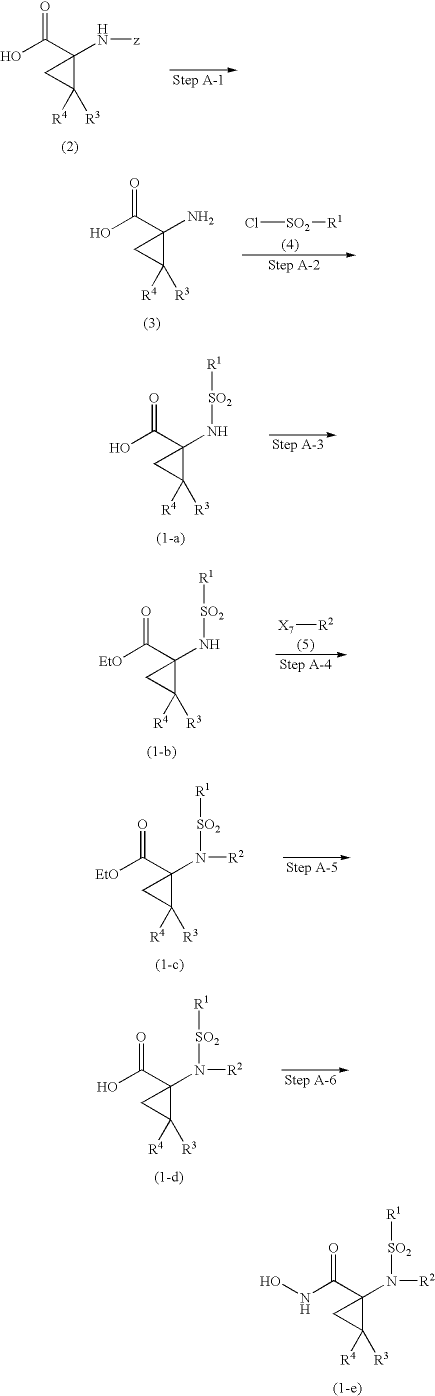
wherein R¹ R² R³ and R⁴ are as defined above, Z is a protective group of amino (e.g., benzyloxycarbonyl, tert-butoxycarbonyl, etc.) and X₇ is halogen atom.
Step A-1

General deprotection is performed. A compound of the formula (2) is reacted in the presence of an acid in a solvent to give a compound of the formula (3).

As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, dichloroethane, etc.; ester solvents such as ethyl acetate, methyl acetate, butyl acetate, etc.; polar solvents such as acetone, N,N-dimethylformamide, etc.; etc. can be mentioned, which may be used alone or in combination. A preferable solvent in this reaction is dioxane.

As the acid to be used for the reaction is, for example, inorganic acids such as hydrochloric acid, sulfuric acid, nitric acid, etc.; and organic acids such as trifluoroacetic acid, trichloroacetic acid, acetic acid, methanesulfonic acid, p-toluenesulfonic acid, etc. can be mentioned, with preference given to hydrochloric acid.

The reaction temperature is generally −30° C. to 60° C., preferably 0° C. to room temperature.

The reaction time is generally 1 hr to 24 hr, preferably 2 hrs to 12 hrs.

Thus obtained compound (3) can be used in the next reaction without isolation.

Step A-2

General sulfonylation is performed. A compound of the formula (3) is reacted with a compound of the formula (4) in a solvent in the presence of a base to give a compound of the formula (1-a), which is one of the objective compounds.

As the base to be used for the reaction is, for example, alkali metal hydrides such as sodium hydride, potassium hydride, etc.; alkali metal carbonate such as sodium carbonate, potassium carbonate, cesium carbonate, sodium hydrogen carbonate, potassium hydrogen carbonate, etc.; alkali metal carboxylate such as sodium acetate, potassium acetate, etc.; alkali metal phosphate such as sodium phosphate, potassium phosphate, etc.; organic base such as triethylamine, diisopropylethylamine, pyridine, N-methylmorpholine, N,N-dimethylaminopyridine, etc. can be mentioned, with preference given to triethylamine and N,N-dimethylaminopyridine.

As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, dichloroethane, etc.; ester solvents such as ethyl acetate, methyl acetate, butyl acetate, etc.; polar solvents such as acetone, N,N-dimethylformamide, water, etc.; etc. can be mentioned, which may be used alone or in combination. A preferable solvent in this reaction is a mixed solvent of dioxane and water.

The reaction temperature is generally −30° C. to 60° C., preferably 0° C. to room temperature.

The reaction time is generally 2 hrs to 24 hr, preferably 4 hrs to 12 hrs.

Step A-3

General esterification is performed. A compound of the formula (1-a) is reacted with an activator for carboxylic acid or an acid catalyst in a solvent to give a compound of the formula (1-b).

As the solvent, for example, ether solvents such as diethyl ether, tetrahydrofuran, dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, dichloroethane, etc.; alcohol solvents such as methanol, ethanol, isopropanol, tert-butanol, etc.; and ester solvents, etc. such as ethyl acetate, methyl acetate, butyl acetate, etc. can be mentioned, which may be used alone or in combination. A preferable solvent in this reaction is ethanol.

As the activator for carboxylic acid, for example, thionyl chloride, etc. can be mentioned.

As the acid catalyst, sulfuric acid, p-toluenesulfonic acid, etc. can be mentioned.

The reaction temperature is generally 80° C. to 150° C., preferably 100° C. to 120° C.

The reaction time is generally 10 hrs to 48 hr, preferably 12 hrs to 24 hrs.

The compound (1-b) obtained in this reaction can be used for the next reaction without isolation.

Step A-4

General alkylation is performed. A compound of the formula (1-b) is reacted with a compound of the formula (5) in the presence of a base in a solvent to give one of the objective compounds of the formula (1-c).

As the solvent to be used for this reaction, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, dichloroethane, etc.; alcohol solvents such as methanol, ethanol, isopropanol, tert-butanol, etc.; ester solvents such as ethyl acetate, methyl acetate, butyl acetate, etc.; and polar solvents such as acetone, N,N-dimethylformamide, dimethyl sulfoxide, etc.; etc. can be mentioned, which may be used alone or in combination. A preferable solvent in this reaction is N,N-dimethylformamide.

As the base, for example, alkali metal hydrides such as sodium hydride, potassium hydride, etc.; alkali metal alkoxides such as sodium ethoxide, sodium methoxide, potassium t-butoxide, etc.; alkylithiums such as n-butylithium, sec-butylithium, etc.; alkali metal amides such as lithium diisopropylamide, sodium amide, lithium bistrimethylsilylamide, etc.; alkali metal carbonates such as sodium carbonate, potassium carbonate, sodium hydrogen carbonate, potassium hydrogen carbonate, etc.; alkali metal hydroxides such as lithium hydroxide, sodium hydroxide, potassium hydroxide, etc.; alkali metal phosphates such as sodium phosphate, potassium phosphate, etc.; and organic bases such as triethylamine, pyridine, N-methylmorpholine, etc. can be mentioned, with preference given to potassium carbonate.

The reaction temperature is generally 0° C. to 90° C., preferably 80° C.

The reaction time is generally 1 hrs to 24 hr. preferably 2 hrs to 12 hrs.

Step A-5

General hydrolysis is performed. A compound of the formula (1-c) is reacted in the presence of a base in a solvent to give a compound of the formula (1-d), which is one of the objective compounds.

As the base to be used for the reaction, for example, alkali metal hydrides such as sodium hydride, potassium hydride, etc.; alkali metal alkoxides such as potassium tert-butoxide, etc.; alkali metal amides such as lithium diisopropylamide, etc.; alkali metal carbonates such as sodium carbonate, potassium carbonate, cesium carbonate, sodium hydrogen carbonate, potassium hydrogen carbonate, etc.; alkali metal hydroxides such as lithium hydroxide, sodium hydroxide, potassium hydroxide, etc.; and the like can be mentioned, with preference given to sodium hydroxide.

As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, dichloroethane, etc.; alcohol solvents such as methanol, ethanol, isopropanol, tert-butanol, etc.; and polar solvents such as water, etc. can be mentioned, which may be used alone or in combination. Preferable solvents in this reaction are tetrahydrofuran and methanol.

The reaction temperature is generally 0° C. to 60° C., preferably room temperature.

The reaction time is generally 1 hr to 24 hr, preferably 2 hrs to 12 hrs.

Step A-6

General amidation is performed. A compound of the formula (I-d) is reacted with a hydroxylamine derivative using a condensing agent in a solvent in the presence of a base to give a compound of the formula (I-e), which is one of the objective compounds.

As the base to be used for the reaction, for example, alkali metal carbonates such as sodium carbonate, potassium carbonate, cesium carbonate, sodium hydrogen carbonate, potassium hydrogen carbonate, etc.; alkali metal carboxylates such as sodium acetate, potassium acetate, etc.; alkali metal phosphates such as sodium phosphate, potassium phosphate, etc., and organic bases such as triethylamine, diisopropylethylamine, pyridine, N-methylmorpholine, etc. can be mentioned, with preference given to N-methylmorpholine.

As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, dichloroethane, etc.; ester solvents such as ethyl acetate, methyl acetate, butyl acetate, etc.; and polar solvents such as acetone, N,N-dimethylformamide, acetonitrile, etc.; etc. can be mentioned, which may be used alone or in combination. Preferable solvents in this reaction are tetrahydrofuran and N,N-dimethylformamide.

As the condensing agent, any condensing agent used for general peptide condensation method (e.g., acyl chloride method, mixed acid anhydride method, etc.) can be used, with preference given to a combination of ethyl chlorocarbonate and N-methylmorpholine.

As the hydroxylamine derivative to be used for the reaction, for example, O-(trimethylsilyl)hydroxylamine, etc. can be mentioned.

The reaction temperature is generally 0° C. to 100° C., preferably room temperature to 60° C.

The reaction time is generally 1 hr to 24 hr, preferably 2 hrs to 12 hrs.

The compound 13, 29 or 34, which is a synthetic intermediate or starting material for the following production method 2, is commercially available or easily synthesized by a conventionally known method, such as a method introduced in the general theory of Stammer et al. and the like (Tetrahedron 1990, 46, 2231; Tetrahedron 1989, 45, 6091; U.S. Pat. No. 3,313,842). Furthermore, examples of the production method of compound 13 are shown in steps 1-1 to 1-3 and 2-1 to 2-6.

[Production Method 2]

This production method is a production method of compound (1) wherein R⁵ is a carboxyl group. 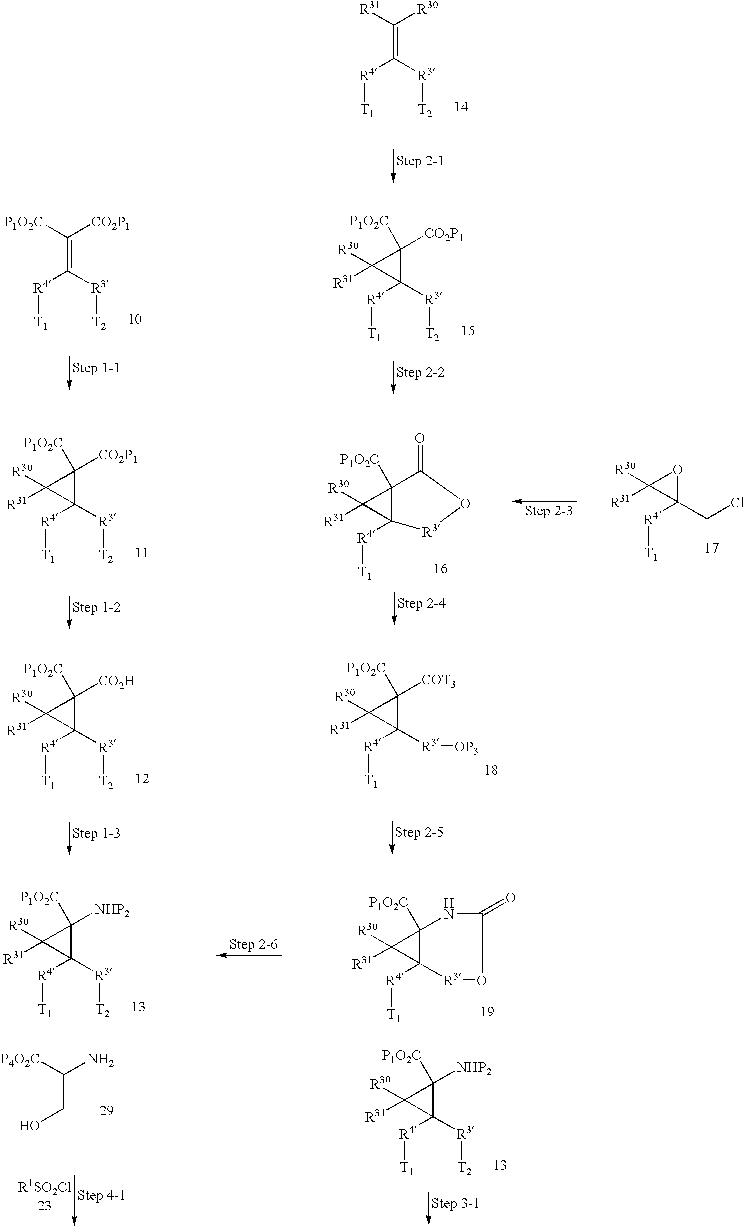 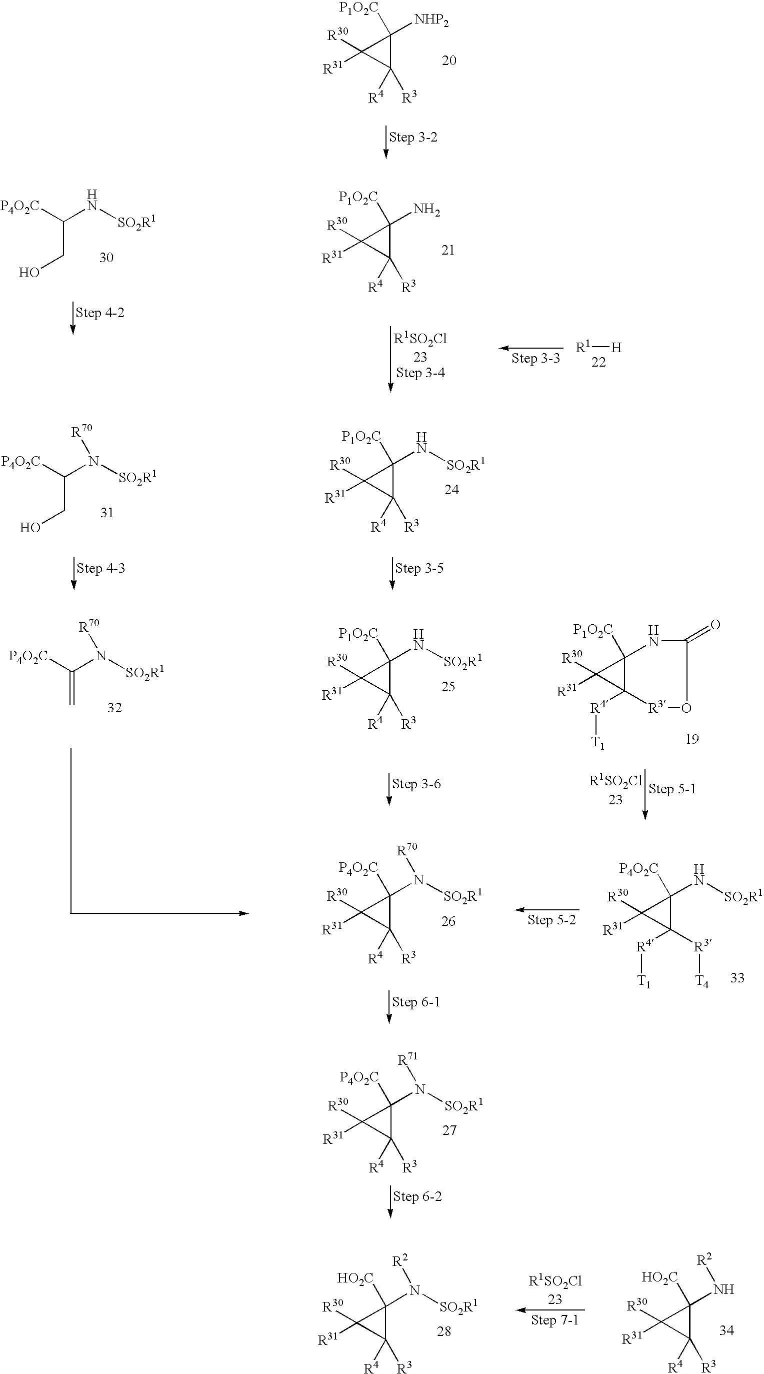
wherein R¹, R², R³, R⁴, R³⁰ and R³¹ are as defined above;
as R³′, the same substituents as for R³ can be mentioned;
as R⁴′, the same substituents as for R⁴ can be mentioned;
as R⁷⁰ and R⁷¹, the same substituents as for R² can be mentioned;
T₁, T₂, T₃ and T₄ are substituents used for later conversion of the functional group and, for example, a hydrogen atom, an alkyl group, a halogen atom, a haloalkyl group, an amino group, a hydroxyl group, a formyl group, an alkylcarbonyl group, an alkylboranyl group, an alkoxyboranyl group, a hydroxyboranyl group, a methylthio group, a benzenesulfonyloxy group, a p-toluenesulfonyloxy group, methanesulfonyloxy group, a trifluoromethanesulfonyloxy group, a nitro group, a cyano group, an alkoxycarbonyl group, an amide group, an azide group, an alkoxy group, a carboxyl group and the like can be mentioned, wherein, T₁ and T₂ remain in the molecules R⁴ and R³, respectively, in the compound in the claims when the conversion of the functional group is not necessary;
P₁ and P₄ are general carboxyl-protecting groups, and as the protecting group, for example, a methyl group, an ethyl group, a t-butyl group, a benzyl group, a p-methoxybenzyl group, an allyl group, a t-butyldimethylsilyl group and the like can be mentioned, wherein, depending on the step, P₁ may be a hydrogen atom;
P₂ is a general amino-protecting group, and as the protecting group, for example, a t-butoxycarbonyl group, a benzyloxycarbonyl group, a fluorenylmethyloxycarbonyl group and the like can be mentioned;
P₃ is a general hydroxyl-protecting group, and as the protecting group, for example, ethers such as a tetrahydropyranyl group, a benzyl group, a methoxymethyl group, a benzyloxymethyl group, a trimethylsilylethyloxymethyl group and the like, esters such as a pivaloyl group, an acetyl group, a benzoyl group and the like, silyl ether-protecting groups such as a trimethylsilyl group, a t-butyldimethylsilyl group, a t-butyldiphenylsilyl group and the like, and the like can be mentioned, wherein, depending on the step, P₃ may be a hydrogen atom.
   Step 1-1

In this Step, the alkylidenemalonic acid diester of the formula 10 is reacted with sulfonium methilide based on the method known in literature (J. Med. Chem. 1992, 35, 1410-1417) to give a compound of the formula 11. Sulfonium methilide is produced by treating trimethylsulfoxonium or trimethylsulfonium halide with a base.

As the base, for example, alkyl lithiums such as butyl lithium, t-butyl lithium, s-butyl lithium etc.; alkali metal hydrides such as sodium hydride, potassium hydride etc.; metal alcoholates such as potassium t-butoxide, sodium ethoxide, sodium methoxide etc.; alkali metal amides such as lithium diisopropyl amide, sodium bis(trimethylsilyl)amide, lithium bis(trimethylsilyl)amide etc. and the like can be mentioned, A preferable base is alkali metal hydride, and sodium hydride is more preferabe. As the solvent, for example, ether solvents such as diethyl ether, tetrahydrofuran (THF), dioxane, 1,2-dimethoxyethane, diglyme etc.; hydrocarbon solvents such as benzene, toluene, hexane, xylene etc.; polar solvents such as N,N-dimethylformamide, dimethyl sulfoxide etc. and the like can be mentioned, which may be used alone or in combination. A preferable solvent in this reaction is a polar solvent and dimethyl sulfoxide is more preferable. The reaction temperature is generally −78° C. to 100° C., preferably 0° C. to 60° C. The reaction time is 30 min to 48 hr, preferably 1 hr to 12 hrs.

Thus obtained compound of the formula 1 can be used in the next reaction without isolation.

Step 1-2

In this Step, one of the esters of cyclopropane dicarboxylic acid diester of the formula 11 and obtained in Step 1-1 is selectively hydrolyzed to give a monoester of the formula 12. While the selectivity varies depending on R⁴′, R³′, R³⁰, R³¹, T₁ and T₂, one of the two esters of less hindered or of being assisted by neighboring functional groups is preferentially hydrolyzed. While the hydrolysis conditions vary depending on the kind of P₁, when, for example, P₁ is a methyl group, the base includes, for example, alkali metal carbonates such as sodium carbonate, potassium carbonate, etc.; alkali metal hydroxides such as lithium hydroxide, sodium hydroxide, potassium hydroxide etc. and the like with preference given to sodium hydroxide. As the solvent, for example, ether solvents such as diethyl ether, tetrahydrofuran (THF), dioxane, 1,2-dimethoxyethane, diglyme etc.; alcohol solvents such as methanol, ethanol, isopropyl alcohol, t-butyl alcohol etc.; polar solvents such as water etc. and the like can be mentioned, which may be used alone or in combination. A preferable solvent in this reaction is an alcohol solvent and a mixed solvent of ethanol or methanol and water is more preferable. The reaction temperature is generally 0° C. to 100° C., preferably 0° C. to room temperature. The reaction time is 1 hr to 48 hr, preferably 6 hrs to 24 hrs.

Thus obtained compound of the formula 12 can be used in the next reaction without isolation.

Step 1-3

In this Step, the dicarboxylic acid monoester of the formula 12 and obtained in Step 1-2 is led to a compound of the formula 13. In this Curtius rearrangement reaction, carboxylic acid azide obtained by converting compound 12 to an activated ester by a conventional method and then reacting the ester with metal azide may be used as a starting material. However, compound 13 can also be obtained from compound 12 via carboxylic acid azide by the use of diphenylphosphoryl azide in the presence of a base. In this case, as the base, organic bases such as triethylamine, pyridine, N-methylmorpholine, 2,6-lutidine, 1,8-diazabicyclo[5.4.0]7 -undecene etc. and the like can be mentioned, with preference given to triethylamine or diisopropylethylamine. As the solvent, for example, ether solvents such as diethyl ether, tetrahydrofuran (THF), dioxane, 1,2-dimethoxyethane, diglyme etc.; hydrocarbon solvents such as benzene, toluene, hexane, xylene etc., alcohol solvents such as benzyl alcohol, fluorenylmethyl alcohol, t-butyl alcohol etc.; polar solvents such as N,N-dimethylformamide, dimethyl sulfoxide etc. and the like can be mentioned, which may be used alone or in combination. The solvent is appropriately chosen depending on P₂. For example, when P₂ is t-butoxycarbonyl, t-butyl alcohol is used. The reaction temperature is generally 0° C. to 150° C., preferably room temperature to 120° C. The reaction time is 1 hr to 96 hr, preferably 6 hrs to 48 hrs.

Thus obtained compound of the formula 13 can be used in the next reaction without isolation.

Step 2-1

In this Step, the alkene of the formula 14 is led to a cyclopropane derivative of the formula 15 by the method known in literature (Synlett 2001, 12, 1843-1846) or a method using diazomalonic acid diester derived from malonic acid diester by a conventional method and a catalyst. In the formula of this Step, T₂ is a protected hydroxyl group. For example, when diazomalonic acid diester is used, the catalyst is preferably rhodium complex, copper complex etc., and rhodium (II) acetate dimer is more preferable. As the malonic acid diester, diethyl malonate, dimethyl malonate, dibenzyl malonate, di-t-butyl malonate etc. can be mentioned, with preference given to dimethyl malonate.

As the solvent, for example, ether solvents such as diethyl ether, tetrahydrofuran (THF), dioxane, 1,2-dimethoxyethane, diglyme etc.; hydrocarbon solvents such as benzene, toluene, hexane, xylene etc.; halogenated solvents such as dichloromethane, chloroform, carbon tetrachloride, 1,2-dichloroethane etc.; ester solvents such as ethyl acetate, methyl acetate, butyl acetate etc.; polar solvents such as acetone, N,N-dimethylformamide, dimethyl sulfoxide, acetonitrile etc. and the like can be mentioned, which may be used alone or in combination. A preferable solvent in this reaction is a hydrocarbon solvent, and no solvent is more preferable. The reaction temperature is generally room temperature to 150° C., preferably 50° C. to 120° C. The reaction time is 1 min to 48 hr, preferably 10 min to 3 hrs.

Thus obtained compound of the formula 15 can be used in the next reaction without isolation.

Step 2-2

In this Step, the protecting group of the substituent T₂ (protected hydroxyl group) of the compound of the formula 15 obtained in Step 2-1 is deprotected to give a lactone of the formula 16. While the reaction conditions are appropriately chosen depending on the kind of the protecting group in T₂, when, for example, the protecting group is a t-butyldiphenylsilyl group, deprotection is possible with an acid or a fluoride source. As the acid, hydrochloric acid, sulfuric acid, phosphoric acid, acetic acid, trifluoroacetic acid, methanesulfonic acid, trifluoromethanesulfonic acid, etc. can be mentioned, with preference given to trifluoroacetic acid. As the fluoride source, hydrogen fluoride, hydrogen fluoride-pyridine, tetrabutylammonium fluoride, potassium fluoride, cesium fluoride, etc. can be mentioned, with preference given to tetrabutylammonium fluoride.

As the solvent, for example, ether solvents such as diethyl ether, tetrahydrofuran (THF), dioxane, 1,2-dimethoxyethane, diglyme etc.; hydrocarbon solvents such as benzene, toluene, hexane, xylene etc.; halogenated solvents such as dichloromethane, chloroform, carbon tetrachloride, 1,2-dichloroethane etc.; alcohol solvents such as methanol, ethanol, isopropyl alcohol, t-butyl alcohol etc.; ester solvents such as ethyl acetate, methyl acetate, butyl acetate etc.; polar solvents such as acetone, N,N-dimethylformamide, dimethyl sulfoxide, acetonitrile, water etc. and the like can be mentioned, which may be used alone or in combination. A preferable solvent in this reaction is an ether solvent, and THF is more preferable. The reaction temperature is generally 0° C. to 100° C., preferably room temperature to 50° C. The reaction time is 1 hr to 48 hr, preferably 1 hr to 12 hrs. Thus obtained compound of the formula 16 can be used in the next reaction without isolation.

Step 2-3

In this Step, the epichlorohydrin derivative of the formula 17 is reacted with malonic acid diester to give a lactone derivative condensed with the cyclopropane of the formula 16. R³′ of the compound of the formula 16 obtained by this Step is methylene. The reaction is carried out in the presence of a base. The malonic acid diester is appropriately chosen depending on P₁, and dimethyl malonate, diethyl malonate, di-t-butyl malonate, dibenzyl malonate, etc. can be mentioned, with preference given to di-t-butyl malonate. As the base, for example, alkyl lithiums such as butyl lithium, t-butyl lithium, s-butyl lithium etc.; alkali metal hydrides such as sodium hydride, potassium hydride etc.; metal alcoholates such as potassium t-butoxide, sodium ethoxide, sodium methoxide, etc.; alkali metal amides such as lithium diisopropyl amide, sodium bis(trimethylsilyl)amide, lithium bis(trimethylsilyl)amide, etc.; alkali metal carbonates such as sodium carbonate, potassium carbonate, sodium hydrogen carbonate, potassium hydrogen carbonate, etc. and the like can be mentioned, with preference given to potassiumt-butoxide. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; alcohol solvents such as methanol, ethanol, isopropyl alcohol, t-butyl alcohol, etc.; polar solvents such as acetone, N,N-dimethylformamide, dimethyl sulfoxide, acetonitrile, etc. and the like can be mentioned, which may be used alone or in combination. A preferable solvent in this reaction is a mixed solvent of t-butyl alcohol and THF. The reaction temperature is generally 0° C. to 150° C., preferably room temperature to 80° C. The reaction time is 1 hr to 48 hr, preferably 6 hrs to 24 hrs. Thus obtained compound of the formula 16 can be used in the next reaction without isolation.

Where necessary, deprotection of carboxylic-protecting group, optical resolution and protection of carboxylic acid may be performed in this Step.

For example, the ester of the formula 16 is led to a carboxylic acid derivative by a conventional method. While the reaction conditions are appropriately chosen depending on P₁, when, for example, P₁ is a methyl group or an ethyl group, conventional hydrolysis with a base is performed. When, for example, P₁ is a t-butyl group, deprotection with an acid is performed.

As the base, for example, alkali metal carbonates such as cesium carbonate, sodium carbonate, potassium carbonate etc.; alkali metal hydroxides such as lithium hydroxide, sodium hydroxide, potassium hydroxide, etc. and the like can be mentioned, with preference given to alkali metal hydroxide.

As the acid to be used for deprotection under acidic conditions, mineral acids such as hydrochloric acid, sulfuric acid, phosphoric acid, nitric acid, etc.; organic acids such as trifluoroacetic acid, methanesulfonic acid, p-toluenesulfonic acid, trifluoromethanesulfonic acid, etc. and the like can be mentioned, with preference given to hydrochloric acid or trifluoroacetic acid. As the solvent for hydrolysis with a base, for example, ether solvents such as diethyl ether, tetrahydrofuran (THF), dioxane, 1,2-dimethoxyethane, diglyme etc.; alcohol solvents such as methanol, ethanol, isopropyl alcohol, t-butyl alcohol etc.; polar solvents such as water etc. and the like can be mentioned, which may be used alone or in combination. A preferable solvent in this reaction is a mixed solvent of an ether solvent and an alcohol solvent, more preferably a mixed solvent of methanol, THF and water. The reaction temperature is generally room temperature to 100° C., preferably room temperature to 80° C. The reaction time is 1 hr to 48 hr, preferably 2 hrs to 24 hrs. In the case of deprotection with an acid, as the solvent, for example, ether solvents such as diethyl ether, tetrahydrofuran (THF), dioxane, 1,2-dimethoxyethane, diglyme etc.; hydrocarbon solvents such as benzene, toluene, hexane, xylene etc.; halogenated solvents such as dichloromethane, chloroform, carbon tetrachloride, 1,2-dichloroethane etc.; ester solvents such as ethyl acetate, methyl acetate, butyl acetate etc.; polar solvents such as acetone, N,N-dimethylformamide, acetonitrile, water, etc. and the like can be mentioned, with preference given to ethyl acetate, dioxane, dichloromethane, chloroform or no solvent.

In addition, thus obtained racemic carboxylic acid is led to a diastereomic salt of a chiral amine and recrystallized. As the chiral amine, alkaloids such as cinchonine, quinidine, cinchonidine, quinine, brucine, strychnine, etc.; amino acids or alcohols derived from amino acids such as alanine, phenylalanine, alaminol, phenylalaminol, etc.; phenethylamine, naphthylethylamine, etc. and the like can be mentioned, with preference given to quinidine or cinchonidine. As the solvent used for recrystallization, for example, ether solvents such as diethyl ether, tetrahydrofuran (THF), dioxane, 1,2-dimethoxyethane, etc.; hydrocarbon solvents such as benzene, toluene, etc.; alcohol solvents such as methanol, ethanol, isopropyl alcohol, t-butyl alcohol, etc.; ester solvents such as ethyl acetate, methyl acetate, butyl acetate, etc.; polar solvents such as acetone, 2-butanone, acetonitrile, water, etc. and the like can be mentioned, which may be used alone or in combination. A preferable solvents in this recrystallization are isopropyl alcohol, acetone, ethyl acetate, and a mixed solvent thereof.

Thus obtained chiral acid is subjected to esterification again to give an chiral carboxylic acid of the compound 16. For protection of a carboxylic acid derivative with P1, which is a protecting group, by a conventional method, P₁ is appropriately chosen depending on T₁. When, for example, P₁ is a t-butyl group, a method using isobutene in the presence of an acid catalyst to give t-butyl ester, and a method using N,N-dimethylformamide di-t-butylacetal can be mentioned.

When, for example, N,N-dimethylformamide di-t-butylacetal is used, as the solvent, for example, ether solvents such as diethyl ether, tetrahydrofuran (THF), dioxane, 1,2-dimethoxyethane, diglyme etc.; hydrocarbon solvents such as benzene, toluene, hexane, xylene etc.; halogenated solvents such as dichloromethane, chloroform, carbon tetrachloride, 1,2-dichloroethane, etc., ester solvents such as ethyl acetate, methyl acetate, butyl acetate etc.; polar solvents such as acetone, N,N-dimethylformamide, dimethyl sulfoxide, acetonitrile, etc. and the like can be mentioned, which may be used alone or in combination. A preferable solvent in this reaction is a hydrocarbon solvent, and toluene is more preferable. The reaction temperature is generally room temperature to 150° C., preferably room temperature to 110° C. The reaction time is 1 hr to 24 hr, preferably 2 hrs to 12 hrs. Thus obtained compound of the formula 16 can be used in the next reaction without isolation.

Step 2-4

In this Step, the lactone of the formula 16 and obtained in Step 2-2 or 2-3 is subjected to ring opening, and a hydroxyl group is protected as necessary. The reaction conditions are appropriately chosen depending on the kind of R³′, P₃ and T₃. For example, when P₃ is a t-butyldimethylsilyl group and T₃ is OH, this Step comprises three reactions including hydrolysis of compound 16 with alkali metal carbonate or alkali metal hydroxide to give a carboxylic acid alkali metal salt, and subsequent protection of newly formed hydroxyl group and carboxyl group with t-butyldimethylsilyl chloride, and selective hydrolysis of carboxylic acid silyl ester with a base. As the alkali metal carbonates used in the hydrolysis of lactone, potassium carbonate, sodium carbonate and the like can be mentioned, and as the alkali metal hydroxides, sodium hydroxide, potassium hydroxide and the like can be mentioned, with preference given to sodium hydroxide.

As the solvent used in the hydrolysis, for example, ether solvents such as diethyl ether, tetrahydrofuran (THF), dioxane, 1,2-dimethoxyethane, diglyme, etc.; alcohol solvents such as methanol, ethanol, isopropyl alcohol, t-butyl alcohol etc.; polar solvents such as water etc. and the like can be mentioned, which may be used alone or in combination. A preferable solvent in this reaction is ether solvent, and a mixed solvent of THF and water is more preferable. The reaction temperature is generally 0° C. to 100° C., preferably room temperature to 80° C. The reaction time is 1 hr to 48 hr, preferably 1 hr to 12 hrs.

The subsequent protection of the newly formed hydroxyl group and carboxyl group with t-butyldimethylsilyl group is performed in the presence of a base. As the base, for example, organic bases such as triethylamine, pyridine, N-methylmorpholine, imidazole, etc. and the like can be mentioned, with preference given to imidazole. As the solvent, for example, ether solvents such as diethyl ether, tetrahydrofuran (THF), dioxane, 1,2-dimethoxyethane, diglyme etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, dimethyl sulfoxide, acetonitrile, etc. and the like can be mentioned, which may be used alone or in combination. A preferable solvent in this reaction is a polar solvent, and N,N-dimethylformamide is more preferable.

The hydrolysis of the carboxylic acid silyl ester can be performed in one-pot with the above-mentioned reaction. That is, after the completion of the above-mentioned reaction, water, an alcohol solvent and a base are added to the reaction, whereby carboxylic acid silyl ester can be selectively hydrolyzed. As the alcohol solvent, methanol is preferably used. As the base, alkali metal carbonates such as sodium carbonate, potassium carbonate, sodium hydrogen carbonate, potassium hydrogen carbonate, etc.; alkali metal hydroxides such as lithium hydroxide, sodium hydroxide, potassium hydroxide, etc. and the like can be mentioned, with preference given to alkali metal carbonate, and potassium carbonate is more preferable. The reaction temperature is generally 0° C. to 100° C., preferably 0° C. to 50° C. The reaction time is 1 hr to 48 hr, preferably 1 hr to 12 hrs. Thus obtained compound of the formula 18 can be used in the next reaction without isolation.

A compound of the formula 18 wherein T₃ is an NH₂ group and P₃ is a hydrogen can be obtained by, for example, treating the lactone of the formula 16 and obtained in Step 2-4 with ammonia.

As the solvent, for example, ether solvents such as diethyl ether, tetrahydrofuran (THF), dioxane, 1,2-dimethoxyethane, diglyme, etc.; alcohol solvents such as methanol, ethanol, isopropyl alcohol, t-butyl alcohol, etc.; polar solvents such as N,N-dimethylformamide, dimethyl sulfoxide, acetonitrile, water, etc. and the like can be mentioned, which may be used alone or in combination. A preferable solvent in this reaction is a mixed solvent of methanol, THF and water. The reaction temperature is generally 0° C. to 100° C., preferably 0° C. to 50° C. The reaction time is 1 hr to 48 hr, preferably 6 hrs to 24 hrs. Thus obtained compound of the formula 18 can be used in the next reaction without isolation.

Step 2-5

In this Step, the compound of the formula 18 obtained in Step 2-4 is led to a cyclic urethane of the formula 19. When, for example, T₃ is OH and P₃ is a trialkylsilyl-protecting group, compound 19 can be obtained by Curtius rearrangement reaction and subsequent deprotection of a trialkylsilyl protecting group. That is, compound 19 is treated with diphenylphosphoryl azide in the presence of a base to give a isocyanate, which is then led to compound 19 by addition of a fluoride source to the reaction to deprotect the silyl-protecting group. As the base, organic bases such as triethylamine, pyridine, N-methylmorpholine, 2,6-lutidine, 1,8-diazabicyclo[5.4.0]7-undecene, etc. and the like can be mentioned, with preference given to triethylamine. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, acetonitrile, water, etc. and the like can be mentioned, which may be used alone or in combination. A preferable solvent in this reaction is a polar solvent, and N,N-dimethylformamide is more preferable. The reaction temperature is generally room temperature to 150° C., preferably room temperature to 80° C. The reaction time is 10 min to 48 hr, preferably 10 min to 6 hrs. As the fluoride source to be added after the completion of the Curtius rearrangement reaction, hydrogen fluoride, hydrogen fluoridepyridinecomplex, tetrabutylammonium fluoride, potassium fluoride, cesium fluoride, and the like can be mentioned, with preference given to cesium fluoride. The reaction temperature after addition of the fluoride source is generally 0° C. to 100° C., preferably room temperature to 80° C. The reaction time is 1 hr to 48 hr, preferably 1 hr to 6 hrs. Thus obtained compound of the formula 19 can be used in the next reaction without isolation.

In addition, for example, a Hoffman rearrangement reaction can be used for the compound of the formula 18, wherein T₃ is NH₂ and P₃ is a hydrogen atom. As the oxidizing reagent to be used for the Hoffman rearrangement, N-bromosuccinimide, N-chlorosuccinimide, sulfuryl chloride, bromine, iodobenzene diacetate, and the like can be mentioned, with preference given to iodobenzene diacetate. The reaction may be carried out in the presence of a base, and as the base, alkali metal carbonates such as sodium carbonate, potassium carbonate, sodium hydrogen carbonate, potassium hydrogen carbonate, etc.; alkali metal hydroxides such as lithium hydroxide, sodium hydroxide, potassium hydroxide, etc. and the like can be mentioned, with preference given to sodium hydroxide.

As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, acetonitrile, water, etc. and the like can be mentioned, which may be used alone or in combination. A preferable solvent in this reaction is a mixed solvent of acetonitrile, ethyl acetate and water. The reaction temperature is generally −20° C. to 100° C., preferably 0° C. to room temperature. The reaction time is 1 hr to 48 hrs, preferably 1 hr to 12 hrs. Thus obtained compound of the formula 19 can be used in the next reaction without isolation.

Step 2-6

In this Step, the cyclic urethane of the formula 19 obtained in Step 2-5 is subjected to ring opening reaction to give N-protected alcohol of the formula 13. In the compound of the formula 13 obtained by this Step, T₂ is OH.

When, for example, R³′ is methylene and P₂ is a t-butoxycarbonyl group, this Step comprises two sequential reactions. The first step is protection of a nitrogen atom of compound 19 with a t-butoxycarbonyl group, and the second step is hydrolysis of a cyclic urethane. In this case, as the butoxycarbonylation reagent to be used in the first step, for example, di-t-butyl carbonate is used, and the reaction is carried out in the presence of a base as necessary.

As the base to be used in the first step, for example, alkyl lithiums such as butyl lithium, t-butyl lithium, s-butyl lithium, etc.; alkali metal hydrides such as sodium hydride, potassium hydride, etc.; alkali metal amides such as lithium diisopropyl amide, sodium bis(trimethylsilyl)amide, lithium bis(trimethylsilyl)amide, etc. and the like can be mentioned. A preferable base is an alkali metal hydride and sodium hydride is more preferable. As the solvent, for example, ether solvents such as diethyl ether, tetrahydrofuran (THF), dioxane, 1,2-dimethoxyethane, diglyme, etc.; polar solvents such as N,N-dimethylformamide, dimethyl sulfoxide, etc. and the like can be mentioned, which may be used alone or in combination. A preferable solvent in this reaction is ether solvent and THF is more preferable. The reaction temperature is generally −20° C. to 100° C., preferably 0° C. to 50° C. The reaction time is 1 hr to 48 hrs, preferably 1 hr to 24 hrs.

The second step is hydrolysis with a base.

As the base to be used in the second step, for example, alkali metal carbonates such as sodium carbonate, potassium carbonate, cesium carbonate, sodium hydrogen carbonate, potassium hydrogen carbonate, etc.; alkali metal hydroxides such as lithium hydroxide, sodium hydroxide, potassium hydroxide, etc. and the like can be mentioned, with preference given to alkali metal carbonates, and cesium carbonate is more preferable. As the solvent, for example, ether solvents such as diethyl ether, tetrahydrofuran (THF), dioxane, 1,2-dimethoxyethane, diglyme, etc.; alcohol solvents such as methanol, ethanol, isopropyl alcohol, t-butyl alcohol, etc.; polar solvents such as N,N-dimethylformamide, dimethyl sulfoxide, water, etc. and the like can be mentioned, which may be used alone or in combination. A preferable solvent in this reaction is an alcohol solvent, and methanol is more preferable. The reaction temperature is generally 0° C. to 100° C., preferably room temperature to 50° C. The reaction time is 10 min to 24 hrs, preferably 30 min to 6 hrs. Thus obtained compound of the formula 13 can be used in the next reaction without isolation.

Step 3-1

In this Step, substituent T₁ on R⁴′ and/or substituent T₂ on R³′ of a compound of the formula 13 obtained by Steps 1-3 and 2-6 are/is led to functional groups/a functional group under conventional conditions to lead to a compound of the formula 20. In this case, R⁴′ and T₁ on compound 13 are together led to R⁴ on compound 20, and R³′ and T₂ on compound 13 are together led to R³ on compound 20. When, for example, R⁴′ is an aromatic ring and T₁ is a halogen atom, so called Negishi reaction, Suzuki-Miyaura reaction (Metal-catalyzed Cross Coupling Reactions; WILEY-VCH; New York, 1998), Buchwald reaction, Ullmann reaction (Tetrahedron 2002, 11, 2041-2075; J. Am. Chem. Soc. 2003, 125, 6653-6655) and the like can be applied, whereby a compound of the formula 24 wherein T₄ is alkoxycarbonylalkylaryl group, carbonylaminoaryl group, alkoxycarbonylaryl group, biaryl group, arylaminoaryl group, alkylaminoaryl group or arylalkoxyaryl group can be obtained respectively. When, for example, R³′ is an alkyl chain and T₂ is a hydroxyl group, for example, a compound of the formula 20 wherein T₃ is an aminoalkyl group or alkylaminoalkyl group can be obtained by a conventional method. Thus obtained compound of the formula 20 can be used in the next reaction without isolation.

Where necessary, deprotection of carboxylic-protecting group, optical resolution and protection of carboxylic acid may be performed in this Step.

For example, when the ester of the formula 13 is led to a carboxylic acid derivative by a conventional method. While the reaction conditions are appropriately chosen depending on P₁, when, for example, P₁ is a methyl group or an ethyl group, conventional hydrolysis with a base is performed. When, for example, P₁ is a t-butyl group, deprotection with an acid is performed.

As the base, for example, alkali metal carbonates such as cesium carbonate, sodium carbonate, potassium carbonate, etc.; alkali metal hydroxides such as lithium hydroxide, sodium hydroxide, potassium hydroxide, etc. and the like can be mentioned, with preference given to sodium hydroxide. As the acid to be used for deprotection with an acid, mineral acids such as hydrochloric acid, sulfuric acid, phosphoric acid, nitric acid, etc.; organic acids such as trifluoroacetic acid, methanesulfonic acid, p-toluenesulfonic acid, trifluoromethanesulfonic acid etc. and the like can be mentioned, with preference given to hydrochloric acid or trifluoroacetic acid. As the solvent for hydrolysis with a base, for example, ether solvents such as diethyl ether, tetrahydrofuran (THF), dioxane, 1,2-dimethoxyethane, diglyme, etc.; alcohol solvents such as methanol, ethanol, isopropyl alcohol, t-butyl alcohol, etc.; polar solvents such as water, etc. and the like can be mentioned, which may be used alone or in combination. A preferable solvent in this reaction is a mixed solvent of an ether solvent and an alcohol solvent, more preferably a mixed solvent of methanol, THF and water. The reaction temperature is generally room temperature to 100° C., preferably room temperature to 80° C. The reaction time is 1 hr to 48 hrs, preferably 2 hrs to 24 hrs. In the case of deprotection with an acid, As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, acetonitrile, water, etc. and the like can be mentioned, with preference given to ethyl acetate, dioxane, dichloromethane, chloroform or no solvent.

In addition, thus obtained racemic carboxylic acid is led to a diastereomeric salt of a chiral amine and recrystallized. As the chiral amine, alkaloids such as cinchonine, quinidine, cinchonidine, quinine, brucine, strychnine, etc.; amino acids or alcohols derived from amino acids such as alanine, phenylalanine, alaminol, phenylalaminol, etc.; phenethylamine, naphthylethylamine, etc. and the like can be mentioned, with preference given to quinidine or cinchonidine. As the solvent used for recrystallization, for example, ether solvents such as diethyl ether, tetrahydrofuran (THF), dioxane, 1,2-dimethoxyethane, etc.; hydrocarbon solvents such as benzene, toluene, etc.; alcohol solvents such as methanol, ethanol, isopropyl alcohol, t-butyl alcohol, etc.; ester solvents such as ethyl acetate, methyl acetate, butyl acetate, etc.; polar solvents such as acetone, 2-butanone, acetonitrile, water, etc. and the like can be mentioned, which may be used alone or in combination. A preferable solvents in this recrystallization are isopropyl alcohol, acetone, ethyl acetate, and a mixed solvent thereof.

Thus obtained chiral acid is subjected to esterification again to give an chiral carboxylic acid of the compound 16. For protection of a carboxylic acid derivative with P₁, which is a protecting group, by a conventional method, P₁ is appropriately chosen depending on P₂, or T₁, T₂. For example, when P₁ is a t-butyl group, a method using isobutene in the presence of an acid catalyst to give t-butyl ester, and a method using N,N-dimethylformamide di-t-butylacetal can be mentioned.

When, for example, N,N-dimethylformamide di-t-butylacetal is used, as the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, dimethyl sulfoxide, acetonitrile, etc. and the like can be mentioned, which may be used alone or in combination. A preferable solvent in this reaction is a hydrocarbon solvent, and toluene is more preferable. The reaction temperature is generally room temperature to 150° C., preferably room temperature to 110° C. The reaction time is 1 hr to 24 hrs, preferably 2 hrs to 12 hrs. Thus obtained compound of the formula 20 can be used in the next reaction without isolation.

When T₁ and/or T₂ are/is hydrogen atom or subsequent conversion is not necessary, this Step does not need to be performed and the compound of the formula 13 can be treated as the compound of the formula 20.

Step 3-2

In this Step, P₂, which is a nitrogen-protecting group in a compound of the formula 20, is deprotected by a conventional method. The reaction conditions are appropriately chosen depending on P₁, or P₂. For example, when P₂ is a t-butoxycarbonyl group and P₁ is a methyl group or proton, deprotection can be performed under acidic conditions.

As the acid, mineral acids such as hydrochloric acid, sulfuric acid, phosphoric acid etc., organic acids such as acetic acid, trifluoroacetic acid, methanesulfonic acid, p-toluenesulfonic acid etc. can be mentioned, with preference givem to hydrochloric acid. As the solvent, for example, ether solvents such as diethyl ether, tetrahydrofuran (THF), dioxane, 1,2-dimethoxyethane, diglyme, etc., hydrocarbon solvents such as benzene, toluene, hexane, xylene, etc.; halogenated solvents such as dichloromethane, chloroform, carbon tetrachloride, 1,2-dichloroethane, etc.; alcohol solvents such as methanol, ethanol, isopropyl alcohol, t-butyl alcohol, etc.; ester solvents such as ethyl acetate, methyl acetate, butyl acetate, etc.; polar solvents such as acetone, N,N-dimethylformamide, dimethyl sulfoxide, acetonitrile, water, etc. and the like can be mentioned, which may be used alone or in combination. A preferable solvent in this reaction is an ether solvent or an ester solvent, an alcohol solvent or acetonitrile. The reaction temperature is generally −30° C. to 60° C., preferably 0° C. to 50° C. The reaction time is generally 1 hr to 72 hrs, preferably 1 hr to 48 hrs.

Thus obtained compound of the formula 21 can be used in the next reaction without isolation.

Step 3-3

In this Step, a hydrogen atom of a compound of the formula 22 is replaced with a chlorosulfonyl group. After conversion the compound of the formula 22 to a sulfonic acid derivative, the derivative is subsequently chlorinated to give the sulfonyl chloride derivative of the formula 23. As the sulfonylation agent, sulfuric acid, chlorosulfonic acid, chlorosulfonic acid trimethylsilyl ester can be mentioned. As the solvent, no solvent, or halogenated solvents such as dichloromethane, chloroform, carbon tetrachloride, 1,2-dichloroethane, etc.; ester solvents such as ethyl acetate, methyl acetate, butyl acetate, etc.; polar solvents such as acetic acid, sulfuric acid, etc. and the like can be mentioned, which may be used alone or in combination. A preferable solvent in this reaction is halogenated solvent, and chloroform is more preferable. The reaction temperature is generally −20° C. to 100° C., preferably 0° C. to 50° C. The reaction time is 1 hr to 96 hrs, preferably 1 hr to 72 hrs.

Subsequent chlorination reaction is a conventional synthetic method for a sulfonyl chloride derivative, and as the chlorinating agent to be used for the reaction, for example, thionyl chloride, phosphorus oxychloride, phosphorus pentachloride, chlorosulfonic acid can be mentioned, with preference given to thionyl chloride. As the solvent, no solvent, or hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, etc., and the like can be mentioned, which may be used alone or in combination. A preferable solvent is no solvent, and a mixed solvent of thionyl chloride, which is a chlorinating agent, and a catalytic amount of N,N-dimethylformamide is more preferable. The reaction temperature is generally 0° C. to 100° C., preferably room temperature to 80° C. The reaction time is 1 hr to 48 hrs, preferably 3 hrs to 24 hrs.

Thus obtained compound of the formula 23 can be used in the next reaction without isolation.

Step 3-4

In this Step, the amine of the formula 21 and obtained in Step 3-2 is led to sulfonamide derivative or a sulfamide derivative of the formula 24.

When the compound of formula 24 is a sulfonamide derivative, for example, the derivative can be obtained by a reaction with the ClSO₂—R¹ of the formula 22 obtained in step 3-3 or O(SO₂—R¹)₂ in the presence of a base. As the base, for example, organic bases such as triethylamine, diisopropylethylamine, pyridine, N-methylmorpholine, 2,6-lutidine, 1,8-diazabicyclo[5.4.0]7-undecene, N,N-dimethylaminopyridine, etc. and the like can be mentioned, with preference given to pyridine, 2,6-lutidine, N,N-dimethylaminopyridine or triethylamine, which may be used as a solvent. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as N,N-dimethylformamide, acetonitrile, etc. and the like can be mentioned, which may be used alone or in combination. A preferable solvent in this reaction is a halogenated solvent or an ether solvent, or a mixed solvent of ether solvent and water, and a mixed solvent of dioxane and water is more preferable. The reaction temperature is generally −30° C. to 100° C., preferably room temperature to 50° C. The reaction time is 1 hr to 72 hrs, preferably 1 hr to 48 hrs.

In addition, when the formula 24 is a sulfamide derivative, the derivative can be synthesized by two consecutive reactions based on the method known in literature (Tetrahedron 1996,52,14217-14227). The first step is a reaction of 2-haloethanol with chlorosulfonyl isocyanate and then with the compound of the formula 21 in the presence of a base to give an oxazolidin-2-on-3-ylsulfamide, and the second step is a reaction of the compound obtained above with a desired amine to give a sulfamide of the formula 24.

As 2-haloethanol, for example, 2-chloroethanol, 2-bromoethanol and 2-iodoethanol can be mentioned, with preference given to 2-chloroethanol. As the base, for example, organic bases such as triethylamine, pyridine, N-methylmorpholine, 2,6-lutidine, 1,8-diazabicyclo[5.4.0]7-undecene, etc. and the like can be mentioned. A preferable base is an organic base, and N-methylmorpholine is more preferable. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, N,N-dimethylacetamide, dimethyl sulfoxide, acetonitrile, etc. and the like can be mentioned, which may be used alone or in combination. A preferable solvent in this reaction is a polar solvent, and acetonitrile is more preferable. The reaction temperature is generally −20° C. to 100° C., preferably 0° C. to 50° C. The reaction time is 1 hr to 48 hrs, preferably 1 hr to 24 hrs.

The second step is a nucleophilic substitution reaction with amine. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogennated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, dimethyl sulfoxide, acetonitrile, etc. and the like can be mentioned, which may be used alone or in combination. A preferable solvent in this reaction is a polar solvent, and acetonitrile is more preferable. The reaction temperature is generally −20° C. to 100° C., preferably 0° C. to 100° C. The reaction time is 1 hr to 48 hrs, preferably 1 hr to 24 hrs.

Thus obtained compound of the formula 24 can be used in the next reaction without isolation.

Step 3-5

In this Step, the carboxylic acid derivative of the formula 24 and obtained in Step 3-4 (compound wherein P₁ is proton) is protected using a protecting group, P₄, by a conventional method. While P₄ is appropriately chosen depending on R³, R⁴, for example, when P₄ is a t-butyl group, a method using isobutene in the presence of an acid catalyst, and a method using N,N-dimethylformamide di-t-butylacetal can be mentioned. For example, when N,N-dimethylformamide di-t-butylacetal is used, as the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, dimethyl sulfoxide, acetonitrile, etc. and the like can be mentioned, which may be used alone or in combination. A preferable solvent in this reaction is hydrocarbon solvent, and toluene is more preferable. The reaction temperature is generally room temperature to 150° C., preferably room temperature to 11° C. The reaction time is 1 hr to 24 hrs, preferably 2 hrs to 12 hrs.

When, for example, P₄ is a methyl group, an ethyl group or a benzyl group, carboxylic acid is led to activated ester or acyl chloride in a solvent, and subsequently an alcohol is added in the presence of a base, or carboxylic acid is reacted with an alcohol in the presence of acid catalyst to give a compound of the formula 25.

As the activated ester, acyl imidazole, mixed acid anhydride, hydroxybenzotriazole ester, hydroxysuccinimide ester and the like can be mentioned, which are prepared by known methods. For preparation of acyl chloride, thionyl chloride, oxalyl chloride and the like are used. The reaction temperature for preparation of the activated ester or acyl chloride is generally −78° C. to 50° C., preferably −20° C. to room temperature.

The reaction time is 10 min to 6 hrs, preferably 30 min to 6 hrs.

The temperature of the reaction with the alcohol equivalent wherein a hydroxylamine or hydroxyl group is protected is generally −78° C. to 50° C., preferably −20° C. to room temperature.

The reaction time is 10 min to 6 hrs, preferably 30 min to 6 hrs.

As the base, organic base such as triethylamine, pyridine, N-methylmorpholine, 2,6-lutidine, 1,8-diazabicyclo[5.4.0]undec-7-ene, etc.; etc. can be mentioned, with preference given to N-methylmorpholine.

As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc., etc. can be mentioned, which may be used alone or in combination. A preferable solvent in this reaction is one of ether solvents, and THF is more preferable.

When carboxylic acid is reacted with an alcohol in the presence of an acid catalyst, of as the acid, for example, p-toluenesulfonic acid, pyridinium p-toluenesulfonate, camphorsulfonic acid, methanesulfonic acid, benzenesulfonic acid, hydrochloric acid and the like can be mentioned. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as N,N-dimethylformamide, etc. and the like can be mentioned, which may be used alone or in combination. When, for example, P₄ is an ethyl group, a preferable solvent is ethanol. The reaction temperature is −78° C. to 100° C., preferably room temperature to 120° C. The reaction time is 1 hr to 48 hrs, preferably 12 hrs to 24 hrs.

Thus obtained compound of the formula 25 can be used in the next reaction without isolation. This Step is necessary only when P₁ is a hydrogen atom. When P₁═P₄, this Step can be omitted and the compound of the formula 24 can be treated as the compound of the formula 25.

Step 3-6

In this Step, a general alkylation reaction is performed. The compound of the formula 25 obtained in Step 3-5 is reacted with an alkylating agent in a solvent in the presence of a base to give a compound of the formula 26. While the alkylating agent is appropriately chosen depending on the desired R⁷⁰, for example, alkyl bromide, alkyl iodide, alkyl methanesulfonate, alkyl p-toluenesulfonate, alkyl trifluoromethanesulfonate can be mentioned, with preference given to alkyl iodide or bromide. The compound 26 can be obtained by performing so called Mitsunobu reaction (J. Org. Chem. 1981, 46, 2381-2383) with an alcohol derivative appropriately determined depending on desired R⁷⁰. In the case of, for example, alkylation reaction in the presence of a base, as the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; ester solvents such as ethyl acetate, methyl acetate, butyl acetate, etc.; acetone, polar solvents such as N,N-dimethylformamide, dimethyl sulfoxide, etc. and the like can be mentioned, which may be used alone or in combination. A preferable solvent in this reaction is N,N-dimethylformamide. As the base, for example, alkyl lithiums such as butyl lithium, t-butyl lithium, s-butyl lithium, etc.; alkali metal hydrides such as sodium hydride, potassium hydride, etc.; metal alcoholates such as potassium t-butoxide, sodium ethoxide, sodium methoxide, etc.; alkali metal amides such as lithium diisopropyl amide, sodium bis(trimethylsilyl)amide, lithium bis(trimethylsilyl)amide etc.; alkali metal carbonates such as sodium carbonate, potassium carbonate, sodium hydrogen carbonate, potassium hydrogen carbonate, etc.; alkali metal hydroxides such as lithium hydroxide, sodium hydroxide, potassium hydroxide, etc.; alkali metal carboxylates such as sodium acetate, potassium acetate etc.; alkali metal phosphates such as sodium phosphate, potassium phosphate, etc., organic bases such as triethylamine, diisopropylethylamine, pyridine, N-methylmorpholine, 2,6-lutidine, 1,8-diazabicyclo[5.4.0]7-undecene, etc. and the like can be mentioned, with preference given to potassium carbonate. The reaction temperature is generally 0° C. to 90° C., preferably room temperature to 80° C. The reaction time is generally 1 hr to 24 hrs, preferably 2 hrs to 12 hrs.

Thus obtained compound of the formula 26 can be used in the next reaction without isolation.

Step 4-1

In this Step, a conventional sulfonylation is performed. In this Step, the compound of the formula 29 is led to a sulfonamide derivative or a sulfamide derivative of the formula 30 in the same manner as in Step 3-4.

When the compound of the formula 30 is a sulfonamide derivative, for example, ClSO₂—R¹ of the formula 23 or O(SO₂—R¹)₂ reacted with the compound of the formula 29, and when the formula 30 is a sulfamide derivative, for example, it can be obtained from the compound of the formula 29 in the same manner as in Step 3-4.

Thus obtained compound of the formula 30 can be used in the next reaction without isolation.

Step 4-2

In this Step, a conventional alkylation reaction is performed. The compound of the formula 30 obtained in Step 4-1 is reacted with an alkylating agent in a solvent in the presence of a base to give a compound of the formula 31. While the alkylating agent is appropriately chosen depending on the desired R⁷⁰ for example, alkyl bromide, alkyl iodide, alkyl methanesulfonate, alkyl p-toluenesulfonate, alkyl trifluoromethanesulfonate and the like can be mentioned, with preference given to alkyl iodide or bromide, and bromoacetic acid t-butyl ester is more preferable. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; alcohol solvents such as methanol, ethanol, isopropyl alcohol, t-butyl alcohol, etc.; ester solvents such as ethyl acetate, methyl acetate, butyl acetate, etc.; polar solvents such as acetone, N,N-dimethylformamide, dimethyl sulfoxide, etc. and the like can be mentioned, which may be used alone or in combination. A preferable solvent is N,N-dimethylformamide. As the base, for example, alkyl lithiums such as butyl lithium, t-butyl lithium, s-butyl lithium, etc.; alkali metal hydrides such as sodium hydride, potassium hydride, etc.; metal alcoholates such as potassium t-butoxide, sodium ethoxide, sodium methoxide, etc.; alkali metal amides such as lithium diisopropyl amide, sodium bis(trimethylsilyl)amide, lithium bis(trimethylsilyl)amide, etc.; alkali metal carbonates such as sodium carbonate, potassium carbonate, sodium hydrogen carbonate, potassium hydrogen carbonate, etc.; alkali metal hydroxides such as lithium hydroxide, sodium hydroxide, potassium hydroxide, etc.; alkali metal carboxylates such as sodium acetate, potassium acetate, etc.; organic bases such as triethylamine, diisopropylethylamine, pyridine, N-methylmorpholine, 2,6-lutidine, 1,8-diazabicyclo[5.4.0]7-undecene, etc. and the like can be mentioned, with preference given to potassium carbonate. The reaction temperature is generally 0° C. to 100° C., preferably room temperature to 70° C. The reaction time is generally 1 hr to 24 hrs, preferably 2 hrs to 12 hrs.

Thus obtained compound of the formula 31 can be used in the next reaction without isolation.

Step 4-3

In this Step, a conventional dehydration reaction is performed. For example, the compound of the formula 31 obtained in Step 4-2 is reacted with a sulfonyl halide or a sulfonic anhydride in a solvent in the presence of a base to give the compound of the formula 32. As sulfonyl halide or sulfonic anhydride, for example, methanesulfonyl chloride, p-toluenesulfonyl chloride, benzenesulfonylchloride, methanesulfonic anhydride, trifluoromethanesulfonic anhydride, and the like can be mentioned, with preference given to methanesulfonyl chloride. As the base, for example, alkyl lithiums such as butyl lithium, t-butyl lithium, s-butyl lithium, etc.; alkali metal hydrides such as sodium hydride, potassium hydride, etc.; metal alcoholates such as potassium t-butoxide, sodium ethoxide, sodium methoxide, etc.; alkali metal amides such as lithium diisopropyl amide, sodium bis(trimethylsilyl)amide, lithium bis(trimethylsilyl)amide, etc.; alkali metal hydroxides such as lithium hydroxide, sodium hydroxide, potassium hydroxide, etc.; organic bases such as triethylamine, diisopropylethylamine, pyridine, N-methylmorpholine, 2,6-lutidine, 1,8-diazabicyclo[5.4.0]7-undecene, etc. and the like can be mentioned, with preference given to a combined use of N-methylmorpholine and 1,8-diazabicyclo[5,4,0]-7-undecene (DBU). As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, acetonitrile, etc. and the like can be mentioned, which may be used alone or in combination. A preferable solvent is THF. The reaction temperature is generally −30° C. to 120° C., preferably 0° C. to room temperature. The reaction time is generally 2 hrs to 24 hrs, preferably 2 hrs to 12 hrs.

Thus obtained compound of the formula 32 can be used in the next reaction without isolation.

Step 4-4

In this Step, a conventional cyclopropanation reaction is performed. The compound of the formula 32 obtained in Step 4-3 is reacted with a ylide compound in a solvent in the presence of a base to give the compound of the formula 26. As the ylide compound to be used for the reaction can be easily synthesized according to the method known in literature (J. Org. Chem., 1992, 57, 6265-6270). As the base, for example, alkyl lithiums such as butyl lithium, t-butyl lithium, s-butyl lithium, etc.; alkali metal hydrides such as sodium hydride, potassium hydride, etc.; metal alcoholates such as potassium t-butoxide, sodium ethoxide, sodium methoxide, etc.; alkali metal amides such as lithium diisopropyl amide, sodium bis(trimethylsilyl)amide, lithium bis(trimethylsilyl)amide, etc. and the like can be mentioned, with preference given to sodium hydride. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; polar solvents such as N,N-dimethylformamide, dimethyl sulfoxide, etc. and the like can be mentioned, which may be used alone or in combination. A preferable solvent is THF. The reaction temperature is generally −80° C. to 120° C., preferably 0° C. to room temperature. The reaction time is generally 2 hrs to 24 hrs, preferably 2 hrs to 12 hrs.

Thus obtained compound of the formula 26 can be used in the next reaction without isolation.

Step 5-1

In this Step, the cyclic urethane derivative of the formula 19 obtained in Step 2-5 is reacted with sulfonyl chloride of the formula 23 and sequentially subjected to ring opening reaction with a nucleophilic agent to give a sulfonamide derivative of the formula 33. When, for example, the nucleophilic agent is a base (hydroxy anion), T₄ in the compound of the formula 33 obtained by this Step is a hydroxyl group. When, for example, the nucleophilic agent is an alkylamine, T₄ in a compound of the formula 33 obtained by this Step is an alkylcarbamoyloxy group. In this Step, moreover, a protecting group P₁ of carboxylic acid does not change and correspond to P₄. The reaction is carried out in the presence of a base. As the base, for example, alkyl lithiums such as butyl lithium, t-butyl lithium, s-butyl lithium, etc.; alkali metal hydrides such as sodium hydride, potassium hydride, etc.; alkali metal amides such as lithium diisopropyl amide, sodium bis(trimethylsilyl)amide, lithium bis(trimethylsilyl)amide, etc. and the like can be mentioned, preferably sodium hydride. As the solvent, for example, ether solvents such as diethyl ether, tetrahydrofuran (THF), dioxane, 1,2-dimethoxyethane, diglyme, 15-crown-5-ether, etc.; polar solvents such as N,N-dimethylformamide, dimethyl sulfoxide, etc. and the like can be mentioned, which may be used alone or in combination. A preferable solvent in this reaction is an ether solvent, more preferably a mixed solvent of THF and 15-crown-5-ether. The reaction temperature is generally −20° C. to 100° C., preferably 0° C. to 50° C. The reaction time is 1 hr to 48 hrs, preferably 1 hr to 24 hrs.

When, for example, the nucleophilic agent in the subsequent ring opening reaction is a base (hydroxyl anion), this reaction is conventional hydrolysis in the presence of a base, and the base to be used for the reaction, for example, alkali metal carbonates such as sodium carbonate, potassium carbonate, cesium carbonate, sodium hydrogen carbonate, potassium hydrogen carbonate, etc.; alkali metal hydroxides such as lithium hydroxide, sodium hydroxide, potassium hydroxide, etc. and the like can be mentioned, with preference given to alkali metal hydroxides and sodium hydroxide is more preferable. As the solvent, for example, ether solvents such as diethyl ether, tetrahydrofuran (THF), dioxane, 1,2-dimethoxyethane, diglyme, etc.; alcohol solvents such as methanol, ethanol, isopropyl alcohol, t-butyl alcohol, etc.; polar solvents such as N,N-dimethylformamide, dimethyl sulfoxide, water, etc. and the like can be mentioned, which may be used alone or in combination. A preferable solvent in this reaction is a polar solvent, and a mixed solvent of THF, methanol and water is more preferable. The reaction temperature is generally 0° C. to 100° C., preferably room temperature to 50° C. The reaction time is 10 min to 48 hr, preferably 30 min to 24 hrs. Thus obtained compound of the formula 33 can be used in the next reaction without isolation.

For example, when the nucleophilic agent is alkylamine, as the alkylamine, isopropylamine, morpholine, benzylamine, and the like can be mentioned. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; polar solvents such as N,N-dimethylformamide, dimethyl sulfoxide, acetonitrile, etc., and the like can be mentioned, which may be used alone or in combination. A preferable solvent in this reaction is an ether solvent, and THF is more preferable. The reaction temperature is generally 0° C. to 100° C., preferably room temperature to 80° C. The reaction time is 1 hr to 24 hr, preferably 1 hr to 12 hrs. Thus obtained compound of the formula 33 can be used in the next reaction without isolation.

Step 5-2

In this Step, a sulfonamide group of a compound of the formula 33 and obtained in Step 5-1 is alkylated under conventional conditions to give a compound of the formula 26. In compound 26 obtained in this Step, R³ and R⁷⁰ may be taken together to form a ring. For example, when T₄ is a hydroxyl group, treatment with an aldehyde using a conventional method provides cyclic acetal which includes the nitrogen atom of the sulfonamide.

As the aldehyde, for example, paraformaldehyde, trioxane, acetaldehyde, benzaldehyde and the like can be mentioned. For example, when aldehyde is paraformaldehyde, dehydrating reaction in the presence of an acid catalyst affords cyclic acetal. As the acid, for example, p-toluenesulfonic acid, pyridium p-toluenesulfonate, camphorsulfonic acid, methanesulfonic acid, benzenesulfonic acid, hydrochloric acid, sulfuric acid, and the like can be mentioned. A preferable acid catalyst in this reaction is p-toluenesulfonic acid. As the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as N,N-dimethylformamide, etc. and the like can be mentioned, which may be used alone or in combination. A preferable solvent in this reaction is a hydrocarbon solvent and benzene is more preferable. The reaction temperature is 0° C. to 150° C., preferably room temperature to 120° C. The reaction time is 10 min to 24 hr, preferably 20 min to 12 hrs.

Thus obtained compound of the formula 26 can be used in the next reaction without isolation. In this Step, the substituent T₁ on R⁴′ does not change and R⁴′ and T₁ on compound 33 are taken together and corresponds to R⁴ on compound 26.

Step 6-1

In this Step, conventional functional group conversion reaction of the substituent R⁷⁰ on sulfonamide on the compound of the formula 26 obtained in Step 3-6, 4-4 or 5-2 to R⁷¹. The compound of the formula 26 is subjected to a combination of various reactions such as hydrolysis, amidation, reduction, C—C bond formation, cyclization, nucleophilic substitution, and the like as necessary in a solvent to give the compound of the formula 27. When, for example, R⁷⁰ is an alkoxycarbonylmethyl group and R⁷¹ is a carboxymethyl group, the compound of the formula 27 can be obtained by conventional hydrolysis, and when R⁷¹ is a carbamoylmethyl group, it can be obtained by subsequent amidation. When, for example, R⁷⁰ is a cyanomethyl group, an oxadiazole ring is constructed by a conventional method (J. Med. Chem. 1996, 39, 5228-5235) to give a compound of the formula 27 wherein R⁷¹ is an oxadiazolylmethyl group.

Thus obtained compound of the formula 27 can be used in the next reaction without isolation. This Step may be performed as necessary, and may be omitted, and a compound of the formula 26 can be treated as a compound of the formula 27.

Step 6-2

In this Step, the carboxy-protecting group in the compound of the formula 27 obtained in Step 6-1, is deprotected to give the carboxylic acid derivative of the formula 28 by conventional reactions. When R⁷¹ is not subject to any structural change by this reaction conditions, R² on compound 28 corresponds to R⁷¹ on compound 27. As a case of structual change of R⁷¹ by the reaction conditions, for example, a case when R⁷¹ is an alkoxycarbonylalkyl group, and the like can be mentioned. In this case, R² of a compound of the formula 28 is a carboxyalkyl group. While the reaction conditions are appropriately chosen depending on P₄, when, for example, P₄ is a methyl group or an ethyl group, this Step is achieved by hydrolysis with a base. When, for example, P₄ is a methyl group, deprotection using a halogen salt of alkali metal can be also performed. In addition, when, for example, P₄ is a t-butyl group, deprotection with an acid can be performed.

As the base to be used for hydrolysis, for example, alkali metal carbonates such as cesium carbonate, sodium carbonate, potassium carbonate, etc.; alkali metal hydroxides such as lithium hydroxide, sodium hydroxide, potassium hydroxide, etc. and the like can be mentioned, with preference given to alkali metal hydroxide. As the acid to be used for deprotection under acidic conditions, mineral acids such as hydrochloric acid, sulfuric acid, phosphoric acid, nitric acid, etc., organic acids such as trifluoroacetic acid, methanesulfonic acid, p-toluenesulfonic acid, trifluoromethanesulfonic acid, and the like can be mentioned, with preference given to hydrochloric acid or trifluoroacetic acid. As the halogen salt of alkali metal, for example, lithium iodide, sodium iodide, potassium iodide, lithium bromide, and the like can be mentioned, with preference given to lithium iodide. As the solvent for hydrolysis with a base, for example, ether solvents such as diethyl ether, tetrahydrofuran (THF), dioxane, 1,2-dimethoxyethane, diglyme, etc.; alcohol solvents such as methanol, ethanol, isopropyl alcohol, t-butyl alcohol, etc.; polar solvents such as water, etc. and the like can be mentioned, which may be used alone or in combination. A preferable solvent in this reaction is a mixed solvent of an ether solvent and an alcohol solvent, and a mixed solvent of methanol, THF and water is more preferable. The reaction temperature is generally room temperature to 120° C., preferably 50° C. to 100° C. The reaction time is 1 hr to 96 hr, preferably 6 hrs to 48 hrs. In the case of deprotection with an acid, as the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; ester solvents such as ethyl acetate, methyl acetate, butyl acetate, etc.; polar solvents such as acetone, N,N-dimethylformamide, water, etc. and the like can be mentioned, with preference given to ethyl acetate, dioxane, dichloromethane, chloroform or no solvent. The reaction temperature is generally room temperature to 100° C., preferably room temperature. The reaction time is 1 hr to 96 hr, preferably 6 hrs to 48 hrs. In the case of deprotection with a halogen salt of alkali metal, as the solvent, for example, ether solvents such as diethyl ether, tetrahydrofuran (THF), dioxane, 1,2-dimethoxyethane, diglyme, etc.; hydrocarbon solvents such as benzene, toluene, hexane, xylene, etc.; halogenated solvents such as dichloromethane, chloroform, carbon tetrachloride, 1,2-dichloroethane, etc.; alcohol solvents such as methanol, ethanol, isopropyl alcohol, t-butyl alcohol, etc.; polar solvents such as acetone, N,N-dimethylformamide, dimethyl sulfoxide, water, pyridine, etc. can be mentioned, which may be used alone or in combination. A preferable solvent in this reaction is pyridine. The reaction temperature is generally room temperature to 150° C., preferably 80° C. to 120° C. The reaction time is generally 1 hr to 96 hr, preferably 4 hrs to 48 hrs.

Step 7-1

In this Step, a conventional sulfonylation is performed. In this Step, the compound of the formula 34 synthesized by the method known in literature (Tetrahedron 1989, 45, 6091-6100) and the like is led to sulfonamide or sulfamide derivative of the formula 28, in the same manner as in Step 3-4.

When the compound of the formula 28 is a sulfonamide derivative, for example, ClSO₂—R¹ of the formula 23 or O(SO₂—R¹)₂ may be reacted with the compound of the formula 34, and when the formula 28 is a sulfamide derivative, for example, it can be obtained from the compound of the formula 34 in the same manner as in Step 3-4.

The production methods described in the specification are among the examples of the production method of the compound of the present invention, and compounds other than those explained in the above can be produced by combining conventional methods known in the field of organic synthetic chemistry.

The compound of the formula (1) and production method thereof of the present invention is explained in detail in the following by way of Examples. It is needless to say that the present invention is not limited by these Examples.

### PRODUCTION EXAMPLE 1-1

### 2-(3-benzyloxyphenyl)-cyclopropane-1,1-dicarboxylic acid dimethyl ester (step 1-1)

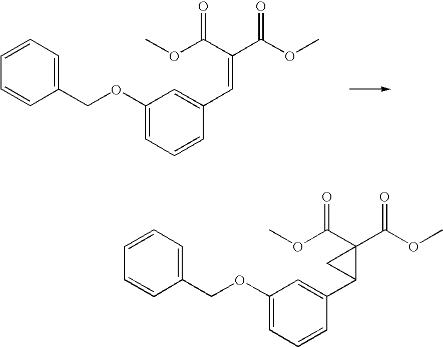

This procedure was performed according to the method described in J. Med. Chem. 1992, 35, 1410-1417.

While water-bathing, to a suspension of sodium hydride (liquid paraffin 40% added, 5.0 g, 0.13 mol) in dimethyl sulfoxide (180 mL) was gradually added trimethylsulfoxonium iodide (28 g, 0.13 mmol) under argon atmosphere, and the mixture was stirred for 30 min. Then dimethyl 2-(3-benzyloxybenzylidene)malonate (37 g, 0.11 mol) synthesized by the method described in the above-mentioned reference was added dropwise. After stirring for 1 hr at 50° C., saturated aqueous ammonium chloride solution (200 mL) and toluene (100 mL) were added to the obtained solution. The mixture was separated into layers and extracted with toluene (100 mL). The organic layer was sequentially washed with water (100 mL) and saturated aqueous sodium chloride solution (20 mL) and dried over magnesium sulfate. After filtration and evaporation, the obtained residue was purified by silica gel chromatography (hexane:chloroform=4:1) to give the title compound (31 g, 79%) as a pale-yellow oil.

### PRODUCTION EXAMPLE 1-2

### (1R*,2R*, 3R*)-2-methyl-3-phenylcyclopropane-1,1-dicarboxylic acid monomethyl ester (step 1-2)

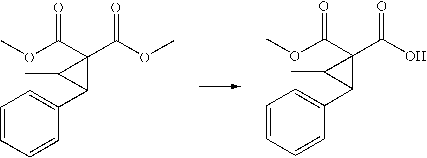

To a solution of (2R*,3R*)-2-methyl-3-phenyl-cyclopropane-1,1-dicarboxylic acid dimethyl ester (39 g, 0.16 mol) obtained in Preparation Example 1-7-2 in methanol (390 mL) was added 4N aqueous sodium hydroxide solution (160 mL, 0.62 mol) at 0° C., and the mixture was stirred for 18 hrs at room temperature. After the mixture was concentrated under reduced pressure, diethyl ether and water were added and the mixture was stirred. After the organic layer was removed, concentrated hydrochloric acid was added to the aqueous layer at 0° C. until the pH level read about 1. The organic layer was extracted with ethyl acetate, washed with saturated aqueous sodium chloride solution, and dried over sodium sulfate. The solution was filtrated and the solvent was evaporated. The obtained crude product was azeotroped with toluene, and diethyl ether and hexane were added gradually. The precipitated crystals were filtrated and dried under reduced pressure to give the title compound (35 g, yield 96%) as white crystals.

### PRODUCTION EXAMPLE 1-3

### (1R*,2S*,3S*)-1-t-butoxycarbonylamino-2-methyl-3-phenyl-cyclopropanecarboxylic acid methyl ester (step 1-3)

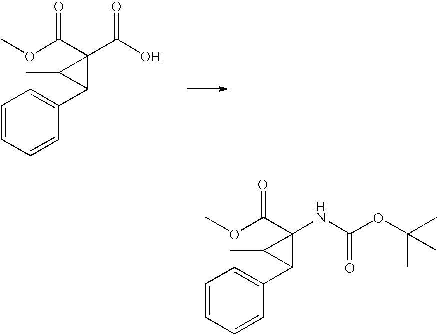

To a solution of 2-methyl-3-phenylcyclopropane-1,1-dicarboxylic acid mono-methyl ester (36 g, 0.16 mol) obtained in Preparation Example 1-11 and triethylamine (35 mL, 0.25 mol) in t-butylalcohol (370 mL) was added diphenylphosphoryl azide (44 mL, 0.20 mol). After stirring for 2 hrs at room temperature, the mixture was warmed gradually and refluxed for 7 hrs. After the solvent was evaporated under reduced pressure, a mixed solvent of hexane:ethyl acetate=4:1 (750 mL) and silica gel (200 g) were added and the mixture was stirred for 30 min. Then silica gel was removed, and the mixture was concentrated under reduced pressure. Hexane was added to the obtained residue, and the precipitated crystals were filtrated to give the title compound (35 g, yield 74%) as a white solid.

### PRODUCTION EXAMPLE 2-1

### 2-[2-(t-butyldiphenylsilanyloxy)ethyl]-2-phenyl-cyclopropane-1,1-dicarboxylic acid dimethyl ester (step 2-1)

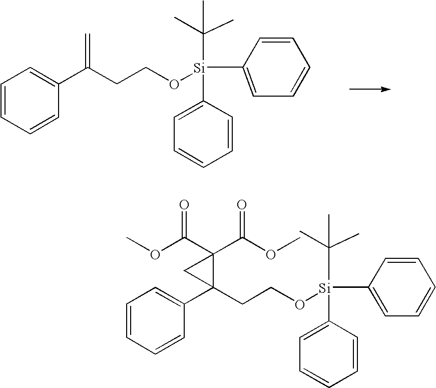

To a mixture of t-butyldiphenyl-(3-phenyl-3-butenyloxy)-silane (3.0 g, 7.0 mmol) and dimethyl diazomalonate (1.1 g, 7.0 mmol), which was synthesized by the method described in Synth. Commun. (1987, 17, 1709-1716), was added rhodium (II) acetate dimmer (62 mg, 0.14 mmol) under argon atmosphere, and the mixture was heated at 100° C. for 10 min. After cooling to room temperature, the mixture was diluted with chloroform (4 mL), purified by silica gel chromatography (hexane:ethyl acetate=100:0 to 4:1) to give the title compound (2.5 g, yield 70%) as a colorless oil.

### PRODUCTION EXAMPLE 2-2

### (1R*,6R*)-2-oxo-6-phenyl-3-oxa-bicyclo[4.1.0]heptane-1-carboxylic acid methyl ester (step 2-2)

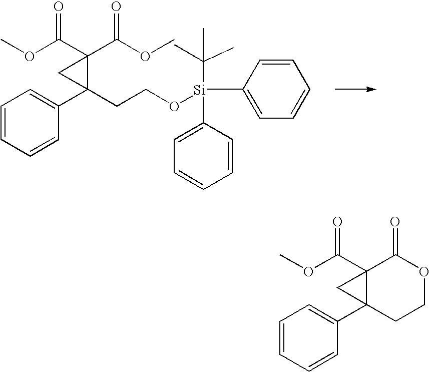

To a solution of 2-[2-(t-butyldiphenylsilanyloxy)ethyl]-2-phenyl-cyclopropane-1,1-dicarboxylic acid dimethyl ester (1.3 g, 2.5 mmol) obtained in Preparation Example 2-1 in tetrahydrofuran (13 mL) was added tetrabutylammonium fluoride trihydrate (1.2 g, 3.7 mmol) under argon atmosphere at 0° C., and the mixture was stirred at room temperature for 12 hrs. The obtained solution was diluted with ethyl acetate and washed with saturated aqueous sodium chloride solution. The aqueous layer was extracted twice with ethyl acetate, and the combined organic layers were dried over sodium sulfate. After filtration and evaporation, the obtained residue was purified by silica gel chromatography (hexane:ethyl acetate=10:1 to 1:1) to give the title compound (0.41 g, yield 67%) as a white solid.

### PRODUCTION EXAMPLE 2-3

### (1R*,5S*)-2-oxo-5-phenyl-3-oxa-bicyclo[3.1.0] hexane-1-carboxylic acid t-butyl ester (step 2-3)

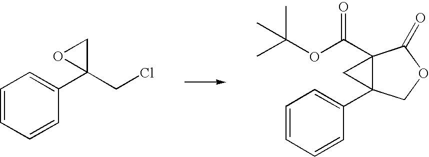

Under nitrogen atmosphere, potassium t-butoxide (110 g, 0.78 mol) was added to a solution of di-t-butyl malonate (170 g, 0.78 mol) in t-butyl alcohol (1.5 L) in 3 steps at room temperature. After stirring for 1 hr at room temperature, the mixture was heated to 70° C. Then a solution of 2-chloromethyl-2-phenyloxirane (120 g) in tetrahydrofuran (500 mL) synthesized by the method described in J. Org. Chem. (1962, 27, 2241-2243) was added dropwise over 90 min. After stirring for 12 hrs at 70° C., the mixture was cooled to room temperature and the solvent was evaporated. 10% Aqueous citric acid solution (500 mL) was added to the residue. The mixture was extracted with ethyl acetate (2.0 L), sequentially washed with water (500 mL) and saturated aqueous sodium chloride solution (200 mL), and dried over magnesium sulfate. After filtration and evaporation, the title compound (120 g, 3 steps, yield 54%) was recrystallized from a mixed solution of hexane:diisopropyl ether=1:1 (600 mL) as a white solid.

### PRODUCTION EXAMPLE 2-3-2

### a) (1R,5S)-2-oxo-5-phenyl-3-oxa-bicyclo[3.1.0]hexane-1-carboxylic acid

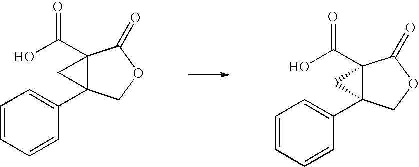

To a suspension of the starting material (7.0 g, 32 mmol) obtained by deprotection of t-butyl ester group of (1R*, 5S*)-2-oxo-5-phenyl-3-oxa-bicyclo[3.1.0]hexane-1-carboxylic acid t-butyl ester obtained in Production Example 2-3 in ethanol (210 mL) was added quinidine (10 g, 32 mmol) at room temperature and the mixture was stirred at room temperature for 5 hrs. The resulting crystals were collected by filtration to give an optically active form as a quinidine salt. The quinidine salt was suspended in ethyl acetate (80 mL) and water (60 mL). 1N Aqueous hydrochloric acid solution (20 mL, 20 mmol) was added at 0° C. and the mixture was stirred. The organic layer was washed with saturated aqueous sodium chloride solution, and dried over magnesium sulfate. After filtration and solvent removal, An optically active carboxylic acid compound was obtained (3.3 g, yield 47%, optical purity 96% ee) as a white amorphous form.

### b) (1R,5S)-2-oxo-5-phenyl-3-oxa-bicyclo[3.1.0]hexane-1-carboxylic acid t-butyl ester (step 2-3)

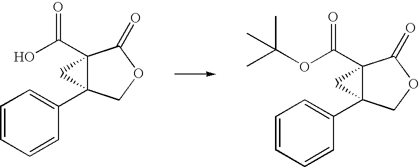

Under argon atomsphere, to a solution of (1R,5S)-2-oxo-5-phenyl-3-oxa-bicyclo[3.1.0]hexane-1-carboxylic acid (3.3 g, 15 mmol) obtained in Production Example 2-3-2 a) in toluene (33 mL) was added dropwise N,N-dimethylformamide di-t-butyl acetal (7.2 mL, 30 mmol) at room temperature for 5 min and the mixture was stirred at 80° C. for 1 hr. This operation was repeated three times and, after confirmation of the completion of the reaction, the reaction mixture was diluted with toluene. The mixture was allowed to cool to room temperature and washed successively with saturated aqueous sodium hydrogen carbonate solution (x2), water (x4) and saturated aqueous sodium chloride solution. After drying over sodium sulfate, filtration and solvent removal, the title compound was obtained (3.9 g, yield 95%, [α]²⁵_{D} −62.9° (c0.275, MeOH)) as white crystals.

### PRODUCTION EXAMPLE 2-4

### a) sodium (1R*,2S*)-1-t-butoxycarbonyl-2-hydroxymethyl-2-phenyl-cyclopropanecarboxylate

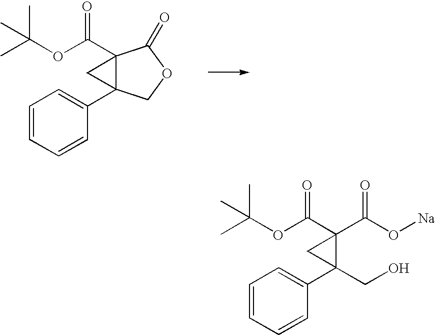

To a solution of (1R*,5S*)-2-oxo-5-phenyl-3-oxa-bicyclo[3.1.0]hexane-1-carboxylic acid t-butyl ester (30 g, 0.11 mol) obtained in Preparation Example 2-3 in tetrahydrofuran (300 mL) was added 4N aqueous sodium hydroxide solution (29 mL, 0.11 mol) at room temperature. After stirring at 60° C. for 2.5 hrs, the mixture was concentrated under reduced pressure. Then the mixture was azeotroped with toluene to remove water. The title compound (39 g) was obtained as a white amorphous form. The obtained product was used in the next step without purification.

### b) (1R*,2S*)-2-(t-butyldimethylsilanyloxymethyl)-2-phenyl-cyclopropane-1,1-dicarboxylic acid mono-t-butyl ester (step 2-4)

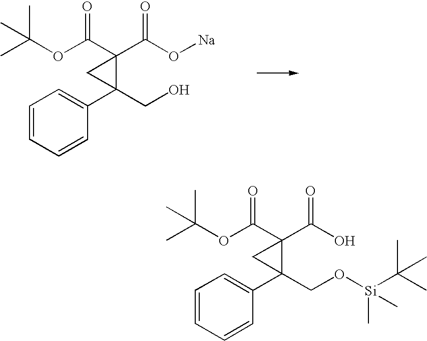

Imidazol (18 g, 0.27 mol) was added to a suspension of sodium (1R*,2S*)-1-t-butoxycarbonyl-2-hydroxymethyl-2-phenyl-cyclopropanecarboxylate (38 g, 0.11 mol) obtained in the above-mentioned Example a) in N,N-dimethylformamide (190 mL) under argon atmosphere at 0° C., and t-butyldimethylsilyl chloride (35 g, 0.24 mol) was further added in 2 steps. After warming to room temperature, the mixture was stirred for 12 hrs. Then water (76 mL) and methanol (76 mL) were added to the mixture at 0° C., which was followed by addition of potassium carbonate (30 g, 0.21 mol). After the obtained suspension was stirred for 3 hrs at room temperature, toluene (190 mL) was added and the mixture was separated into layers using 10% aqueous citric acid solution (400 mL) while adjusting pH to about 5. The aqueous layer was extracted twice with toluene, and the combined organic layer was sequentially washed with 10% aqueous citric acid solution and saturated aqueous sodium chloride solution, and dried over sodium sulfate. After filtration and evaporation, the product was azeotroped with xylene to remove t-butyldimethylsilanol. The title compound (44 g) was obtained as white crystals. The obtained product was used in the next step without purification.

### PRODUCTION EXAMPLE 2-4-2

### (1R*,2S*)-cis-1-carbamoyl-2-(2-hydroxyethyl)-2-phenylcyclopropanecarboxylic acid methyl ester (step 2-4)

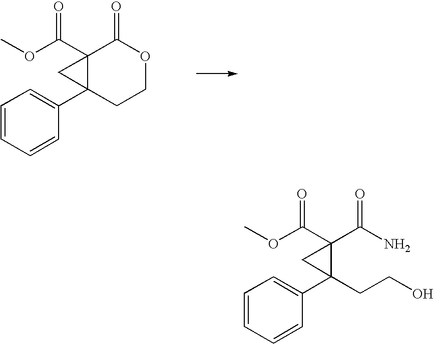

To a solution of (1R*,6R*)-2-oxo-6-phenyl-3-oxa-bicyclo[4.1.0]heptan-1-carboxylic acid methyl ester (0.29 g, 1.2 mmol) obtained in Preparation Example 2-2 in a tetrahydrofuran:methanol=1:1 mixture (6 mL) was added 28% ammonia water (6 mL) at room temperature, and the mixture was stirred for 12 hrs. The obtained solution was concentrated under reduced pressure to give the title compound (0.32 g) as a colorless oil. The obtained product was used in the next step without purification.

### PRODUCTION EXAMPLE 2-5

### (1R*,6R*)-3-oxo-6-phenyl-4-oxa-2-aza-bicyclo[4.1.0]heptane-1-carboxylic acid t-butyl ester (step 2-5)

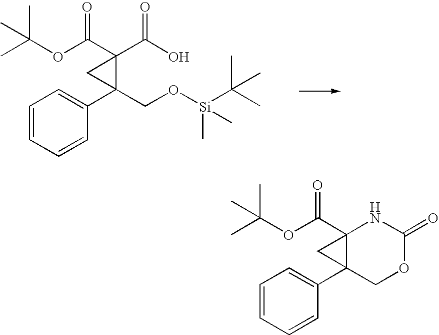

To a solution of (1R*,2S*)-2-(t-butyldimethylsilanyloxymethyl)-2-phenyl-cyclopropane-1,1-dicarboxylic acid mono-t-butyl ester (42 g, 0.10 mol) obtained in Preparation Example 2-4 in N,N-dimethylformamide (310 mL) were sequentially added triethylene (15 mL, 0.11 mol) and diphenylphosphoryl azide (24 mL, 0.11 mol) under argon atmosphere. After stirring at 80° C. for 30 min., the mixture was cooled to room temperature over 1 hr or more. Then, cesium fluoride (30 g, 0.20 mol) was added at once, and the mixture was stirred at 50° C. for 1.5 hrs. To the obtained suspension were added water (300 mL), toluene (150 mL), diethyl ether (150 mL) and tetrahydrofuran (100 mL), and the obtained crystals were filtrated. The filtrate was separated into layers, and the aqueous layer was extracted twice with toluene. The residue was sequentially washed with 1N aqueous sodium hydroxide solution and water and dried over sodium sulfate. After filtration and evaporation, the obtained residue and the above-mentioned filtrate were combined. A mixed solvent of hexane:diisopropyl ether=2:1 (150 mL) was added and the mixture was stirred at room temperature for 30 min. After the obtained crystal was filtrated, the residue was dried under reduced pressure to give the title compound (21 g, 3 steps, yield 73%) as a white solid.

### PRODUCTION EXAMPLE 2-5-2

### (1R*,7R*)-3-oxo-7-phenyl-4-oxa-2-azabicyclo[5.1.0]octane-1-carboxylic acid methyl ester (step 2-5)

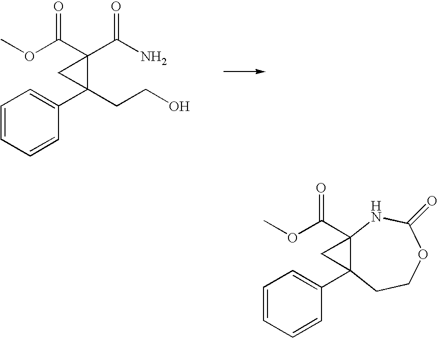

To a solution of (1R*,2S*)-cis-1-carbamoyl-2-(2-hydroxyethyl)-2-phenylcyclopropanecarboxylic acid methyl ester (0.30 g, 1.1 mmol) obtained in Preparation Example 2-4-2 in an ethyl acetate:acetonitrile:water=1:2:1 mixture (12 mL) was added iodobenzene diacetate (0.48 g, 1.5 mmol) at 0° C. After stirring at room temperature for 1.5 hrs, iodobenzene diacetate (64 mg, 0.23 mmol) was further added, and the mixture was stirred for 1.5 hrs. After the obtained solution was diluted with ethyl acetate and separated into layers, the aqueous layer was extracted twice with ethyl acetate. The combined organic layers were washed with saturated aqueous sodium chloride solution, and dried over sodium sulfate. After filtration and evaporation, the obtained residue was purified by silica gel chromatography (hexane:ethyl acetate=40:1 to 1:2) to give the title compound (0.11 g, 35%) as a white solid.

### PRODUCTION EXAMPLE 2-6

### a) (1R*,2R*)-1-t-butoxycarbonylamino-2-hydroxymethyl-2-phenylcyclopropanecarboxylic acid t-butyl ester

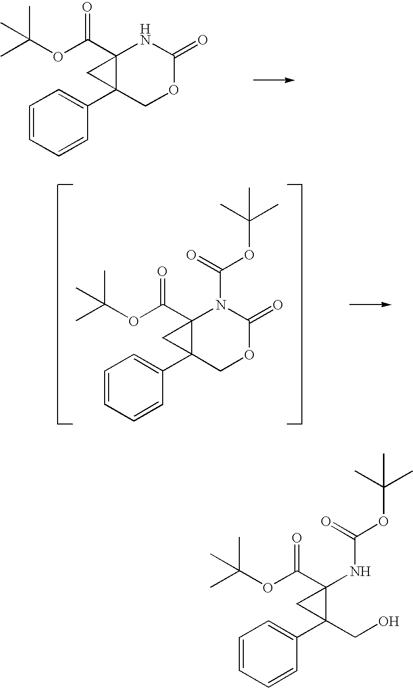

To a solution of (1R*,6R*)-3-oxo-6-phenyl-4-oxa-2-aza-bicyclo[4.1.0]heptan-1-carboxylic acid t-butyl ester (2.0 g, 6.9 mmol) obtained in Preparation Example 2-5 in tetrahydrofuran (40 mL) was added sodium hydride (liquid paraffin 40% added, 0.61 g, 15 mmol) under nitrogen atmosphere at 0° C., and the mixture was stirred for 30 min. Then a solution of di-t-butyl dicarbonate (2.4 g, 11 mmol) in tetrahydrofuran (20 mL) was added dropwise to the obtained solution. After stirring at 0° C. for 5 min., the mixture was warmed to room temperature and stirred for 20 hrs. Then, acetic acid (1 mL) and water (30 mL) were added to the obtained solution, and the solution was extracted three times with ethyl acetate (50 mL). The combined organic extracts were washed with water (30 mL) and saturated aqueous sodium chloride solution (30 mL), and dried over sodium sulfate. After filtration and evaporation, hexane (20 mL) was added to the obtained residue to allow precipitation of crystals. The crystals were collected and dried under reduced pressure to give (1R*,6R*)-3-oxo-6-phenyl-4-oxa-2-aza-bicyclo[4.1.0]heptan-1,2-dicarboxylic acid di-t-butyl (2.1 g) as a crude product.

To the obtained solution of (1R*,6R*)-3-oxo-6-phenyl-4-oxa-2-aza-bicyclo[4.1.0]heptan-1,2-dicarboxylic acid di-t-butyl ester (2.1 g, 5.5 mmol) in methanol (42 mL) was added cesium carbonate (0.54 g, 1.7 mmol) at room temperature. After stirring for 30 min., the mixture was concentrated to about half the amount under reduced pressure, and then saturated aqueous sodium chloride solution (40 mL) was added to the obtained residue. The mixture was extracted three times with ethyl acetate (30 mL), washed with water (50 mL) and saturated aqueous sodium chloride solution (50 mL), then dried over sodium sulfate. After filtration and evaporation, hexane (20 mL) was added to the obtained residue to allow precipitation of crystals. The crystals were filtrated and dried under reduced pressure to give the title compound (1.9 g, yield 74%) as a colorless amorphous form.

### b) (1R*,2R*)-1-t-butoxycarbonylamino-2-methanesulfonylmethyl-2-phenyl-cyclopropanecarboxylic acid t-butyl ester

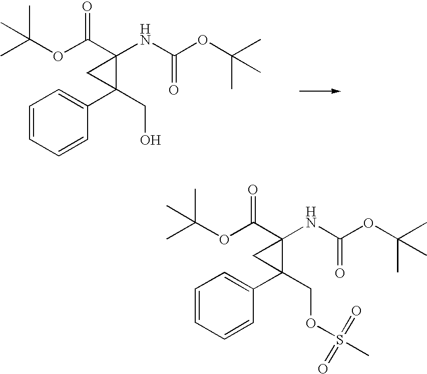

To a solution of (1R*,2S*)-1-t-butoxycarbonylamino-2-hydroxymethyl-2-phenyl-cyclopropanecarboxylic acid t-butyl ester (95 mg, 0.26 mmol) obtained in Production Example 2-6 a) in dichloromethane (1.0 mL) were successively added triethylamine (43 μL, 0.31 mmol) and methanesulfonyl chloride (22 μL, 0.29 mmol) at 0° C. and the mixture was stirred for 30 min. The obtained reaction mixture was purified by silica gel chromatography (hexane:ethyl acetate=3:2) to give the title compound (0.12 g, yield 100%) as a pale-yellow oil.

### c) (1R*,2S*)-1-t-butoxycarbonylamino-2-morpholin-4-ylmethyl-2-phenyl-cyclopropanecarboxylic acid t-butyl ester (step 2-6)

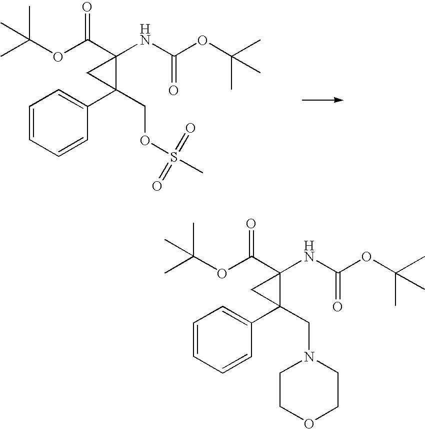

A solution of (1R*,2R*)-1-t-butoxycarbonylamino-2-methanesulfonylmethyl-2-phenyl-cyclopropanecarboxylic acid t-butyl ester (58 mg, 0.13 mmol) obtained in Production Example 2-6 b) in morpholine (0.32 mL) was stirred at 100° C. for 3 hrs. To this reaction mixture were added ethyl acetate (2.0 mL) and saturated aqueous sodium hydrogen carbonate solution (2.0 mL). The mixture was extracted 3 times with ethyl acetate, washed with water (3.0 mL) and saturated aqueous sodium chloride solution (3.0 mL) and dried over sodium sulfate. After filtration and solvent removal, the obtained residue was purified by silica gel chromatography (chloroform:ethyl acetate=7:3) to give the title compound (26 mg, yield 46%) as a colorless oil.

### PRODUCTION EXAMPLE 3-1

### (1R*,2S*)-1-t-butoxycarbonylamino-2-[2-(2-ethoxycarbonyl-ethyl)-phenyl]-cyclopropanecarboxylic acid methyl ester (step 3-1)

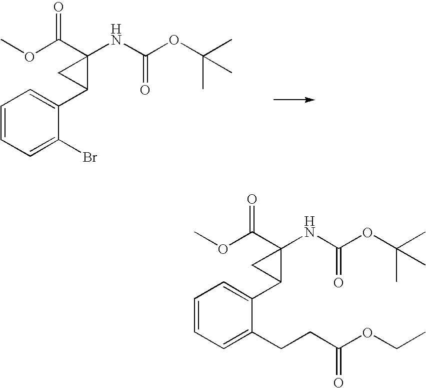

To a solution of (1R*,2S*)-2-(2-bromo-phenyl)-1-t-butoxycarbonylamino-cyclopropanecarboxylic acid methyl ester (50 mg, 0.14 mmol) obtained in a similar manner as described in Preparation Example 1-12 in tetrahydrofuran (0.5 mL) were added dibenzylidine acetone palladium (7.8 mg, 14 μmol), 1,2,3,4,5-pentaphenyl-1′-(di-t-butylphosphino)ferrocene (9.6 mg, 14 μmol) and 0.5M solution of 3-ethoxy-3-oxopropylzinc bromide in tetrahydrofuran (0.81 mL, 0.41 mmol), and the mixture was stirred at room temperature for 2 hrs. To the mixture were added 1N aqueous hydrochloric acid solution (0.5 mL) and water (5.0 mL), and the mixture was extracted twice with ethyl acetate (10 mL). Then the organic layer was sequentially washed with water (5.0 mL) and saturated aqueous sodium chloride solution (5.0 mL), and dried over magnesium sulfate. After filtration and evaporation, the obtained residue was purified by silica gel chromatography (hexane:ethyl acetate=5:1) to give the title compound (50 mg, yield 95%) as a brown oil.

### PRODUCTION EXAMPLE 3-1-2

### a) (1R*,2S*, 3S*)-1-t-butoxycarbonylamino-2-methyl-3-phenyl-cyclopropanecarboxylic acid

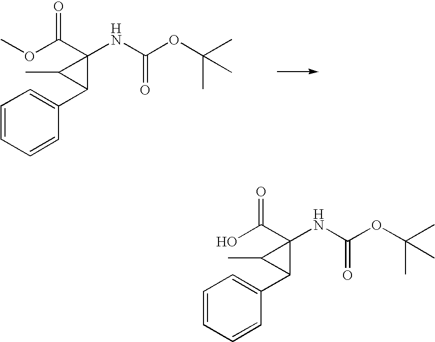

To a solution of (1R*,2S*,3S*)-1-t-butoxycarbonylamino-2-methyl-3-phenyl-cyclopropanecarboxylic acid methyl ester (37 g, 0.12 mol) obtained in Preparation Example 1-12 in a methanol:tetrahydrofuran=15:1 mixture (610 mL) was added aqueous solution of 4N sodium hydrate (95 mL, 0.38 mol), and the mixture was refluxed for 6 hrs. The mixture was allowed to cool to room temperature and the solvent was evaporated. 4N aqueous hydrochloric acid solution was added to the residue at 0° C. until the pH level read about 3. After the aqueous layer was extracted with ethyl acetate (800 mL), the organic layer was washed with saturated aqueous sodium chloride solution. The solution was dried over magnesium sulfate and the solvent was evaporated under reduced pressure to give the title compound (38 g) as a crude product of a pale-yellow oil. The obtained product was used in the next step without purification.

### b) (1S,2R,3R)-1-t-butoxycarbonylamino-2-methyl-3-phenyl-cyclopropanecarboxylic acid

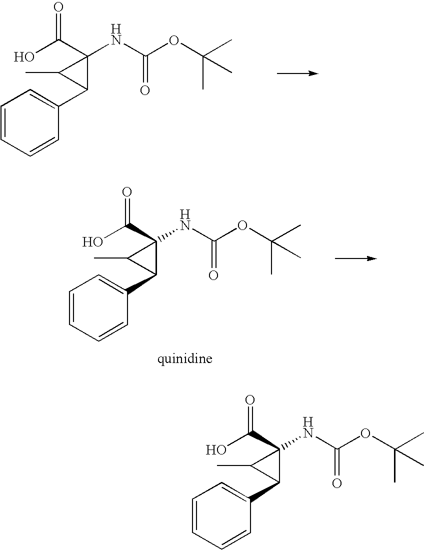

To a solution of (1R*,2S*,3S)-1-t-butoxycarbonylamino-2-methyl-3-phenyl-cyclopropanecarboxylic acid (38 g) obtained in Preparation Example 5-1 in isopropylalcohol (380 mL) was added quinidine (40 g, 0.12 mmol), and the mixture was stirred at room temperature for 20 hrs. The obtained crystal was filtrated to give an optical active quinidine salt (28 g, 44 mmol) as a white solid. The quinidine salt was suspended in ethyl acetate (250 mL) and water (250 mL), and the suspension was stirred after addition of 1N aqueous hydrochloric acid solution (88 mL, 88 mmol) at 0° C. The organic layer was washed with saturated aqueous sodium chloride solution, and dried over magnesium sulfate. The title compound [13 g, 2 steps, yield 37%, [α]^{25D}+111° (c1.00, MeOH), optical purity 97% ee] was obtained as a white amorphous form by filtration and evaporation.

### c) (1S,2R,3R)-1-t-butoxycarbonylamino-2-methyl-3-phenyl-cyclopropanecarboxylic acid t-butyl ester (step 3-1)

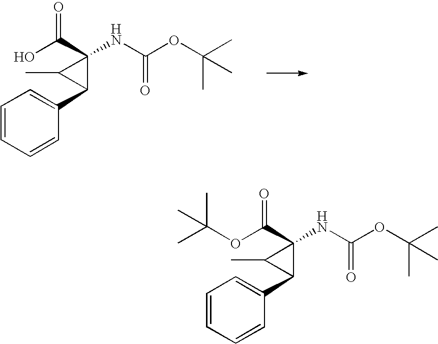

Under argon atmosphere, N,N-dimethylformamide di-t-butylacetal (5.0 mL, 21 mmol) was added dropwise to a solution of (1S,2R,3R)-1-t-butoxycarbonylamino-2-methyl-3-phenyl-cyclopropanecarboxylic acid (1.5 g, 5.2 mmol) obtained in Preparation Example 5-1 in toluene (15 mL) at 80° C. over 15 min, and the mixture was stirred for 1 hr. The obtained solution was cooled to 0° C. After saturated aqueous sodium hydrogen carbonate solution (15 mL) was added to the mixture, the organic layer was washed three times with water (10 mL) and dried over magnesium sulfate. Then, the title compound (1.8 g, yield 99%) was obtained as a pale-yellow oil by filtration and evaporation. The obtained product was used in the next step without purification.

### PRODUCTION EXAMPLE 3-2

### (1S,2R)-1-amino-2-phenylcyclopropanecarboxylic acid (step 3-2)

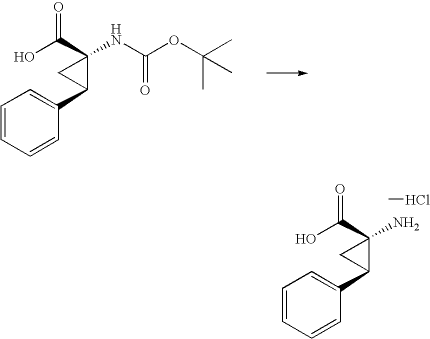

To commercially available (1S,2R)-1-t-butoxycarbonylamino-2-phenylcyclopropanecarboxylic acid (130 mg, 0.45 mmol) was added 4N hydrochloric acid dioxane solution (2.0 mL) and the mixture was stirred at room temperature for 1 hr. Diethyl ether (1.0 mL) was added and the mixture was stirred for 5 min. The resulting crystals were collected by filtration, washed with diethyl ether (1.0 mL) and dried under reduced pressure to give the title compound (81 mg, yield 84%) as a white powder.

### PRODUCTION EXAMPLE 3-3

### a) 1-(3-thiophen-2-yl-isoxazol-5-yl)-ethanone

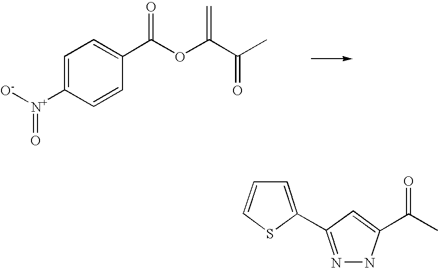

The title compound was synthesized according to the method described in known reference (Heterocycles 1993, 35, 591-598).

To a solution of 4-nitro-benzoic acid 1-methylene-2-oxo-propyl ester (10 g, 43 mmol) synthesized by the method of Helv. Chim. Acta (1981, 64, 188-197) and 2-thiophenecarbohydroxymoyl chloride (10 g, 62 mmol) synthesized by the method of Bioorg. Med. Chem. Lett. (2003, 13, 1795-1799) in chloroform (100 mL) was added dropwise triethylamine (9.0 mL, 62 mmol) under argon atmosphere at 0° C. over 30 min., and the mixture was stirred for 30 min. Then triethylamine (6.0 mL, 42 mmol) was added dropwise rapidly and the mixture was warmed gradually to room temperature.

After stirring at room temperature for 12 hrs, water (100 mL) was added to the obtained solution, and the mixture was filtrated through celite. The filtrate was separated into layers and extracted with chloroform (100 mL). After the organic layer was washed with 1N aqueous sodium hydroxide solution (40 mL), 1N aqueous hydrochloric acid solution (80 mL) and saturated aqueous sodium chloride solution (40 mL) were added sequentially, and the mixture was dried over sodium sulfate. After filtration and evaporation, the obtained residue was purified by silica gel chromatography (hexane:chloroform=1:1) to give the title compound (5.4 g, yield 66%) as light brown crystals.

### b) 5-(1,1-difluoroethyl)-3-thiophen-2-yl-isoxazole

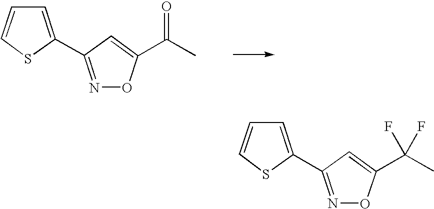

Under argon atmosphere, to a suspension of 1-(3-thiophene-2-yl-isoxazole-5-yl)-ethanone (6.0 g, 31 mmol) obtained in Preparation Example 5-10-a) in dichloromethane (30 mL) was added dropwise diethylaminosulfur trifluoride (DAST) (16 mL, 0.12 mol) over 5 min. at 0° C., and the suspension was warmed gradually to room temperature. After stirring at room temperature for 23 hrs, the obtained solution was transferred to a separatory funnel and added dropwise over 30 min to 4N aqueous sodium hydroxide solution (105 mL) cooled at 0° C. Then the obtained solution was warmed gradually to room temperature and filtrated through celite. The filtrate was separated into layers and extracted with chloroform (60 mL). Then the organic layer was sequentially washed with saturated aqueous sodium hydrogen carbonate solution (90 mL), 1N aqueous hydrochloric acid solution (60 mL) and saturated aqueous sodium chloride solution (30 mL), and dried over magnesium sulfate. After filtration and evaporation, the obtained residue was purified by silica gel chromatography (hexane:ethyl acetate=15:1) to give the title compound (6.2 g, 94%) as a brown oil.

### c) 5-[5-(1,1-difluoroethyl)-isoxazol-3-yl]-thiophene-2-sulfonic acid

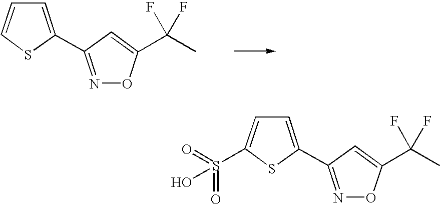

Under argon atmosphere, to a solution of 5-(1,1-difluoroethyl)-3-thiophene-2-yl-isoxazole (6.2 g, 31 mmol) obtained in Preparation Example 5-10-b) in chloroform (100 mL) was added chlorosulfonic acid (2.5 mL, 38 mmol), and the mixture was stirred at room temperature for 3 days. The obtained solution was filtrated and dried under reduced pressure to give the title compound (7.9 g, yield 93%) as a light brown powder.

### d) 5-[5-(1,1-difluoroethyl)-isoxazol-3-yl]-thiophene-2-sulfonic acid chloride (step 3-3)

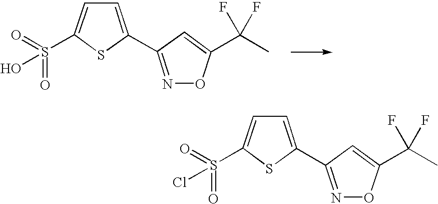

To a suspension of 5-[5-(1,1-difluoroethyl)-isoxazol-3-yl]-thiophene-2-sulfonic acid (9.5 g, 32 mmol) obtained in Preparation Example 5-10-c) in thionyl chloride (50 mL) was added dimethylformamide (1.0 mL) under argon atmosphere, and the suspension was stirred at 80° C. for 16 hrs. The obtained solution was concentrated, and chloroform (100 mL) was added. The mixture was concentrated twice, and chloroform (50 mL) was added to the residue. The obtained mixture was extracted twice, sequentially washed with water (20 mL) and saturated aqueous sodium chloride solution (10 mL), and dried over magnesium sulfate. After filtration and evaporation, the obtained residue was purified by silica gel chromatography (hexane:ethyl acetate=15:1) to give the title compound (6.9 g, 68%) as a yellow solid.

### PRODUCTION EXAMPLE 3-4

### (1S,2R)-1-(4′-chlorobiphenyl-4-sulfonylamino)-2-phenylcyclopropanecarboxylic acid (step 3-4)

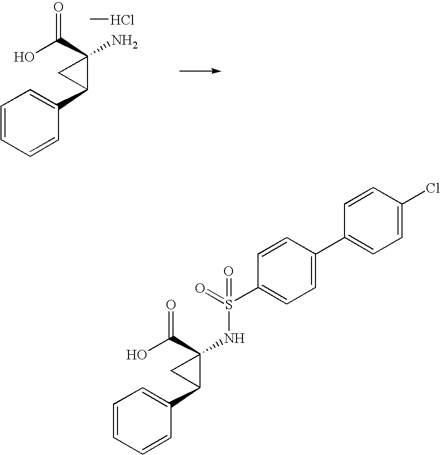

To a suspension of (1S,2R)-1-amino-2-phenylcyclopropanecarboxylic acid (80 mg, 0.38 mol) obtained in Production Example 3-2 in dioxane:water=1:1 (3.2 mL) mixture were successively added triethylamine (0.18 mL, 1.3 mmol), 4-chlorobiphenylsulfonyl chloride (110 mg, 1.1 mol) and N,N-dimethylaminopyridine (9.0 mg, 0.20 mmol) at 0° C. After stirring at room temperature for 12 hrs, 1N aqueous hydrochloric acid solution was added until the pH reached about 1 and the mixture was extracted twice with ethyl acetate (4.0 mL). After concentration, the obtained crude product was purified by thin layer silica gel chromatography (chloroform:methanol=7:1) to give the title compound (60 mg, yield 37%) as a white amorphous form.

### PRODUCTION EXAMPLE 3-5

### (1S,2R)-1-(4′-chlorobiphenyl-4-sulfonylamino)-2-phenylcyclopropanecarboxylic acid ethyl ester (step 3-5)

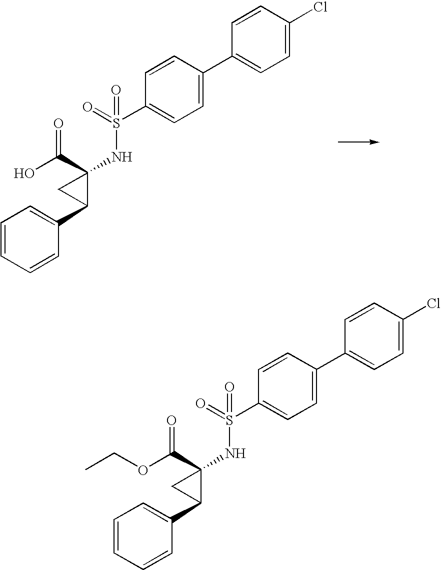

Under argon atomsphere, to a suspension of (1S,2R)-1-(4′-chlorobiphenyl-4-sulfonylamino)-2-phenylcyclopropanecarboxylic acid (40 mg, 0.094 mmol) obtained in Production Example 3-4 in ethanol (0.80 mL) was added dropwise thionyl chloride (0.014 mL, 0.19 mmol) at −20° C., and the mixture was warmed to room temperature. After stirring at 90° C. for 8 hrs, the solvent was removed under reduced pressure. Water (2.0 mL) was added to the residue and the mixture was extracted twice with ethyl acetate (4.0 mL), washed with a mixed solution of saturated aqueous sodium hydrogen carbonate solution:saturated aqueous sodium chloride solution=1:1 (2.0 mL) and dried over magnesium sulfate.

After filtration and solvent removal, the crude product of the title compound (39 mg, yield 91%) was obtained as a pale-brown solid.

### PRODUCTION EXAMPLE 3-6

### (1S,2R)-1-[(4′-chlorobiphenyl-4-sulfonyl)ethoxycarbonylmethylamino]-2-phenylcyclopropanecarboxylic acid ethyl ester (step 3-6)

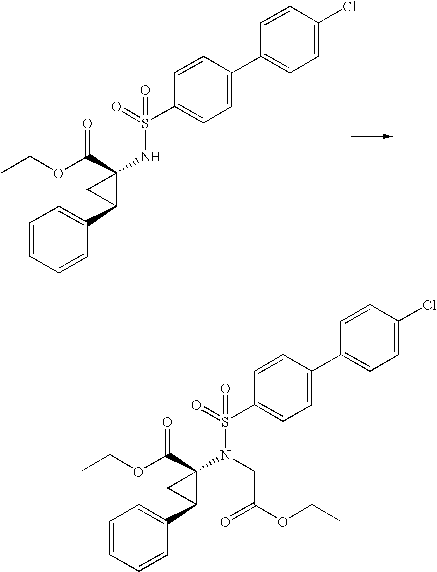

Under argon atomsphere, to a solution of (1S,2R)-1-(4′-chlorobiphenyl-4-sulfonylamino)-2-phenylcyclopropanecarboxylic acid ethyl ester (39 mg, 0.086 mmol) obtained in Production Example 3-5 in N,N-dimethylformamide (0.5 mL) were successively added bromoethyl acetate (0.011 mL, 0.090 mmol) and potassium carbonate (14 mg, 0.10 mmol) at room temperature and the mixture was stirred at 60° C. for 4 hrs. Water (1.0 mL) was added to the obtained reaction mixture at room temperature. The mixture was extracted with ethyl acetate (2.0 mL) and the extract was washed successively with water (2.0 mL) and saturated aqueous sodium chloride solution (0.50 mL) and dried over magnesium sulfate. After filtration and solvent removal, the obtained crude product was purified by thin layer silica gel chromatography (chloroform:ethyl acetate=10:1) to give the title compound (38 mg, yield 82%) as a pale-yellow oil.

### PRODUCTION EXAMPLE 3-6-2

### (1R*,2S*)-1-[(4′-chlorobiphenyl-4-sulfonyl)-cyanomethylamino]-2-phenylcyclopropanecarboxylic acid methyl ester (step 3-6)

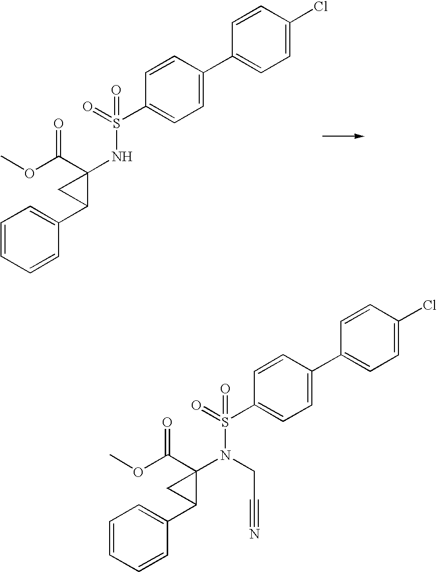

Under argon atomsphere, to a solution of (1R*,2S*)-1-(4′-chlorobiphenyl-4-sulfonylamino)-2-phenylcyclopropanecarboxylic acid methyl ester (2.0 g, 4.5 mmol) obtained by the same method as in Production Example 3-5 in N,N-dimethylformamide (20 mL) were successively added potassium carbonate (0.76 g, 5.5 mmol) and bromoacetonitrile (0.38 mL, 5.5 mmol) at room temperature and the mixture was stirred for 12 hrs. To a reaction suspension were added diethyl ether and water for layer separation, and the aqueous layer was extracted twice with diethyl ether. The combined organic layers were washed with water and saturated aqueous sodium chloride solution, and dried over sodium sulfate. After filtration and solvent removal, the obtained crude product was purified by silica gel chromatography (hexane:ethyl acetate=4:1-2:1) to give the title compound (2.2 g, yield>99%) as a white amorphous form.

### PRODUCTION EXAMPLE 4-1

### (S)-2-(4′-chlorobiphenyl-4-sulfonylamino)-3-hydroxy-propionic acid t-butyl ester (step 4-1)

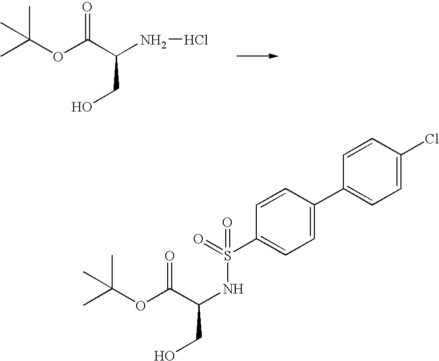

Under argon atomsphere, to a solution of L-serine t-butyl hydrochloride (5.0 g, 26 mmol) in tetrahydrofuran (50 mL) were successively added water (50 mL), sodium hydrogen carbonate (12 g, 150 mmol) and 4-chlorobiphenylsulfonyl chloride (8.1 g, 28 mmol). After stirring at room temperature for 16 hrs, the organic solvent was removed under reduced pressure and diisopropyl ether was added to the residue. The resulting crystals were collected by filtration and dried under reduced pressure to give the title compound (12 g, yield>99%) as a white solid.

### PRODUCTION EXAMPLE 4-2

### (S)-2-[(4′-chlorobiphenyl-4-sulfonyl)-t-butoxycarbonylmethylamino]-3-hydroxy-propionic acid t-butyl ester (step 4-2)

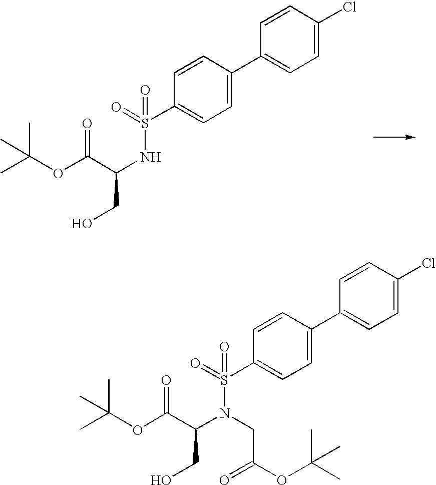

Under argon atomsphere, to a solution of (S)-2-(4′-chlorobiphenyl-4-sulfonylamino)-3-hydroxypropionic acid t-butyl ester (4.2 g, 10 mmol) obtained in Production Example 4-1 in N,N-dimethylformamide (42 mL) were successively added potassium carbonate (1.9 g, 13 mmol) and t-butyl bromoacetate (1.8 mL, 12 mmol) at room temperature and the mixture was stirred at 70° C. for 3 hrs. Water (100 mL) was added to the obtained reaction mixture at room temperature. The mixture was extracted with ethyl acetate (80 mL) and washed with water (40 mL) and saturated aqueous sodium chloride solution (20 mL), then dried over sodium sulfate. After filtration and solvent removal, the obtained crude product was purified by silica gel chromatography (hexane:diethyl ether=2:1) to give the title compound (4.5 g, yield 83%) as a white amorphous form.

### PRODUCTION EXAMPLE 4-3

### (S)-2-[(4′-chlorobiphenyl-4-sulfonyl)-t-butoxycarbonylmethylamino]-acrylic acid t-butyl ester (step 4-3)

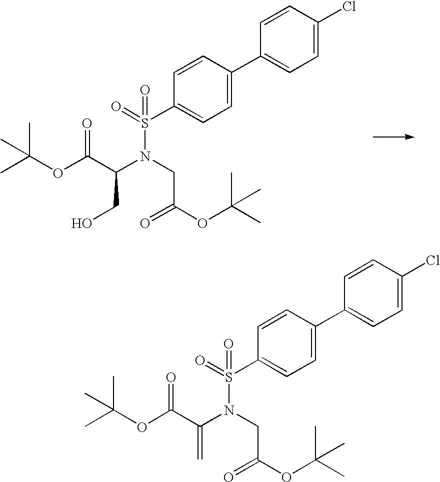

Under argon atomsphere, to a solution of (S)-2-[(4′-chlorobiphenyl-4-sulfonyl)-t-butoxycarbonylmethylamino]-3-hydroxy-propionic acid t-butyl ester (4.5 g, 8.5 mmol) obtained in Production Example 4-2 in tetrahydrofuran (45 mL) was added N-methylmorpholine (2.2 mL, 20 mmol) at room temperature, methanesulfonyl chloride (1.5 mL, 19 mmol) was added at 0° C. and the temperature was gradually raised to room temperature with stirring. The reaction mixture was cooled to 0° C. again, then 1,8-Diazabicyclo[5.4.0]-7-undecene (3.0 mL, 20 mmol) was added and the reaction temperature was gradually raised to room temperature with stirring. 1N aqueous potassium bisulfate solution (ca. 20 mL) was added to the obtained reaction mixture until the pH reached about 2. The mixture was extracted twice with ethyl acetate (40 mL) and dried over magnesium sulfate. After filtration and solvent removal, the obtained crude product was purified by silica gel chromatography (hexane:diethyl ether=4:1) to give the title compound (3.9 g, yield 89%) as a pale-yellow oil.

### PRODUCTION EXAMPLE 4-4

### (1R*,2S*)-1-[(4′-chlorobiphenyl-4-sulfonyl)-t-butoxycarbonylmethylamino]-2-(4′-cyano-phenyl)-cyclopropanecarboxylic acid t-butyl ester (step 4-4)

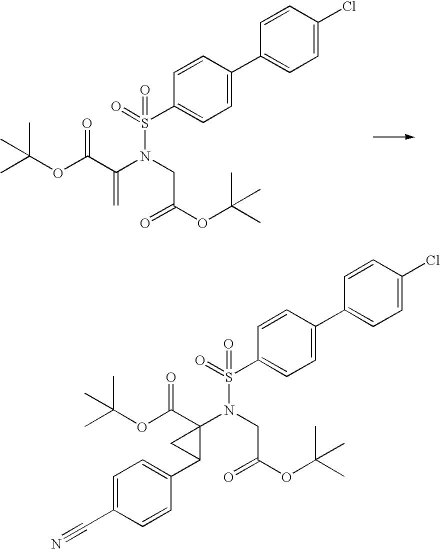

Under argon atomsphere, to a mixture of (S)-2-[(4′-chlorobiphenyl-4-sulfonyl)-t-butoxycarbonylmethylamino]-acrylic acid t-butyl ester (100 mg, 0.20 mmol) obtained in Production Example 4-3 and 1-(4-cyano-benzyl)-tetrahydro-thiophenium bromide (110 mg, 0.39 mmol) in tetrahydrofuran (2.0 mL) was added sodium hydride (16 mg, 0.40 mmol) at −40° C. The reaction temperature was gradually raised to room temperature and stirred at room temperature for 12 hrs. Saturated aqueous ammonium chloride solution (5.0 mL) was added to the reaction mixture and the mixture was extracted twice with diethyl ether (5.0 mL). The organic layer was washed three times with water (2.0 mL) and dried over magnesium sulfate. After filtration and solvent removal, the obtained crude product was purified by thin layer silica gel chromatography (hexane:acetone=3:1) to give the title compound (25 mg, yield 20%) as a pale-yellow amorphous form.

### PRODUCTION EXAMPLE 5-1

### (1R*,2R*)-1-[5-(4-chloro-phenyl)-thiophene-2-sulfonylamino]-2-hydroxymethyl-2-phenyl-cyclopropanecarboxylic acid t-butyl ester (step 5-1)

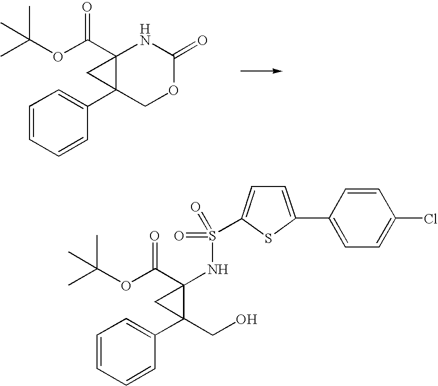

To a solution of (1R*,6R*)₃-oxo-6-phenyl-4-oxa-2-aza-bicyclo[4.1.0]heptan-1-carboxylic acid t-butyl ester (5.0 g, 17 mmol) obtained in Preparation Example 2-5 in tetrahydrofuran (50 mL) was sequentially added 15-crown-5 (0.34 mL, 1.7 mmol) and sodium hydride (liquid paraffin 40% added, 1.7 g, 41 mmol) at 0° C. under nitrogen atmosphere. After stirring for 5 min., the mixture was further stirred at room temperature for 30 min. The obtained solution was cooled to 0° C., and 5-(4-chlorophenyl)-thiophene-2-sulfonyl chloride (6.1 g, 21 mmol) was added. After stirring at 0° C. for 15 min., the mixture was stirred at room temperature for 6 hrs. To the obtained solution were sequentially added tetrahydrofuran (50 mL), methanol (100 mL) and 2N aqueous sodium hydroxide solution (17 mL, 69 mmol). After stirring for 15 hrs, the mixture was concentrated to about half the amount under reduced pressure. To the obtained solution was added 5% aqueous potassium hydrogen sulfate solution until the pH level read about 6. Then the solution was extracted three times with ethyl acetate (50 mL), washed with water (30 mL) and saturated aqueous sodium chloride solution (30 mL), and dried over sodium sulfate. After filtration and evaporation, the obtained residue was purified by silica gel chromatography (hexane:ethyl acetate=7:3) to give the title compound (3.6 g, yield 40%) as a pale-yellow amorphous form.

### PRODUCTION EXAMPLE 5-2

### (1R*,6S*)-2-[5-(4-chlorophenyl)-thiophene-2-sulfonyl]-6-phenyl-4-oxa-2-aza-bicyclo[4.1.0]heptane-1-carboxylic acid methyl ester (step 5-2)

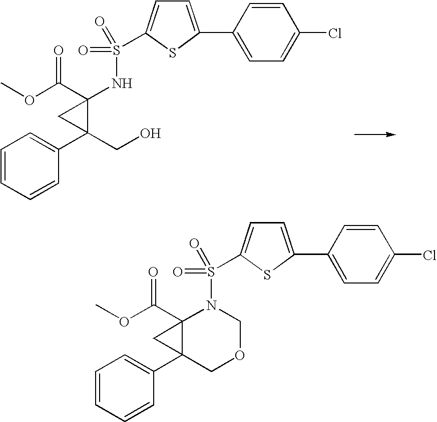

To a solution of (1R*,2R*)-1-[5-(4-chloro-phenyl)-thiophene-2-sulfonylamino]-2-hydroxymethyl-2-phenyl-cyclopropanecarboxylic acid methyl ester (28 mg, 0.059 mmol) obtained by the same method as in Production Example 5-1 in benzene (2.0 mL) were successively added p-formaldehyde (purity 95%, 19 mg, 0.59 mmol) and a catalytic amount of p-toluenesulfonic acid monohydrate (2.0 mg) at room temperature. The mixture was heated to reflux to remove water with a Dean Stark trap for 30 min. The mixture was cooled to room temperature and ethyl acetate and aqueous sodium hydrogen carbonate solution were added to the mixture. The mixture was extracted twice with ethyl acetate and the combined organic layers were washed with water and dried over sodium sulfate. After filtration and solvent removal, the residue was purified by silica gel chromatography (hexane:ethyl acetate=20:1-1:1) to give the title compound (12 mg, yield 42%) as a yellow solid.

### PRODUCTION EXAMPLE 6-1

### a) (1R*,2S*)-1-[(4′-chlorobiphenyl-4-sulfonyl)carboxymethylamino]-2-phenylcyclopropanecarboxylic acid methyl

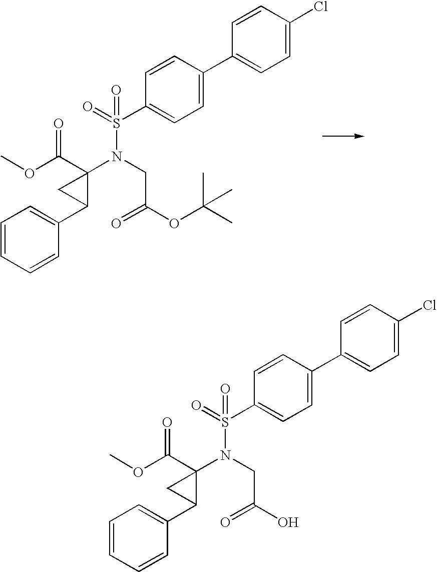

Under argon atomsphere, to a solution of (1R*,2S*)-1-[(4′-chlorobiphenyl-4-sulfonyl)-t-butoxycarbonylmethylamino]-2-phenylcyclopropanecarboxylic acid methyl ester (1.8 g, 3.3 mmol) obtained by the same method as in Production Example 3-6 in dichloromethane (24 mL) was added trifluoroacetic acid (8.0 mL) at 0° C. and the mixture was stirred at room temperature for 1 hr. The organic solvent was removed under reduced pressure to give the title compound (1.7 g, yield>99%) as a pale-yellow solid.

### b) (1R*,2S*)-1-[(4-chlorobiphenyl-4-sulfonyl)carbamoylmethylamino]-2-phenylcyclopropanecarboxylic acid methyl ester (step 6-1)

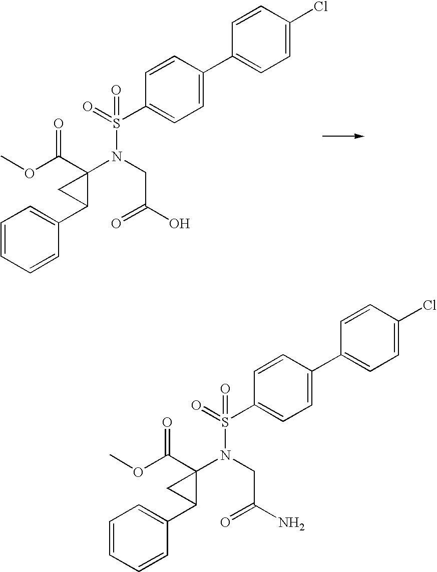

Under argon atomsphere, to a solution of (1R*,2S*) 1[(4′-chlorobiphenyl-4-sulfonyl)carboxymethylamino]-2-phenylcyclopropanecarboxylic acid methyl ester (500 mg, 1.0 mmol) obtained in Production Example 6-1 a) in tetrahydrofuran (5.0 mL) was added N,N′-carbonyldiimidazole (180 mg, 1.1 mmol) and the mixture was stirred at room temperature for 1 hr. 28% Aqueous ammonia (2.5 mL) was added to the reaction mixture and the mixture was stirred at room temperature for 1 hr. The organic solvent was removed under reduced pressure and the residue was extracted with ethyl acetate (5.0 mL), washed with water (2.0 mL) and saturated aqueous sodium chloride solution (1.0 mL) and dried over sodium sulfate. After filtration and solvent removal, the title compound was obtained (490 mg, yield 98%) as a pale-yellow solid.

### PRODUCTION EXAMPLE 6-1-2

### a) (1R*,2S*)-1-4′-chlorobiphenyl-4-sulfonyl-(N-hydroxycarbamimidoylmethyl)-amino]-2-phenylcyclopropanecarboxylic acid methyl ester

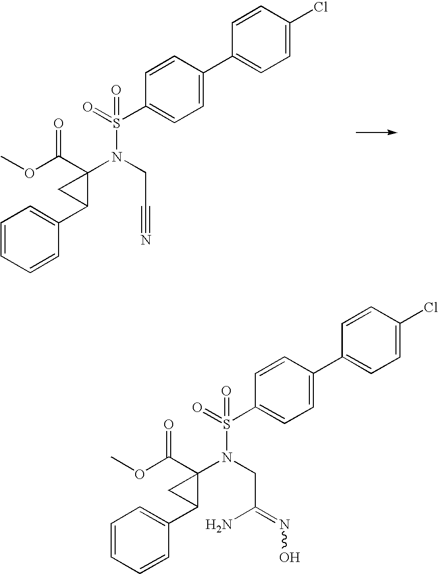

To a solution of (1R*,2S*)-1-[(4′-chlorobiphenyl-4-sulfonyl)-cyanomethylamino]-2-phenylcyclopropanecarboxylic acid methyl ester (1.6 g, 3.3 mmol) obtained in Production Example 3-6-2 in ethanol:dioxane=2:1 (24 mL) mixture was added dropwise an aqueous hydroxylamine solution (prepared by adding potassium carbonate (2.2 g, 16 mmol) to aqueous solution (8.0 mL) of hydroxylamine hydrochloride (1.1 g, 16 mmol) at 0° C.) at room temperature. The reaction mixture was diluted with ethanol (8.0 mL), and heated to reflux at 90° C. for 1.5 hrs. After the mixture was cooled to room temperature, ethyl acetate and water were added to the reaction mixture. The mixture was extracted with ethyl acetate, and the combined organic layers were washed with saturated aqueous sodium chloride solution, then dried over sodium sulfate. After filtration and solvent removal, the residue was azeoproped with toluene and dried under reduced pressure to give the title compound (1.6 g, yield 94%) as a white solid.

### b) (1R*,2S*)-1-[(4′-chlorobiphenyl-4-sulfonyl)-(5-oxo-4,5-dihydro[1.2.4]oxadiazol-3-ylmethyl)-amino]-2-phenylcyclopropanecarboxylic acid methyl ester (step 6-1)

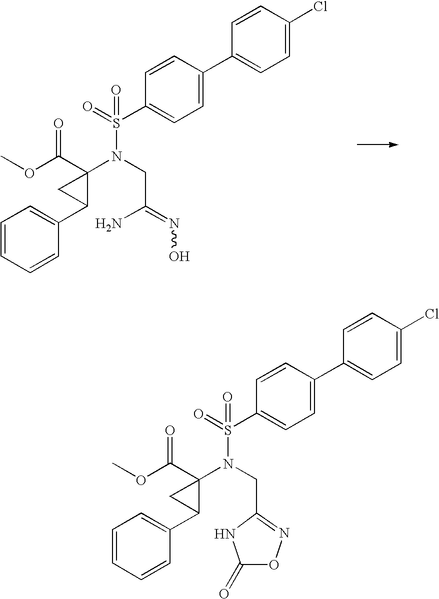

Under argon atomsphere, to a solution of (1R*,2S*)-1-[(4′-chlorobiphenyl-4-sulfonyl)-(N-hydroxycarbamimidoylmethyl)-amino]-2-phenylcyclopropanecarboxylic acid methyl ester (0.51 g, 1.0 mmol) obtained in Production Example 6-1-2 a) in N,N-dimethylformamide (5.0 mL) were successively added pyridine (0.085 mL, 1.1 mmol) and isobutyl chlorocarbonate (0.14 mL, 1.1 mmol) at 0° C., then the mixture was stirred at 0° C. for 30 min. Diethyl ether and water were added to the reaction mixture and the mixture was extracted with diethyl ether. The combined organic layers were washed with water and saturated aqueous sodium chloride solution and dried over sodium sulfate. After filtration and solvent removal, the residue was azeoproped with toluene and dried under reduced pressure to give a white amorphous form. Under argon atomsphere, this amorphous form was dissolved in xylene (15 mL) and the solution was heated to reflux at 150° C. for 12 hrs. After the mixture was cooled to room temperature, the solvent was removed, then the obtained crude product was purified by silica gel chromatography (hexane:ethyl acetate=4:1-1:1) to give the title compound (0.23 g, yield 43%) as a brown viscous oil.

### EXAMPLE 1

### a) (1S,2R)-1-Amino-2-phenylcyclopropanecarboxylic acid

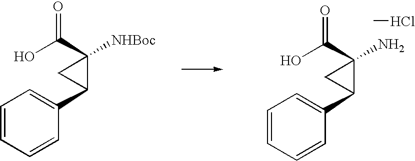

To commercially available (1R,2S)-1-tert-butoxycarbonylamino-2-phenylcyclopropanecarboxylic acid (130 mg, 0.45 mmol) was added 4N hydrochloric acid-1,4-dioxane solution (2.0 mL, 16 v/w) and the mixture was stirred for 1 hr at room temperature. Diethyl ether (1.0 mL) was added thereto and the mixture was stirred for 5 min, after which the resulting crystals were collected by filtration. The crystals were washed with diethyl ether (1.0 mL) and dried under reduced pressure to give the title compound (81 mg, white powder, yield 84%).

¹H-NMR (DMSO, 300 MHz): 1.84(dd, J=6.0, 9.0 Hz, 1H), 2.03(dd, J=6.0, 9.0 Hz, 1H), 2.99(t, J=10.5 Hz, 1H), 7.20-7.40(m, 5H), 8.29(br, 3H)

### b) (1S,2R)-1-(4′-Chlorobiphenyl-4-sulfonylamino)-2-phenylcyclopropanecarboxylic acid

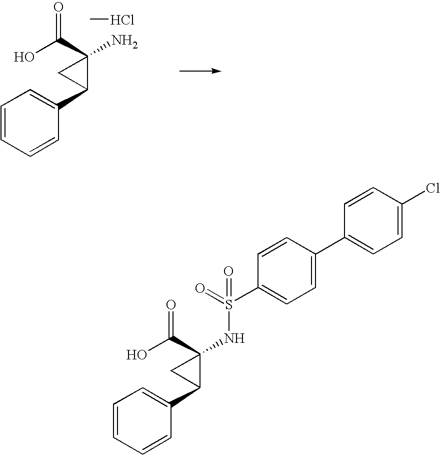

To a suspension of (1S,2R)-1-amino-2-phenylcyclopropanecarboxylic acid (80 mg, 0.38 mol) obtained in the above a) in 1,4-dioxane:water=1:1 (3.2 mL, 40 v/w) were successively added triethylamine (0.18 mL, 1.3 mmol), 4-chlorobiphenylsulfonic acid chloride (110 mg, 1.1 mol) and N,N-dimethylaminopyridine (9.0 mg, 0.20 mmol) at 0° C. The mixture was stirred for 12 hrs at room temperature, and 1N hydrochloric acid was added thereto until its pH reached approximately 1. The organic layer was extracted twice with ethyl acetate (4.0 mL) and concentrated. Then, the obtained crude product was purified by thin-layer silica gel chromatography (chloroform:methanol=7:1) to give the title compound (60 mg, white amorphous solid, 37%).

### c) (1S,2R)-1-(4′-Chlorobiphenyl-4-sulfonylamino)-2-phenylcyclopropanecarboxylic acid ethyl ester

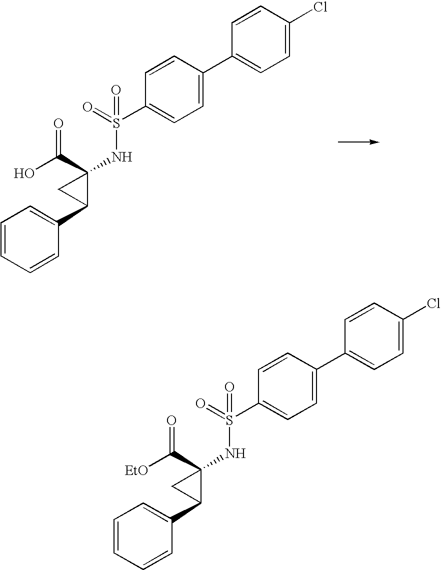

Under argon atmosphere, to a suspension of (1S,2R)-1-(4′-chlorobiphenyl-4-sulfonylamino)-2-phenylcyclopropanecarboxylic acid (40 mg, 0.094 mmol) obtained in the above b) in ethanol (0.80 mL, 20 v/w) was added dropwise thionyl chloride (0.014 mL, 0.19 mmol) at −20° C. After warming to room temperature, the reaction mixture was stirred at 90° C. for 8 hrs. The solvent was concentrated under reduced pressure and water (2.0 mL) was added to the residue. The organic layer was extracted twice with ethyl acetate (4.0 mL), washed with a mixed solution of a saturated aqueous sodium hydrogen carbonate solution:saturated brine=1:1 (2.0 mL) and dried over magnesium sulfate. After filtration, the solvent was evaporated off to give a crude product (39 mg, 91%) of the title compound as a pale brown solid.

¹H-NMR(CDCl₃, 300 MHz): 0.61 (t, J=7.5 Hz, 3H), 2.14-2.28(m, 2H), 2.86(t, J=9.0 Hz, 1H), 3.25-3.33(m, 1H), 3.39-3.47(m, 1H), 5.85(br, 1H), 7.13-7.24(m, 4H), 7.41-7.55(m, 5H), 7.67(dd, J=3.0, 6.0 Hz, 2H), 7.97(dd, J=3.0, 6.0 Hz, 2H)

### d) (1S,2R)-1-[(4′-Chlorobiphenyl-4-sulfonyl)ethoxycarbonylmethylamino]-2-phenylcyclopropanecarboxylic acid ethyl ester

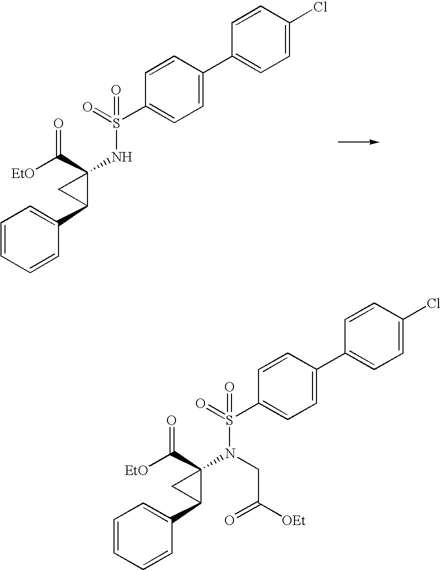

Under argon atmosphere, to a solution of (1S,2R)-1-(4′-chlorobiphenyl-4-sulfonylamino)-2-phenylcyclopropanecarboxylic acid ethyl ester (39 mg, 0.086 mmol) obtained in the above c) in N,N-dimethylformamide (0.5 mL, 13 v/w) were successively added bromoethyl acetate (0.011 mL, 0.090 mmol) and potassium carbonate (14 mg, 0.10 mmol) at room temperature, and the mixture was stirred at 60° C. for 4 hrs. To the obtained reaction solution was added water (1 mL) at room temperature. The organic layer was extracted with ethyl acetate (2.0 mL), washed successively with water (2.0 mL) and saturated brine (0.50 mL), and dried over magnesium sulfate. The residue was filtered and the solvent was evaporated off. The obtained crude product was purified by thin-layer silica gel chromatography (chloroform:ethyl acetate=10:1) to give the title compound (38 mg, 82%) as a pale yellow oil.

¹H-NMR(CDCl₃, 300 MHz): 0.73 (t, J=7.5 Hz, 3H), 1.33(t, J=4.5 Hz, 3H), 1.98-2.18(m, 1H), 2.23-2.44(m, 1H), 2.88-3.77(m, 2H), 4.18-4.89(m, 2H), 7.04-7.34(m, 5H), 7.45(d, J=9.0 Hz, 2H), 7.53(d, J=9.0 Hz, 2H), 7.68(d, J=9.0 Hz, 2H), 8.00(d, J=9.0 Hz, 2H)

### e) (1S,2R)-1-[Carboxymethyl-(4′-chloro-biphenyl-4-sulfonyl)-amino]-2-phenylcyclopropanecarboxylic acid

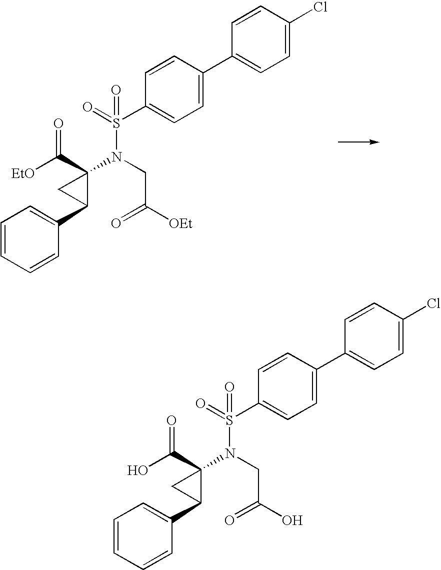

Under argon atmosphere, to a solution of (1S,2R)-1-[(4′-chlorobiphenyl-4-sulfonyl)ethoxycarbonylmethylamino]-2-phenylcyclopropanecarboxylic acid ethyl ester (38 mg, 0.070 mmol) obtained in the above d) in tetrahydrofuran (0.40 mL, 10 v/w) were successively added methanol (0.4 mL, 10 v/w) and 4N aqueous sodium hydroxide solution (0.40 mL, 10 v/w), and the mixture was stirred at 90° C. for 12 hrs. Then, the organic solvent was concentrated under reduced pressure and 1N hydrochloric acid was added to the residue until its pH reached approximately 1. The organic layer was extracted twice with ethyl acetate (2.0 mL) and concentrated, and to the obtained crude product were gradually added diethyl ether and hexane. The precipitated crystals were collected by filtration and washed successively with a mixed solution of diethyl ether:hexane=1:2 and water, and dried under reduced pressure to give the title compound (26 mg, pale brown powder, yield 76%).

Melting point 191.0-196.6° C. (decomposition)

### EXAMPLE 1-30

(1R*,2S*)-1-[carboxymethyl-(4′-chloro-biphenyl-4-sulfonyl)-amino]-2-(4-cyano-phenyl)-cyclopropanecarboxylic acid

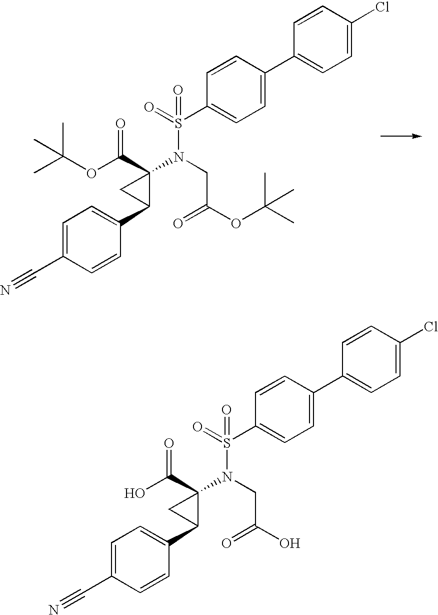

Under argon atmosphere, to (1R*,2S*)-1-[(4′ chlorobiphenyl-4-sulfonyl)ₜ-butoxycarbonylmethylamino]-2-(4-cyano-phenyl)-cyclopropanecarboxylic acid t-butyl ester (20 mg, 0.040 mmol) obtained in Production Example 4-4 was added trifluoroacetic acid (0.50 mL) and the mixture was stirred at room temperature for 1 hr. The solvent was removed under reduced pressure, and the residue was azeotroped with chloroform and dissolved in a small amount of diethyl ether. Hexane was added and the precipitated crystals were collected by filtration and dried to give the title compound (7.0 mg, yield 34%) as a pale-yellow powder.

### EXAMPLE 1-36

(1R*,2S*)-1-[(4′-chlorobiphenyl-4-sulfonyl)carboxymethylamino]-2-phenylcyclopropanecarboxylic acid (step 6-2)

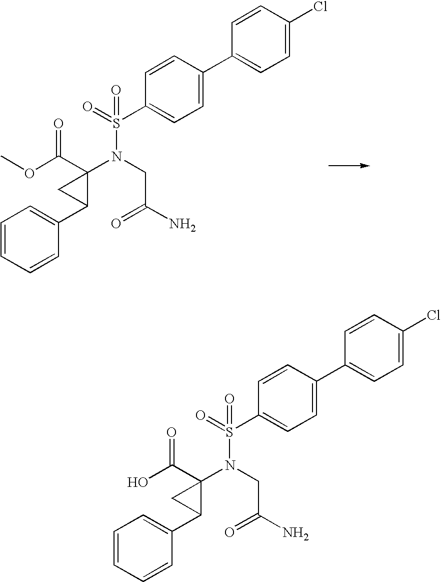

Under argon atmosphere, to a solution of (1R*,2S*)-1-[(4′-chlorobiphenyl-4-sulfonyl) carbamoylmethylamino]-2-phenylcyclopropanecarboxylic acid methyl ester (50 mg, 0.10 mmol) obtained in Production Example 6-1 in pyridine (0.5 mL) was added lithium iodide (67 mg, 0.50 mmol) and the mixture was stirred at 120° C. for 12 hrs. The organic solvent was concentrated under reduced pressure, and the residue was extracted with ethyl acetate (2.0 mL), washed successively with 1N aqueous hydrochloric acid solution (2.0 mL), water (1.0 mL) and saturated aqueous sodium chloride solution (0.50 mL) and dried over magnesium sulfate. After filtration and solvent removal, methanol was added to the obtained crude product. The precipitated crystals were filtered and vacuum dried to give the title compound (31 mg, yield 67%) as a white powder.

### EXAMPLE 1-64

(R*,2S*)-1-[(4′-chlorobiphenyl-4-sulfonyl)-(5-oxo-4,5-dihydro[1.2.4]oxadiazol-3-ylmethyl)-amino]-2-phenylcyclopropanecarboxylic acid (step 6-2)

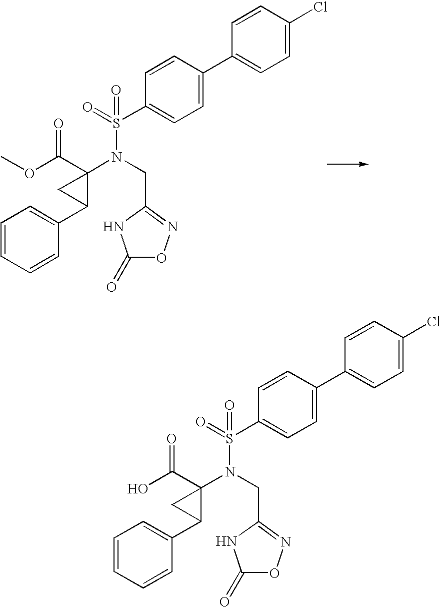

To a solution of (1R*,2S*)-1-(4′-chlorobiphenyl-4-sulfonyl)-(5-oxo-4,5-dihydro[1.2.4]oxadiazol-3-ylmethyl)-amino]-2-phenylcyclopropanecarboxylic acid methyl ester (0.12 g, 0.21 mmol) obtained in Production Example 6-1-2 in tetrahydrofuran (2.1 mL) were successively added methanol (2.1 mL), water (1.6 mL) and 4N aqueous lithium hydroxide solution (0.53 mL, 2.1 mmol). The reaction solution was refluxed at 90° C. for 14 hrs. The mixture was allowed to cool to room temperature and concentrated under reduced pressure. 2N aqueous hydrochloric acid solution was added until the pH reached about 2, and the precipitate was collected by filtration. After the filtered solid was purified by silica gel chromatography (chloroform:methanol=100:0-7:1), Hexane and chloroform were added to the residue. The precipitated crystals were collected by filtration and vacuum dried to give the title compound (64 mg, yield 57%) as a white solid.

melting point: 168-172° C. (decom)

### (Examples 1-2 to 1-115)

In the same manner as in Examples 1, 1-30, 1-36 and 1-64, the compounds of Examples 1-2 to 1-29, 1-31 to 1-35, 1-37 to 1-63 and 1-65 to 1-115 were obtained.

The structural formulas of the compounds of Examples 1 to 1-115 are shown in Tables 1-1 to 1-23.

### EXAMPLE 2

### (1R*,6S*)-2-[5-(4-chlorophenyl)-thiophene-2-sulfonyl]-6-phenyl-4-oxa-2-aza-bicyclo[4.1.0]heptane-1-carboxylic acid (step 6-2)

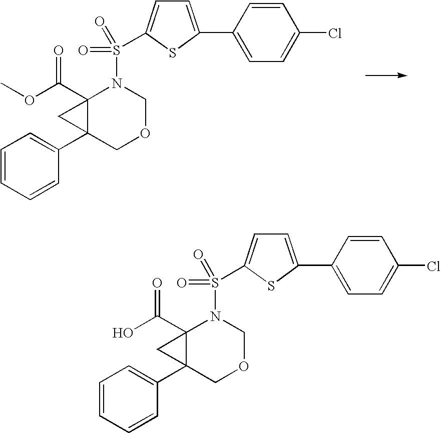

To a solution of (1R*,6S*)-2-[5-(4-chlorophenyl)-thiophene-2-sulfonyl]-6-phenyl-4-oxa-2-aza-bicyclo[4.1.0]heptane-1-carboxylic acid methyl ester (12 mg, 0.025 mmol) obtained in Production Example 5-2 in isopropyl alcohol (0.24 mL) were successively added dioxane (0.24 mL) and 4N aqueous sodium hydroxide solution (0.12 mL) and the mixture was stirred at 90° C. for 24 hrs. 4N Aqueous sodium hydroxide solution (0.12 mL), isopropyl alcohol (0.24 mL) and dioxane (0.24 mL) were supplemented and the mixture was refluxed at 100° C. for 12 hrs. The mixture was allowed to cool to room temperature, and acidified with 1N aqueous hydrochloric acid solution (0.96 mL) and extracted three times with ethyl acetate. The combined organic layers were washed with water and dried over sodium sulfate. After filtration and solvent removal, the residue was purified by silica gel chromatography (chloroform:methanol=100:0-7:1) to give the title compound (2.0 mg, yield 17%) as a yellow viscous oil.

### EXAMPLE 2-2

### (1R*, 5R*,6S*)-2-(4′-chlorobiphenyl-4-sulfonyl)-6-phenyl-2-aza-bicyclo[3.1.0]hexane-1-carboxylic acid (step 7-1)

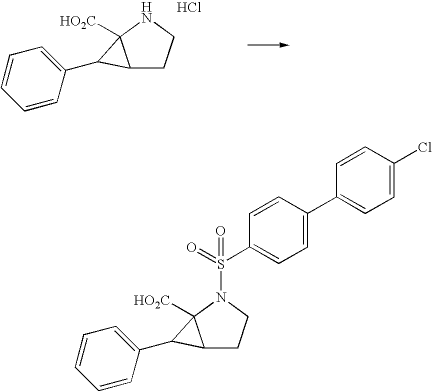

Under argon atmosphere, to a solution of (1R*,5R*,6S*)-6-phenyl-2-aza-bicyclo[3.1.0]hexane-1-carboxylic acid hydrochloride (11 mg, 0.047 mmol) synthesized by the method known in literature (Tetrahedron 1989, 45, 6091-6100) in water (0.30 mL) were successively added dioxane (0.30 mL), 4-chlorobiphenylsulfonyl chloride (14 mg, 0.049 mmol), triethylamine (23 μL, 0.17 mmol) and N,N-dimethylaminopyridine (1.0 mg, 0.0080 mmol). After stirring at room temperature for 12 hrs, 1N aqueous hydrochloric acid solution was added until the pH reached about 1. The organic layer was extracted twice with ethyl acetate (1.0 mL), washed with saturated aqueous sodium chloride solution and concentrated. The obtained crude product was purified by thin layer silica gel chromatography (chloroform:methanol=15:1) to give the title compound (8.0 mg, yield 38%) as a white amorphous form.

### (Examples 2-2 to 2-27)

In the same manner as in Examples 2 and 2-2, the compounds of Examples 2-3 to 2-27 were obtained.

The structural formulas of the compounds of Examples 2 to 2-27 are shown in Tables 2-1 to 2-6.

| | | |
|---|---|---|
| Example | Structural formula | NMR |

TABLE 1-1

| | | |
|---|---|---|
| 1 | 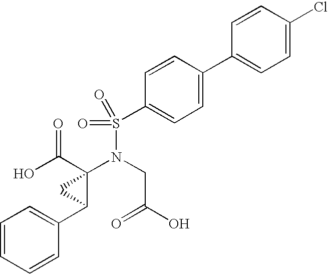 | (DMSO-d6-300) 1.76-1.90(m, 1H), 2.15-2.32(m, 1H), 2.91-3.07(m, 1H), 4.15-4.30(m, 1H), 4.41-4.61(m, 1H), 7.11-7.33(m, 5H), 7.58(d, J=9.0 Hz, 2H), 7.80(d, J=9.0 Hz, 2H), 7.87-7.96(m, 4H) |
| 1-2 | 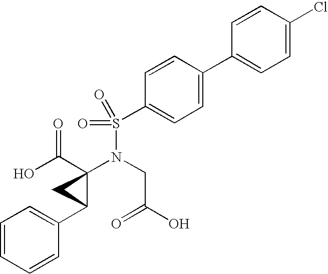 | (DMSO-d6-300) 1.76-1.90(m, 1H), 2.15-2.32(m, 1H), 2.91-3.07(m, 1H), 4.15-4.30(m, 1H), 4.41-4.61(m, 1H), 7.11-7.33(m, 5H), 7.58(d, J=9.0 Hz, 2H), 7.80(d, J=9.0 Hz, 2H), 7.87-7.96(m, 4H) |
| 1-3 | 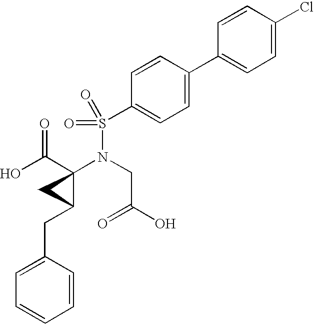 | (DMSO-d6-300) 1.54-1.72(m, 1H), 1.88-2.05(m, 1H), 2.59-2.80(m, 1H), 3.45-3.59(m, 1H), 4.15-4.34(m, 1H), 7.05-7.32(m, 5H), 7.58(d, J=6.0 Hz, 2H), 7.79(d, J=9.0 Hz, 2H), 7.86(br, 4H) |
| 1-4 | 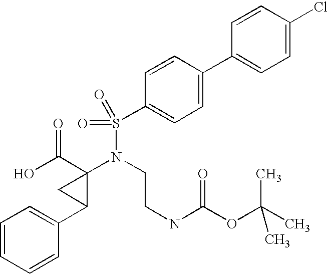 | (DMSO-d6-300) 1.37(s, 9H), 1.73-1.83(m, 1H), 2.16-2.31(m, 1H), 2.90-3.05(m, 1H), 3.08-3.78(m, 4H), 7.05-7.34(m, 5H), 7.58(d, J=9.0 Hz, 2H), 7.79(d, J=9.0 Hz, 2H), 7.84-7.95(m, 4H), 12.25(brs, 1H) |
| 1-5 | 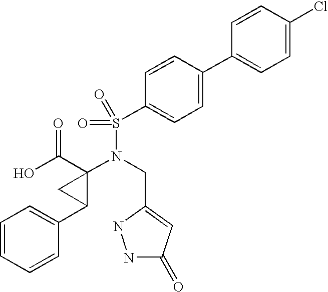 | (DMSO-d6-300(120° C.)) 1.72(dd, J=6.0, 9.0 Hz, 1H), 2.09(dd, J=6.0, 9.0 Hz, 1H), 3.02(t, J=9.0 Hz, 1H), 4.58(d, J=18.0 Hz, 1H), 4.69(d, J=18.0 Hz, 1H), 5.33(s, 1H), 7.11-7.28(m, 5H), 7.52(d, J=9.0 Hz, 2H), 7.73(d, J=9.0 Hz, 2H), 7.80(d, J=9.0 Hz, 2H), 7.86(d, J=9.0 Hz, 2H) |

TABLE 1-2

| | | |
|---|---|---|
| 1-6 | 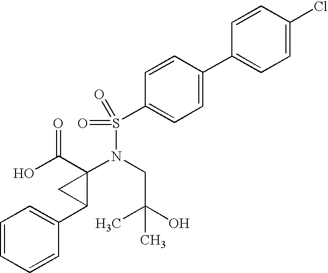 | (DMSO-d6-300 (120° C.)) 1.22(s, 6H), 1.82(dd, J=6.0, 9.0 Hz, 1H), 2.11(dd, J=6.0, 9.0 Hz, 1H), 3.38(t, J=9.0 Hz, 1H), 3.51-3.66(m, 2H), 7.11-7.26(m, 3H), 7.34(d, J=6.0 Hz, 2H), 7.52(d, J=9.0 Hz, 2H), 7.72(d, J=6.0 Hz, 2H), 7.81(d, J=6.0 Hz, 2H), 7.92(d, J=6.0 Hz, 2H) |
| 1-7 | 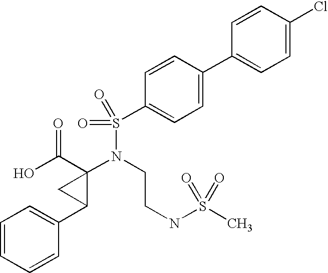 | (DMSO-d6-300) 1.18-1.28(m, 1H), 1.75-1.85(m, 1H), 2.20-2.33(m, 1H), 2.97(s, 3H), 3.03-3.46(m, 2H), 3.48-3.68(m, 1H), 3.71-3.87(m, 1H), 7.16-7.42(m, 5H), 7.58(d, J=9.0 Hz, 2H), 7.79(d, J=9.0 Hz, 2H), 7.85-7.96(m, 4H), 12.26(br, 1H) |
| 1-8 | 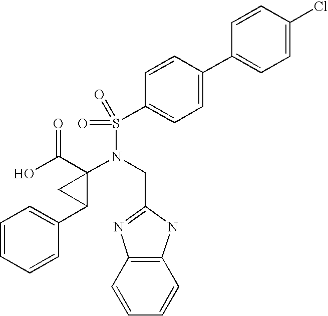 | (DMSO-d6-300) 1.73-1.82(m, 1H), 2.25-2.36(m, 1H), 3.11-3.24(m, 1H), 4.89-5.00(m, 1H), 5.05-5.22(m, 1H), 7.04-7.24(m, 7H), 7.44-7.59(m, 4H), 7.61-7.79(m, 4H), 7.84(d, J=9.0 Hz, 2H) |
| 1-9 | 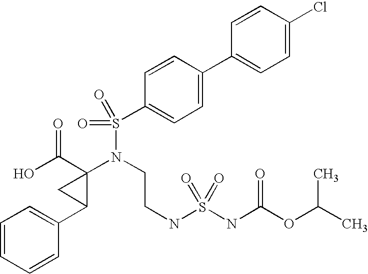 | (DMSO-d6-300) 1.09(s, 3H), 1.14(s, 3H), 1.21-1.34(m, 1H), 1.70-1.82(m, 1H), 2.19-2.31(m, 1H), 2.90-3.17(m, 2H), 3.49-3.85(m, 2H), 4.61-4.80(m, 1H), 7.13-7.38(m, 5H), 7.58(d, J=9.0 Hz, 2H), 7.79(d, J=9.0 Hz, 2H), 7.85-7.93(m, 4H) |
| 1-10 | 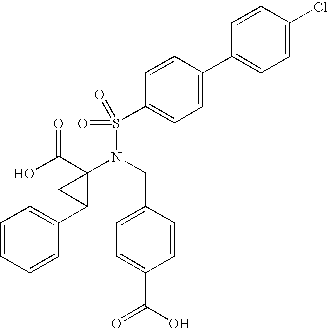 | (DMSO-d6-300) 1.80-2.13(m, 1H), 2.20-2.35(m, 1H), 2.56-2.80(m, 1H), 4.58-5.23(m, 2H), 6.85-8.18(m, 17H), 12.11-13.07(m, 2H) |

TABLE 1-3

| | | |
|---|---|---|
| 1-11 | 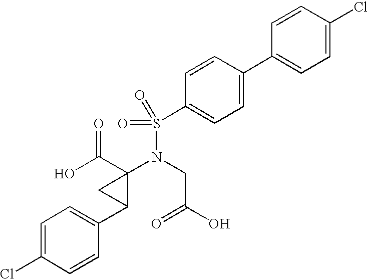 | (DMSO-d6-300) 1.21-1.30(m, 1H), 2.13-2.21(m, 1H), 2.71-2.83(m, 1H), 3.86(d, J=18.0 Hz, 1H), 4.04(d, J=18.0 Hz, 1H), 7.09-7.18(m, 2H), 7.27(d, J=9.0 Hz, 2H), 7.58(d, J=9.0 Hz, 2H), 7.81(d, J=9.0 Hz, 2H), 7.83-7.95(m, 4H) |
| 1-12 | 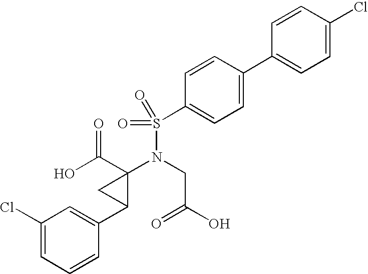 | (DMSO-d6-300) 1.80-1.93(m, 1H), 2.17-2.35(m, 1H), 2.94-3.09(m, 1H), 4.18-4.35(m, 1H), 4.41-4.60(m, 1H), 7.09-7.36(m, 4H), 7.58(d, J=9.0 Hz, 2H), 7.80(d, J=9.0 Hz, 2H), 7.87-7.97(m, 4H) |
| 1-13 | 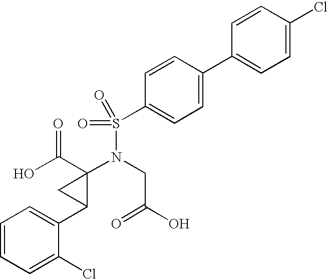 | (DMSO-d6-300) 2.03(dd, J=6.0, 9.0 Hz, 1H), 2.25-2.40(m, 1H), 3.12(t, J=9.0 Hz, 1H), 4.47(d, J=18.0 Hz, 1H), 4.60(d, J=18.0 Hz, 1H), 7.21-7.30(m, 3H), 7.35-7.43(m, 1H), 7.58(d, J=9.0 Hz, 2H), 7.80(d, J=9.0 Hz, 2H), 7.91(d, J=9.0 Hz, 2H), 7.95(d, J=9.0 Hz, 2H) |
| 1-14 | 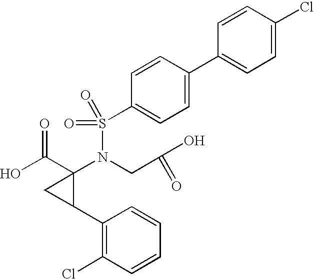 | (DMSO-d6-300) 1.99(dd, J=6.0, 9.0 Hz, 1H), 2.12(dd, J=6.0, 9.0 Hz, 1H), 2.80(t, J=9.0 Hz, 1H), 3.49(d, J=18.0 Hz, 2H), 4.15-4.29(m, 2H), 6.85-6.94(m, 1H), 7.25-7.34(m, 2H), 7.37-7.44(m, 1H), 7.61(d, J=9.0 Hz, 2H), 7.86(d, J=9.0 Hz, 2H), 7.95(d, J=9.0 Hz, 2H), 8.04(d, J=6.0 Hz, 2H) |
| 1-15 | 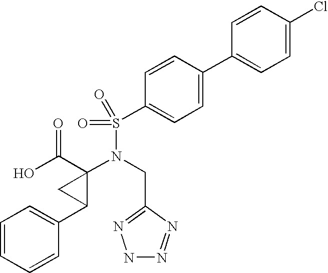 | (DMSO-d6-400) 1.64-1.90(m, 1H), 2.19-2.34(m, 1H), 3.08-3.24(m, 1H), 4.99(d, J=20.0 Hz, 1H), 5.10-5.24(m, 1H), J=8.0 Hz, 2H), 7.71-7.95(m, 6H) |

TABLE 1-4

| | | |
|---|---|---|
| 1-16 | 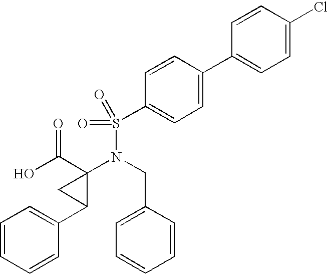 | (DMSO-d6-400) 1.81-2.00(m, 1H), 2.13-2.32(m, 1H), 2.54-2.74(m, 1H), 4.46-4.67(m, 1H), 4.90-5.14(m, 1H), 6.56-6.72(m, 1H), 6.78-6.89(m, 1H), 6.94-7.05(m, 1H), 7.10-7.40(m, 6H), 7.57(d, J=12.0 Hz, 2H), 7.76(d, J=8.0 Hz, 2H), 7.83(brs, 4H), 9.43(brs, 1H), 12.33(brs, 1H) |
| 1-17 | 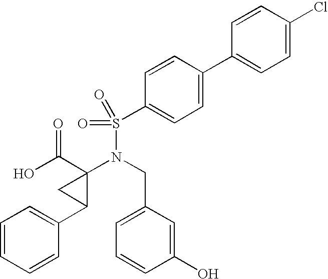 | (DMSO-d6-400) 1.81-2.00(m, 1H), 2.13-2.32(m, 1H), 2.54-2.74(m, 1H), 4.46-4.67(m, 1H), 4.90-5.14(m, 1H), 6.56-6.72(m, 1H), 6.78-6.89(m, 1H), 6.94-7.05(m, 1H), 7.10-7.40(m, 6H), 7.57(d, J=12.0 Hz, 2H), 7.76(d, J=8.0 Hz, 2H), 7.83(brs, 4H), 9.43(brs, 1H), 12.33(brs, 1H) |
| 1-18 | 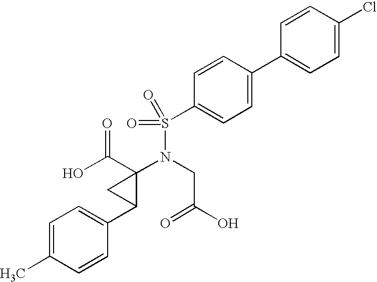 | (DMSO-d6-400) 1.21-1.29(m, 1H), 1.71-1.85(m, 1H), 2.23(s, 3H), 2.51-2.57(m, 1H), 4.11-4.26(m, 2H), 6.99-7.07(m, 4H), 7.57(d, J=12.0 Hz, 2H), 7.78(d, J=12.0 Hz, 2H), 7.87-7.94(m, 4H) |
| 1-19 | 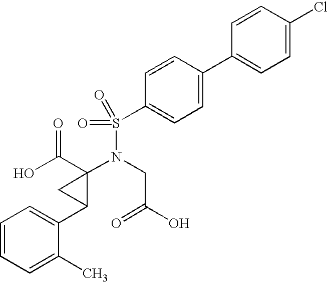 | (DMSO-d6-400) 1.20-1.29(m, 1H), 2.25(s, 3H), 2.31-2.36(m, 1H), 2.51-2.55(m, 1H), 4.18-4.35(m, 2H), 7.01-7.12(m, 4H), 7.57(d, J=6.0 Hz, 2H), 7.79(d, J=6.0 Hz, 2H), 7.88-7.96(m, 4H) |
| 1-20 | 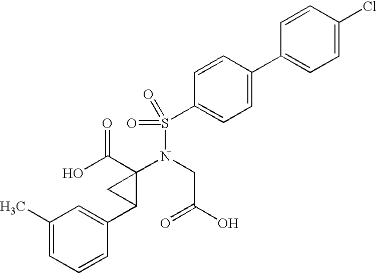 | (DMSO-d6-400) 1.21-1.29(m, 1H), 1.74-1.86(m, 1H), 2.23(s, 3H), 2.85-3.00(m, 1H), 4.13-4.31(m, 2H), 6.90-7.18(m, 4H), 7.57(d, J=6.0 Hz, 2H), 7.78(d, J=6.0 Hz, 2H), 7.85-7.95(m, 4H) |

TABLE 1-5

| | | |
|---|---|---|
| 1-21 | 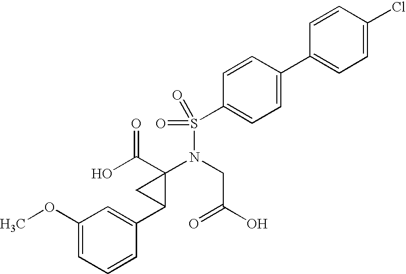 | (DMSO-d6-400) 1.74-1.86(m, 1H), 2.15-2.23(m, 1H), 2.89-3.00(m, 1H), 3.69(s, 3H), 4.16-4.27(m, 1H), 4.46-4.59(m, 1H), 6.66-6.79(m, 3H), 7.11-7.20(m, 1H), 7.57(d, J=6.0 Hz, 2H), 7.77(d, J=6.0 Hz, 2H), 7.86-7.94(m, 4H) |
| 1-22 | 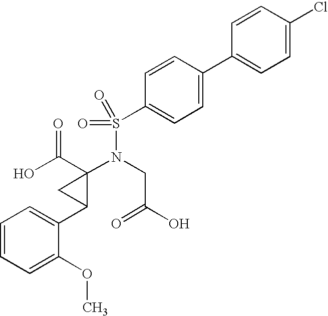 | (DMSO-d6-400) 1.81-1.91(m, 1H), 2.15-2.22(m, 1H), 2.87-2.96(m, 1H), 3.71(s, 3H), 4.41-4.58(m, 2H), 6.82(t, J=6.0 Hz, 1H), 6.88(d, J=6.0 Hz, 1H), 7.01(d, J=6.0 Hz, 1H), 7.18(t, J=6.0 Hz, 1H), 7.57(d, J=9.0 Hz, 2H), 7.78(d, J=9.0 Hz, 2H), 7.86-7.94(m, 4H) |
| 1-23 | 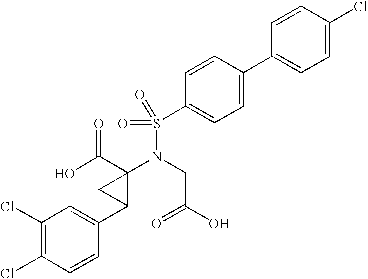 | (DMSO-d6-400) 1.83-1.91(m, 1H), 2.21-2.30(m, 1H), 2.94-3.07(m, 1H), 4.23-4.32(m, 1H), 4.41-4.53(m, 1H), 7.17-7.27(m, 1H), 7.49-7.54(m, 1H), 7.57(d, J=6.0 Hz, 2H), 7.78(d, J=6.0 Hz, 2H), 7.86-7.96(m, 4H) |
| 1-24 | 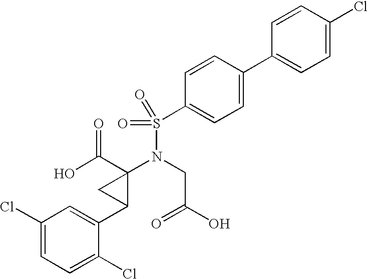 | (DMSO-d6-400) 1.99-2.10(m, 1H), 2.33-2.43(m, 1H), 3.05-3.13(m, 1H), 4.36-4.45(m, 1H), 4.51-4.63(m, 1H), 7.28-7.36(m, 2H), 7.42(d, J=8.0 Hz, 1H), 7.57(d, J=8.0 Hz, 2H), 7.78(d, J=8.0 Hz, 2H), 7.87-7.97(m, 4H) |
| 1-25 | 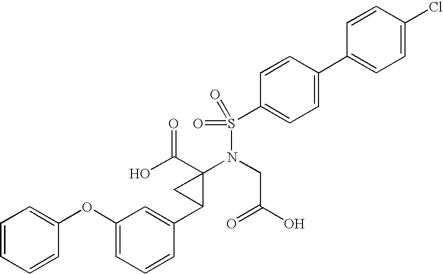 | (DMSO-d6-300) 2.09-2.24(m, 1H), 2.81-3.04(m, 1H), 3.87(br, 2H), 6.74-7.01(m, 4H), 7.11(t, J=7.5 Hz, 1H), 7.19-7.40(m, 3H), 7.58(d, J=9.0 Hz, 2H), 7.80(d, J=9.0 Hz, 2H), 7.85(br, 4H) |

TABLE 1-6

| | | |
|---|---|---|
| 1-26 | 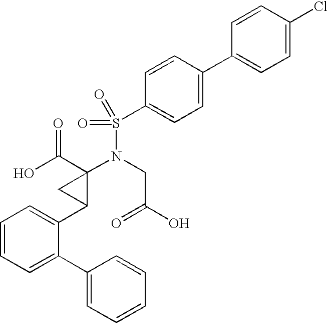 | (DMSO-d6-300) 1.70-1.87(m, 1H), 1.90-2.17(m, 1H), 3.01-3.21(m, 1H), 3.61-4.05(m, 2H), 7.03-7.12(m, 1H), 7.16-7.29(m, 5H), 7.29-7.45(m, 4H), 7.58(d, J=9.0 Hz, 2H), 7.77(d, J=9.0 Hz, 4H), 7.86(d, J=9.0 Hz, 2H) |
| 1-27 | 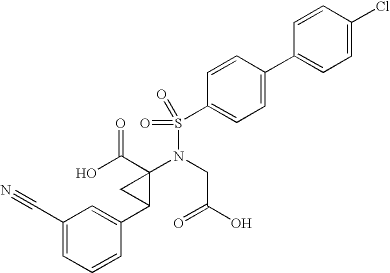 | (DMSO-d6-300) 1.91(br, 1H), 2.32(br, 1H), 3.05(br, 1H), 4.32(d, J=16.0 Hz, 1H), 4.48(d, J=16.0 Hz, 1H), 7.47(br, 1H), 7.51-7.61(m, 3H), 7.64-7.81(m, 4H), 7.90(br, 4H) |
| 1-28 | 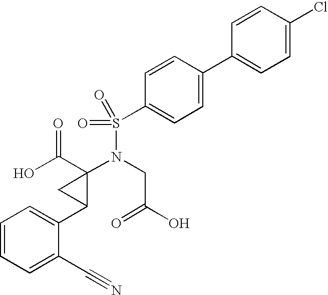 | (DMSO-d6-300) 2.09(br, 1H), 2.40(br, 1H), 3.25(br, 1H), 4.49(br, 2H), 6.99(br, 1H), 7.16(br, 1H), 7.31(br, 1H), 7.43(t, J=7.5 Hz, 1H), 7.58(d, J=9.0 Hz, 2H), 7.74-7.82(m, 3H), 7.92(br, 4H) |
| 1-29 | 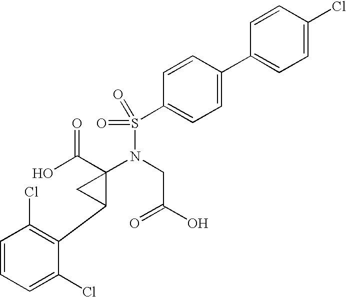 | (DMSO-d6-300) 1.71-1.77(m, 1H), 2.10-2.16(m, 1H), 2.60-2.68(m, 1H), 3.98(d, J=18.0 Hz, 1H), 4.22(d, J=18.0 Hz, 1H), 7.13-7.28(m, 2H), 7.29-7.37(m, 1H), 7.58(d, J=9.0 Hz, 2H), 7.82(d, J=9.0 Hz, 2H), 7.84-7.95(m, 4H) |
| 1-30 | 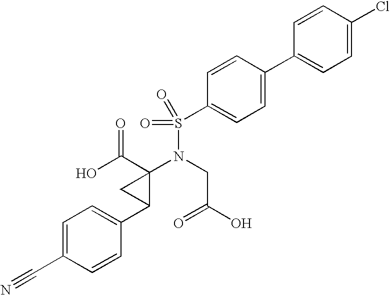 | (DMSO-d6-400) 1.84-1.97(m, 1H), 2.25-2.36(m, 1H), 3.10(br, 1H), 4.17-4.30(m, 1H), 4.39-4.60(m, 1H), 7.36-7.46(m, 2H), 7.57(d, J=8.0 Hz, 2H), 7.78(d, J=8.0 Hz, 2H), 7.70-7.81(m, 2H), 7.86-7.95(m, 4H) |

TABLE 1-7

| | | |
|---|---|---|
| 1-31 | 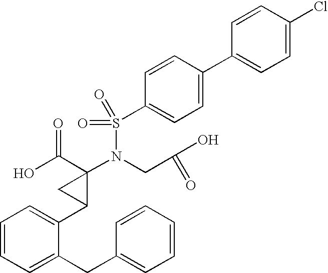 | (DMSO-d6-400) 1.88-2.02(m, 1H), 2.10-2.23(m, 1H), 2.86-3.01(m, 1H), 3.91-4.27(m, 4H), 6.89-6.97(m, 1H), 7.03(d, J=8.0 Hz, 2H), 7.07-7.20(m, 5H), 7.20-7.32(m, 3H), 7.57(d, J=8.0 Hz, 2H), 7.78(d, J=8.0 Hz, 2H), 7.88(br, 4H) |
| 1-32 | 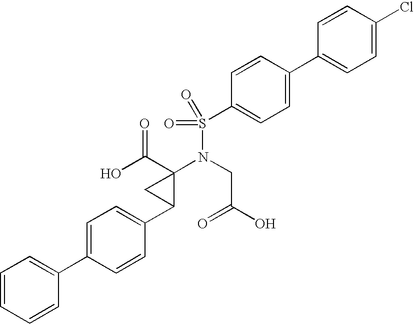 | (DMSO-d6-300) 1.81-1.94(m, 1H), 2.21-2.35(m, 1H), 2.97-3.10(m, 1H), 4.18-4.35(m, 1H), 4.47-4.63(m, 1H), 7.21-7.36(m, 3H), 7.43(t, J=6.0 Hz, 2H), 7.57(d, J=6.0 Hz, 2H), 7.51-7.67(m, 5H), 7.79(d, J=6.0 Hz, 2H), 7.90(d, J=6.0 Hz, 2H), 7.94(d, J=6.0 Hz, 2H) |
| 1-33 | 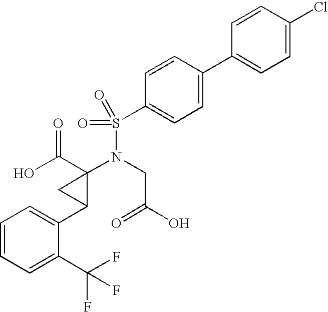 | (DMSO-d6-300) 2.06-2.15(m, 1H), 2.24-2.41(m, 1H), 3.15-3.27(m, 1H), 4.14-4.79(m, 2H), 7.41-7.48(m, 2H), 7.54-7.62(m, 3H), 7.68(d, J=9.0 Hz, 1H), 7.80(d, J=9.0 Hz, 2H), 7.91(d, J=9.0 Hz, 2H), 7.94(d, J=9.0 Hz, 2H) |
| 1-34 | 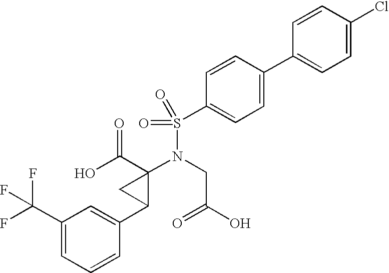 | (DMSO-d6-300) 1.85-1.98(m, 1H), 2.24-2.41(m, 1H), 3.01-3.17(m, 1H), 4.19-4.66(m, 2H), 7.45-7.62(m, 6H), 7.79(d, J=9.0 Hz, 2H), 7.90(d, J=9.0 Hz, 2H), 7.94(d, J=9.0 Hz, 2H) |
| 1-35 | 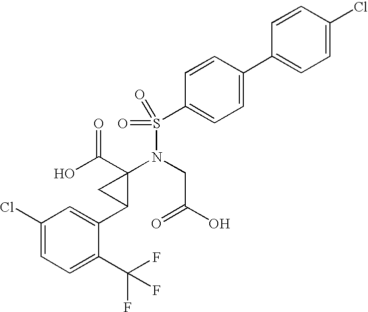 | (DMSO-d6-300) 2.07-2.17(m, 1H), 2.21-2.38(m, 1H), 3.20(t, J=9.0 Hz, 1H), 4.11-4.62(m, 2H), 7.44-7.61(m, 3H), 7.71(d, J=6.0 Hz, 1H), 7.80(d, J=9.0 Hz, 2H), 7.90-7.95(m, 4H) |

TABLE 1-8

| | | |
|---|---|---|
| 1-36 | 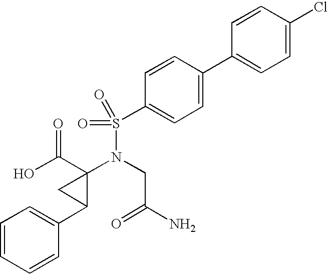 | (DMSO-d6-400) 1.61-1.72(m, 1H), 2.13-2.25(m, 1H), 3.04-3.17(m, 1H), 4.15(d, J=20.0 Hz, 1H), 4.49(d, J=24.0 Hz, 1H), 7.10-7.43(m, 5H), 7.57(d, J=8.0 Hz, 2H), 7.79(d, J=8.0 Hz, 2H), 7.89(d, J=8.0 Hz, 2H), 7.95(d, J=8.0 Hz, 2H) |
| 1-37 | 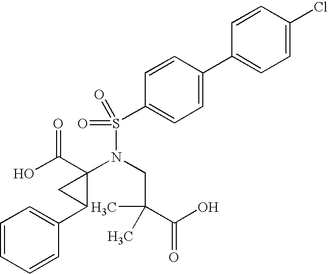 | (DMSO-d6-400) 0.94-1.37(m, 6H), 1.59-1.76(m, 1H), 1.83-2.01(m, 1H), 2.11-2.25(m, 1H), 3.40-3.66(m, 2H), 7.11-7.33(m, 4H), 7.36-7.47(m, 1H), 7.56(d, J=8.0 Hz, 2H), 7.77(d, J=8.0 Hz, 2H), 7.85-8.01(m, 4H) |
| 1-38 | 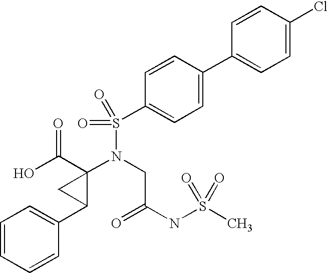 | (DMSO-d6-400) 1.07-1.35(m, 1H), 2.10-2.27(m, 1H), 2.82(s, 3H), 2.95-3.10(m, 1H), 3.88-4.31(m, 2H), 7.08-7.14(m, 7.16-7.25(m, 3H), 7.57(d, J=8.0 Hz, 2H), 7.79(d, J=8.0 Hz, 2H), 7.87-7.98(m, 4H), 8.53-8.59(m, 1H) |
| 1-39 | 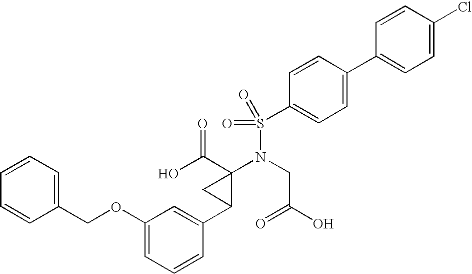 | (DMSO-d6-300) 1.77-1.88(m, 1H), 2.11-2.31(m, 1H), 2.85-3.05(m, 1H), 4.16-4.33(m, 1H), 4.49-4.67(m, 1H), 5.03(s, 2H), 6.71-6.91(m, 3H), 7.13-7.48(m, 7H), 7.58(d, J=9.0 Hz, 2H), 7.80(d, J=9.0 Hz, 2H), 7.86-7.97(m, 4H) |
| 1-40 | 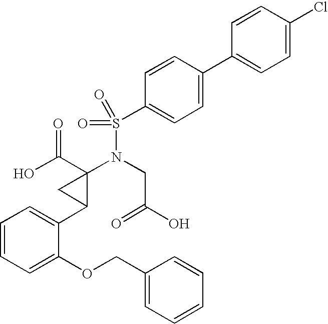 | (DMSO-d6-300) 1.17-1.34(m, 1H), 1.79-1.98(m, 1H), 2.14-2.34(m, 1H), 3.32(brs, 2H), 4.27-4.57(m, 1H), 5.03-5.31(m, 1H), 6.73-6.86(m, 2H), 6.97-7.14(m, 2H), 7.26-7.41(m, 5H), 7.59(d, J=9.0 Hz, 2H), 7.80(d, J=9.0 Hz, 2H), 7.91(brs, 4H), 12.05-12.94(m, 1H) |

TABLE 1-9

| | | |
|---|---|---|
| 1-41 | 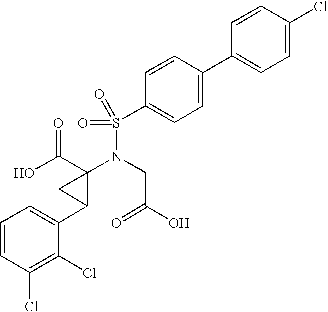 | (DMSO-d6-300) 1.94-2.12(m, 1H), 2.20-2.39(m, 1H), 3.06-3.23(m, 1H), 4.44(d, J=21.0 Hz, 1H), 4.53-4.71(m, 1H), 7.24-7.31(m, 2H), 7.46-7.55(m, 1H), 7.58(d, J=9.0 Hz, 2H), 7.80(d, J=9.0 Hz, 2H), 7.91(d, J=9.0 Hz, 2H), 7.95(d, J=9.0 Hz, 2H), 12.80(brs, 1H) |
| 1-42 | 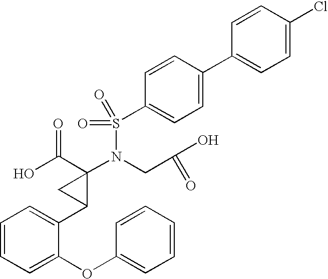 | (DMSO-d6-300) 1.91-2.07(m, 1H), 2.20-2.33(m, 1H), 3.08(t, J=9.0 Hz, 1H), 4.29(d, J=18.0 Hz, 1H), 4.38(d, J=18.0 Hz, 1H), 6.60(d, J=9.0 Hz, 1H), 6.91(d, J=9.0 Hz, 2H), 7.01(t, J=7.5 Hz, 1H), 7.10-7.26(m, 3H), 7.38(t, J=9.0 Hz, 2H), 7.58(d, J=9.0 Hz, 2H), 7.79(d, J=9.0 Hz, 2H), 7.89(brs, 4H), 12.38(brs, 1H), 12.67(brs, 1H) |
| 1-43 | 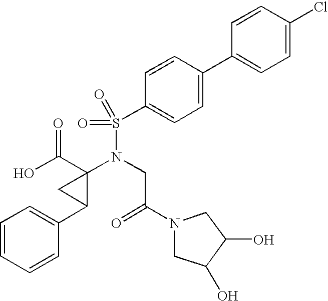 | (DMSO-d6-400) 1.41-1.62(m, 1H), 2.10-2.23(m, 1H), 3.03-3.18(m, 1H), 3.36-3.53(m, 3H), 3.70-3.82(m, 1H), 3.97-4.07(m, 1H), 4.11-4.19(m, 1H), 4.31-4.45(m, 1H), 4.58-4.75(m, 1H), 4.97-5.07(m, 1H), 5.11-5.17(m, 1H), 7.03-7.49(m, 5H), 7.57(d, J=8.0 Hz, 2H), 7.79(d, J=8.0 Hz, 2H), 7.90(d, J=12.0 Hz, 2H), 7.97(t, J=8.0 Hz, 2H) |
| 1-44 | 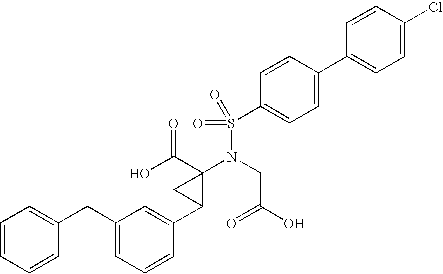 | (DMSO-d6-300) 1.74-1.98(m, 1H), 2.10-2.34(m, 1H), 2.87-3.10(m, 1H), 3.89(brs, 2H), 4.15-4.33(m, 1H), 4.40-4.70(m, 1H), 6.96-7.34(m, 9H), 7.59(d, J=6.0 Hz, 2H), 7.80(d, J=6.0 Hz, 2H), 7.92(brs, 4H), 12.39(brs, 1H), 12.92(brs, 1H) |
| 1-45 | 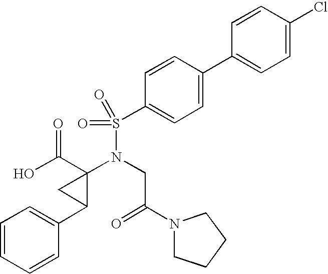 | (DMSO-d6-400) 1.43-1.63(m, 1H), 1.77-1.89(m, 2H), 1.90-2.02(m, 2H), 2.11-2.20(m, 1H), 3.13(t, J=12.0 Hz, 1H), 3.35-3.45(m, 2H), 3.51-3.67(m, 2H), 4.26-4.47(m, 1H), 4.59-4.78(m, 1H), 7.05-7.49(m, 5H), 7.57(d, J=8.0 Hz, 2H), 7.79(d, J=8.0 Hz, 2H), 7.90(d, J=8.0 Hz, 2H), 7.98(d, J=8.0 Hz, 2H) |

TABLE 1-10

| | | |
|---|---|---|
| 1-46 | 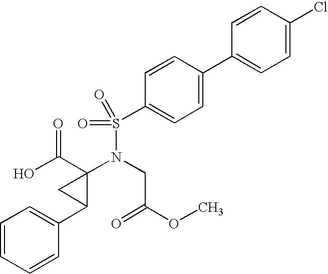 | (DMSO-d6-400) 1.76-1.95(m, 1H), 2.11-2.33(m, 1H), 2.83-3.02(m, 1H), 3.70(brs, 3H), 4.36(d, J=16.0 Hz, 1H), 4.50-4.71(m, 1H), 7.11-7.33(m, 5H), 7.57(d, J=8.0 Hz, 2H), 7.78(d, J=8.0 Hz, 2H), 7.86-7.95(m, 4H), 12.40(brs, 1H) |
| 1-47 | 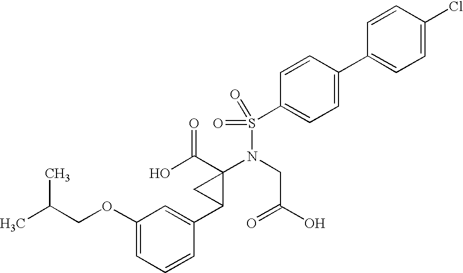 | (DMSO-d6-300) 0.96(d, J=6.0 Hz, 6H), 1.74-1.85(m, 1H), 1.91-2.05(m, 1H), 2.11-2.25(m, 1H), 2.87-3.01(m, 1H), 3.68(d, J=6.0 Hz, 2H), 4.14-4.31(m, 1H), 4.41-4.63(m, 1H), 6.64-6.80(m, 3H), 7.15(t, J=7.5 Hz, 1H), 7.58(d, J=9.0 Hz, 2H), 7.80(d, J=9.0 Hz, 2H), 7.87-7.96(m, 4H) |
| 1-48 | 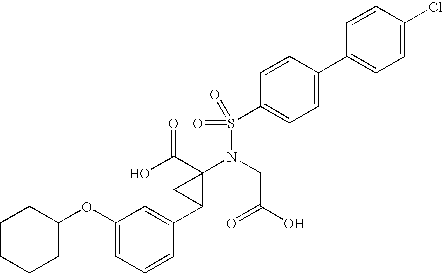 | (DMSO-d6-300) 1.19-1.97(m, 11H), 2.11-2.29(m, 1H), 2.86-3.05(m, 1H), 4.13-4.32(m, 2H), 4.45-4.69(m, 1H), 6.63-6.83(m, 3H), 7.14(t, J=7.5 Hz, 1H), 7.58(d, J=9.0 Hz, 2H), 7.80(d, J=9.0 Hz, 2H), 7.87-7.97(m, 4H) |
| 1-49 | 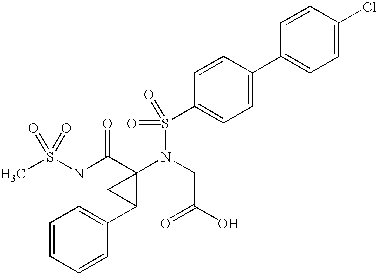 | (DMSO-d6-400) 1.20-1.30(m, 1H), 2.22(s, 3H), 2.86(t, J=10.0 Hz, 1H), 3.87(d, J=20.0 Hz, 1H), 4.11(d, J=16.0 Hz, 1H), 7.10-7.25(m, 5H), 7.56(d, J=8.0 Hz, 2H), 7.79(d, J=12.0 Hz, 2H), 7.86-7.91(m, 4H) |
| 1-50 | 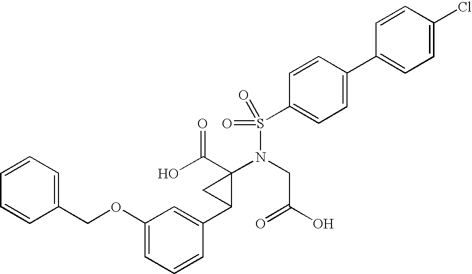 | (DMSO-d6-300) 1.46(dd, J=6.0, 9.0 Hz, 1H), 1.96(dd, J=6.0, 9.0 Hz, 1H), 2.66(t, J=9.0 Hz, 1H), 5.02(s, 2H), 6.78-6.88(m, 3H), 7.16(t, J=9.0 Hz, 1H), 7.28-7.46(m, 5H), 7.57(d, J=9.0 Hz, 2H), 7.78(d, J=9.0 Hz, 2H), 7.83-7.91(m, 4H), 8.90(br, 1H), 12.12(br, 1H) |

TABLE 1-11

| | | |
|---|---|---|
| 1-51 | 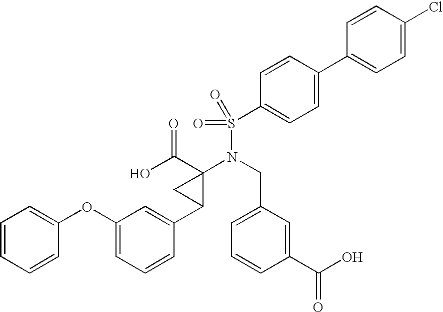 | (DMSO-d6-300 (120° C.) 1.67-1.77(m, 1H), 2.01-2.08(m, 1H), 4.73(d, J=15.8 Hz, 1H), 4.83(d, J=15.8 Hz, 1H), 6.78-6.83(m, 1H), 6.84-6.88(m, 1H), 6.91-7.01(m, 3H), 7.06-7.13(m, 1H), 7.09(t, J=7.3Hz, 1H), 7.21(t, J=7.9 Hz, 1H), 7.30-7.39(m, 3H), 7.49-7.55(m, 3H), 7.67-7.85 (7H, m) 7.86-7.90(m, 1H) |
| 1-52 | 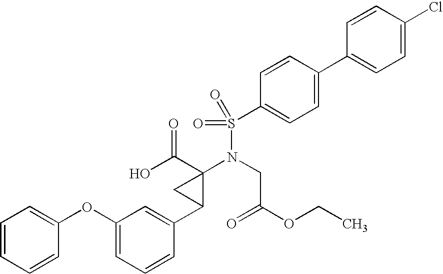 | (DMSO-d6-300 (120° C.)) 1.18(t, J=7.0 Hz, 3H), 1.86(dd, J=10.3, 5.5 Hz, 1H), 2.08(dd, J=8.8, 5.9 Hz, 1H), 3.05(t, J=9.0 Hz, 1H), 4.12(q, J=7.0 Hz, 2H), 4.29(d, J=18.0 Hz, 1H), 4.42(d, J=18.0 Hz, 1H), 6.91-6.98(m, 3H), 7.01-7.13(m, 3H), 7.25(t, J=7.8 Hz, 1H), 7.30-7.37(m, 2H), 7.49-7.55(m, 2H), 7.70-7.76(m, 2H), 7.80-7.86(m, 2H), 7.88-7.93(m, 2H) |
| 1-53 | 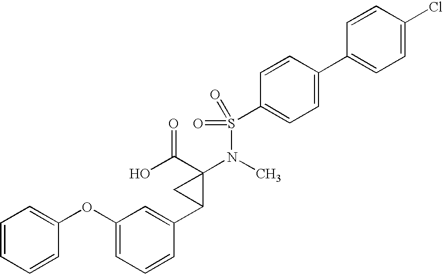 | (DMSO-d6-300) 1.48-1.82(m, 1H), 1.96-2.24(m, 1H), 2.84-3.16(m, 4H), 6.77-6.86(m, 1H), 6.91-7.03(m, 3H), 7.12(t, J=7.5 Hz, 1H), 7.27(t, J=7.5 Hz, 1H), 7.37(t, J=7.5 Hz, 2H), 7.57(d, J=6.0 Hz, 2H), 7.78(d, J=9.0 Hz, 2H), 7.85(brs, 4H) |
| 1-54 | 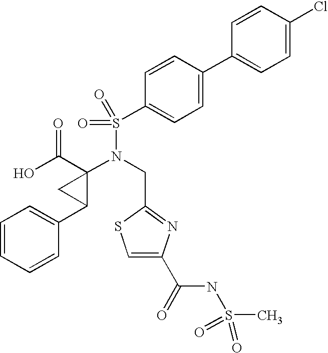 | (DMSO-d6-300 (120° C.)) 1.46-1.59(m, 1H), 1.94-2.02(m, 1H), 2.66-2.75(m, 1H), 2.84(s, 3H), 4.99(d, J=15.0 Hz, 1H), 5.24(d, J=15.0 Hz, 1H), 6.98-7.15(m, 3H), 7.25(d, J=9.0 Hz, 2H), 7.50(d, J=9.0 Hz, 2H), 7.73(d, J=6.0 Hz, 2H), 7.76(d, J=6.0 Hz, 2H), 7.99(d, J=9.0 Hz, 2H) |
| 1-55 | 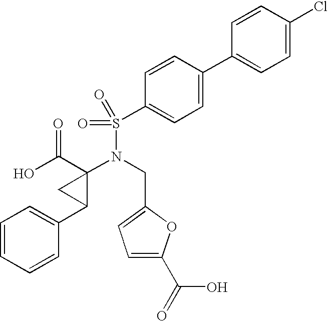 | (METHANOL-d4-400) 1.91-2.11(m, 1H), 2.30-2.39(m, 1H), 2.99-3.09(m, 1H), 4.41-4.58(m, 1H), 5.18-5.28(m, 1H), 6.38-6.62(m, 1H), 6.99-7.33(m, 5H), 7.43-7.56(m, 1H), 7.47(d, J=8.0 Hz, 2H), 7.65(d, J=8.0 Hz, 2H), 7.68-7.88(m, 4H) |

TABLE 1-12

| | | |
|---|---|---|
| 1-56 | 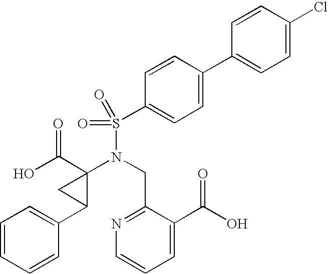 | (METHANOL-d4-400) 1.66-1.78(m, 1H), 2.41-2.52(m, 1H), 3.04-3.17(m, 1H), 5.16-5.32(m, 1H), 5.51-5.67(m, 1H), 6.88-6.99(m, 1H), 7.06-7.25(m, 2H), 7.32(t, J=8.0 Hz, 2H), 7.49(d, J=8.0 Hz, 2H), 7.67(d, J=8.0 Hz, 1H), 7.77(d, J=8.0 Hz, 1H), 7.92(d, J=8.0 Hz, 1H), 8.13(d, J=8.0 Hz, 2H), 8.19-8.49(m, 2H), 8.44(d, J=8.0 Hz, 2H) |
| 1-57 | 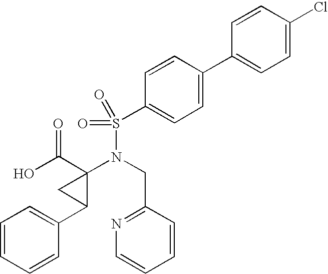 | (METHANOL-d4-400) 1.99-2.13(m, 1H), 2.36-2.47(m, 1H), 2.91-3.03(m, 1H), 5.05-5.25(m, 2H), 6.98-7.43(m, 8H), 7.48(d, J=8.0 Hz, 2H), 7..67(d, J=8.0 Hz, 2H), 7.77(d, J=8.0 Hz, 2H), 7.88(d, J=8.0 Hz, 2H), 8.44-8.53(m, 1H) |
| 1-58 | 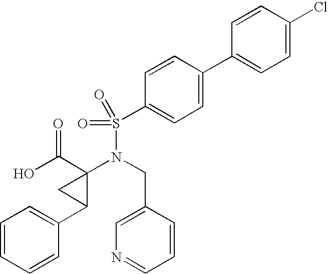 | (METHANOL-d4-400) 1.86-2.47(m, 3H), 4.91-5.20(m, 2H), 7.01-7.28(m, 5H), 7.34-7.44(m, 1H), 7.47(d, J=8.0 Hz, 2H), 7.68(d, J=8.0 Hz, 2H), 7.73(d, J=8.0 Hz, 2H), 7.90(d, J=8.0 Hz, 2H), 7.91-8.00(m, 1H), 8.40-8.46(m, 1H), 8.54-8.66(m, 1H) |
| 1-59 | 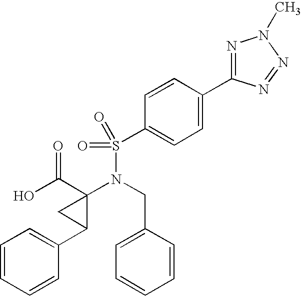 | (ACETONE-d6-400) 2.09-2.23(m, 1H), 2.30-2.43(m, 1H), 2.66-2.81(m, 1H), 4.49(s, 3H), 4.77-5.32(m, 2H), 7.00-7.58(m, 10H), 7.96-8.14(m, 2H), 8.25(d, J=8.0 Hz, 2H) |
| 1-60 | 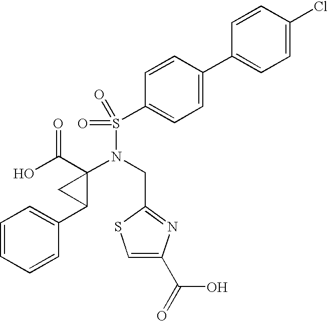 | (DMSO-d6-300) 1.16-1.37(m, 1H), 2.18-2.37(m, 1H), 2.86-3.05(m, 1H), 5.01(d, J=18.0 Hz, 1H), 5.21-5.46(m, 1H), 7.15-7.45(m, 5H), 7.58(d, J=9.0 Hz, 2H), 7.78(d, J=9.0 Hz, 2H), 7.86-7.97(m, 4H), 8.43(s, 1H), 12.95(brs, 1H) |

TABLE 1-13

| | | |
|---|---|---|
| 1-61 | 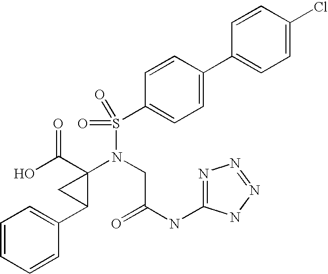 | (DMSO-d6-300) 1.91-2.00(m, 1H), 2.25-2.33(m, 1H), 3.07-3.19(m, 1H), 4.40-4.52(m, 1H), 4.80-4.93(m, 1H), 7.14-7.31(m, 5H), 7.59(d, J=9.0 Hz, 2H), 7.79(d, J=9.0 Hz, 2H), 7.87-7.98(m, 4H), 12.34(brs, 2H), 16.01(brs, 1H) |
| 1-62 | 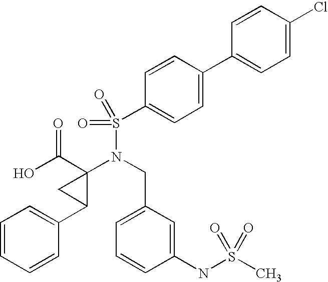 | (DMSO-d6-300) 1.41-1.59(m, 0.3H), 1.73-2.03(m, 1H), 2.18-2.35(m, 0.7H), 2.61-2.76(m, 0.7H), 2.87-2.98(m, 0.3H), 2.93(s, 3H), 4.27-4.81(m, 1.3H), 4.87-5.14(m, 0.7H), 6.91-7.52(m, 9H), 7.58(d, J=8.7 Hz, 2H), 7.77(d, J=8.7 Hz, 2H), 7.83(s, 4H), 9.64-9.85(brs, 1H), 12.15-12.49(brs, 1H) |
| 1-63 | 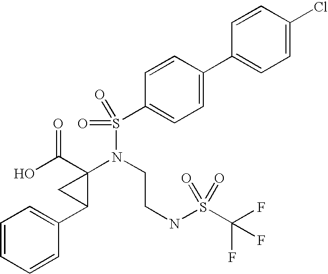 | (DMSO-d6-300) 1.77-1.91(m, 1H), 2.23-2.34(m, 1H), 2.99-3.11(m, 1H), 3.49-3.87(m, 4H), 7.15-7.35(m, 5H), 7.58(d, J=9.0 Hz, 2H), 7.79(d, J=9.0 Hz, 2H), 7.84-7.93(m, 4H), 9.76(brs, 1H), 12.34(brs, 1H) |
| 1-64 | 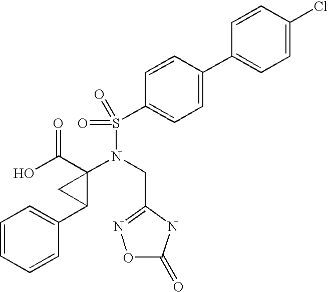 | (METHANOL-d4-300) 1.88-2.01(m, 1H), 2.31-2.44(m, 1H), 2.91-3.04(m, 1H), 4.61-5.00(m, 2H), 7.11-7.30(m, 5H), 7.48(d, J=9.0 Hz, 2H), 7.68(d, J=9.0 Hz, 2H), 7.81(d, J=9.0 Hz, 2H), 7.96(d, J=9.0 Hz, 2H) |
| 1-65 | 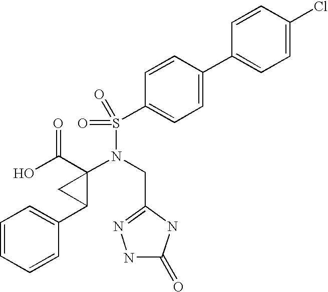 | (METHANOL-d4-300) 2.08-2.20(m, 1H), 2.33-2.43(m, 1H), 3.03-3.14(m, 1H), 4.57-4.71(m, 1H), 4.87-4.96(m, 1H), 7.11-7.34(m, 5H), 7.43-7.53(m, 2H), 7.61-7.71(m, 2H), 7.75-7.84(m, 2H), 7.93(d, J=9.0 Hz, 2H) |

TABLE 1-14

| | | |
|---|---|---|
| 1-66 | 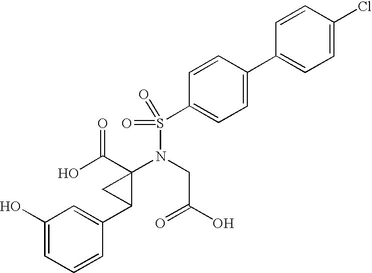 | (DMSO-d6-300) 1.11-1.31(m, 1H), 1.98-2.18(m, 1H), 2.60-2.80(m, 1H), 3.80(d, J=18.0 Hz, 1H), 4.02(d, J=18.0 Hz, 1H), 6.49-6.59(m, 3H), 6.97(t, J=9.0 Hz, 1H), 7.57(d, J=6.0 Hz, 2H), 7.81(d, J=9.0 Hz, 2H), 7.86(d, J=9.0 Hz, 2H), 7.91(d, J=9.0 Hz, 2H), 9.22(s, 1H) |
| 1-67 | 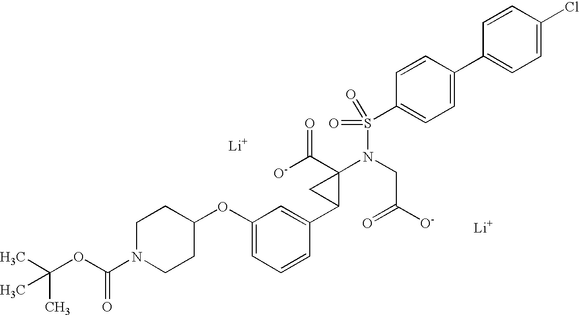 | (DMSO-d6-300) 1.40(s, 9H), 1.43-1.57(m, 2H), 1.70-1.97(m, 4H), 2.65-2.77(m, 1H), 3.08-3.21(m, 2H), 3.58-3.72(m, 2H), 4.34-4.51(m, 1H), 6.58-7.08(m, 4H), 7.57(d, J=9.0 Hz, 2H), 7.79(d, J=9.0 Hz, 2H), 7.84(d, J=9.0 Hz, 2H), 7.99(d, J=9.0 Hz, 2H) |
| 1-68 | 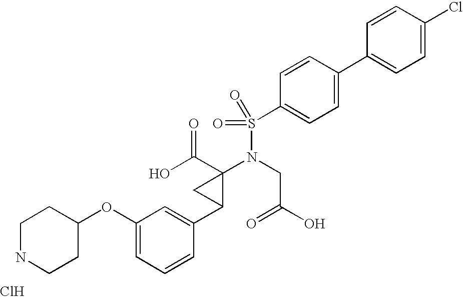 | (DMSO-d6-300) 1.69-1.90(m, 3H), 1.99-2.12(m, 2H), 2.15-2.32(m, 1H), 2.85-3.23(m, 3H), 3.41-3.54(m, 1H), 3.63-3.77(m, 2H), 4.09-4.32(m, 1H), 4.51-4.71(m, 1H), 6.71-6.91(m, 3H), 7.11-7.24(m, 1H), 7.58(d, J=9.0 Hz, 2H), 7.80(d, J=9.0 Hz, 2H), 7.91(brs, 4H), 8.61-9.01(m, 2H), 12.31(brs, 1H) |
| 1-69 | 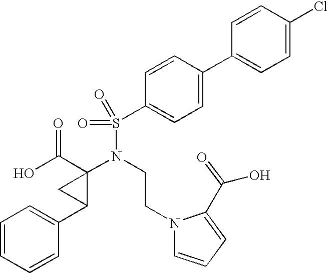 | (METHANOL-d4-400) 1.95-2.00(m, 1H), 2.19(dd, J=4.0, 8.0 Hz, 1H), 3.29-3.32(m, 1H), 3.65-3.75(m, 1H), 4.10-4.15(m, 1H), 4.21-4.27(m, 1H), 4.46-4.60(m, 1H), 6.05(t, J=2.0 Hz, 1H), 6.81-6.85(m, 1H), 7.01-7.37(m, 6H), 7.48(d, J=8.0 Hz, 2H), 7.68(d, J=8.0 Hz, 2H), 7.81(d, J=8.0 Hz, 2H), 7.95(d, J=8.0 Hz, 2H) |
| 1-70 | 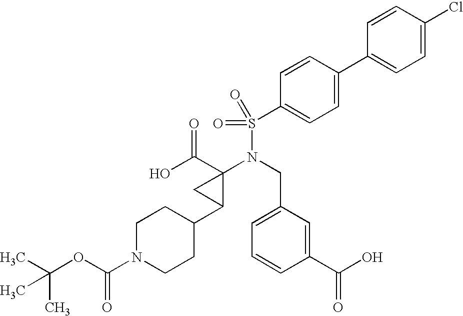 | (DMSO-d6-300 (120° C.) 0.97-1.65(m, 16H), 2.50-2.72(m, 3H), 3.71-3.88(m, 2H), 4.59(d, J=15.0 Hz, 1H), 4.71-4.86(m, 1H), 7.36(t, J=7.5 Hz, 1H), 7.47(d, J=9.0 Hz, 1H), 7.52(d, J=9.0 Hz, 2H), 7.70(d, J=9.0 Hz, 2H), 7.73(s, 4H), 7.76-7.82(m, 2H) |

TABLE 1-15

| | | |
|---|---|---|
| 1-71 | 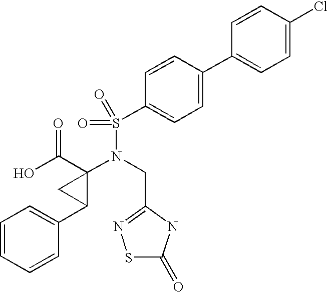 | (METHANOL-d4-300) 2.09-2.20(m, 1H), 2.34-2.45(m, 1H), 3.11-3.21(m, 1H), 4.54-4.67(m, 1H), 4.75-4.97(m, 1H), 7.11-7.33(m, 5H), 7.49(dd, J=3.0, 6.0 Hz, 2H), 7.68(dd, J=3.0, 6.0 Hz, 2H), 7.81(d, J=9.0 Hz, 2H), 7.94(d, J=9.0 Hz, 2H) |
| 1-72 | 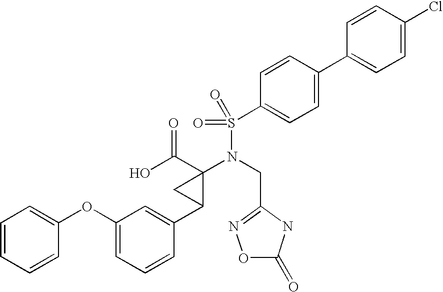 | (DMSO-d6-300) 1.79-2.07(m, 1.3H), 2.19-2.35(m, 0.7H), 2.86-3.17(m, 1H), 4.38-4.57(m, 0.6H), 4.63-4.83(m, 1.4H), 6.76-7.44(m, 9H), 7.59(d, J=8.6 Hz, 2H), 7.70-7.97(m, 6H), 12.13-12.77(brs, 2H) |
| 1-73 | 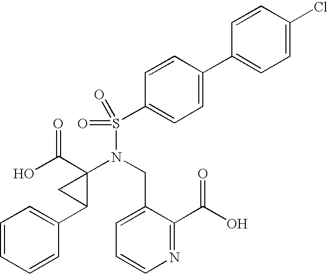 | (METHANOL-d4-400) 2.31-2.48(m, 1H), 2.51-2.68(m, 1H), 3.30-3.62(m, 1H), 5.21-5.60(m, 2H), 6.71-8.70(m, 16H) |
| 1-74 | 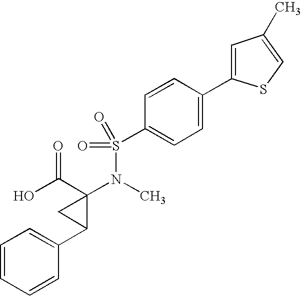 | (ACETONE-d6-500) 1.75-2.30(m, 2H), 2.28(s, 3H), 2.96(br, 1H), 3.30(brs, 3H), 7.13-7.29(m, 5H), 7.35(brs, 1H), 7.46(s, 1H), 7.81(d, J=10.0 Hz, 2H), 7.87(d, J=10.0 Hz, 2H) |
| 1-75 | 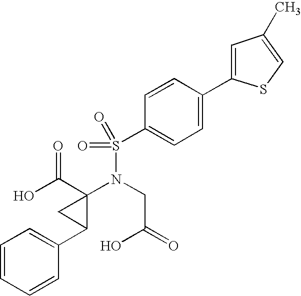 | (ACETONE-d6-500) 1.93(dd, J=5.0, 10.0 Hz, 1H), 2.01(dd, J=5.0, 10.0 Hz, 1H), 2.29(s, 3H), 2.78(t, J=10.0 Hz, 1H), 4.38-4.46(m, 1H), 4.69-4.78(m, 1H), 7.11-7.31(m, 6H), 7.84(d, J=5.0 Hz, 2H), 7.96(d, J=5.0 Hz, 2H), 11.23(brs, 1H) |

TABLE 1-16

| | | |
|---|---|---|
| 1-76 | 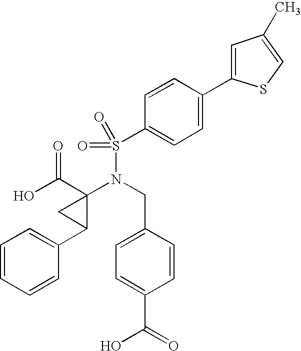 | (ACETONE-d6-500) 2.11-2.26(m, 1H), 2.29(s, 3H), 2.30-2.44(m, 1H), 2.61-2.77(m, 1H), 4.86-4.97(m, 1H), 5.23-5.35(m, 1H), 7.05-7.33(m, 6H), 7.46-7.59(m, 2H), 7.61-7.72(m, 1H), 7.80(d, J=10.0 Hz, 2H), 7.81-7.92(m, 2H), 8.01-8.09(m, 2H) |
| 1-77 | 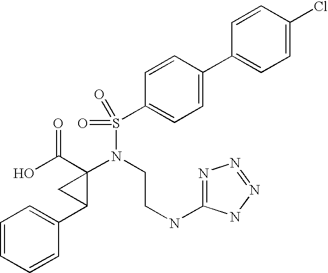 | (METHANOL-d4-400) 1.83-1.92(m, 1H), 2.01-2.12(m, 1H), 2.24-2.30(m, 1H), 3.99-4.39(m, 2H), 4.78-5.13(m, 2H), 7.13-7.25(m, 5H), 7.48(dd, J=2.0, 8.0 Hz, 2H), 7.69(dd, J=2.0, 8.0 Hz, 2H), 7.82(d, J=8.0 Hz, 2H), 7.97(d, J=8.0 Hz, 2H) |
| 1-78 | 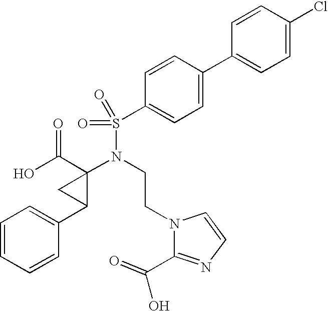 | (METHANOL-d4-400) 2.05-2.14(m, 1H), 2.30-2.37(m, 1H), 2.50-2.58(m, 1H), 4.11-4.35(m, 2H), 4.99-5.20(m, 2H), 7.15-7.26(m, 5H), 7.48(d, J=8.0 Hz, 2H), 7.64-7.66(m, 1H), 7.67(d, J=8.0 Hz, 2H), 7.80(d, J=8.0 Hz, 2H), 7.90(d, J=12.0 Hz, 2H), 7.91-7.94(m, 1H) |
| 1-79 | 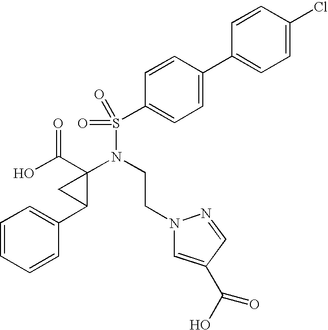 | (ACETONE-d6-500) 2.17(dd, J=5.0, 10.0 Hz, 1H), 2.39(dd, J=5.0, 10.0 Hz, 1H), 2.67(t, J=8.0 Hz, 1H), 4.30(d, J=15.0 Hz, 2H), 4.65(d, J=15.0 Hz, 2H), 7.15-7.34(m, 5H), 7.55(d, J=5.0 Hz, 2H), 7.77(d, J=5.0 Hz, 2H), 7.89(d, J=5.0 Hz, 2H), 7.93(s, 1H), 8.04(d, J=5.0 Hz, 2H), 8.23(s, 1H) |
| 1-80 | 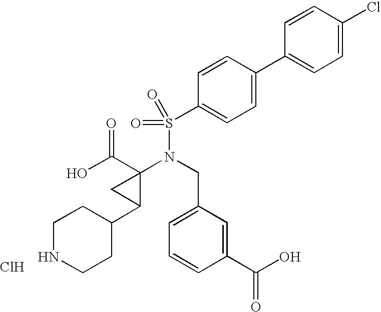 | (DMSO-d6-300 (120° C.)) 1.33-1.85(m, 8H), 3.13-3.24(m, 3H), 4.59(d, J=18.0 Hz, 1H), 4.67-4.83(m, 1H), 7.37(t, J=9.0 Hz, 1H), 7.44-7.50(m, 1H), 7.53(d, J=9.0 Hz, 2H), 7.71(d, J=9.0 Hz, 2H), 7.75(s, 4H), 7.77-7.83(m, 2H) |

TABLE 1-17

| | | |
|---|---|---|
| 1-81 | 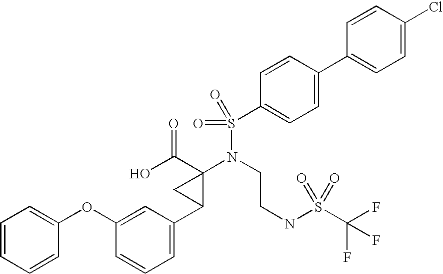 | (DMSO-d6-300 (120° C.)) 1.76-1.86(m, 1H), 2.07-2.17(m, 1H), 3.01-3.10(m, 1H), 3.41-3.74(m, 5H), 6.83(d, J=6.0 Hz, 1H), 6.91-7.02(m, 3H), 7.04-7.14(m, 2H), 7.25(d, J=9.0 Hz, 1H), 7.27-7.38(m, 2H), 7.53(d, J=9.0 Hz, 2H), 7.73(d, J=9.0 Hz, 2H), 7.79-7.90(m, 4H) |
| 1-82 | 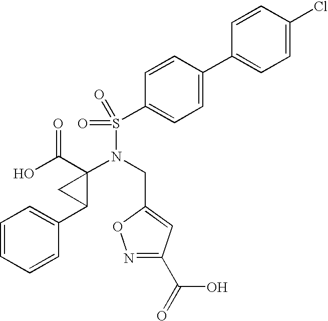 | (METHANOL-d4-400) 2.13(brs, 1H), 2.39(brs, 1H), 2.97(brs, 1H), 4.88-4.96(m, 1H), 5.23-5.38(m, 1H), 6.77(brs, 1H), 7.10-7.36(m, 5H), 7.48(d, J=8.0 Hz, 2H), 7.68(d, J=8.0 Hz, 2H), 7.79(d, J=8.0 Hz, 2H), 7.90(d, J=8.0 Hz, 2H) |
| 1-83 | 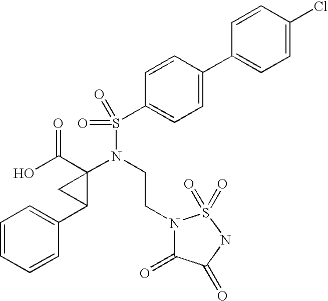 | (METHANOL-d4-400) 2.09(brs, 1H), 2.41(brs, 1H), 2.96-3.12(m, 1H), 3.76-3.92(m, 1H), 3.94-4.06(m, 2H), 4.16-4.29(m, 1H), 7.11-7.42(m, 5H), 7.47(d, J=8.0 Hz, 2H), 7.68(d, J=8.0 Hz, 2H), 7.81(d, J=8.0 Hz, 2H), 7.97(d, J=8.0 Hz, 2H) |
| 1-84 | 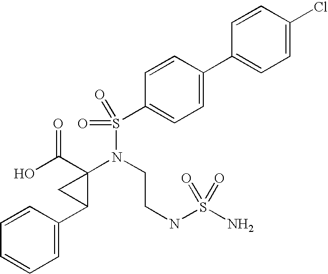 | (METHANOL-d4-400) 2.05(brs, 1H), 2.37(brs, 1H), 3.05-3.18(m, 1H), 3.40-3.65(m, 2H), 3.67-3.83(m, 1H), 3.84-3.99(m, 1H), 7.09-7.38(m, 5H), 7.48(d, J=8.0 Hz, 2H), 7.68(d, J=8.0 Hz, 2H), 7.81(d, J=8.0 Hz, 2H), 7.95(d, J=8.0 Hz, 2H) |
| 1-85 | 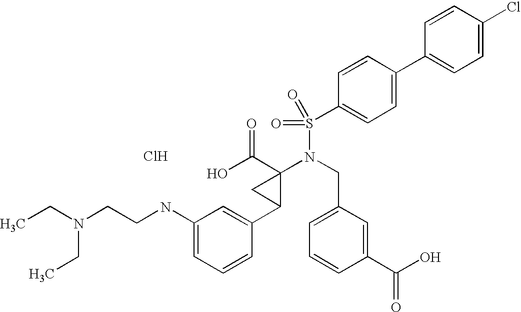 | (DMSO-d6-300) 1.65-1.75(m, 1H), 2.01-2.12(m, 1H), 3.10-3.24(m, 8H), 3.30-3.45(m, 1H), 4.70-5.25(m, 2H), 6.26-7.07(m, 4H), 7.40-7.62(m, 2H), 7.58(d, J=9.0 Hz, 2H), 7.78(d, J=21.0 Hz, 2H), 7.75-7.96(m, 6H) |

TABLE 1-18

| | | |
|---|---|---|
| 1-86 | 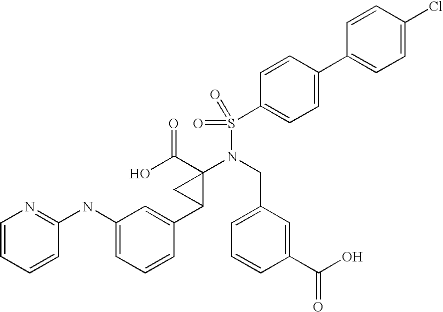 | (DMSO-d6-300) 1.37-1.60(m, 0.3H), 1.68-2.08(m, 1H), 2.14-2.36(m, 0.7H), 2.44-2.66(m, 1H), 4.28-5.29(m, 6.50-6.62(m, 1H), 6.71(dd, J=5.3, 7.1 Hz, 1H), 6.77-6.88(m, 1H), 6.91-7.22(m, 1H), 7.33-8.03(m, 7H), 7.57(d, J=8.6 Hz, 2H), 7.76(d, J=8.7 Hz, 2H), 7.81(s, 4H), 8.12(d, J=4.9 Hz, 1H), 8.93(s, 1H) |
| 1-87 | 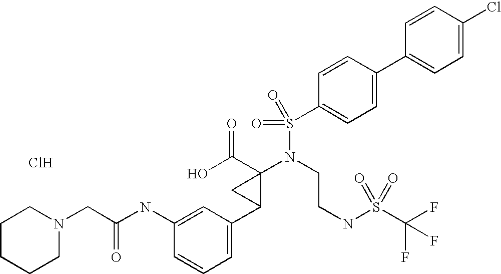 | (DMSO-d6-300) 0.70-0.88(m, 2H), 1.29-1.52(m, 1H), 1.64-1.93(m, 5H), 2.01-2.27(m, 1H), 2.93-3.17(m, 2H), 3.34-3.67(m, 5H), 3.73-3.90(m, 1H), 3.95-4.16(m, 2H), 6.97-7.10(m, 1H), 7.13-7.34(m, 2H), 7.37-7.64(m, 4H), 7.68-7.81(m, 2H), 7.68-7.99(m, 3H), 9.57-9.90(m, 2H), 10.45-10.81(m, 1H), 12.13-12.61(m, 1H) |
| 1-88 | 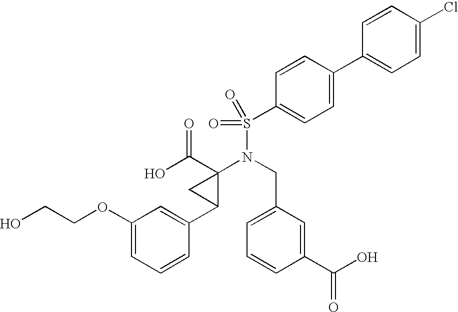 | (DMSO-d6-300 (120° C.)) 1.58-1.76(m, 1H), 1.96-2.13(m, 1H), 2.67-2.87(m, 1H), 3.69(t, J=4.5 Hz, 2H), 3.93(t, J=6.0 Hz, 2H), 4.81(s, 1H), 6.68-6.79(m, 3H), 7.07(t, J=9.0 Hz, 1H), 7.39(t, J=7.5 Hz, 1H), 7.47-7.57(m, 3H), 7.65-7.94(m, 8H) |
| 1-89 | 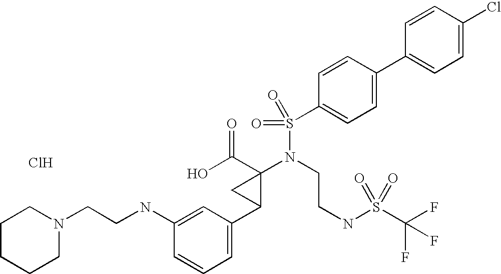 | (DMSO-d6-300) 1.17-1.48(m, 2H), 1.61-1.86(m, 6H), 2.15-2.29(m, 1H), 2.81-2.99(m, 3H), 3.07-3.21(m, 3H), 3.56-3.63(m, 2H), 3.73-3.89(m, 2H), 6.41-6.60(m, 2H), 6.94-7.06(m, 1H), 7.59(d, J=9.0 Hz, 2H), 7.79(d, J=9.0 Hz, 2H), 7.83-7.97(m, 4H), 9.79(brs, 1H) |
| 1-90 | 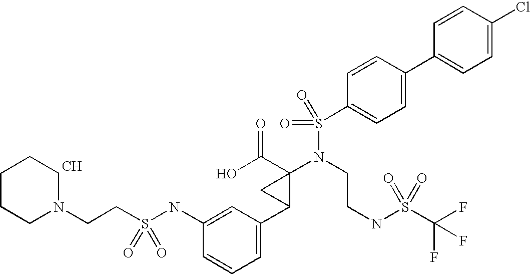 | (DMSO-d6-300) 1.23-1.44(m, 2H), 1.51-1.81(m, 5H), 1.81-1.98(m, 1H), 2.11-2.34(m, 1H), 2.76-2.96(m, 2H), 2.99-3.13(m, 2H), 3.53-3.68(m, 4H), 3.77-3.89(m, 2H), 7.04-7.17(m, 2H), 7.21-7.35(m, 1H), 7.59(d, J=9.0 Hz, 2H), 7.79(d, J=9.0 Hz, 2H), 7.85-8.02(m, 4H), 10.04-10.21(m, 1H) |

TABLE 1-19

| | | |
|---|---|---|
| 1-91 | 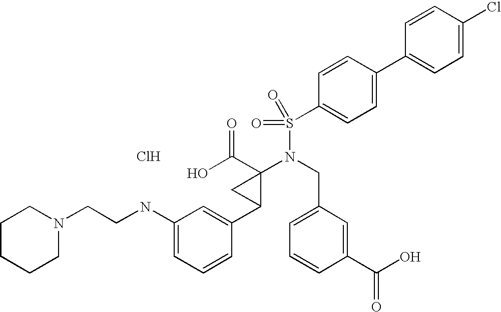 | (DMSO-d6-300) 1.50-1.62(m, 2H), 1.65-1.75(m, 1H), 1.75-1.86(m, 4H), 2.01-2.11(m, 1H), 2.77-2.85(m, 1H), 3.10-3.45(m, 8H), 4.71-4.90(m, 2H), 6.44-6.53(m, 3H), 6.91-6.97(m, 1H), 7.37-7.61(m, 2H), 7.58(d, J=9.0 Hz, 2H), 7.76(d, J=9.0 Hz, 2H), 7.73-7.97(m, 6H) |
| 1-92 | 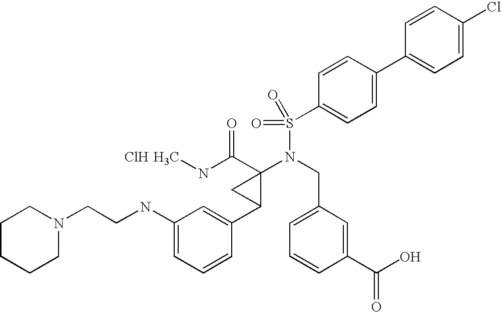 | (DMSO-d6-300) 1.29-1.42(m, 2H), 1.63-1.83(m, 5H), 2.16-2.33(m, 1H), 2.60 3.48(m, 9H), 2.99(s, 3H), 4.58-5.20(m, 2H), 6.17-7.00(m, 4H), 7.41-7.63(m, 2H), 7.53(d, J=9.0 Hz, 2H), 7.72(d, J=9.0 Hz, 2H), 7.73-8.05(m, 6H) |
| 1-93 | 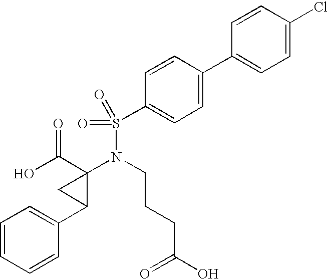 | (METHANOL-d4-400) 1.80-2.26(m, 3H), 2.28-2.56(m, 2H), 2.91-3.17(m, 1H), 3.54-3.92(m, 2H), 7.11-7.38(m, 5H), 7.48(d, J=8.0 Hz, 2H), 7.68(d, J=8.0 Hz, 2H), 7.81(d, J=8.0 Hz, 2H), 7.89-8.10(m, 2H) |
| 1-94 | 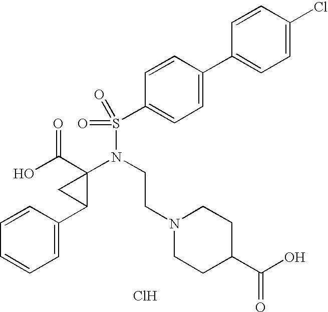 | (DMSO-d6-300 (120° C.)) 1.57-1.73(m, 3H), 1.75-1.87(m, 2H), 2.16(dd, J=6.0, 9.0 Hz, 1H), 2.21-2.44(m, 3H), 2.61-3.28(m, 5H), 3.60-3.78(m, 2H), 7.14-7.34(m, 5H), 7.52(d, J=9.0 Hz, 2H), 7.73(d, J=9.0 Hz, 2H), 7.84(d, J=9.0 Hz, 2H), 7.93(d, J=9.0 Hz, 2H) |
| 1-95 | 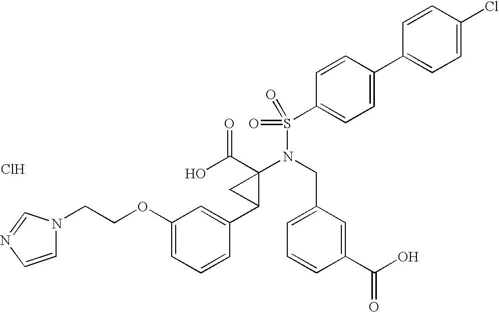 | (DMSO-d6-300 (90° C.)) 1.65-1.83(m, 1H), 1.98-2.20(m, 1H), 2.70-2.93(m, 1H), 4.30(t, J=6.0 Hz, 2H), 4.55(t, J=4.5 Hz, 2H), 4.68-4.95(m, 2H), 6.65-6.89(m, 3H), 7.13(t, J=9.0 Hz, 1H), 7.42(t, J=7.5 Hz, 1H), 7.49-7.62(m, 5H), 7.65-7.96(m, 8H), 8.91(brs, 1H) |

TABLE 1-20

| | | |
|---|---|---|
| 1-96 | 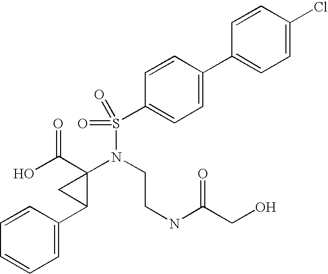 | (DMSO-d6-300) 1.71-1.82(m, 1H), 2.24-2.32(m, 1H), 2.95-3.09(m, 1H), 3.75-3.89(m, 2H), 5.40-5.55(m, 1H), 7.11-7.39(m, 4H), 7.58(d, J=9.0 Hz, 2H), 7.78(d, J=6.0 Hz, 2H), 7.86-7.97(m, 4H), 8.01-8.17(m, 1H), 12.02(brs, 1H) |
| 1-97 | 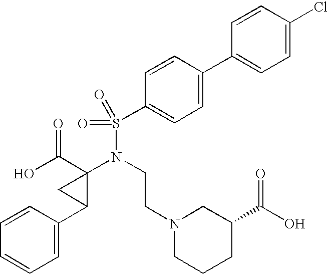 | (DMSO-d6-400) 1.31-2.70(m, 8H), 2.87-3.58(m, 6H), 3.75-3.97(m, 2H), 7.15-7.50(m, 5H), 7.53-7.62(m, 2H), 7.74-7.80(m, 2H), 7.85-8.00(m, 5H) |
| 1-98 | 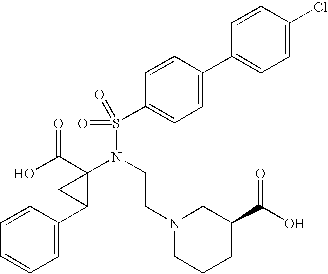 | (DMSO-d6-400) 1.24-2.54(m, 8H), 2.64-3.45(m, 6H), 3.61-3.95(m, 2H), 7.09-7.45(m, 5H), 7.49-7.58(m, 2H), 7.71-7.78(m, 2H), 7.81-7.97(m, 4H) |
| 1-99 | 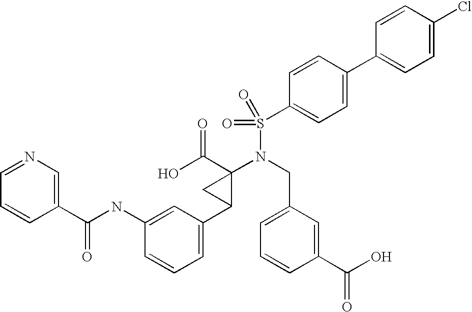 | (DMSO-d6-300) 1.44-1.58(m, 0.4H), 1.79-1.88(m, 0.4H), 1.99-2.09(m, 0.6H), 2.21-2.35(m, 0.6H), 2.61-2.73(m, 0.6H), 3.20-3.34(m, 0.4H), 4.43-4.87(m, 1.4H), 5.11-5.26(m, 0.6H), 6.76-6.87(m, 0.6H), 7.13-7.33(m, 1.4H), 7.35-8.03(m, 15H), 8.37-8.52(m, 1H), 8.78-8.84(m, 1H), 9.10-9.22(m, 1H), 10.41-10.62(m, 1H) |
| 1-100 | 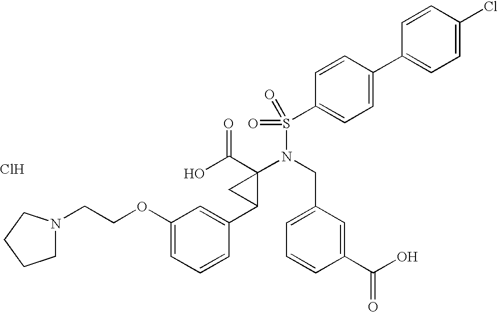 | (DMSO-d6-300 (120° C.)) 1.61-1.83(m, 1H), 1.93-2.01(m, 4H), 2.03-2.17(m, 1H), 2.65-3.12(m, 5H), 3.49(t, J=6.0 Hz, 2H), 4.29(t, J=6.0 Hz, 2H), 4.64-4.98(m, 2H), 6.76-6.90(m, 3H), 7.16(t, J=7.5 Hz, 1H), 7.41(t, J=7.5 Hz, 1H), 7.52(d, J=9.0 Hz, 2H), 7.58(d, J=9.0 Hz, 1H), 7.67-7.88(m, 8H), 7.90(brs, 1H) |

TABLE 1-21

| | | |
|---|---|---|
| 1-101 | 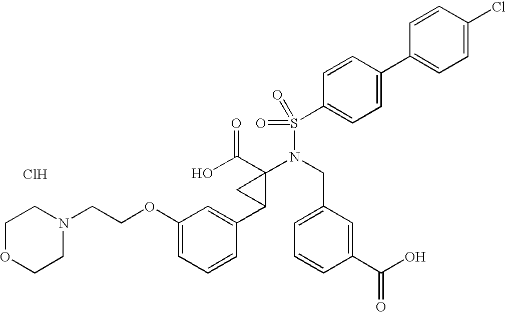 | (DMSO-d6-300 (120° C.)) 1-61-1.78(m, 1H), 1.99-2.14(m, 1H), 2.67(t, J=6.0 Hz, 2H), 2.77-2.91(m, 1H), 3.48-3.60(m, 4H), 4.02(t, J=4.5 Hz, 2H), 4.64-4.94(m, 2H), 6.63-6.81(m, 3H), 7.09(t, J=9.0 Hz, 1H), 7.40(t, J=7.5 Hz, 1H), 7.44-7.61(m, 3H), 7.64-7.95(m, 8H) |
| 1-102 | 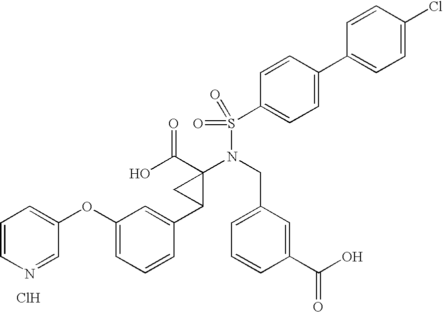 | (DMSO-d6-300 (120° C.)) 1.67-1.78(m, 1H), 2.01-2.11(m, 1H), 4.73(d, J=15.0 Hz, 1H), 4.82(d, J=15.0 Hz, 1H), 6.84-6.92(m, 2H), 7.00-7.06(m, 1H), 7.21-7.40(m, 4H), 7.49-7.55(m, 3H), 7.67-7.89(m, 8H), 8.27-8.34(m, 2H) |
| 1-103 | 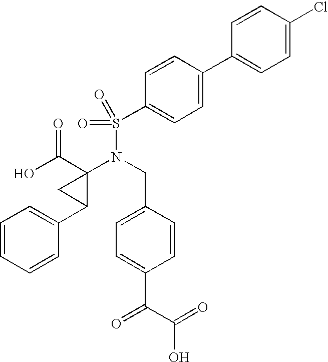 | (METHANOL-d4-400) 2.15(brs, 1H), 2.35(brs, 1H), 2.64(brs, 1H), 4.31-4.63(m, 1H), 4.68-5.01(m, 2H), 5.11-5.39(m, 1H), 6.90-7.28(m, 5H), 7.48(d, J=8.0 Hz, 2H), 7.53-7.65(m, 1H), 7.68(d, J=8.0 Hz, 2H), 7.76(d, J=8.0 Hz, 2H), 7.81-8.05(m, 3H), 8.24-8.66(m, 2H) |
| 1-104 | 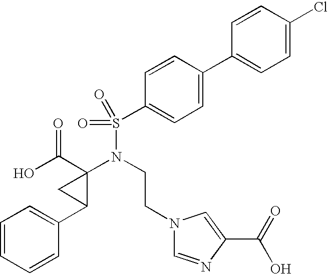 | (METHANOL-d4-400) 1.90-2.05(m, 1H), 2.27-2.42(m, 1H), 2.49-2.65(m, 1H), 4.05-4.35(m, 2H), 4.95-5.20(m, 2H), 7.11-7.35(m, 5H), 7.48(d, J=8.0 Hz, 2H), 7.67(d, J=8.0 Hz, 2H), 7.81(d, J=12.0 Hz, 2H), 7.90(d, J=8.0 Hz, 2H), 8.18(brs, 1H), 9.15(brs, 1H) |
| 1-105 | 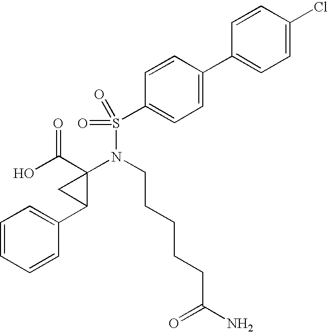 | (DMSO-d6-400) 1.08-1.85(m, 6H), 1.94-2.09(m, 2H), 2.84-3.15(m, 2H), 7.14-7.32(m, 5H), 7.56-7.58(d, 2H), 7.78(d, J=8.6 Hz, 2H), 7.84-7.99(m, 4H) |

TABLE 1-22

| | | |
|---|---|---|
| 1-106 | 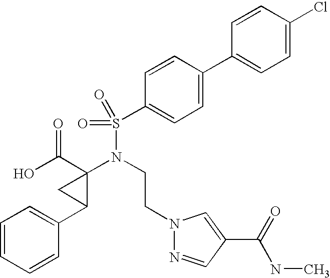 | (METHANOL-d4-400) 1.53-1.62(m, 1H), 1.94-2.00(m, 1H), 2.21-2.30(m, 1H), 2.81(s, 1.5H), 2.82(s, 1.5H), 3.89-4.01(m, 1H), 4.15-4.26(m, 1H), 4.54(dt, J=4.3, 14.5 Hz, 1H), 4.74-4.82(m, 1H), 7.09-7.28(m, 5H), 7.48(d, J=9.0 Hz, 2H), 7.68(d, J=8.7 Hz, 2H), 7.81(d, J=8.2 Hz, 2H), 7.91(s, 1H), 7.96(d, J=8.2 Hz, 2H), 8.07(s, 1H), 8.15(s, 1H) |
| 1-107 | 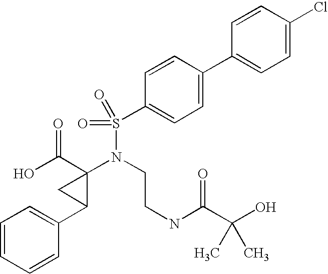 | (DMSO-d6-300) 1.23(s, 3H), 1.25(s, 3H), 1.57-1.80(m, 1H), 2.07-2.35(m, 1H), 2.81-3.05(m, 1H), 5.21-5.42(m, 1H), 7.14-7.41(m, 4H), 7.57(d, J=9.0 Hz, 2H), 7.78(d, J=9.0 Hz, 2H), 7.83-7.95(m, 4H), 7.98-8.13(m, 1H) |
| 1-108 | 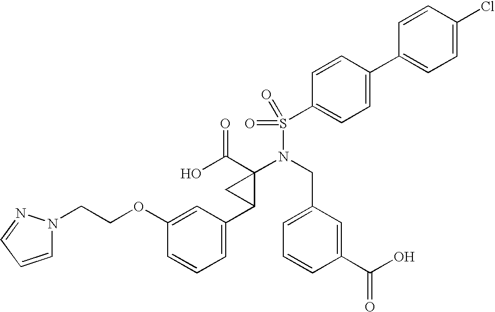 | (DMSO-d6-300 (120° C.)) 1.66-1.75(m, 1H), 2.01-2.10(m, 2H), 4.27(t, J=6.0 Hz, 2H), 4.42(t, J=6.0 Hz, 2H), 4.71-4.91(m, 2H), 6.17-6.21(m, 1H), 6.65-6.80(m, 3H), 7.08(t, J=9.0 Hz, 1H), 7.34-7.42(m, 2H), 7.47-7.59(m, 3H), 7.61-7.92(m, 9H) |
| 1-109 | 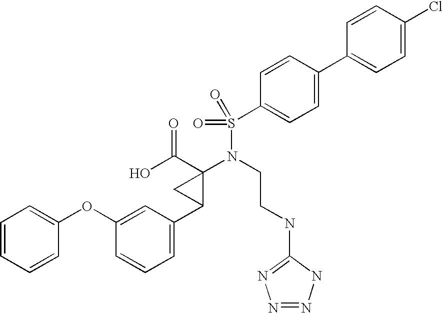 | (DMSO-d6-300 (120° C.)) 1.71(dd, J=6.0, 12.0 Hz, 1H), 2.09(dd, J=6.0, 9.0 Hz, 1H), 3.89-4.12(m, 2H), 4.68-4.83(m, 2H), 5.52(brs, 2H), 6.83(dd, J=3.0, 9.0 Hz, 1H), 6.91-6.98(m, 3H), 7.04-7.12(m, 2H), 7.23-7.37(m, 3H), 7.52(dt, J=9.0, 6.0 Hz, 2H), 7.73(dt, J=9.0, 7.5 Hz, 2H), 7.83(d, J=9.0 Hz, 2H), 7.89(d, J=9.0 Hz, 2H) |
| 1-110 | 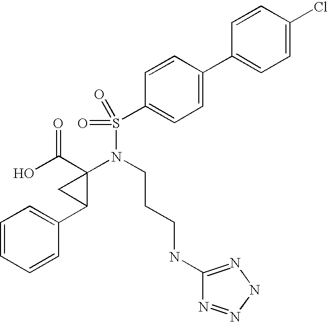 | (METHANOL-d4-400) 1.56-3.06(m, 4H), 3.24-3.68(m, 2H), 3.73-4.01(m, 1H), 4.31-4.69(m, 2H), 7.01-7.32(m, 6H), 7.44-7.60(m, 2H), 7.63-7.72(m, 2H), 7.74-7.84(m, 2H), 7.85-8.00(m, 2H) |

TABLE 1-23

| | | |
|---|---|---|
| 1-111 | 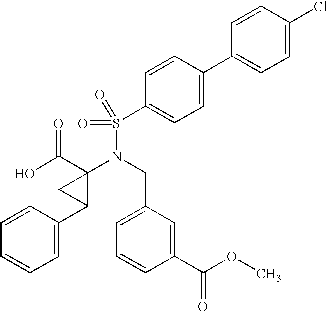 | (METHANOL-d4-400) 1.08-2.76(m, 3H), 3.58-3.96(m, 3H), 4.34-5.33(m, 2H), 6.81-8.07(m, 17H) |
| 1-112 | 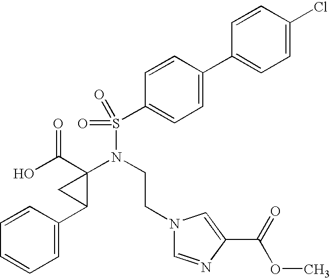 | (METHANOL-d4-400) 1.94-2.03(m, 1H), 2.27-2.36(m, 1H), 2.38-2.48(m, 1H), 3.97(s, 3H), 4.09-4.30(m, 2H), 4.97-5.06(m, 2H), 7.11-7.33(m, 5H), 7.48(d, J=8.0 Hz, 2H), 7.67(d, J=8.0 Hz, 2H), 7.81(d, J=8.0 Hz, 2H), 7.90(d, J=8.0 Hz, 2H), 8.16(br, 1H), 8.97(br, 1H) |
| 1-113 | 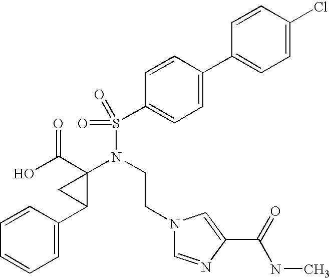 | (CD3OD-400) 1.94-2.01(m, 1H), 2.28-2.38(m, 1H), 2.40-2.50(m, 1H), 2.93(s, 3H), 4.19-4.29(m, 2H), 4.95-5.15(m, 2H), 7.14-7.29(m, 5H), 7.48(d, J=8.0 Hz, 2H), 7.67(d, J=8.0 Hz, 2H), 7.81(d, J=8.0 Hz, 2H), 7.90(d, J=8.0 Hz, 2H), 8.01(br, 1H), 9.11(br, 1H) |
| 1-114 | 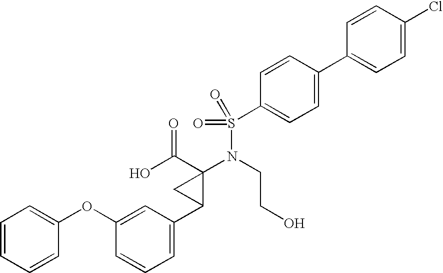 | (DMSO-d6-300 (120° C.)) 1.80(dd, J=6.0, 9.0 Hz, 1H), 2.09(dd, J=6.0, 9.0 Hz, 1H), 3.18(t, J=10.5 Hz, 1H), 3.44-3.57(m, 1H), 3.58-3.70(m, 3H), 6.83(dd, J=3.0, 9.0 Hz, 1H), 6.91-7.02(m, 3H), 7.05-7.13(m, 2H), 7.26(t, J=7.5 Hz, 1H), 7.30-7.38(m, 2H), 7.52(d, J=9.0 Hz, 2H), 7.73(d, J=9.0 Hz, 2H), 7.81(d, J=9.0 Hz, 2H), 7.89(d, J=9.0 Hz, 2H) |
| 1-115 | 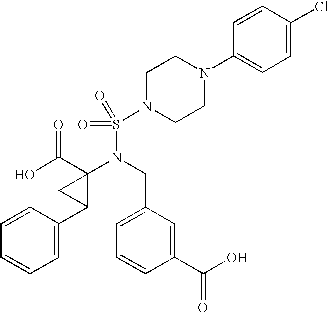 | (METHANOL-d4-400) 1.44-1.56(m, 1H), 2.07-2.25(m, 1H), 2.36-2.70(m, 1H), 3.00-3.15(m, 4H), 3.30-3.42(m, 4H), 6.83-6.94(m, 2H), 7.11-7.25(m, 5H), 7.36-7.50(m, 2H), 7.60-7.76(m, 1H), 7.90-8.01(m, 1H), 7.90-8.01(m, 1H), 7.90-8.01(m, 1H) |

| | | |
|---|---|---|
| Example | Structural formula | NMR |

Table 2-1

| | | |
|---|---|---|
| 2 | 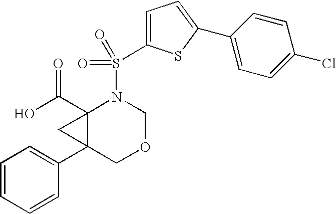 | (MeOH-d4-300) 1.37(d, J=5.7 Hz, 1H), 2.27(d, J=5.7 Hz, 1H), 3.80(d, J=11.7 Hz, 1H), 3.96(d, J=11.7 Hz, 1H), 4.66(d, J=11.7 Hz, 1H), 5.41(d, J=11.7 Hz, 1H), 7.21-7.34 (m, 5H), 7.47 (d, J=6.6 Hz, 2H), 7.50 (d, J=4.1 Hz, 1H), 7.71-7.72 (m, 3H) |
| 2-2 | 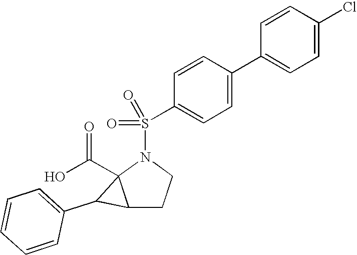 | (DMSO-d6-300) 1.96-2.02 (m, 1H), 2.22-2.30 (m, 2H), 2.54-2.63 (m, 1H), 3.36-3.51 (m, 2H), 7.08-7.20 (m, 5H), 7.52 (d, J=8.7 Hz, 2H), 7.73 (d, J=8.7 Hz, 2H), 7.86 (d, J=8.3 Hz, 2H), 8.01 (d, J=8.3 Hz, 2H). |
| 2-3 | 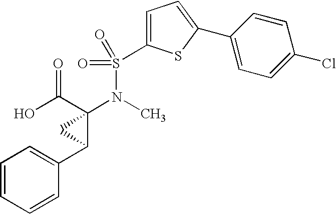 | (DMSO-d6-300) 1.74-2.39(m, 2H), 2.81-3.28(m, 1H), 3.32(brs, 3H), 7.16-7.41(m, 5H), 7.53(d, J=8.7 Hz, 2H), 7.62(d, J=3.8 Hz, 1H), 7.65(d, J=3.8 Hz, 1H), 7.77(d, J=8.7 Hz, 2H), 12.41(s, 1H) |
| 2-4 | 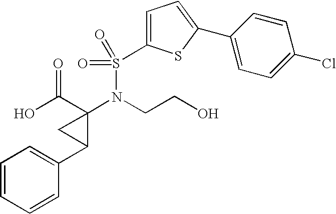 | (MeOH-d4-400) 2.13(br s, 1H), 2.39 (br s, 1H), 3.22-3.35(m, 1H), 3.62-3.98(m, 4H), 7.16-7.71(m, 11H) |
| 2-5 | 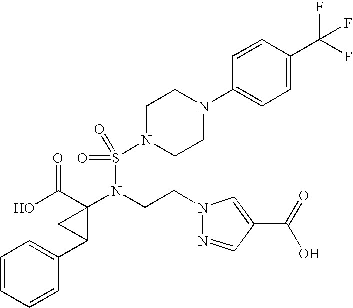 | (MeOH-d4-400) 1.67(br s, 1H), 1.92 (br s, 1H), 2.21(br s, 1H), 3.33-3.41(m, 4H), 3.41-3.50(m, 4H), 3.74-4.72(m, 4H), 7.05 (d, J=8.7 Hz, 2H), 7.14-7.39 (m, 5H), 7.48(d, J=8.7 Hz, 2H), 7.90(s, 1H), 8.18(s, 1H). |

Table 2-2

| | | |
|---|---|---|
| 2-6 | 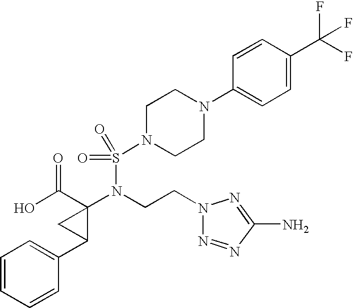 | (MeOH-d4-400) 1.88-2.30(m, 2H), 2.74(t, J=8.0Hz, 1H), 3.40-3.50(m, 4H), 3.34-3.40(m, 4H), 3.95-4.12(m, 2H), 4.25-4.50(m, 2H), 7.07(d, J=8.7 Hz, 2H), 7.13-7.36(m, 5H), 7.49(d, J=8.7 Hz, 2H). |
| 2-7 | 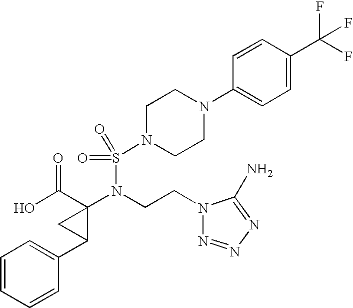 | (MeOH-d4-400) 1.96(dd, J=5.4, 9.9 Hz, 1H), 2.24(dd, J=5.5, 8.5 Hz, 1H), 3.01(t, J=9.2 Hz, 1H), 3.34-3.44(m, 6H), 3.71-4.40(m, 4H), 4.69-5.00(m, 2H), 7.07 (d, J=8.7 Hz, 2H), 7.17-7.36 (m, 5H), 7.50(d, J=8.7 Hz, 2H). |
| 2-8 | 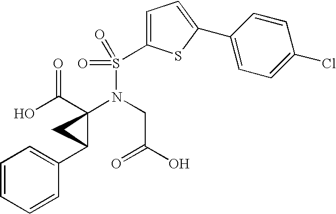 | (DMSO-d6-300) 1.94-2.32(m, 1H), 2.97-3.13 (m, 1H), 3.58-3.67(m, 1H), 4.16-4.49(m, 2H), 7.19-7.25 (m, 5H), 7.52(d, J=8.8 Hz, 2H), 7.62(d, J=4.0 Hz, 1H), 7.69(d, J=4.0 Hz, 1H), 7.76(d, J=8.8 Hz, 2H). |
| 2-9 | 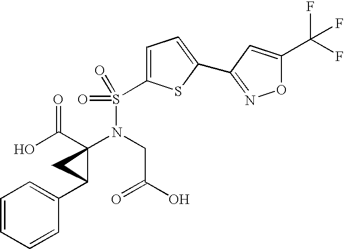 | (DMSO-d6-300) 1.90-2.07(m, 1H), 2.26-2.32 (m, 1H), 3.01-3.12(m, 1H), 4.36-4.49(m, 2H), 7.22-7.40 (m, 5H), 7.87(d, J=3.8 Hz, 1H), 7.92(d, J=3.8 Hz, 1H), 8.20(s, 1H). |
| 2-10 | 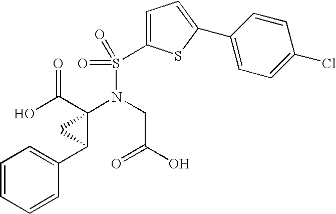 | (DMSO-d6-300) 1.94-2.32 (m, 1H), 2.97-3.13 (m, 1H), 3.58-3.67(m, 1H), 4.16-4.49(m, 2H), 7.19-7.25 (m, 5H), 7.52(d, J=8.8 Hz, 2H), 7.62(d, J=4.0 Hz, 1H), 7.69(d, J=4.0 Hz, 1H), 7.76(d, J=8.8 Hz, 2H). |

Table 2-3

| | | |
|---|---|---|
| 2-11 | 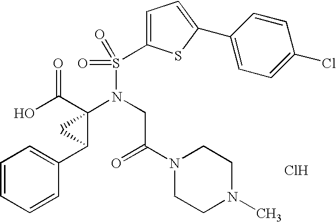 | (DMSO-d6-300) 1.75-2.32(m, 2H), 2.88-3.09 (m, 7H), 3.50-3.57(m, 2H), 4.20-4.43(m, 2H), 7.22-7.31 (m, 5H), 7.55(d, J=8.7 Hz, 2H), 7.65(s, 1H), 7.76-7.79 (m, 3H). |
| 2-12 | 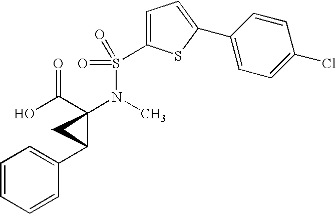 | (DMSO-d6-300) 1.75-2.35(m, 1H), 2.86-3.26(m, 2H), 3.32(brs, 3H), 7.16-7.34(m, 5H), 7.53(d, J=8.7 Hz, 2H), 7.60-7.67(m, 2H), 7.77(d, J=8.7 Hz, 2H), 12.41(brs, 1H) |
| 2-13 | 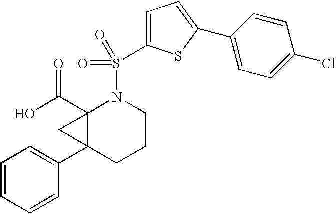 | (Methanol-d4-300) 0.88-0.92(m, 1H), 1.40(d, J=5.8 Hz, 1H), 1.67-1.72(m, 1H), 1.89-2.04(m, 2H), 2.28 (d, J=5.8 Hz, 1H), 3.20-3.25(m, 1H), 3.80(dt, J=13.7, 3.9Hz, 1H), 7.18-7.28 (m, 5H), 7.45-7.48(m, 3H), 7.69-7.72(m, 3H). |
| 2-14 | 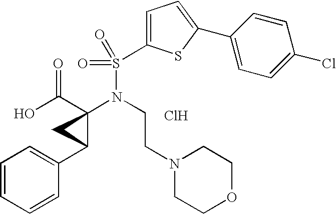 | (DMSO-d6-300) 1.83-2.06(m, 1.4H), 2.24-2.37(m, 0.6H), 2.96-4.22(m, 13H), 7.18-7.49(m, 5H), 7.55(d, J=7.9 Hz, 2H), 7.68(d, J=3.8 Hz, 1H), 7.74-7.86(m, 3H) |
| 2-15 | 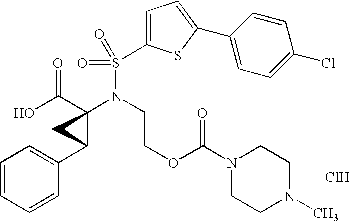 | (DMSO-d6-300) 1.72-2.09(m, 1.3H), 2.26-2.39(m, 0.7H), 2.72(brs, 3H), 2.84-4.49(m, 13H), 7.18-7.36(m, 5H), 7.54(d, J=7.2 Hz, 2H), 7.62-7.73(m, 2H), 7.78(d, J=8.7 Hz, 2H), 10.47(brs, 1H), 12.47(brs, 1H) |

Table 2-4

| | | |
|---|---|---|
| 2-16 | 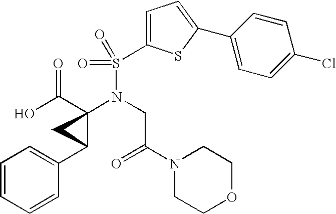 | (DMSO-d6-300) 1.79-1.94(m, 1.3H), 2.18-2.32(m, 0.7H), 3.06-3.78(m, 9H), 4.30-4.48(m, 1H), 4.76-4.93(m, 1H), 7.09-7.32(m, 5H), 7.54(d, J=8.3 Hz, 2H), 7.64(d, J=3.8 Hz, 1H), 7.73-7.83(m, 3H), 12.77(brs, 1H) |
| 2-17 | 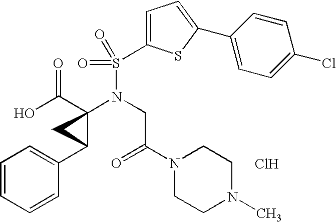 | (DMSO-d6-300) 1.81-1.96(m, 1.3H), 2.13-2.32(m, 0.7H), 2.71-3.74(m, 12H), 3.97-5.10(m, 2H), 7.14-7.36(m, 5H), 7.54(d, J=7.9 Hz, 2H), 7.64(d, J=3.0 Hz, 1H), 7.73-7.83(m, 3H), 10.99(brs, 1H), 12.59(s, 1H) |
| 2-18 | 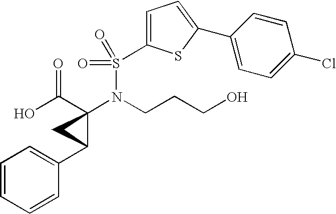 | (DMSO-d6-300) 1.58-2.12(m, 3.3H), 2.22-2.41(m, 0.7H), 2.98-4.06(m, 5H), 4.43-4.71(m, 1H), 7.14-7.47(m, 5H), 7.53(d, J=8.7 Hz, 2H), 7.58-7.72(m, 2H), 7.77(d, J=8.7 Hz, 2H), 12.40(brs, 1H) |
| 2-19 | 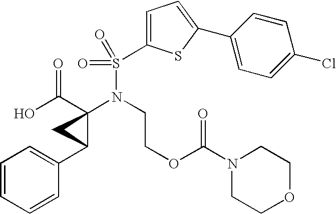 | (DMSO-d6-300) 1.69-2.09(m, 1.3H), 2.24-2.39(m, 0.7H), 2.87-4.51(m, 13H), 7.16-7.48(m, 5H), 7.53(d, J=8.7 Hz, 2H), 7.59-7.74(m, 2H), 7.77(d, J=8.7 Hz, 2H), 12.45(brs, 1H) |
| 2-20 | 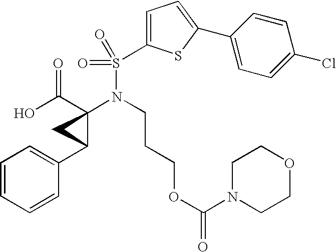 | (DMSO-d6-300) 1.69-2.36(m, 4H), 2.92-4.24(m, 13H), 7.15-7.49(m, 5H), 7.53(d, J=8.7 Hz, 2H), 7.59-7.73(m, 2H), 7.78(d, J=8.7 Hz, 2H), 12.46(brs, 1H) |

Table 2-5

| | | |
|---|---|---|
| 2-21 | 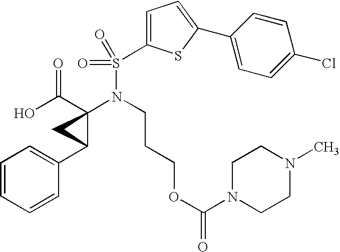 | (DMSO-d6-300) 1.71-2.37(m, 4H), 2.74(s, 3H), 2.86-4.33(m, 13H), 7.16-7.49(m, 5H), 7.54(d, J=8.7 Hz, 2H), 7.61-7.74(m, 2H), 7.78(d, J=8.7 Hz, 2H), 10.72(brs, 1H), 12.45(brs, 1H) |
| 2-22 | 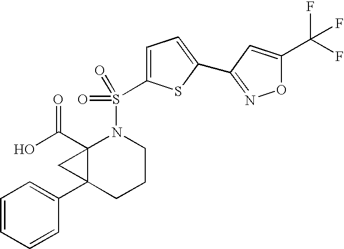 | (MeOH-d4-300) 1.33-1.38(m, 2H), 1.72-1.77 (m, 1H), 1.97-2.04(m, 2H), 2.32(d, J=5.7 Hz, 1H), 3.25-3.27(m, 1H), 3.81-3.86 (m, 1H), 7.22-7.30(m, 5H), 7.72(br s, 1H), 7.78(d, J=4.1 Hz, 1H), 7.81(d, J=4.1 Hz, 1H). |
| 2-23 | 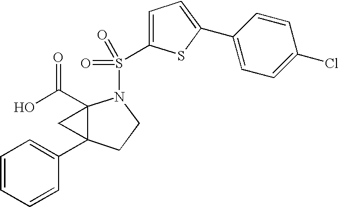 | (DMSO-d6-300) 0.92(t, J=7.2 Hz, 2H), 1.87(d, J=5.3 Hz, 1H), 2.24(t, J=9.4 Hz, 1H), 2.98(d, J=9.4 Hz, 1H), 3.75(t, J=8.9 Hz, 1H), 7.20-7.29(m, 5H), 7.55(d, J=7.9 Hz, 2H), 7.68(d, J=3.8 Hz, 1H), 7.75(d, J=3.8 Hz, 1H), 7.82(d, J=8.7 Hz, 2H), 12.52(br s, 1H). |
| 2-24 | 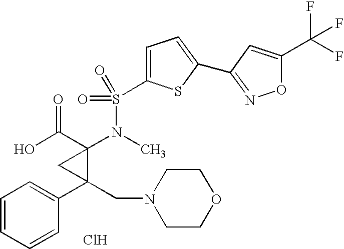 | (DMSO-d6-300) 2.13-2.45(m, 2H), 2.68-3.96(m, 12H), 4.17-4.47(m, 1H), 7.18-7.50(m, 5H), 7.73-7.80(m, 1H), 7.91-8.03(m, 1H), 8.17-8.23(m, 1H), 9.86(brs, 1H), 12.61(brs, 1H) |
| 2-25 | 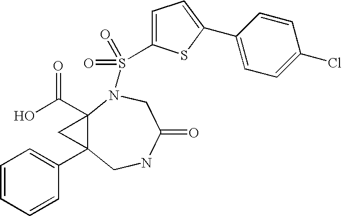 | (MeOH-d4-300) 2.32(d, J=6.0 Hz, 1H), 2.38(d, J=6.0 Hz, 1H), 3.42(d, J=15.0 Hz, 1H), 3.66(d, J=15.0 Hz, 1H), 7.19-7.31(m, 5H), 7.41-7.48(m, 3H), 7.64(d, J=3.0 Hz, 1H), 7.69(d, J=9.0 Hz, 2H) |

Table 2-6

| | | |
|---|---|---|
| 2-26 | 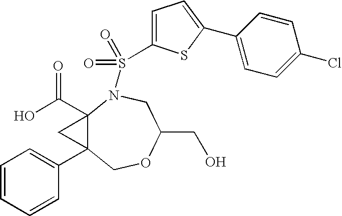 | (CDCL3-300) 2.51(d, J=6.0 Hz, 1H), 2.54 (d, J=6.0 Hz, 1H), 3.70(dd, J=12.6, 2.4 Hz, 1H), 3.90(dd, J=13.0, 3.2 Hz, 1H), 3.94-4.03(m, 2H), 4.28(dd, J=15.3, 12.2 Hz, 1H), 4.41 (dd, J=15.1, 4.9 Hz, 1H), 4.83-4.87(m, 1H), 7.25-7.43(m, 8H), 7.53(d, J=8.7 Hz, 2H), 7.67(d, J=3.8 Hz, 1H) |
| 2-27 | 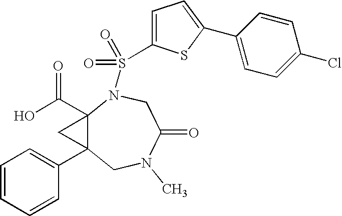 | (MeOH-d4-300) 2.25(d, J=6.0 Hz, 1H), 2.35(d, J=6.0 Hz, 1H), 2.68(s, 3H), 3.56(d, J=15.0 Hz, 1H), 4.03(d, J=15.0 Hz, 1H), 7.15-7.21(m, 2H), 7.26-7.33(m, 3H), 7.43-7.48(m, 3H), 7.65(d, J=3.0 Hz, 1H), 7.69(d, J=9.0 Hz, 2H) |

The following show the results of experiments performed with regard to the aggrecanase-1 inhibitory activity, matrix metalloproteinase-1 (MMP-1) inhibitory activity and MMP-13 inhibitory activity of the compound of the present invention.

(Pharmacological Tests)

### EXPERIMENTAL EXAMPLE 1

### Aggrecanase-1 Inhibitory Action

Particle Assay was used for determination of aggrecanase activity.

The enzyme and substrate were diluted with Tris-HCl buffer, and test compounds were diluted with dimethyl sulfoxide (DMSO).

Test compounds and the enzyme were added into 96-well plate, and polyacrylamide particles containing aggrecan were added as a substrate and the mixture was incubated at 37° C. for 15 hr.

After incubation; the supernatant was transferred to another plate, and mixed with 1,9-dimethylmethylene blue. The absorbance at 595 nm was measured to quantify the amount of glycosaminoglycan (GAG) released in the reaction supernatant. Whale chondroitin sulfate was used as the standard of GAG. The inhibitory activity of the compound in each well (%) was calculated based on the values of enzyme-free well and inhibitor-free well. The inhibitory activity of the compound was represented as IC₅₀ (μM).

### EXPERIMENTAL EXAMPLE 2

### MMP-1 Inhibitory Action

For MMP-1 Assay, Type I collagen Activity Measurement kit (YAGAI YU-72013) modified to a 96-well plate format was used.

The principle of the kit is based on the property of collagen that becomes soluble in ethanol after being cleaved by MMP-1.

The enzyme and substrate were diluted with Tris-HCl buffer, and test compounds were diluted with dimethyl sulfoxide (DMSO).

The enzyme and test compounds were added into 96-well plate, and fluorescein isothiocianate (FITC)-labeled collagen type I was added as a substrate and the mixture was incubated at 37° C. for 3 hr.

The reaction was terminated by addition of Tris-HCl buffer containing ethanol. After centrifugation, the supernatant containing denatured substrate was transferred to another 96-well plate. The collagenase activity of MMP-1 was determined by measuring FITC fluorescence intensity (excitation wavelength 485 nm, emission wavelength 530 nm) of each well. The inhibitory activity of the compound in each well (%) was calculated based on the values of enzyme-free well and inhibitor-free well. The inhibitory activity of the compound was represented as IC₅₀ (μM).

### EXPERIMENTAL EXAMPLE 3

Inhibitory activity of tested compounds on MMP-13 was measured using MMP-13 specific fluorescent substrate with quencher.

The enzyme and substrate were diluted with Tris-HCl buffer, and test compounds were diluted with dimethyl sulfoxide (DMSO).

Test compounds and the enzyme (Recombinant Human MMP-13: R&D systems, 511-MM) were added into 96-well plate. The reaction was initiated by adding synthetic substrate (7-MCA-Pro-CHA-Gly-NVal-His-Ala-DPA: enzyme systems products, Met-06) into the plate. After incubation at 25° C. for 1 h, the reaction was terminated by addition of reaction terminating solution containing acetic acid. Fluorescence intensity of each well was measured (Ex: 325 nm, Em: 405 nm) and the MMP-13 inhibitory activity of the compound in each well (%) was calculated based on the values of enzyme-free well and inhibitor-free well. The inhibitory activity of the compound was represented as IC₅₀ (μM).

The results of the aforementioned Experimental Examples 1 to 3 are shown in Tables 3-1 to 3-3. In the table, +means less than 1 μM, ++means less than 0.1 μM, −means not less than 1 μM, −− means not less than 10 μM and blank column means “not tested”.

TABLE 3

| | Experimental | Experimental | Experimental |
|---|---|---|---|
| | Example 1 | Example 2 | Example 3 |
| Example | IC₅₀ AG1 | IC₅₀ MMP1 | IC₅₀ MMP13 |
| 1-2 | ++ | −− | |
| 1-3 | + | −− | |
| 1-4 | + | −− | |
| 1-5 | + | −− | |
| 1-7 | + | −− | |
| 1-9 | + | −− | |
| 1-10 | ++ | −− | |
| 1-11 | + | −− | |
| 1-12 | + | −− | |
| 1-13 | + | −− | |
| 1-15 | + | −− | |
| 1-17 | + | −− | |
| 1-18 | + | −− | |
| 1-19 | + | −− | |
| 1-20 | + | −− | |
| 1-21 | + | −− | |
| 1-22 | + | −− | |
| 1-23 | + | −− | |
| 1-24 | + | −− | |
| 1-25 | ++ | −− | |
| 1-26 | + | −− | |
| 1-27 | ++ | −− | |
| 1-28 | + | −− | |
| 1-29 | + | −− | |
| 1-30 | + | −− | |
| 1-31 | ++ | −− | |
| 1-32 | + | −− | |
| 1-33 | + | −− | |
| 1-34 | ++ | −− | |
| 1-35 | + | −− | |
| 1-36 | + | −− | |
| 1-37 | + | −− | |
| 1-38 | + | −− | |
| 1-39 | ++ | −− | |
| 1-40 | ++ | −− | |
| 1-41 | ++ | −− | |
| 1-42 | ++ | −− | |
| 1-43 | + | −− | |
| 1-44 | ++ | −− | |
| 1-45 | + | −− | |
| 1-46 | + | −− | |
| 1-47 | ++ | −− | |
| 1-48 | ++ | −− | |
| 1-50 | + | − | |
| 1-51 | ++ | −− | |
| 1-52 | + | −− | |
| 1-53 | + | −− | |
| 1-55 | + | −− | |
| 1-56 | + | −− | |
| 1-60 | + | −− | |
| 1-61 | + | −− | |
| 1-62 | + | −− | |
| 1-63 | + | −− | |
| 1-64 | ++ | −− | |
| 1-66 | ++ | −− | |
| 1-67 | ++ | −− | |
| 1-68 | + | −− | |
| 1-69 | + | −− | |
| 1-70 | + | −− | |
| 1-71 | + | −− | |
| 1-72 | ++ | −− | |
| 1-73 | + | −− | |
| 1-77 | + | −− | |
| 1-78 | + | −− | |
| 1-79 | ++ | −− | |
| 1-81 | + | −− | |
| 1-82 | + | −− | |
| 1-83 | ++ | −− | |
| 1-84 | + | −− | |
| 1-85 | + | −− | |
| 1-86 | ++ | −− | |
| 1-88 | ++ | −− | |
| 1-89 | + | −− | |
| 1-90 | + | −− | |
| 1-91 | + | −− | |
| 1-93 | + | −− | |
| 1-95 | ++ | − | |
| 1-96 | + | −− | |
| 1-99 | ++ | −− | |
| 1-100 | + | −− | |
| 1-101 | + | −− | |
| 1-102 | ++ | −− | |
| 1-103 | ++ | −− | |
| 1-104 | ++ | −− | |
| 1-106 | + | − | |
| 1-107 | + | − | |
| 1-108 | + | −− | |
| 1-109 | ++ | −− | |
| 1-110 | + | −− | |
| 1-112 | + | −− | |
| 1-113 | ++ | −− | |
| 1-114 | + | −− | |
| 1-115 | + | −− | |
| 2-01 | − | −− | − |
| 2-02 | −− | −− | |
| 2-03 | − | −− | −− |
| 2-04 | − | −− | − |
| 2-05 | + | −− | − |
| 2-06 | − | −− | −− |
| 2-07 | − | −− | −− |
| 2-08 | ++ | −− | −− |
| 2-09 | ++ | −− | − |
| 2-10 | + | −− | −− |
| 2-11 | − | −− | −− |
| 2-12 | − | −− | − |
| 2-13 | + | −− | − |
| 2-14 | − | −− | |
| 2-15 | − | −− | − |
| 2-16 | − | −− | − |
| 2-17 | − | −− | − |
| 2-18 | − | −− | + |
| 2-19 | − | −− | + |
| 2-20 | − | −− | − |
| 2-21 | − | −− | − |
| 2-22 | − | −− | − |
| 2-23 | − | −− | − |
| 2-24 | − | −− | −− |
| 2-25 | − | −− | − |
| 2-26 | − | −− | − |
| 2-27 | − | −− | − |

The compound (1) of the present invention described in the results above has superior aggrecanase inhibitory activity and MMP-13 inhibitory activity, and high selectivity to aggrecanase as compared to the activity of MMP-1.

### INDUSTRIAL APPLICABILITY

According to the present invention, a compound useful as a prophylactic or therapeutic agent for diseases mediated by aggrecanase, such as osteoarthritis (OA), rheumatoid arthritis (RA), joint injury, reactive arthritis, cancer, asthma, allergic reaction, chronic pulmonary emphysema, fibroid lung, acute respiratory distress (ARDS), lung infection, interstitial pneumonia, bone resorption disorder, etc. is provided.

1. An N-substituted-N-sulfonylaminocyclopropane compound represented by the formula (1) 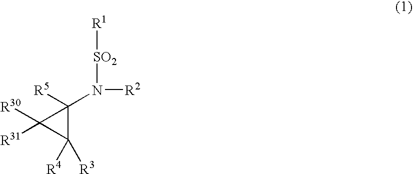
wherein
R¹ is
—W-A¹-W¹-A²
wherein
W is —(CH₂)ₘ—X(CH₂)ₙ—,
W₁ is (CH₂)ₘ₁—X₁-(CH₂)ₙ₁—,
wherein
m, n, m1 and n1 are the same or different and each is selected from 0 and an integer ranging from 1 to 6,
X and X₁ are the same or different and each is a linker selected from the following group A,
group A:
(a) a single bond,
(b) a C₁₋₆ alkylene group,
(c) a C₂₋₆ alkenylene group,
(d) a C₂₋₆ alkynylene group,
(e) —O—,
(f) —N(R⁶)
(g) —S(O)ₘ₃—,
(h) —CO—,
(i) —COO—,
(j) —OCO—,
(k) —CON(R⁶)—,
(l) —N(R⁶)CO—,
(m) —SO₂N(R⁶)—,
(n) —N(R⁶)SO₂—,
(o) —N(R⁶)CON(R⁷)_,
(p) —N(R⁶)SO₂N(R⁷)_,
(q) —OCON(R⁶)_,
(r) —N(R⁶)COO—, and
(s) —S(O)ₘ₃—(CH₂)ₙ₃—CO—,
(wherein R⁶ and R⁷ are the same or different and each is selected from a hydrogen atom, a C₁₋₆ alkyl group optionally substituted by halogen atoms or hydroxyl groups, a C₃₋₁₄ hydrocarbon ring group and a heterocyclic group, m3 is selected from 0 and an integer ranging from 1 to 2, and n3 is an integer ranging from 1 to 2),
A¹ is selected from an optionally substituted C₃₋₁₄ hydrocarbon ring group and an optionally substituted heterocyclic group, and
A² is selected from a substituted C₃₋₁₄ hydrocarbon ring group and a substituted heterocyclic group,
or A¹ and A² may be taken together with a substituent thereof to form an optionally substituted fused C₆₋₁₄ hydrocarbon ring group;
R² is selected from
(CH₂)ₘ₅—X₅-(CH₂)ₙ₅-A⁵ (1) and
(CH₂)ₘ₅—X₅—(CH₂)ₙ₅—R³² (2)
(wherein m5 and n5 are the same or different and each is selected from 0 and an integer ranging from 1 to 6,
X₅ are the same or different and each is a linker selected from the above-mentioned group A,
A⁵ is selected from an optionally substituted C₃₋₁₄ hydrocarbon ring group and an optionally substituted heterocyclic group, and
R³² is a substituent selected from the following group B, provided that when m5 and n5 are 0 and X₅ is a single bond, then R³² should not be is not a hydrogen atom);
group B:
(a) a hydrogen atom,
(b) a halogen atom,
(c) a hydroxyl group,
(d) a nitro group,
(e) a cyano group,
(f) a carboxyl group,
(g) an amino group,
(h) an amide group,
(i) a C₂₋₆acyl group,
(j) a halogenated C₁₋₆alkyl group,
(k) a C₁₋₆alkyl group optionally substituted by hydroxyl groups,
(l) a C₂₋₆alkenyl group optionally substituted by halogen atoms,
(m) a C₂₋₆alkynyl group,
(n) a C₁₋₆alkoxy group optionally substituted by hydroxyl groups
(o) a C₁₋₆alkoxy-C₁₋₁₆alkyl group,
(p) a C₁₋₆alkoxy-carbonyl group,
(q) a C₁₋₆alkyl-aminocarbonyl group optionally substituted by halogen atoms,
(r) a mono(C₁₋₆alkyl)amino group,
(s) a di(C₁₋₆alkyl)amino group,
(t) a C₁₋₆alkyl-carbonylamino group optionally substituted by halogen atoms,
(u) a C₁₋₆alkylsulfonyl group, and
(v) a C₁₋₆alkylsulfonylamino group,
or R² and R³ and the cyclopropane ring may be taken together to form an optionally further substituted fused ring);
R³ and R⁴ are the same or different and each is selected from
—(CH₂)ₘ₂—X₂—(CH₂)ₙ₂-A⁴ (1)
(wherein m2 and n2 are the same or different and each is selected from 0 and an integer ranging from 1 to 6, X₂ is a linker selected from the above-mentioned group A, and A⁴ is selected from an optionally substituted C₃₋₁₄ hydrocarbon ring group and an optionally substituted heterocyclic group) and
—(CH₂)ₘ₆—X₆—(CH₂)ₙ₆—R³ (2)
(wherein m6 and n6 are the same or different and each is selected from 0 and an integer ranging from 1 to 6, X₆ is a linker selected from the above-mentioned group A, and R³³ is a substituent selected from the above-mentioned group B);
or A⁴ and R³³ may be taken together to form an optionally substituted fused ring group, and R³ and R⁴ may be taken together with a carbon atom bonded thereto to form the following ring 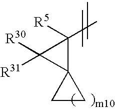
(wherein m10 is an integer ranging from 1 to 6),
provided that R³ and R⁴ are not hydrogen atoms at the same time;
R⁵ is selected from
(1) —CO₂R²¹,
(2) —C(O)NHOR²¹,
(3) —C(O)NH—SO₂—R²¹,
(4) —C(O)NHR²¹,
(5) —SH,
(6) —CH₂CO₂R²¹,
(7) —C(O)R²¹,
(8) —N(OH)COR²¹,
(9) —SN₂H₂R²¹,
(10) —SONHR²¹,
(11) —CH₂CO₂H,
(12) —PO(OH)₂,
(13) —PO(OH)NHR²¹,
(14) —CH₂SH,
(15) —CH₂OH,
(16) —(CH₂)ᵣ, —PO(OH)—(CH₂)ᵣ₂R²¹,
(17) —NHR²¹,
(18) —NH—NHR²¹′ and
(19) —(CH₂)ᵣ₁—R⁵⁰
(wherein r1 and r2 are the same or different and each is selected from 0 and an integer ranging from 1 to 6,
R²¹ is selected from
(1) a hydrogen atom,
(2) an optionally substituted C₁₋₁₀ alkyl group,
(3) an optionally substituted C₆₋₁₄ aryl-C₁₋₆ alkyl group and
—(CH₂)ₘ₇—X₇—(CH₂)ₙ₇—R³⁴ (4)
(wherein m7 and n7 are the same or different and each is selected from 0 and an integer ranging from 1 to 6, X₇ is a linker selected from the above-mentioned group A, R³⁴ is a substituent selected from the following group C));
group C:
(a) a hydrogen atom,
(b) a halogen atom,
(c) a hydroxyl group,
(d) a nitro group,
(e) a cyano group,
(f) a carboxyl group,
(g) an amino group,
(h) an amide group,
(i) a C₂₋₆ acyl group,
(o) a halogenated C₁₋₆ alkyl group,
(k) a C₁₋₆ alkyl group optionally substituted by hydroxyl groups,
(l) a C₂₋₆ alkenyl group optionally substituted by halogen atoms,
(m) a C₂₋₆ alkynyl group,
(n) a C₁₋₆ alkoxy group optionally substituted by hydroxyl groups,
(o) a C₁₋₆alkoxy-C₁₋₆alkyl group,
(p) a C₁₋₆ alkoxy-carbonyl group,
(q) a C₁₋₆ alkyl-aminocarbonyl group optionally substituted by halogen atoms,
(r) a mono(C₁₋₆alkyl)amino group,
(s) a di(C₁₋₆ alkyl)amino group,
(t) a C₁₋₆ alkyl-carbonylamino group optionally substituted by halogen atoms,
(u) a C₁₋₆alkylsulfonyl group,
(v) a C₁₋₆alkylsulfonylamino group,
(w) a C₃₋₁₄ hydrocarbon ring group optionally substituted by 1 to 5 substituents selected from the above-mentioned group B, and
(x) a heterocyclic group optionally substituted by 1 to 5 substituents selected from the above-mentioned group B), and
R⁵⁰ is selected from an optionally substituted C₃₋₁₄ hydrocarbon ring group and an optionally substituted heterocyclic group;
or R²¹ of —C(O)NHR²¹, A⁴ and the cyclopropane ring may be taken together to form an optionally further substituted fused ring;
R³⁰ and R³¹ are the same or different and each is selected from
—(CH₂)ₘ₈—X₈—(CH₂)ₙ₈-A⁶ (1)
(wherein m8 and n8 are the same or different and each is 0 or an integer ranging from 1 to 6, X₈ is a linker selected from the above-mentioned group A, and A⁶ is selected from an optionally substituted C₃₋₁₄ hydrocarbon ring group and an optionally substituted heterocyclic group), and
—(CH₂)ₘ₉—X₉—(CH₂)ₙ₉—R³⁶ (2)
(wherein m9 and n9 are the same or different and each is selected from 0 and an integer ranging from 1 to 6, X₉ is a linker selected from the above-mentioned group A, and R³⁶ is a substituent selected from the above-mentioned group B);
or A⁴, R³⁶ and the cyclopropane ring may be taken together to form an optionally further substituted fused ring,
or R²¹ of —CO₂R²¹, R³⁰ and the cyclopropane ring may be taken together to form an optionally further substituted fused ring,
or further, R³⁰ and R³¹ may be taken together with a carbon atom bonded thereto to form the following ring 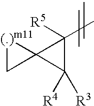
(wherein m11 is an integer ranging from 1 to 6);
or a pharmaceutically acceptable salt thereof.

2. The compound of claim 1, wherein A² is 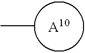
(wherein ring A¹⁰ is selected from a C₃₋₁₄ hydrocarbon ring group and a heterocyclic group, and further the ring A₁₀ is substituted by 1 to 5 groups of “—(CH₂)ₘ₁₂—X₁₂—(CH₂)ₙ₁₂—R³⁷”, which are the same or different (wherein m12 and n12 are the same or different and each is selected from 0 and an integer ranging from 1 to 6, X₁₂ is a linker selected from the above-mentioned group A and R³⁷ is a substituent selected from the above-mentioned group C)),
or the ring A₁₀ and A¹ may be taken together with a substituent thereof to form an optionally substituted fused C₆₋₁₄ hydrocarbon ring group,
A⁴, A⁵ and A⁶ may be the same or different and each is 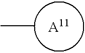
(wherein ring A¹¹ is selected from a C₃₋₁₄ hydrocarbon ring group and a heterocyclic group, and further the ring A¹¹ is optionally substituted by 1 to 5 groups of “—(CH₂)ₘ₁₃—X₁₃—(CH₂)ₙ₁₃—R³⁸”, which are the same or different (wherein m13 and n13 are the same or different and each is selected from 0 and an integer ranging from 1 to 6, X₁₃ is a linker selected from the above-mentioned group A and R³⁸ is a substituent selected from the above-mentioned group C)); or a pharmaceutically acceptable salt thereof.

3. The compound of claim 2, wherein m and n are 0 and X is a single bond; or a pharmaceutically acceptable salt thereof.

4. The compound of claim 3, wherein m1 and n1 are 0 and X, is a single bond; or a pharmaceutically acceptable salt thereof.

5. The compound of claim 4, wherein R⁵ is selected from —CO₂R²¹ and —C(O)NHOR²¹; or a pharmaceutically acceptable salt thereof.

6. The compound of claim 5, wherein R²¹ is a hydrogen atom; or a pharmaceutically acceptable salt thereof.

7. The compound of claim 6, wherein R³ is —(CH₂)ₘ₂—X₂—(CH₂)ₙ₂-A⁴; or a pharmaceutically acceptable salt thereof.

8. The compound of claim 1, which is selected from the group consisting of
(1S*,5S*,6R*)-2-(4′-Chloro-biphenyl-4-sulfonyl)-6-phenyl-2-aza-bicyclo[3.1.0]hexane-1-carboxylic acid,
(1S,2R)-1-{[5-(4-Chloro-phenyl)-thiophene-2-sulfonyl]-methyl-amino}-2-phenyl-cyclopropanecarboxylic acid,
(1R*,2S*)-1-[[5-(4-Chloro-phenyl)-thiophene-2-sulfonyl]-(2-hydroxy-ethyl)-amino]-2-phenyl-cyclopropanecarboxylic acid,
1-(2-{((1R*,2S*)-1-Carboxy-2-phenyl-cyclopropyl)-[4-(4-trifluoromethyl-phenyl)-piperazine-1-sulfonyl]-amino}-ethyl)-1H-pyrazole-4-carboxylic acid,
(1R*,2S*)-1-{[2-(5-Amino-tetrazol-2-yl)-ethyl]-[4-(4-trifluoromethyl-phenyl)-piperazine-1-sulfonyl]-amino}-2-phenyl-cyclopropanecarboxylic acid,
(1R*,2S*)-1-{[2-(5-Amino-tetrazol-1-yl)-ethyl]-[4-(4-trifluoromethyl-phenyl)-piperazine-1-sulfonyl]-amino}-2-phenyl-cyclopropanecarboxylic acid,
(1R,2S)-1-{Carboxymethyl-[5-(4-chloro-phenyl)-thiophene-2-sulfonyl]-amino}-2-phenyl-cyclopropanecarboxylic acid,
(1R,2S)-1-{Carboxymethyl-[5-(5-trifluoromethyl-isoxazol-3-yl)-thiophene-2-sulfonyl]-amino}-2-phenyl-cyclopropanecarboxylic acid,
(1S,2R)-1-{Carboxymethyl-[5-(4-chloro-phenyl)-thiophene-2-sulfonyl]-amino}-2-phenyl-cyclopropanecarboxylic acid,
(1S,2R)-1-{[5-(4-Chloro-phenyl)-thiophene-2-sulfonyl]-[2-(4-methyl-piperazin-1-yl)-2-oxo-ethyl]-amino}-2-phenyl-cyclopropanecarboxylic acid
(1R,2S)-1-{[5-(4-Chloro-phenyl)-thiophene-2-sulfonyl]-methyl-amino}-2-phenyl-cyclopropanecarboxylic acid,
(1R*,6S*)-2-[5-(4-Chloro-phenyl)-thiophene-2-sulfonyl]-6-phenyl-2-aza-bicyclo[4.1.0]heptane-1-carboxylic acid,
(1R,2S)-1-[[5-(4-Chloro-phenyl)-thiophene-2-sulfonyl]-(2-morpholin-4-yl-ethyl)-amino]-2-phenyl-cyclopropanecarboxylic acid,
4-Methyl-piperazine-1-carboxylic acid 2-{((1R,2S)-1-carboxy-2-phenyl-cyclopropyl)-[5-(4-chloro-phenyl)-thiophene-2-sulfonyl]-amino}-ethyl ester,
(1R,2S)-1-[[5-(4-Chloro-phenyl)-thiophene-2-sulfonyl]-(2-morpholin-4-yl-2-oxo-ethyl)-amino]-2-phenyl-cyclopropanecarboxylic acid,
(1R,2S)-1-{[5-(4-Chloro-phenyl)-thiophene-2-sulfonyl]-[2-(4-methyl-piperazin-1-yl)-2-oxo-ethyl]-amino}-2-phenyl-cyclopropanecarboxylic acid,
(1R,2S)-1-[[5-(4-Chloro-phenyl)-thiophene-2-sulfonyl]-(3-hydroxy-propyl)-amino]-2-phenyl-cyclopropanecarboxylic acid,
Morpholine-4-carboxylic acid 2-{((1R,2S)-1-carboxy-2-phenyl-cyclopropyl)-[5-(4-chloro-phenyl)-thiophene-2-sulfonyl]-amino}-ethyl ester,
Morpholine-4-carboxylic acid 3-{((1R,2S)-1-carboxy-2-phenyl-cyclopropyl)-[5-(4-chloro-phenyl)-thiophene-2-sulfonyl]-amino}-propyl ester,
4-Methyl-piperazine-1-carboxylic acid 3-{((1R,2S)-1-carboxy-2-phenyl-cyclopropyl)-[5-(4-chloro-phenyl)-thiophene-2-sulfonyl]-amino}-propyl ester,
(1R*,6R*)-2-[5-(4-Chloro-phenyl)-thiophene-2-sulfonyl]-6-phenyl-4-oxa-2-aza-bicyclo[4.1.0]heptane-1-carboxylic acid,
(1R*,6S*)-6-Phenyl-2-[5-(5-trifluoromethyl-isoxazol-3-yl)-thiophene-2-sulfonyl]-2-aza-bicyclo[4.1.0]heptane-1-carboxylic acid,
(1R*,5S*)-2-[5-(4-Chloro-phenyl)-thiophene-2-sulfonyl]-5-phenyl-2-aza-bicyclo[3.1.0]hexane-1-carboxylic acid,
(1R*,2S*)-1-{Methyl-[5-(5-trifluoromethyl-isoxazol-3-yl)-thiophene-2-sulfonyl]-amino}-2-morpholin-4-ylmethyl-2-phenyl-cyclopropanecarboxylic acid,
1R*,7S*)-2-[5-(4-Chloro-phenyl)-thiophene-2-sulfonyl]-4-oxo-7-phenyl-2,5-diaza-bicyclo[5.1.0]octane-1-carboxylic acid,
(1R,7R*)-2-[5-(4-Chloro-phenyl)-thiophene-2-sulfonyl]-4-hydroxymethyl-7-phenyl-5-oxa-2-aza-bicyclo[5.1.0]octane-1-carboxylic acid, and
(1R*,7R*)-2-[5-(4-Chloro-phenyl)-thiophene-2-sulfonyl]-5-methyl-4-oxo-7-phenyl-2-,5-diaza-bicyclo[5.1.0]octane-1-carboxylic acid;
or a pharmaceutically acceptable salt thereof.

9. The compound of claim 1, which is represented by the formula (1′): 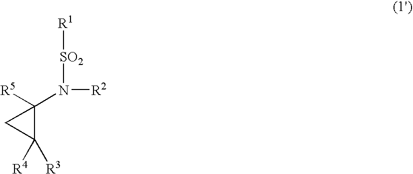
wherein
R¹ is —W-A¹-W¹-A²,
wherein
W is —(CH₂)ₘ—X—(CH₂)ₙ—,
W₁ is (CH₂)ₘ₁—X₁—(CH₂)ₙ₁—,
wherein
m, m1, n and n1 are the same or different and each is selected from 0 and an integer ranging from 1 to 6,
X and X, are the same or different and each is selected from a single bond, a C₁₋₆ alkylene group, a C₂₋₆ alkenylene group, a C₂₋₆ alkynylene group, —O—, —N(R⁶)—, S(O)_{q}—, —CO—, —CON(R⁶)—, —N(R⁶)CO—, —SO₂N(R⁶)—, —N(R⁶)SO₂—, —N(R⁶)CON(R⁷)—, —N(R⁶)SO₂N(R⁷)—, —OCON(R⁶)— and —N(R⁶)COO—,
wherein
R⁶ and R⁷ are the same or different and each is selected from a hydrogen atom, a C₁₋₆ alkyl group, an optionally substituted C₃₋₁₄ hydrocarbon ring group and an optionally substituted heterocyclic group,
q is selected from 0 and an integer ranging from 1 to 2,
A¹ is selected from an optionally substituted C₃₋₁₄ hydrocarbon ring group and an optionally substituted heterocyclic group;
A² is selected from a substituted C₃₋₁₄ hydrocarbon ring group and a substituted heterocyclic group;
R² is selected from
—(CH₂)ᵣ—CO—R⁸ (1)
wherein
r is selected from 0 and an integer ranging from 1 to 6,
R⁸ is selected from a C₁₋₆ alkoxy group and —N(R⁹)(R¹⁰)
wherein
R⁹ and R¹⁰ are the same or different and each is selected from a hydrogen atom, a C₁₋₆ alkyl group, a C₁₋₆ alkylsulfonyl group, —SO₂A³ and A³, or may be taken together with a nitrogen atom to form an optionally substituted nitrogen-containing heterocyclic group, A³ is selected from an optionally substituted C₃₋₁₄ hydrocarbon ring group and an optionally substituted heterocyclic group;
—(CH₂)ᵣ—N(R¹¹)(R¹²) (2)
wherein
r is as defined above,
R¹¹ and R¹² are the same or different and each is selected from a hydrogen atom, a C₁₋₆ alkyl group, —CO—R³, —SO₂—R¹⁴ and A³, or may be taken together with a nitrogen atom to form an optionally substituted nitrogen-containing heterocyclic group,
wherein
R¹³ is selected from a C₁₋₆ alkyl group optionally substituted by C₁₋₆ alkoxy groups or hydroxy groups, and a C₁₋₆ alkoxy group,
R¹⁴ is selected from a C₁₋₆ alkyl group, a halogenated C₁₋₆ alkyl group, —N(R¹⁵)(R¹⁶) and A³,
wherein
R¹⁵ and R¹⁶ are the same or different and each is selected from a hydrogen atom, a C₁₋₆ alkyl group, a C₁₋₆ alkoxy-carbonyl group and A³,
A³ is as defined above; and
—(CH₂)ᵣ—R¹⁷ (3)
wherein
r is as defined above,
R¹⁷ is selected from a C₁₋₆ alkyl group optionally substituted by at least one substituent selected from hydroxy groups and —CO₂R¹⁸ groups, and A³,
wherein
R¹⁸ is selected from a hydrogen atom and a C₁₋₆ alkyl group,
A³ is as defined above;
R³ and R⁴ are the same or different and each is selected from
(1) a hydrogen atom,
(2) a C₁₋₆ alkyl group
(3) a halogenated C₁₋₆ alkyl group, and
—(CH₂)ₘ₂—X₂—(CH₂)ₙ₂-A⁴, (4)
wherein
m2 and n2 are the same or different and each is selected from 0 and an integer ranging from 1 to 6,
X₂ is selected from a single bond, a C₁₋₆ alkylene group, a C₂₋₆ alkenylene group, a C₂₋₆ alkynylene group, —O—, —N(R¹⁹)—, S(O)_{q}—, —CO—, —CON(R¹⁹)—, —N(R¹⁹)CO—, —SO₂N(R¹⁹)—, —N(R¹⁹)SO₂—, —N(R¹⁹)CON(R²⁰)—, —N(R¹⁹)SO₂N(R²⁰)—, —OCON(R¹⁹)— and —N(R¹⁹)COO—,
wherein
R¹⁹ and R²⁰ are the same or different and each is selected from a hydrogen atom, a C₁₋₆ alkyl group, an optionally substituted C₃₋₁₄ hydrocarbon ring group and an optionally substituted heterocyclic group,
q1 is selected from 0 and an integer ranging from 1 to 2,
A⁴ is selected from an optionally substituted C₃₋₁₄ hydrocarbon ring group and an optionally substituted heterocyclic group;
R⁵ is selected from
(1) —CO₂R²¹,
(2) —C(O)NHOR²¹,
(3) —C(O)NH—SO₂—R²¹,
(4) —C(O)NHR²¹,
(5) —SH,
(6) —CH₂CO₂R²¹,
(7) —C(O)R²¹,
(8) —N(OH)COR²¹,
(9) —SN₂H₂R²¹,
(10) —SONHR²¹,
(11) —CH₂CO₂H,
(12) —PO(OH)₂,
(13) —PO(OH)NHR²¹,
(14) —CH₂SH and
(15) —CH₂OH
wherein
R²¹ is selected from a hydrogen atom, an optionally substituted C₁₋₁₀ alkyl group and an optionally substituted C₆₋₁₄ aryl-C₁₋₆ alkyl group;
or a pharmaceutically acceptable salt thereof.

10. The compound of claim 9, wherein m and n are 0, and X is a single bond; or a pharmaceutically acceptable salt thereof.

11. The compound of claim 10, wherein m1 and n1 are 0; or a pharmaceutically acceptable salt thereof.

12. The compound of claim 11, wherein R⁵ is selected from —CO₂R²¹ and —C(O)NHOR²¹; or a pharmaceutically acceptable salt thereof.

13. The compound of claim 12, wherein R²¹ is a hydrogen atom; or a pharmaceutically acceptable salt thereof.

14. The compound of claim 13, wherein R³ is —(CH₂)ₘ₂—X₂—(CH₂)ₙ₂-A⁴; or a pharmaceutically acceptable salt thereof.

15. The compound of claim 9, which is selected from the group consisting of
(1S,2R)-1-[carboxymethyl-(4′-chloro-biphenyl-4-sulfonyl)-amino]-2-phenyl-cyclopropanecarboxylic acid,
(1R,2S)-1-[carboxymethyl-(4′-chloro-biphenyl-4-sulfonyl)-amino]-2-phenyl-cyclopropanecarboxylic acid,
(1S,2R)-2-benzyl-1-[carboxymethyl-(4′-chloro-biphenyl-4-sulfonyl)-amino]-cyclopropanecarboxylic acid,
(1R*,2S*)-1-[(2-tert-butoxycarbonylamino-ethyl)-(4′-chloro-biphenyl-4-sulfonyl)-amino]-2-phenyl-cyclopropanecarboxylic acid,
(1R,2S)-1-[(4′-chloro-biphenyl-4-sulfonyl)-(5-oxo-2,5-dihydro-1H-pyrazol-3-ylmethyl)-amino]-2-phenyl-cyclopropanecarboxylic acid,
(1R*,2S*)-1-[(4′-chloro-biphenyl-4-sulfonyl)-(2-hydroxy-2-methyl-propyl)-amino]-2-phenyl-cyclopropanecarboxylic acid,
(1R*,2S*)-1-[(4′-chloro-biphenyl-4-sulfonyl)-(2-methanesulfonylamino-ethyl)-amino]-2-phenyl-cyclopropanecarboxylic acid,
(1R*,2S*)-1-[(1H-benzoimidazol-2-ylmethyl)-(4′-chloro-biphenyl-4-sulfonyl)-amino]-2-phenyl-cyclopropanecarboxylic acid,
(1R,2S*)-1-[(4′-chloro-biphenyl-4-sulfonyl)-(2-isopropoxycarbonylaminosulfonylamino-ethyl)-amino]-2-phenyl-cyclopropanecarboxylic acid,
(1R*,2S*)-4-{[(1-carboxy-2-phenyl-cyclopropyl)-(4′-chloro-biphenyl-4-sulfonyl)-amino]-methyl}-benzoic acid,
(1R*,2S*)-1-[carboxymethyl-(4′-chloro-biphenyl-4-sulfonyl)-amino]-2-(4-chloro-phenyl)-cyclopropanecarboxylic acid,
(1R*,2S*)-1-[carboxymethyl-(4′-chloro-biphenyl-4-sulfonyl)-amino]-2-(3-chloro-phenyl)-cyclopropanecarboxylic acid,
(1R*,2S*)-1-[carboxymethyl-(4′-chloro-biphenyl-4-sulfonyl)-amino]-2-(2-chloro-phenyl)-cyclopropanecarboxylic acid,
(1R*,2R*)-1-[carboxymethyl-(4′-chloro-biphenyl-4-sulfonyl)-amino]-2-(2-chloro-phenyl)-cyclopropanecarboxylic acid,
(1R*,2S*)-1-[(4′-chloro-biphenyl-4-sulfonyl)-(2H-tetrazol-5-ylmethyl)-amino]-2-phenyl-cyclopropanecarboxylic acid,
(1R*,2S*)-1-[benzyl-(4′-chloro-biphenyl-4-sulfonyl)-amino]-2-phenyl-cyclopropanecarboxylic acid,
(1R*,2S*)-1-[(4′-chloro-biphenyl-4-sulfonyl)-(3-hydroxy-benzyl)-amino]-2-phenyl-cyclopropanecarboxylic acid,
(1R*,2S*)-1-[carboxymethyl-(4′-chloro-biphenyl-4-sulfonyl)-amino]-2-(4-methyl-phenyl)-cyclopropanecarboxylic acid,
(1R*,2S*)-1-[carboxymethyl-(4′-chloro-biphenyl-4-sulfonyl)-amino]-2-(2-methyl-phenyl)-cyclopropanecarboxylic acid,
(1R*,2S*)-1-[carboxymethyl-(4′-chloro-biphenyl-4-sulfonyl)-amino]-2-(3-methyl-phenyl)-cyclopropanecarboxylic acid,
(1R*,2S*)-1-[carboxymethyl-(4′-chloro-biphenyl-4-sulfonyl)-amino]-2-(3-methoxy-phenyl)-cyclopropanecarboxylic acid,
(1R*,2S*)-1-[carboxymethyl-(4′-chloro-biphenyl-4-sulfonyl)-amino]-2-(2-methoxy-phenyl)-cyclopropanecarboxylic acid,
(1R*,2S*)-1-[carboxymethyl-(4′-chloro-biphenyl-4-sulfonyl)-amino]-2-(3,4-dichloro-phenyl)-cyclopropanecarboxylic acid,
(1R*,2S*)-1-[carboxymethyl-(4′-chloro-biphenyl-4-sulfonyl)-amino]-2-(2,5-dichloro-phenyl)-cyclopropanecarboxylic acid,
(1R*,2S*)-1-[carboxymethyl-(4′-chloro-biphenyl-4-sulfonyl)-amino]-2-(3-phenoxy-phenyl)-cyclopropanecarboxylic acid,
(1R*,2S*)-2-biphenyl-2-yl-1-[carboxymethyl-(4′-chloro-biphenyl-4-sulfonyl)-amino]-cyclopropanecarboxylic acid,
(1R*,2S*)-1-[carboxymethyl-(4′-chloro-biphenyl-4-sulfonyl)-amino]-2-(3-cyano-phenyl)-cyclopropanecarboxylic acid,
(1R*,2S*)-1-[carboxymethyl-(4′-chloro-biphenyl-4-sulfonyl)-amino]-2-(2-cyano-phenyl)-cyclopropanecarboxylic acid,
(1R*,2S*)-1-[carboxymethyl-(4′-chloro-biphenyl-4-sulfonyl)-amino]-2-(2,6-dichloro-phenyl)-cyclopropanecarboxylic acid,
(1R*,2S*)-1-[carboxymethyl-(4′-chloro-biphenyl-4-sulfonyl)-amino]-2-(4-cyano-phenyl)-cyclopropanecarboxylic acid,
(1R*,2S*)-2-(2-benzyl-phenyl)-1-[carboxymethyl-(4′-chloro-biphenyl-4-sulfonyl)-amino]-cyclopropanecarboxylic acid,
(1R*,2S*)-2-biphenyl-4-yl-1-[carboxymethyl-(4′-chloro-biphenyl-4-sulfonyl)-amino]-cyclopropanecarboxylic acid,
(1R*,2S*)-1-[carboxymethyl-(4′-chloro-biphenyl-4-sulfonyl)-amino]-2-(2-trifluoromethyl-phenyl)-cyclopropanecarboxylic acid,
(1R*,2S*)-1-[carboxymethyl-(4′-chloro-biphenyl-4-sulfonyl)-amino]-2-(3-trifluoromethyl-phenyl)-cyclopropanecarboxylic acid,
(1R*,2S*)-1-[carboxymethyl-(4′-chloro-biphenyl-4-sulfonyl)-amino]-2-(5-chloro-2-trifluoromethyl-phenyl)-cyclopropanecarboxylic acid,
(1R*,2S*)-1-[carbamoylmethyl-(4′-chloro-biphenyl-4-sulfonyl)-amino]-2-phenyl-cyclopropanecarboxylic acid,
(1R*,2S*)-1-[(2-carboxy-2-methyl-propyl)-(4′-chloro-biphenyl-4-sulfonyl)-amino]-2-phenyl-cyclopropanecarboxylic acid,
(1R*,2S*)-1-[(4′-chloro-biphenyl-4-sulfonyl)-(2-methanesulfonylamino-2-oxo-ethyl)-amino]-2-phenyl-cyclopropanecarboxylic acid,
(1R*,2S*)-2-(3-benzyloxy-phenyl)-1-[carboxymethyl-(4′-chloro-biphenyl-4-sulfonyl)-amino]-cyclopropanecarboxylic acid,
(1R*,2S*)-2-(2-benzyloxy-phenyl)-1-[carboxymethyl-(4′-chloro-biphenyl-4-sulfonyl)-amino]-cyclopropanecarboxylic acid,
(1R*,2S*)-1-[carboxymethyl-(4′-chloro-biphenyl-4-sulfonyl)-amino]-2-(2,3-dichloro-phenyl)-cyclopropanecarboxylic acid,
(1R*,2S*)-1-[carboxymethyl-(4′-chloro-biphenyl-4-sulfonyl)-amino]-2-(2-phenoxy-phenyl)-cyclopropanecarboxylic acid,
(1R*,2S*)-1-{(4′-chloro-biphenyl-4-sulfonyl)-[2-(3,4-dihydroxy-pyrrolidin-1-yl)-2-oxo-ethyl]-amino}-2-phenyl-cyclopropanecarboxylic acid,
(1R*,2S*)-2-(3-benzyl-phenyl)-1-[carboxymethyl-(4′-chloro-biphenyl-4-sulfonyl)-amino]-cyclopropanecarboxylic acid,
(1R*,2S*)-1-[(4′-chloro-biphenyl-4-sulfonyl)-(2-oxo-2-pyrrolidin-1-yl-ethyl)-amino]-2-phenyl-cyclopropanecarboxylic acid,
(1R*,2S*)-1-[(4′-chloro-biphenyl-4-sulfonyl)-methoxycarbonylmethyl-amino]-2-phenyl-cyclopropanecarboxylic acid,
(1R*,2S*)-1-[carboxymethyl-(4′-chloro-biphenyl-4-sulfonyl)-amino]-2-(3-isobutoxy-phenyl)-cyclopropanecarboxylic acid,
(1R*,2S*)-1-[carboxymethyl-(4′-chloro-biphenyl-4-sulfonyl)-amino]-2-(3-cyclohexyloxy-phenyl)-cyclopropanecarboxylic acid,
(1R*,2S*)-[(4′-chloro-biphenyl-4-sulfonyl)-(1-methanesulfonylaminocarbonyl-2-phenyl-cyclopropyl)-amino]-acetic acid,
(1R*,2S*)-2-(3-benzyloxy-phenyl)-1-[carboxymethyl-(4′-chloro-biphenyl-4-sulfonyl)-amino]-cyclopropanecarboxylic acid,
(1R*,2S*)-3-{[[1-carboxy-2-(3-phenoxy-phenyl)-cyclopropyl]-(4′-chloro-biphenyl-4-sulfonyl)-amino]-methyl}-benzoic acid,
(1R*,2S*)-1-[(4′-chloro-biphenyl-4-sulfonyl)-ethoxycarbonylmethyl-amino]-2-(3-phenoxy-phenyl)-cyclopropanecarboxylic acid,
(1R*,2S*)-1-[(4′-chloro-biphenyl-4-sulfonyl)-methyl-amino]-2-(3-phenoxy-phenyl)-cyclopropanecarboxylic acid,
(1R*,2S*)-1-[(4′-chloro-biphenyl-4-sulfonyl)-(4-methanesulfonylaminocarbonyl-thiazol-2-ylmethyl)-amino]-2-phenyl-cyclopropanecarboxylic acid,
5-{[((1R,2S)-1-carboxy-2-phenyl-cyclopropyl)-(4′-chloro-biphenyl-4-sulfonyl)-amino]-methyl}-furan-2-carboxylic acid,
2-{[((1R,2S)-1-carboxy-2-phenyl-cyclopropyl)-(4′-chloro-biphenyl-4-sulfonyl)-amino]-methyl}-nicotinic acid,
(1R,2S)-1-[(4′-chloro-biphenyl-4-sulfonyl)-pyridin-2-ylmethyl-amino]-2-phenyl-cyclopropanecarboxylic acid,
(1R,2S)-1-[(4′-chloro-biphenyl-4-sulfonyl)-pyridin-3-ylmethyl-amino]-2-phenyl-cyclopropanecarboxylic acid,
(1R*,2S*)-1-{benzyl-[4-(2-methyl-2H-tetrazol-5-yl)-benzenesulfonyl]-amino}-2-phenyl-cyclopropanecarboxylic acid,
2-{[((1R,2S)-1-carboxy-2-phenyl-cyclopropyl)-(4′-chloro-biphenyl-4-sulfonyl)-amino]-methyl}-thiazole-4-carboxylic acid,
(1R*,2S*)-1-{(4′-chloro-biphenyl-4-sulfonyl)-[(1H-tetrazol-5-ylcarbamoyl)-methyl]-amino}-2-phenyl-cyclopropanecarboxylic acid,
(1R*,2S*)-1-[(4′-chloro-biphenyl-4-sulfonyl)-(3-methanesulfonylamino-benzyl)-amino]-2-phenyl-cyclopropanecarboxylic acid,
(1R,2S)-1-[(4′-chloro-biphenyl-4-sulfonyl)-(2-trifluoromethanesulfonylamino-ethyl)-amino]-2-phenyl-cyclopropanecarboxylic acid,
(1R*,2S*)-1-[(4′-chloro-biphenyl-4-sulfonyl)-(5-oxo-4,5-dihydro-[1,2,4]oxadiazol-3-ylmethyl)-amino]-2-phenyl-cyclopropanecarboxylic acid,
(1R*,2S*)-1-[(4′-chloro-biphenyl-4-sulfonyl)-(5-oxo-4,5-dihydro-1H-[1,2,4]triazol-3-ylmethyl)-amino]-2-phenyl-cyclopropanecarboxylic acid,
(1R*,2S*)-1-[carboxymethyl-(4′-chloro-biphenyl-4-sulfonyl)-amino]-2-(3-hydroxy-phenyl)-cyclopropanecarboxylic acid,
4-(3-{(1R,2S)-2-Carboxy-2-[carboxymethyl-(4′-chloro-biphenyl-4-sulfonyl)-amino]-cyclopropyl}-phenoxy)-piperidine-1-carboxylic acid tert-butyl ester,
(1R*,2S*)-1-[carboxymethyl-(4′-chloro-biphenyl-4-sulfonyl)-amino]-2-[3-(piperidin-4-yloxy)-phenyl]-cyclopropanecarboxylic acid,
1-{2-[((1R,2S)-1-carboxy-2-phenyl-cyclopropyl)-(4′-chloro-biphenyl-4-sulfonyl)-amino]-ethyl}-1H-pyrrole-2-carboxylic acid,
4-{(1R*,2S*)-2-carboxy-2-[(3-carboxy-benzyl)-(4′-chloro-biphenyl-4-sulfonyl)-amino]-cyclopropyl}-piperidin-1-carboxylic acid tert-butyl ester,
(1R*,2S*)-1-[(4′-chloro-biphenyl-4-sulfonyl)-(5-oxo-4,5-dihydro-[1,2,4]thiadiazol-3-ylmethyl)-amino]-2-phenyl-cyclopropanecarboxylic acid,
(1R*,2S*)-1-[(4′-chloro-biphenyl-4-sulfonyl)-(5-oxo-4,5-dihydro-[1,2,4]oxadiazol-3-ylmethyl)-amino]-2-(3-phenoxy-phenyl)-cyclopropanecarboxylic acid,
3-{[((1R*,2S*)-1-carboxy-2-phenyl-cyclopropyl)-(4′-chloro-biphenyl-4-sulfonyl)-amino]-methyl}-pyridin-2-carboxylic acid,
(1R*,2S*)-1-{methyl-[4-(4-methyl-thiophen-2-yl)-benzenesulfonyl]-amino}-2-phenyl-cyclopropanecarboxylic acid,
(1R*,2S*)-1-{carboxymethyl-[4-(4-methyl-thiophen-2-yl)-benzenesulfonyl]-amino}-2-phenyl-cyclopropanecarboxylic acid,
4-({((1R,2S)-1-carboxy-2-phenyl-cyclopropyl)-[4-(4-methyl-thiophen-2-yl)-benzenesulfonyl]-amino}-methyl)-benzoic acid,
(1R*,2S*)-1-{(4′-chloro-biphenyl-4-sulfonyl)-[2-(1H-tetrazol-5-ylamino)-ethyl]-amino}-2-phenyl-cyclopropanecarboxylic acid,
1-{2-[((1R*,2S*)-1-carboxy-2-phenyl-cyclopropyl)-(4′-chloro-biphenyl-4-sulfonyl)-amino]-ethyl}-1H-imidazole-2-carboxylic acid,
1-{2-[((1R,2S)-1-carboxy-2-phenyl-cyclopropyl)-(4′-chloro-biphenyl-4-sulfonyl)-amino]-ethyl}-1H-pyrazol-4-carboxylic acid,
3-{[((1R,2S)-1-carboxy-2-piperidin-4-yl-cyclopropyl)-(4′-chloro-biphenyl-4-sulfonyl)-amino]-methyl}-benzoic acid,
(1R*,2S*)-1-[(4′-chloro-biphenyl-4-sulfonyl)-(2-trifluoromethanesulfonylamino-ethyl)-amino]-2-(3-phenoxy-phenyl)-cyclopropanecarboxylic acid,
5-{[((1R,2S)-1-carboxy-2-phenyl-cyclopropyl)-(4′-chloro-biphenyl-4-sulfonyl)-amino]-methyl}-isooxazole-3-carboxylic acid,
(1R*,2S*)-1-{(4′-chloro-biphenyl-4-sulfonyl)-[2-(1,1,3,4-tetraoxo-1 lambda*6*-[1,2,5]thiadiazolidin-2-yl)-ethyl]-amino}-2-phenyl-cyclopropanecarboxylic acid,
(1R*,2S*)-1-[(4′-chloro-biphenyl-4-sulfonyl)-(2-aminosulfonylamino-ethyl)-amino]-2-(3-phenoxy-phenyl)-cyclopropanecarboxylic acid,
3-{[{(1R,2S)-1-carboxy-2-[3-(2-diethylamino-ethylamino)-phenyl]-cyclopropyl}-(4′-chloro-biphenyl-4-sulfonyl)-amino]-methyl}-benzoic acid,
3-{[{(1R,2S)-1-carboxy-2-[3-(pyridin-2-ylamino)-phenyl]-cyclopropyl}-(4′-chloro-biphenyl-4-sulfonyl)-amino]-methyl}-benzoic acid,
(1R*,2S*)-1-[(4′-chloro-biphenyl-4-sulfonyl)-(2-trifluoromethanesulfonylamino-ethyl)-amino]-2-[3-(2-piperidin-1-yl-acetylamino)-phenyl]-cyclopropanecarboxylic acid,
3-{[{(1R,2S)-1-carboxy-2-[3-(2-hydroxy-ethoxy)-phenyl]-cyclopropyl}-(4′-chloro-biphenyl-4-sulfonyl)-amino]-methyl}-benzoic acid,
(1R*,2S*)-1-[(4′-chloro-biphenyl-4-sulfonyl)-(2-trifluoromethanesulfonylamino-ethyl)-amino]-2-[3-(2-piperidin-1-yl-ethylamino)-phenyl]-cyclopropanecarboxylic acid,
(1R*,2S*)-1-[(4′-chloro-biphenyl-4-sulfonyl)-(2-trifluoromethanesulfonylamino-ethyl)-amino]-2-[3-(2-piperidin-1-yl-ethanesulfonylamino)-phenyl]-cyclopropanecarboxylic acid,
3-([{(1R,2S)-1-carboxy-2-[3-(2-piperidin-1-yl-ethylamino)-phenyl]-cyclopropyl)-(4′-chloro-biphenyl-4-sulfonyl)-amino]-methyl}-benzoic acid,
3-[((4′-chloro-biphenyl-4-sulfonyl)-{(1R*,2S*)-1-methyl carbamoyl-2-[3-(2-piperidin-1-yl-ethylamino)-phenyl]-cyclopropyl}-amino)-methyl]-benzoic acid,
(1R*,2S*)-1-[(3-carboxy-propyl)-(4′-chloro-biphenyl-4-sulfonyl)-amino]-2-phenyl-cyclopropanecarboxylic acid,
1-{2-[((1R*,2S*)-1-carboxy-2-phenyl-cyclopropyl)-(4′-chloro-biphenyl-4-sulfonyl)-amino]-ethyl}-piperidin-4-carboxylic acid,
3-{[{(1R,2S)-1-carboxy-2-[3-(2-imidazol-1-yl-ethoxy)-phenyl]-cyclopropyl}-(4′-chloro-biphenyl-4-sulfonyl)-amino]-methyl}-benzoic acid,
(1R*,2S*)-1-{(4′-chloro-biphenyl-4-sulfonyl)-[2-(2-hydroxy-acetylamino)-ethyl]-amino}-2-phenyl-cyclopropanecarboxylic acid,
1-{2-[((1R,2S)-1-carboxy-2-phenyl-cyclopropyl)-(4′-chloro-biphenyl-4-sulfonyl)-amino]-ethyl}-piperidin-3R-carboxylic acid,
1-{2-[((1R,2S)-1-carboxy-2-phenyl-cyclopropyl)-(4′-chloro-biphenyl-4-sulfonyl)-amino]-ethyl}-piperidin-3S-carboxylic acid,
3-{[((1R,2S)-1-carboxy-2-{3-[(pyridin-3-carbonyl)-amino]-phenyl}-cyclopropyl)-(4′-chloro-biphenyl-4-sulfonyl)-amino]-methyl}-benzoic acid,
3-{[{(1R*,2S*)-1-carboxy-2-[3-(2-pyrrolidin-1-yl-ethoxy)-phenyl]-cyclopropyl}-(4′-chloro-biphenyl-4-sulfonyl)-amino]-methyl}-benzoic acid,
3-{[{(1R,2S)-1-carboxy-2-[3-(2-morpholin-4-yl-ethoxy)-phenyl]-cyclopropyl}-(4′-chloro-biphenyl-4-sulfonyl)-amino]-methyl}-benzoic acid,
3-{[{(1R*,2S*)-1-carboxy-2-[3-(pyridin-3-yloxy)-phenyl]-cyclopropyl}-(4′-chloro-biphenyl-4-sulfonyl)-amino]-methyl}-benzoic acid,
(1R*,2S*)-1-[(4′-chloro-biphenyl-4-sulfonyl)-(4-oxalyl-benzyl)-amino]-2-phenyl-cyclopropanecarboxylic acid,
1-{2-[((1R,2S)-1-carboxy-2-phenyl-cyclopropyl)-(4′-chloro-biphenyl-4-sulfonyl)-amino]-ethyl}-1H-imidazole-4-carboxylic acid,
(1R*,2S*)-1-[(5-carbamoyl-pentyl)-(4′-chloro-biphenyl-4-sulfonyl)-amino]-2-phenyl-cyclopropanecarboxylic acid,
(1R*,2S*)-1-{(4′-chloro-biphenyl-4-sulfonyl)-[2-(4-methyl carbamoyl-pyrazol-1-yl)-ethyl]-amino}-2-phenyl-cyclopropanecarboxylic acid,
(1R*,2S*)-1-{(4′-chloro-biphenyl-4-sulfonyl)-[2-(2-hydroxy-2-methyl-propionylamino)-ethyl]-amino}-2-phenyl-cyclopropanecarboxylic acid,
3-{[{(1R,2S)-1-carboxy-2-[3-(2-pyrazol-1-yl-ethoxy)-phenyl]-cyclopropyl}-(4′-chloro-biphenyl-4-sulfonyl)-amino]-methyl)-benzoic acid,
(1R,2S)-1-{(4′-chloro-biphenyl-4-sulfonyl)-[2-(1H-tetrazol-5-ylamino)-ethyl]-amino}-2-(3-phenoxy-phenyl)-cyclopropanecarboxylic acid,
(1R,2S)-1-((4′-chloro-biphenyl-4-sulfonyl)-[3-(2H-tetrazol-5-ylamino)-propyl]-amino}-2-phenyl-cyclopropanecarboxylic acid,
3-{[((1R,2S)-1-carboxy-2-phenyl-cyclopropyl)-(4′-chloro-biphenyl-4-sulfonyl)-amino]-methyl}-benzoic acid methyl ester,
1-{2-[((1R,2S)-1-carboxy-2-phenyl-cyclopropyl)-(4′-chloro-biphenyl-4-sulfonyl)-amino]-ethyl}-1H-imidazole-4-carboxylic acid methyl ester,
(1R*,2S*)-1-{(4′-chloro-biphenyl-4-sulfonyl)-[2-(4-methyl carbamoyl-imidazol-1-yl)-ethyl]-amino}-2-phenyl-cyclopropanecarboxylic acid,
(1R*,2S*)-1-[(4′-chloro-biphenyl-4-sulfonyl)-(2-hydroxy-ethyl)-amino]-2-(3-phenoxy-phenyl)-cyclopropanecarboxylic acid, and
3-({((1R,2S)-1-carboxy-2-phenyl-cyclopropyl)-[4-(4-chloro-phenyl)-piperazine-1-sulfonyl]-amino}-methyl)-benzoic acid;
or a pharmaceutically acceptable salt thereof.

16. A pharmaceutical composition comprising a compound of claim 1, or a pharmaceutically acceptable salt thereof, and a pharmaceutically acceptable carrier.

17. A method of inhibiting aggrecanase comprising administering a compound of claim 1, or a pharmaceutically acceptable salt thereof as an active ingredient, to a mammal.

18. A method of inhibiting MMP comprising administering a compound of claim 1, or a pharmaceutically acceptable salt thereof as an active ingredient, to a mammal.

19. The method of claim 18, wherein MMP is MMP-13.

20-21. (canceled)

22. A method for preventing or treating osteoarthritis, which comprises administering a compound of claim 1, or a pharmaceutically acceptable salt thereof to a mammal.

23. A method for preventing or treating rheumatoid arthritis, which comprises administering a compound of claim 1, or a pharmaceutically acceptable salt thereof to a mammal.

24-27. (canceled)

28. The method of claim 22, further comprising administering at least one other therapeutic agent for osteoarthritis.

29. The method of claim 22, further comprising administering at least one other therapeutic agent for rheumatoid arthritis.

30. The method of claim 23, further comprising administering at least one other therapeutic agent for osteoarthritis.

31. The method of claim 23, further comprising administering at least one other therapeutic agent for rheumatoid arthritis.
